(19) United States
(12) Patent Application Publication
(10) Pub. No.: US 2025/0366361 A1
(43) Pub. Date: Nov. 27, 2025

(54) METAL IRIDIUM COMPLEX AND USE THEREOF

(71) Applicant: GUANGDONG AGLAIA OPTOELECTRONIC MATERIALS CO., LTD, Guangdong (CN)

(72) Inventors: Liangliang YAN, Guangdong (CN); Shaofu CHEN, Guangdong (CN); Lei DAI, Guangdong (CN); Lifei CAI, Guangdong (CN)

(73) Assignee: GUANGDONG AGLAIA OPTOELECTRONIC MATERIALS CO., LTD, Guangdong (CN)

(21) Appl. No.: 18/874,414

(22) PCT Filed: May 31, 2023
(86) PCT No.: PCT/CN2023/097311

§ 371 Date: Dec. 12, 2024

(30) Foreign Application Priority Data
Jun. 30, 2022 (CN) 202210754653.3

(51) Int. Cl. H10K 85/30; C07D 215/06; C07F 15/00; C09K 11/06; H10K 50/12; H10K 50/17

(52) U.S. Cl. CPC H10K 85/342; C07D 215/06; C07F 15/0033; C09K 11/06; C09K 2211/1007; C09K 2211/1029; C09K 2211/1088; C09K 2211/1092; C09K 2211/185; H10K 50/12; H10K 50/17

(57) ABSTRACT

The present disclosure relates to a metal iridium complex and use thereof. The metal iridium complex has a general formula Ir(La)(Lb)(Lc), wherein La is the structure represented by formula (1), and Lb is the structure represented by formula (2). The complex has the advantages of low driving voltage, low sublimation temperature, good optical and electrical stability, high luminous efficiency, long service life, high color saturation, and the like, can be used in organic light-emitting devices, and especially as a red luminous phosphorescent material, and has the possibility of being applied to the AMOLED industry.

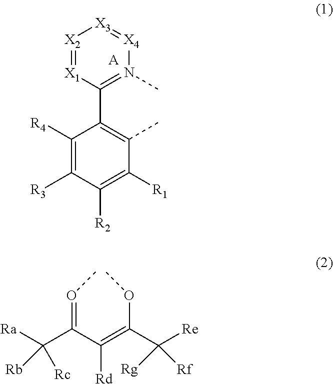

### TECHNICAL FIELD

The present disclosure relates to the technical field of organic electroluminescence, in particular, to an organic light-emitting material, and particularly, to a metal iridium complex and its use in organic electroluminescent devices.

### BACKGROUND

At present, organic electroluminescent devices (OLEDs), as a new generation of display technology, have attracted more and more attention in display and lighting technology, and have broad application prospects. However, compared with market application requirements, the luminous efficiency, driving voltage, service life and other properties of OLEDs need to continue to be strengthened and improved.

Generally speaking, the basic structure of an OLED consists of thin films of various organic functional materials sandwiched between metal electrodes. Driven by current, holes and electrons are injected from the anode and cathode, respectively, and are recombined in a light-emitting layer after moving a certain distance and released in a form of light or heat, resulting in the luminescence of the OLED. However, the organic functional materials are core components of the OLED, and their thermal stability, photochemical stability, electrochemical stability, quantum yield, film formation stability, crystallinity, color saturation and the like are main factors that affect the performance of the device.

Generally, the organic functional materials include fluorescent materials and phosphorescent materials. The fluorescent materials are usually organic small molecule materials, which generally can only utilize 25% singlet excitons for light-emission, thus leading to a relatively low luminous efficiency. Due to the spin-orbit coupling caused by heavy atom effects, the phosphorescent materials can utilize both 25% singlet excitons and the energy of 75% triplet excitons, thus improving their luminous efficiency. However, compared with the fluorescent materials, the phosphorescent materials are of a late start, and their thermal stability, service life, color saturation and the like all need to be improved, which is a challenging topic. Various compounds have been developed as the phosphorescent materials. For example, the patent CN1589307A discloses a metal complex

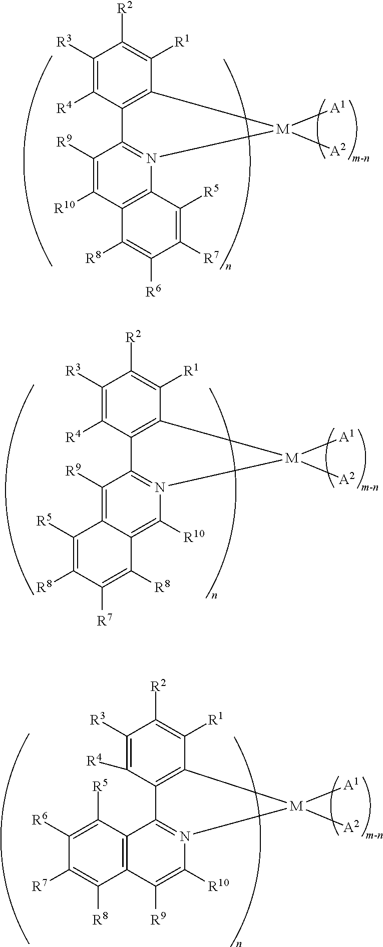

which quinoline, isoquinoline and benzene ring-linked compounds are used as ligands, especially iridium complexes, which can provide a luminescence of 500-700 nm, and states that the color of luminescence for compounds is adjusted upon selection of electron-donating or electron-withdrawing groups at specific positions. The patent CN100375749C discloses an iridium complex

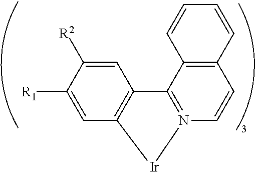

with isoquinoline and benzene ring derivatives as ligands, and has carried out certain selection and screening for the selection of R₁ and R₂ and obtained a higher photoluminescence efficiency relative to Ir(ppy)₃, however, the corresponding device performance, especially device efficiency, needs to be further improved. The patent CN101160369B discloses an iridium complex

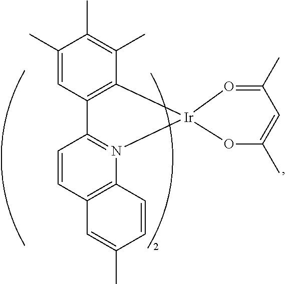

however, the color saturation, device efficiency and service life of such materials need to be improved. The patent CN102603803B discloses a metal complex

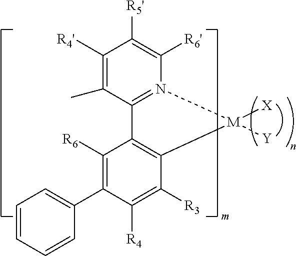

in which isoquinoline is connected to meta-biphenyl. However, the device performance of such materials is lower, especially the device efficiency may be lower than the same complex without biphenyl. The patent CN104885248B discloses an Ir metal complex

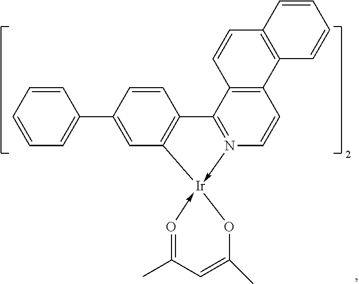

where the applicant points out that high device efficiency and long service life can be provided by adjusting the matching and combination of light-emitting layers. The patent CN110615816A discloses an iridium complex containing triphenyl silicon, specifically disclosing a complex

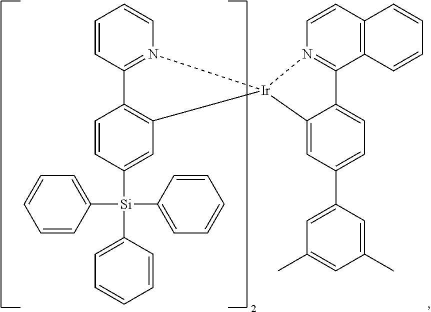

where the applicant points out that such materials have high device efficiency an long service life. The patent TW200848422A discloses an iridium complex with phenylquinoline or isoquinoline

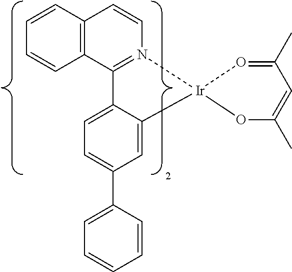

as a ligand, in particular, the performance of the disclosed complex needs to be better improved. The patent TW200423814A discloses

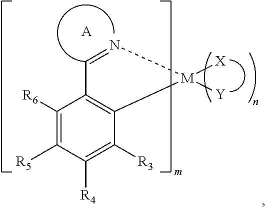

in which R₅ is a substituted or unsubstituted heteroaryl group, and a complex

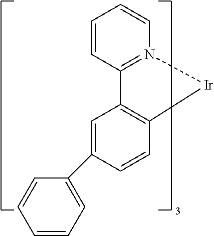

is prepared, showing a red-shifted luminescence spectrum (514 nm to 519 nm) and a slightly improved EQE (6.5% to 7.0%) relative to Ir(ppy)₃. Although the device performance of such materials has been improved to varying degrees, especially in terms of the device efficiency and service life, further improvements are needed to meet the growing market demand.

### SUMMARY

The present disclosure is created to solve the above-mentioned problems, and aims to provide a high-performance organic electroluminescent device and a novel material that can realize such an organic electroluminescent device.

The inventors of the present disclosure have repeatedly conducted in-depth research in order to achieve the aforementioned objective, and have found that a high-performance organic electroluminescent device can be obtained by using an organometallic iridium complex including ligands represented by the following formulas (1) and (2).

The metal iridium complex has a general formula Ir(La)(Lb)(Lc), wherein La is a structure of formula (1) and Lb is a structure of formula (2). The complex provided by the present disclosure has the advantages of low driving voltage, low sublimation temperature, good optical and electrical stability, high luminous efficiency, long service life, high color saturation and the like, can be used in organic light-emitting devices, especially as a red-emitting phosphorescent material, and has the possibility to be applied to the AMOLED industry.

A metal iridium complex has a general formula Ir(La)(Lb)(Lc), where La is a structure of formula (1),

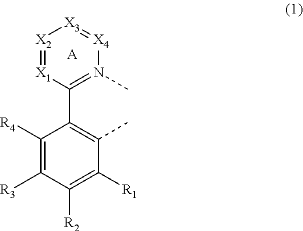

where dotted lines indicate a position connected to a metal Ir;
where X₁-X₄ are independently N or CR₀, and at least two of the X₁-X₄ are CR₀, and two R₀ are connected to each other to form a five- or six-membered substituted or unsubstituted aromatic or heteroaromatic ring;
where R₀ and R₄ are independently selected from hydrogen, deuterium, halogen, cyano, a substituted or unsubstituted C1-C10 alkyl group, a substituted or unsubstituted C1-C10 heteroalkyl group, a substituted or unsubstituted C3-C20 cycloalkyl group, a substituted or unsubstituted C3-C20 heterocycloalkyl group, a substituted or unsubstituted C2-C10 alkenyl group, a substituted or unsubstituted C2-C10 alkynyl group, a substituted or unsubstituted C6-C30 aryl group, a substituted or unsubstituted C2-C30 heteroaryl group, a substituted or unsubstituted (tri-C1-C10 alkyl)silyl group, a substituted or unsubstituted (tri-C6-C12 aryl)silyl group, a substituted or unsubstituted (di-C1-C10 alkyl)(mono-C6-C30 aryl)silyl group, a substituted or unsubstituted (mono-C1-C10 alkyl)(di-C6-C30 aryl)silyl group, amino group, thiol group, or hydroxyl group;
where R₁ and R₃ are independently selected from a substituted or unsubstituted C1-C10 alkyl group, a substituted or unsubstituted C1-C10 heteroalkyl group, a substituted or unsubstituted C3-C20 cycloalkyl group, or a substituted or unsubstituted C3-C20 heterocycloalkyl group;
where R₂ is a substituted or unsubstituted C6-C30 aryl group, or a substituted or unsubstituted C2-C30 heteroaryl group;
where Lb is a structure of formula (2),

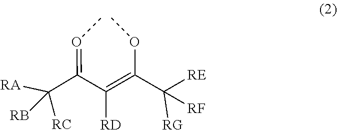

where dotted lines indicate a position connected to the metal Ir,
where RA-RG are independently selected from hydrogen, deuterium, halogen, a substituted or unsubstituted C1-C10 alkyl group, a substituted or unsubstituted C3-C20 cycloalkyl group, a substituted or unsubstituted C1-C10 heteroalkyl group, or a substituted or unsubstituted C3-C20 heterocycloalkyl group, or RA, RB, and RC are connected in pairs to form an aliphatic ring, and RE, RF, and RG are connected in pairs to form an aliphatic ring;
where the heteroaromatic ring, the heteroalkyl group, the heterocycloalkyl group and the heteroaryl group contain at least one 0, N or S heteroatom;
where the substitution is the substitution by deuterium, F, Cl, Br, a C1-C6 alkyl group, a C1-C6 alkoxy group, a C3-C6 cycloalkyl group, or amine group substituted by a C1-C6 alkyl group, cyano, isocyano group or phosphino, where the substitution is from a monosubstitution to a maximum substitution;
where Lc is a monoanionic bidentate ligand; Lc is different from Lb and not OO-type ligand;
where Lc and La are the same or different, and the difference is that a parent core structure is different, or the parent core structure is the same but substituents are different, or the parent core structure is the same, the substituents are the same, but positions of the substituents are different; and
or, two or three of La, Lb and Lc are connected to each other to form a multidentate ligand.

As a preferred metal iridium complex, the aromatic ring structure or the heteroaromatic ring structure is an aromatic ring or heteroaromatic ring in which X₁ and X₂, or X₂ and X₃, or X₁ and X₄ each are CR₀ and two R₀ are connected to each other to form a five-membered ring or a six-membered ring, where the aromatic ring structure or the heteroaromatic ring structure of the five-membered ring or six-membered ring is fused with an A ring to form a fused ring structure, and the fused ring structure is represented b one of the following formulas:

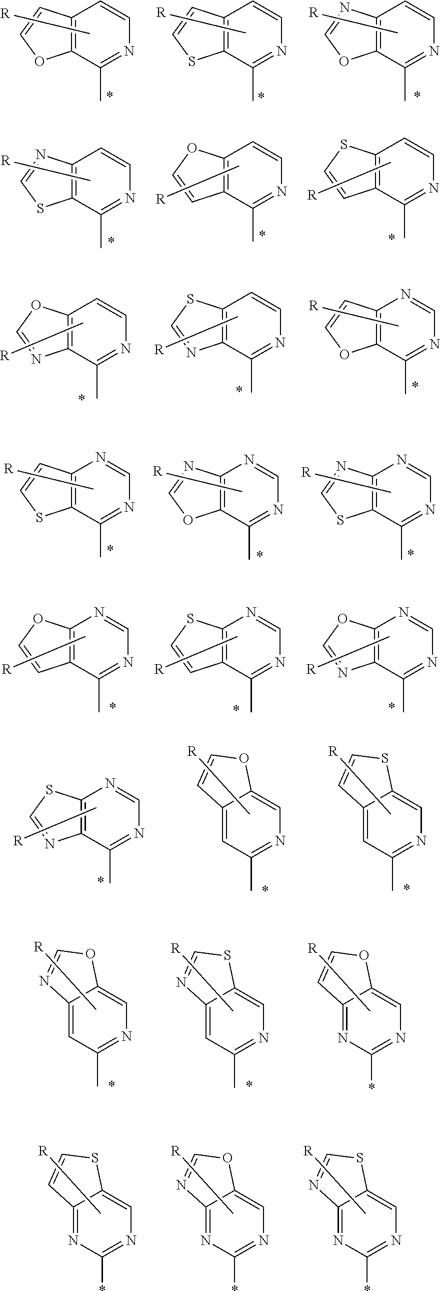

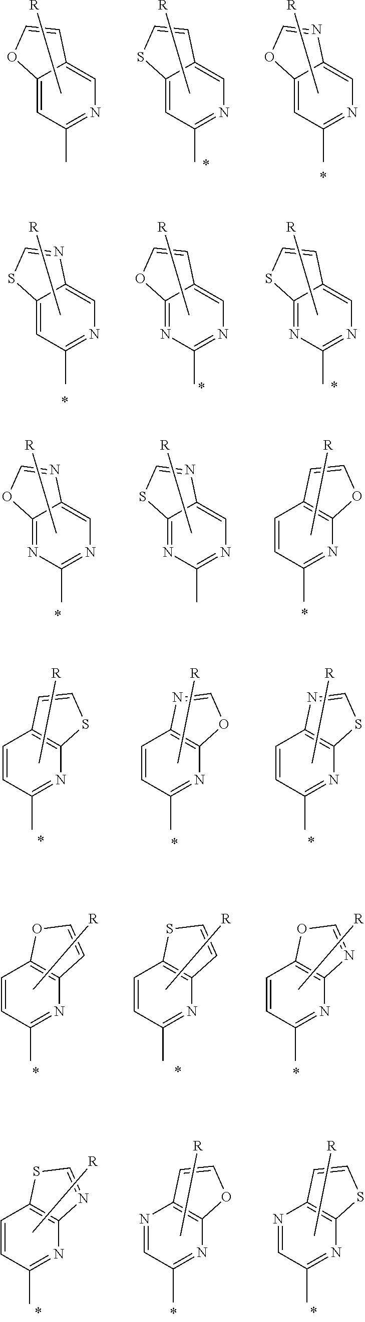

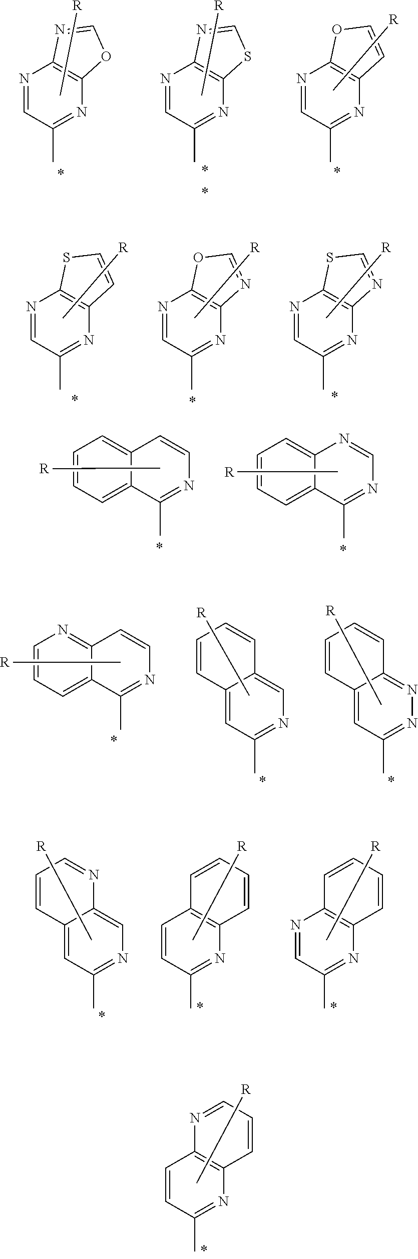

where * represents a position connected to a substituted benzene ring in formula (1);
where R represents no substitution to a maximum possible substitution, or two adjacent R are connected to each other to form an aliphatic or aromatic ring structure, where no substitution means that the positions connected to carbon are all hydrogen atoms, and R is independently selected from deuterium, halogen, cyano, a substituted or unsubstituted C1-C10 alkyl group, a substituted or unsubstituted C1-C10 heteroalkyl group, a substituted or unsubstituted C3-C20 cycloalkyl group, a substituted or unsubstituted C3-C20 heterocycloalkyl group, a substituted or unsubstituted C2-C10 alkenyl group, a substituted or unsubstituted C2-C10 alkynyl group, a substituted or unsubstituted C6-C30 aryl group, a substituted or unsubstituted C2-C30 heteroaryl group, a substituted or unsubstituted (tri-C1-C10 alkyl) silyl group, a substituted or unsubstituted (tri-C6-C12 aryl) silyl group, a substituted or unsubstituted (di-C1-C10 alkyl) (mono-C6-C30 aryl) silyl group, a substituted or unsubstituted (mono-C1-C10 alkyl) (di-C6-C30 aryl) silyl group, amino group, thiol group, or hydroxyl group.

As a preferred metal iridium complex, a structure of formula (3) formed by interconnection between two adjacent R₀ in formula (1) forms a fused ring structure with the A ring:

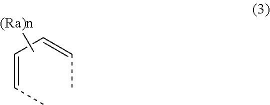

where dotted lines indicate a site connected to the A ring;
where Ra is independently selected from hydrogen, deuterium, halogen, cyano, a substituted or unsubstituted C1-C10 alkyl group, a substituted or a unsubstituted C1-C10 heteroalkyl group, a substituted or unsubstituted C3-C20 cycloalkyl group, a substituted or unsubstituted C3-C20 heterocycloalkyl group, a substituted or unsubstituted C2-C10 alkenyl group, a substituted or unsubstituted C2-C10 alkynyl group, a substituted or unsubstituted C6-C30 aryl group, a substituted or unsubstituted C2-C30 heteroaryl group, a substituted or unsubstituted (tri-C1-C10 alkyl)silyl group, a substituted or unsubstituted (tri-C6-C12 aryl)silyl group, a substituted or unsubstituted (di-C1-C10 alkyl)(mono-C6-C30 aryl)silyl group, and a substituted or unsubstituted (mono-C1-C10 alkyl)(di-C6-C30 aryl)silyl group, or two adjacent Ra are connected to each other to form an alicyclic ring or aromatic ring;
where n is a natural number from 0 to 4.

As a preferred metal iridium complex, the formula (1) has a structure of formula (4):

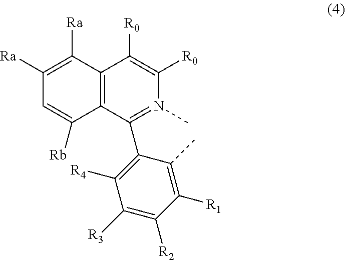

where Rb is H, deuterium, halogen, cyano, a substituted or unsubstituted C1-C10 alkyl group, a substituted or unsubstituted C1-C10 heteroalkyl group, a substituted or unsubstituted C3-C20 cycloalkyl group, or a substituted or unsubstituted C3-C20 heterocycloalkyl group;
where at least one of Ra is not H; and
the symbol definition thereof is the same as mentioned above.

As a preferred metal iridium complex, R₂ is a substituted or unsubstituted C6-C12 aryl group, or a substituted or unsubstituted C4-C12 heteroaryl group.

As a preferred metal iridium complex, it has a structure of formula (5):

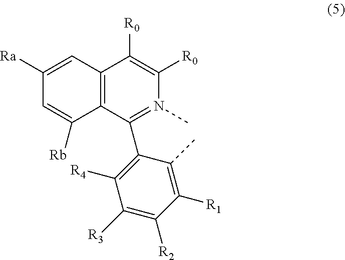

where Ra is independently selected from a substituted or unsubstituted C1-C10 alkyl group, a substituted or unsubstituted C1-C10 heteroalkyl group, a substituted or unsubstituted C3-C20 cycloalkyl group, a substituted or unsubstituted C3-C20 heterocycloalkyl group, a substituted or unsubstituted C2-C10 alkenyl group, a substituted or unsubstituted C2-C10 alkynyl group, a substituted or unsubstituted C6-C30 aryl group, or a substituted or unsubstituted C2-C30 heteroaryl group; and
R₂ is a substituted or unsubstituted phenyl group, or a substituted or unsubstituted C4 heteroaryl group.

As a preferred metal iridium complex, the substitution in R₂ is the substitution by a group containing deuterium; F; CN; C1-C6 alkyl; a C3-C6 cycloalkyl group containing deuterium, F, CN, C1-C6; a deuterium or F substituted C1-C6 alkyl group; or a deuterium or F substituted C3-C6 cycloalkyl group.

As a preferred metal iridium complex, R₁ and R₃ are a deuterium or F substituted or unsubstituted C1-C6 alkyl group, or a deuterium or F substituted or unsubstituted C3-C6 cycloalkyl group.

As a preferred metal iridium complex, R₄ is hydrogen, deuterium, F, CN, or a substituted or unsubstituted C1-C4 alkyl group.

As a preferred metallic iridium complex, R₄ and Rb are connected to each other to form a structure of formula (6):

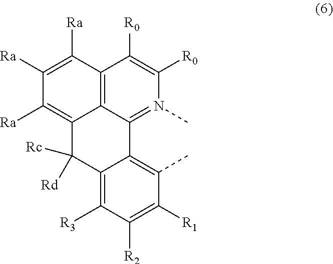

where Rc and Rd are H, deuterium, F, CN, or a substituted or unsubstituted C1-C4 alkyl group, and where the substitution in Rc and Rd is the substitution by deuterium, F or a C1-C4 alkyl group, and at least one of the Ra is not hydrogen.

Further preferably:

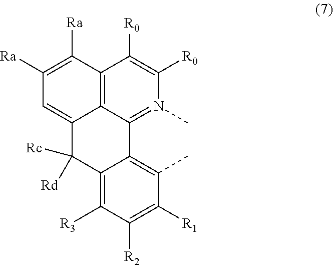

where at least one of the Ra is not hydrogen.

As a preferred metal iridium complex, Lc is the same as La.

As a preferred metal iridium complex, Lc and La are different.

As a preferred metal iridium complex, Lc is a structure of formula (8),

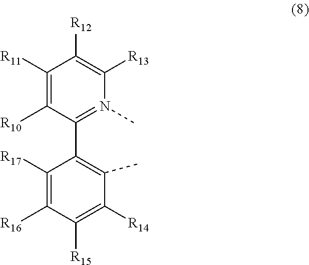

where dotted lines indicate a position connected to the metal Ir;
where R₁₀-R₁₇ are independently selected from hydrogen, deuterium, halogen, cyano, hydroxyl group, amino group, amine group, a substituted or unsubstituted C1-C10 alkyl group, a substituted or unsubstituted C1-C10 heteroalkyl group, a substituted or unsubstituted C3-C20 cycloalkyl group, a substituted or unsubstituted C2-C10 alkenyl group, a substituted or unsubstituted C2-C10 alkynyl group, a substituted or unsubstituted C6-C18 aryl group, a substituted or unsubstituted C2-C17 heteroaryl group, a substituted or unsubstituted (tri-C1-C10 alkyl)silyl group, a substituted or unsubstituted (tri-C6-C12 aryl)silyl group, a substituted or unsubstituted (di-C1-C10 alkyl)(mono-C6-C30 aryl)silyl group, or a substituted or unsubstituted (mono-C1-C10 alkyl)(di-C6-C30 aryl)silyl group;
where at least two of R₁₄-R₁₇ are not hydrogen;
or, at least one group of two adjacent groups in R₁₀-R₁₃ forms an aromatic ring as represented by the following formula (9);

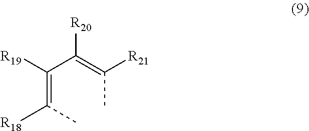

in the formula (9),
where dotted lines indicate a position connected to a pyridine ring;
where R₁₈-R₂₁ are independently selected from hydrogen, deuterium, halogen, cyano, a substituted or unsubstituted C1-C10 alkyl group, a substituted or unsubstituted C1-C10 heteroalkyl group, a substituted or unsubstituted C3-C20 cycloalkyl group, a substituted or unsubstituted C2-C10 alkenyl group, a substituted or unsubstituted C2-C10 alkynyl group, a substituted or unsubstituted C6-C18 aryl group, a substituted or unsubstituted C2-C17 heteroaryl group, a substituted or unsubstituted (tri-C1-C10 alkyl)silyl group, a substituted or unsubstituted tri-C6-C12 arylsilyl, a substituted or unsubstituted (di-C1-C10 alkyl)(mono-C6-C30 aryl)silyl group, and a substituted or unsubstituted (mono-C1-C10 alkyl)(di-C6-C30 aryl)silyl group, or two adjacent groups of Rig-R₂₁ are connected to each other to form an alicyclic ring or aromatic ring;
where the heteroalkyl group and the heteroaryl group contain at least one O, N or S heteroatom;
where the substitution is the substitution by deuterium, F, Cl, Br, a C1-C6 alkyl group, a C3-C6 cycloalkyl group, amine group substituted by a C1-C6 alkyl group, cyano, isocyano group or phosphino, and where the substitution is a monosubstitution to a maximum substitution.

As a preferred metal iridium complex, Lc is one of the following structural formulas, or being correspondingly partially or completely deuterated or fluorinated,

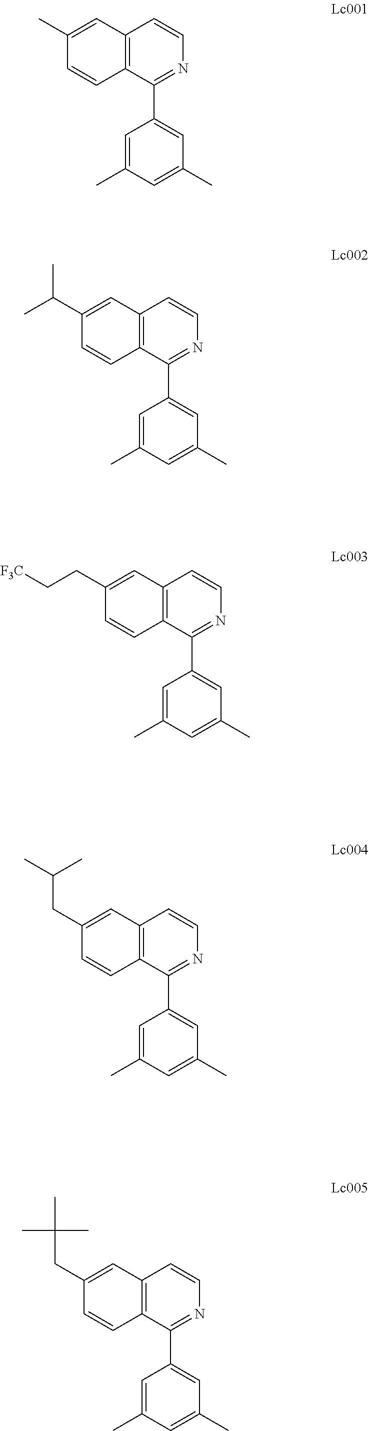

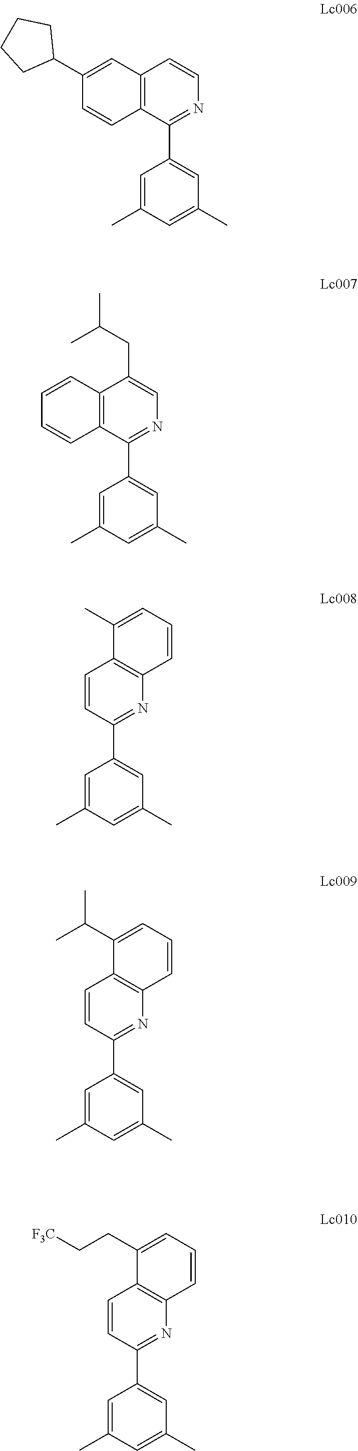

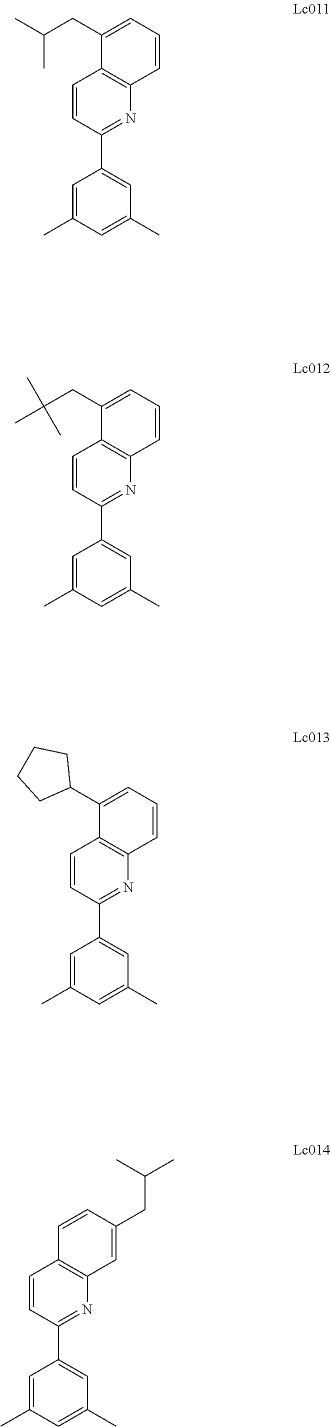 [SMILES: Cc1cc(C)cc(-c2ccc3c(CC(C)C)cccc3n2)c1 ; Cc1cc(C)cc(-c2ccc3c(CC(C)(C)C)cccc3n2)c1 ; Cc1cc(C)cc(-c2ccc3c(C4CCCC4)cccc3n2)c1 ; Cc1cc(C)cc(-c2ccc3ccc(CC(C)C)cc3n2)c1]

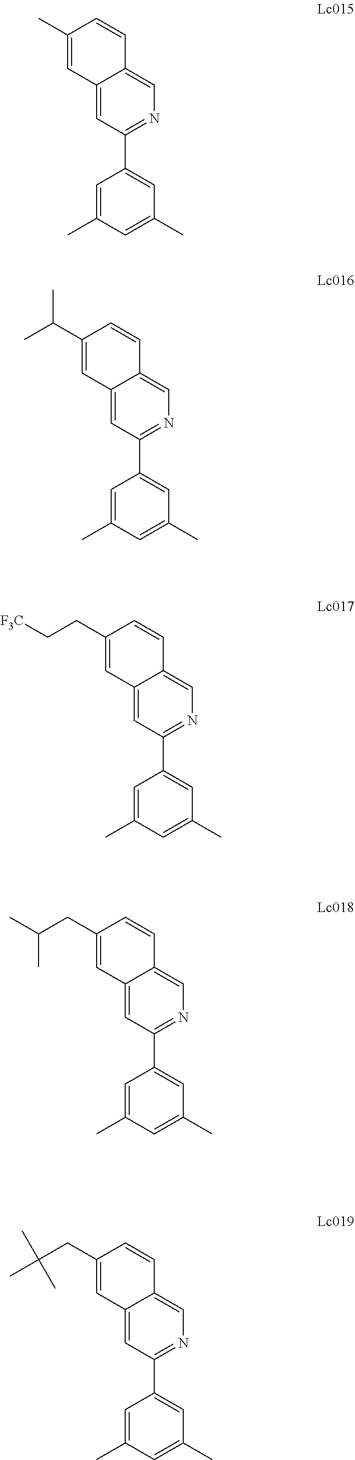

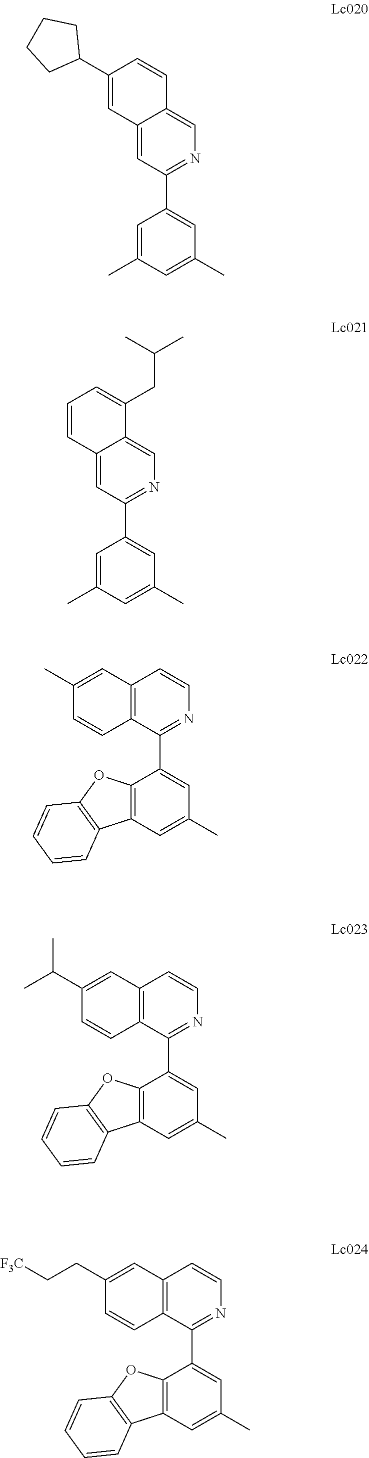

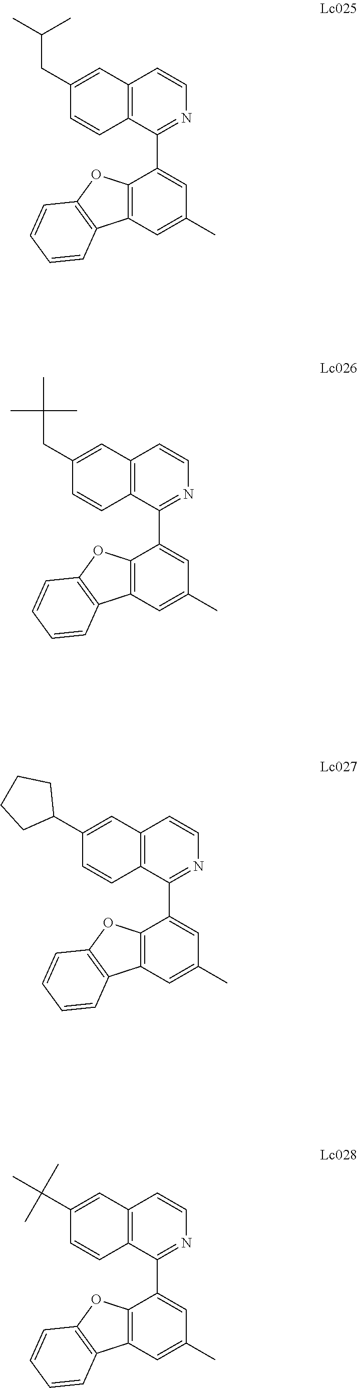

As a preferred metal indium complex, La is one of the following structural formulas, or being correspondingly partially or completely deuterated or fluorinated,

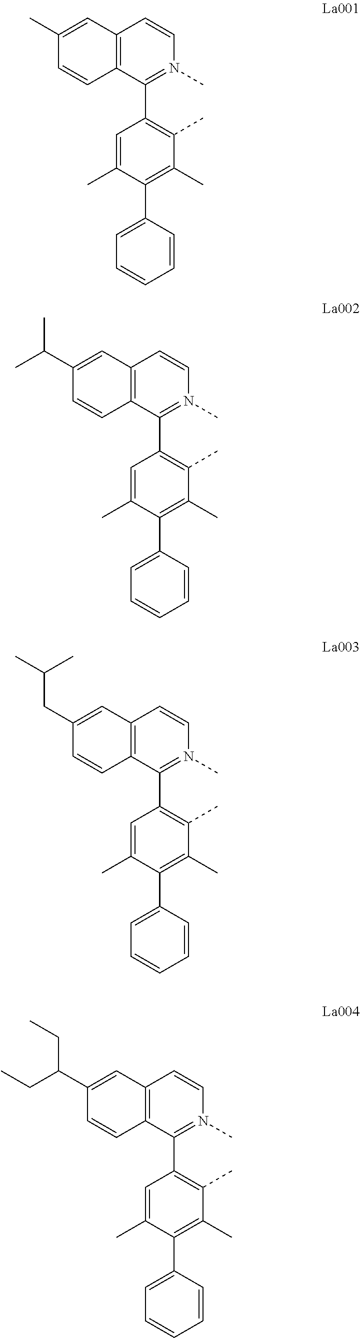

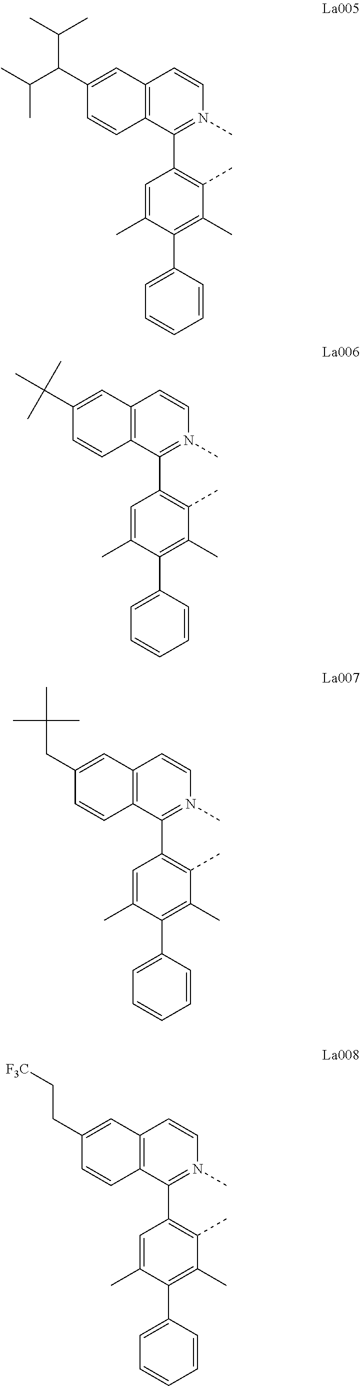

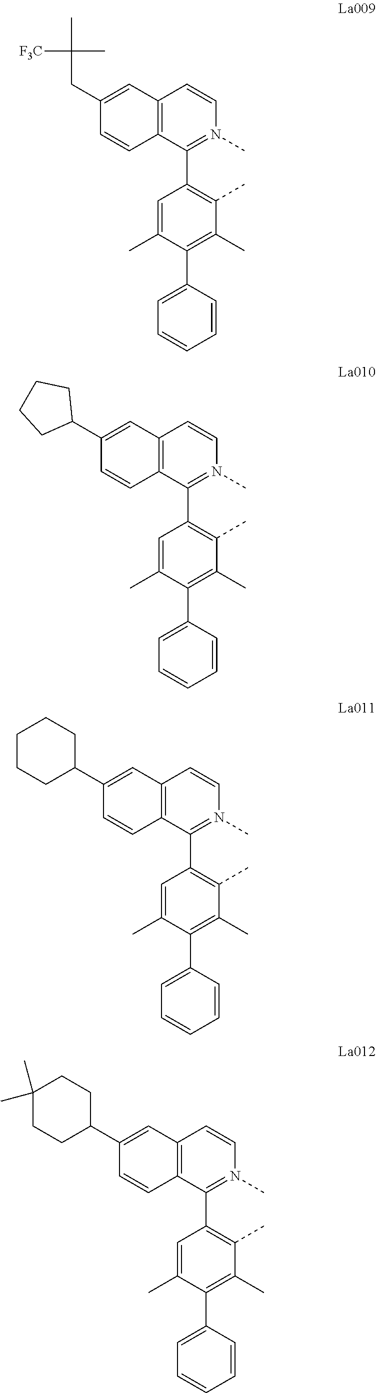

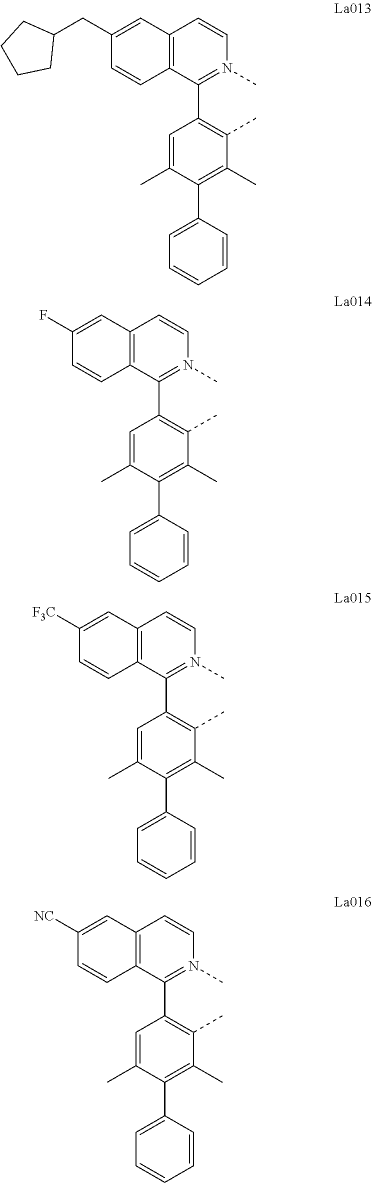

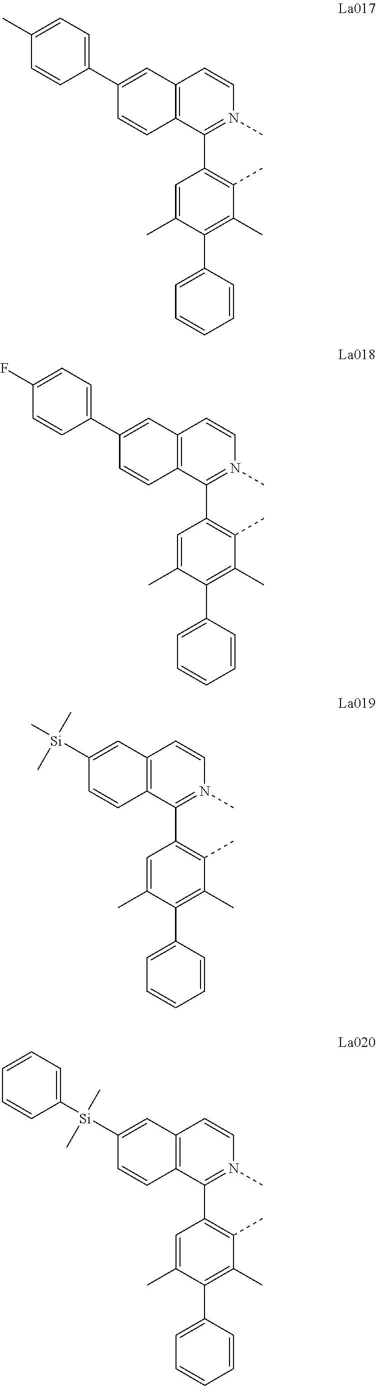

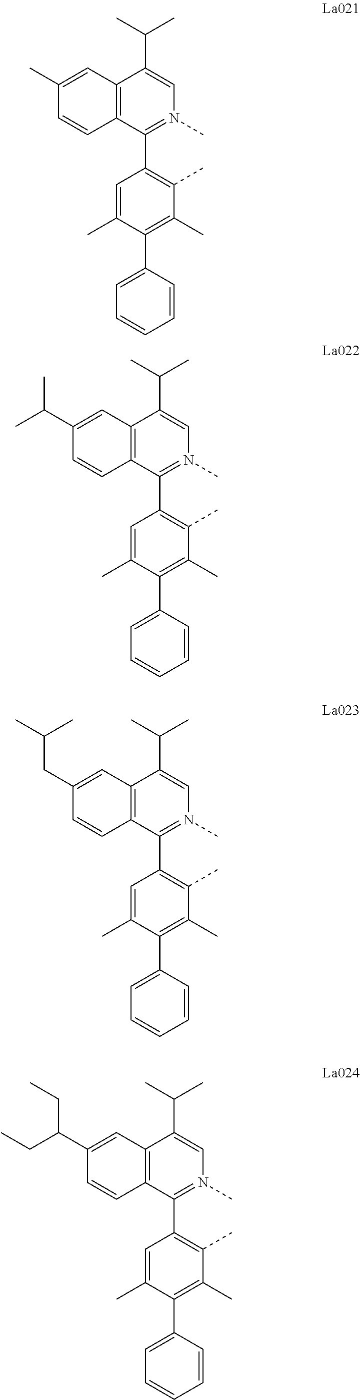

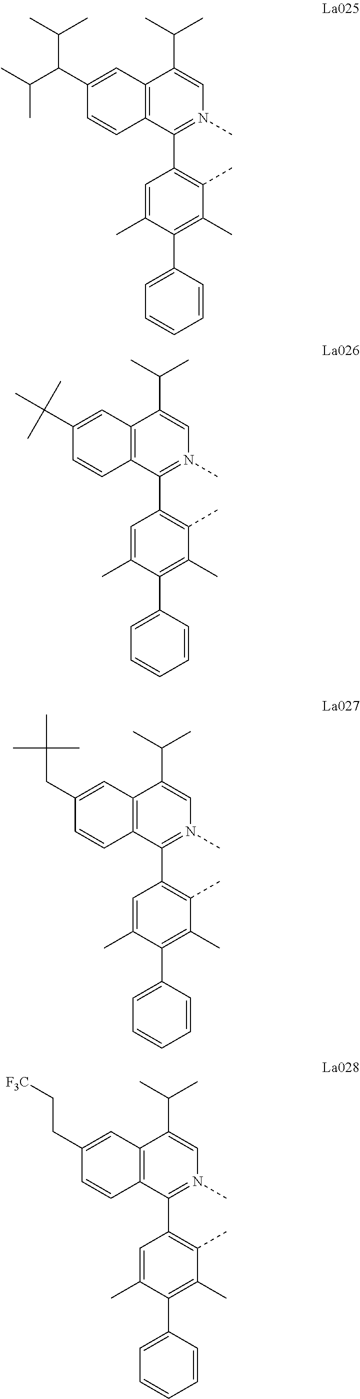

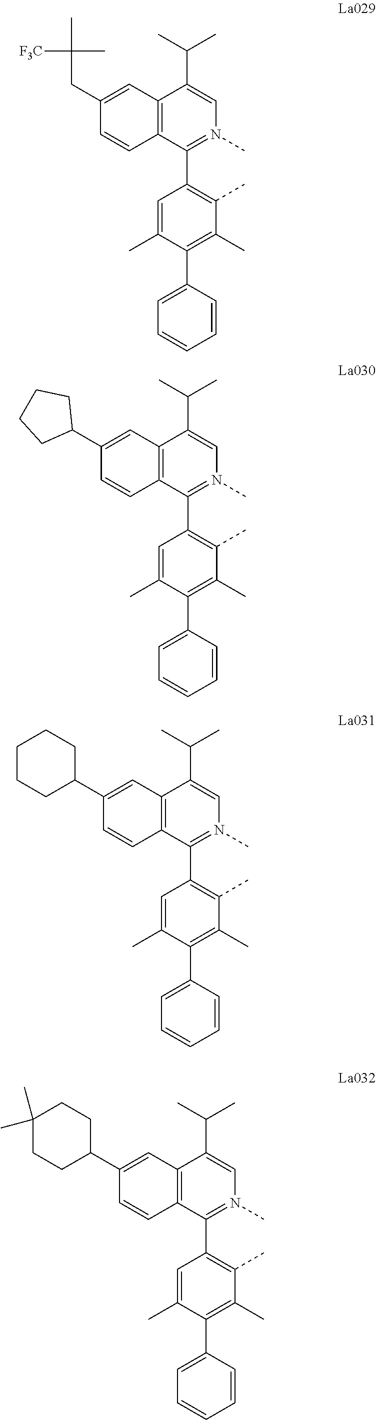

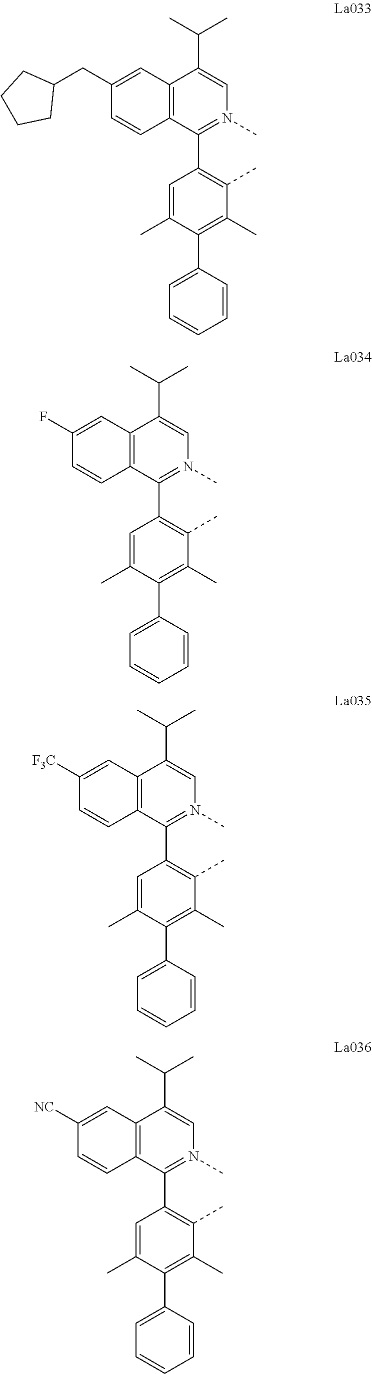

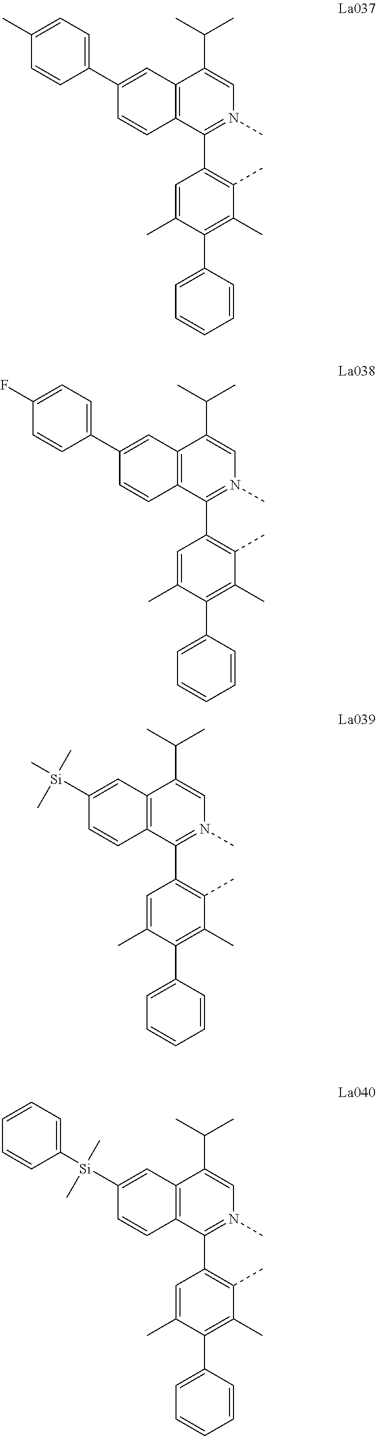

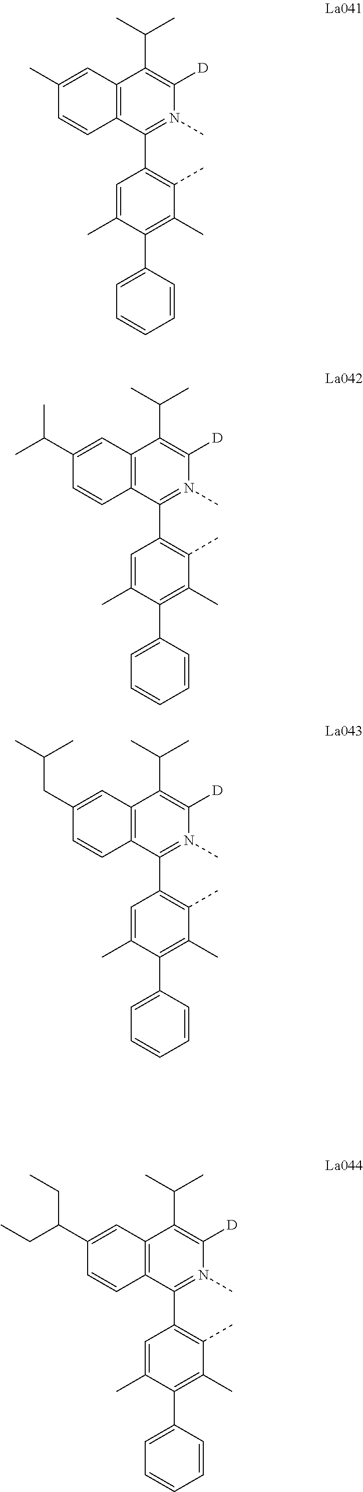

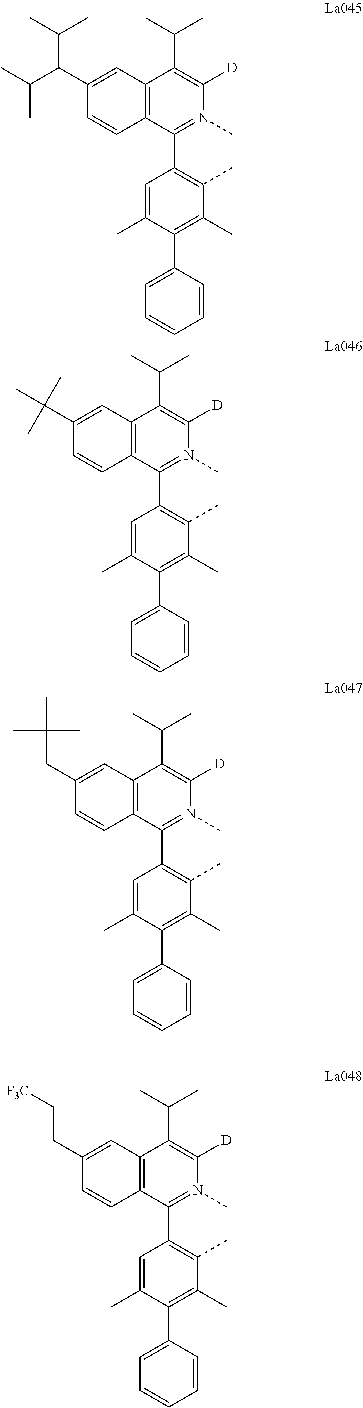

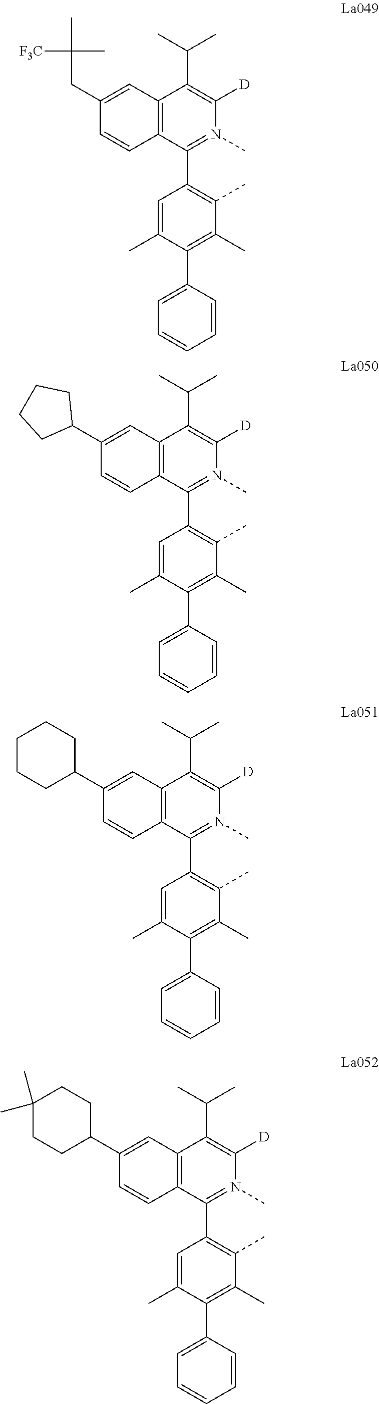

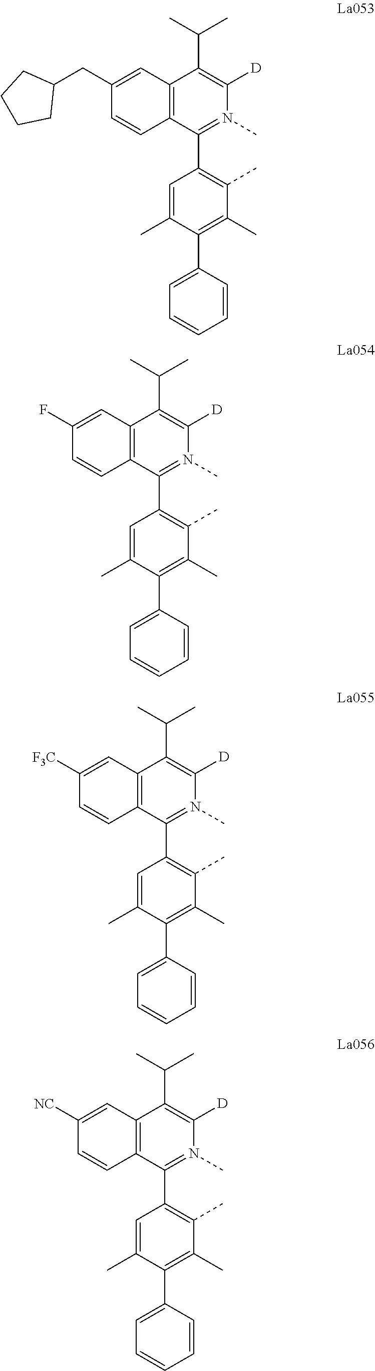

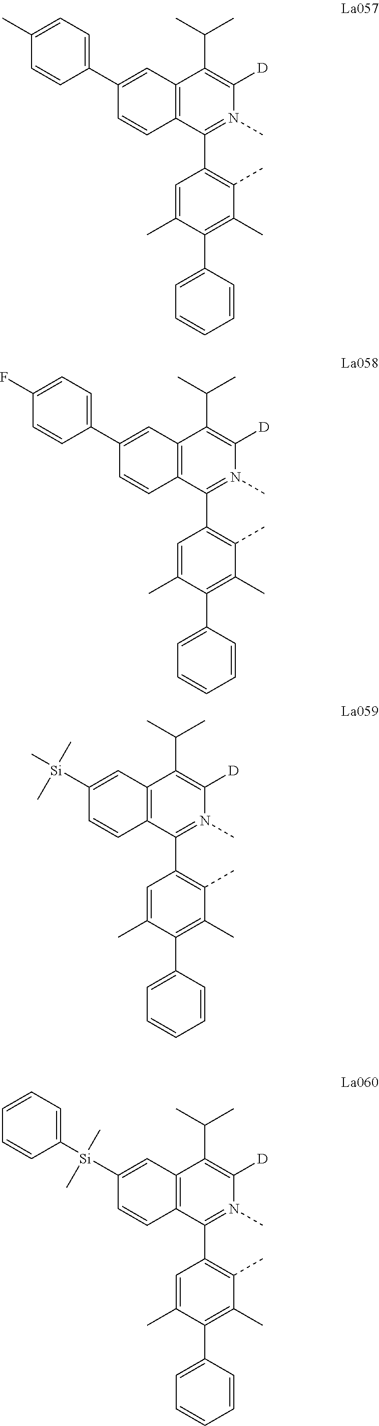

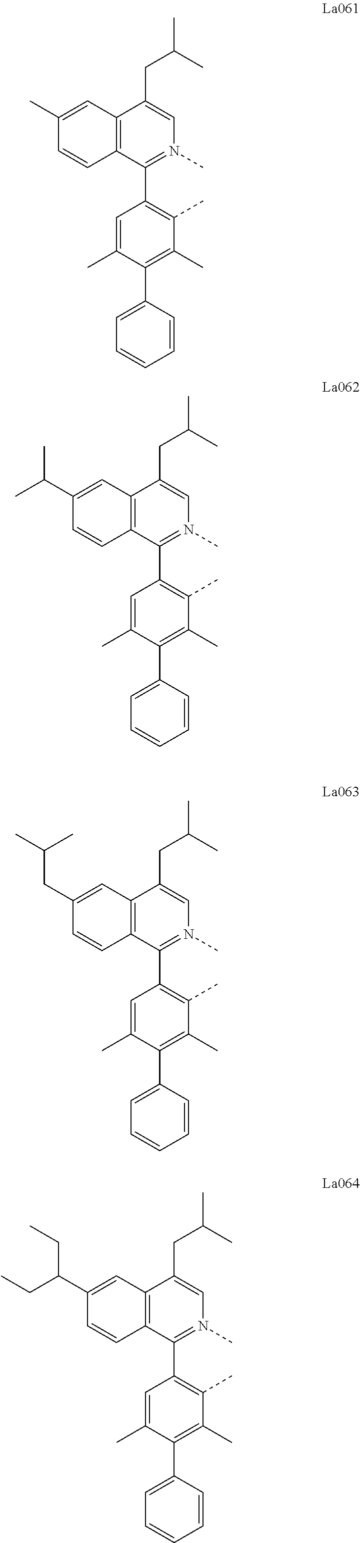

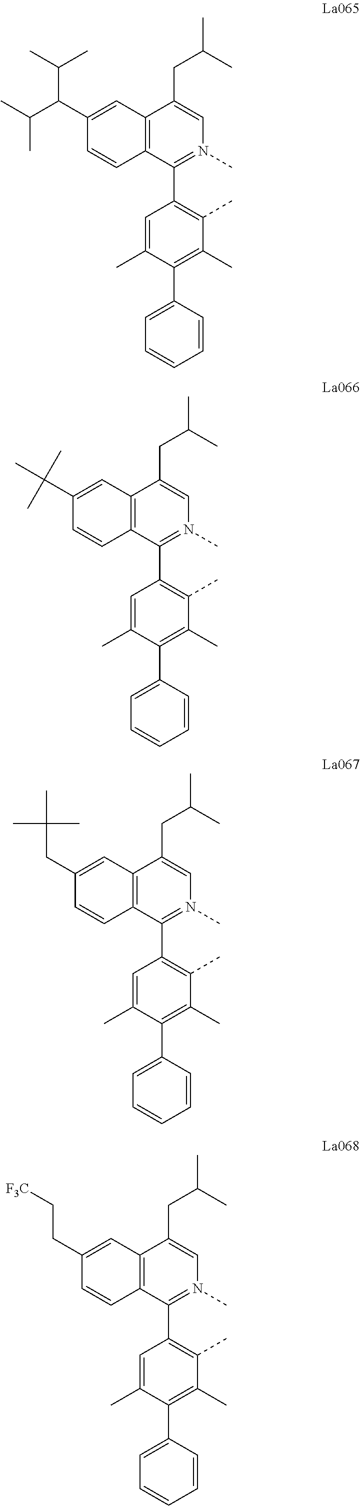

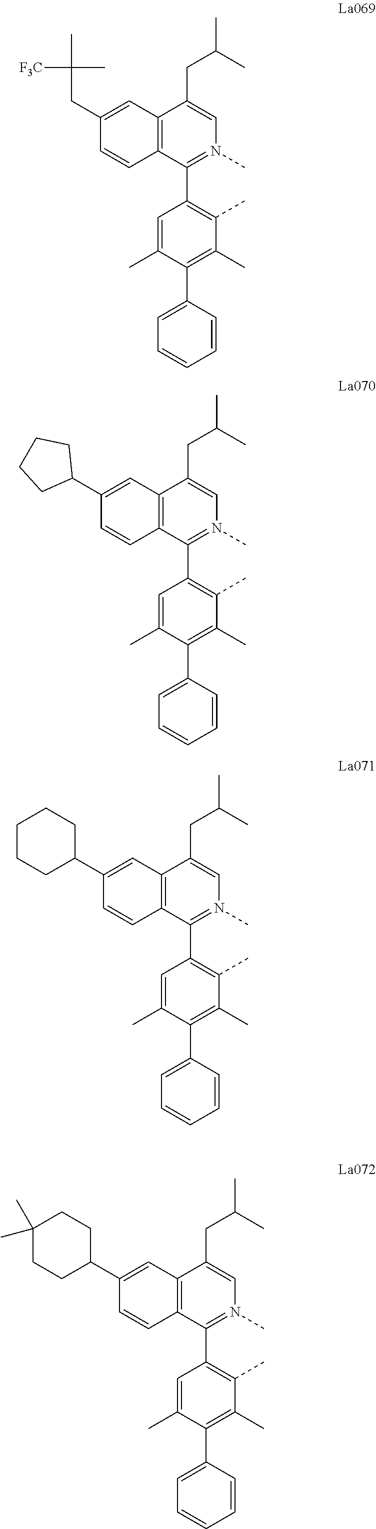

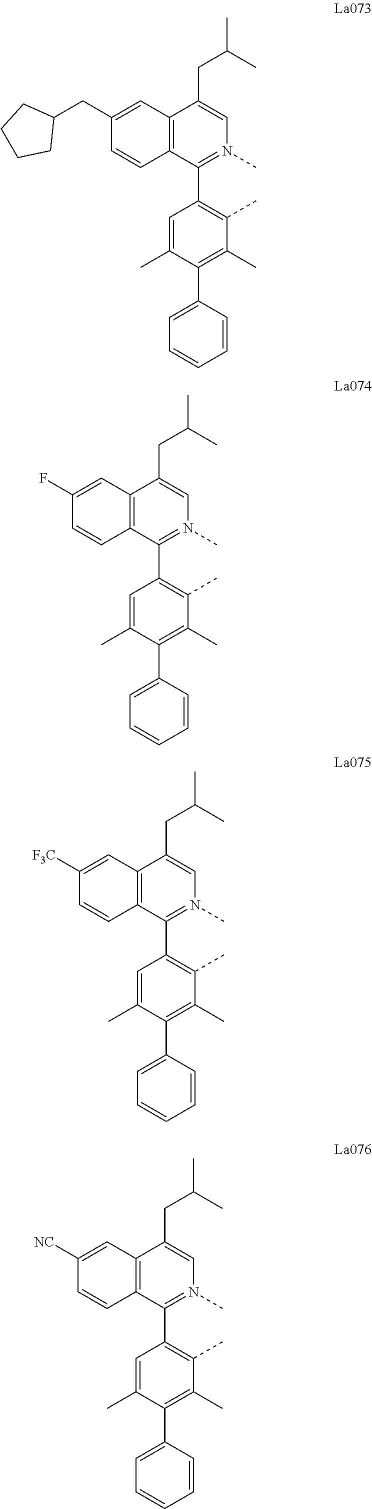

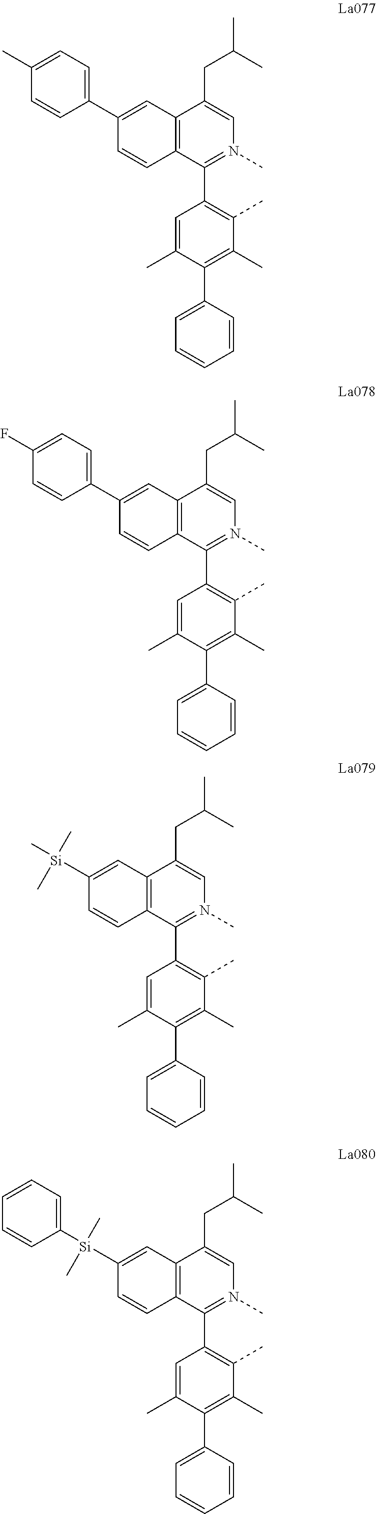

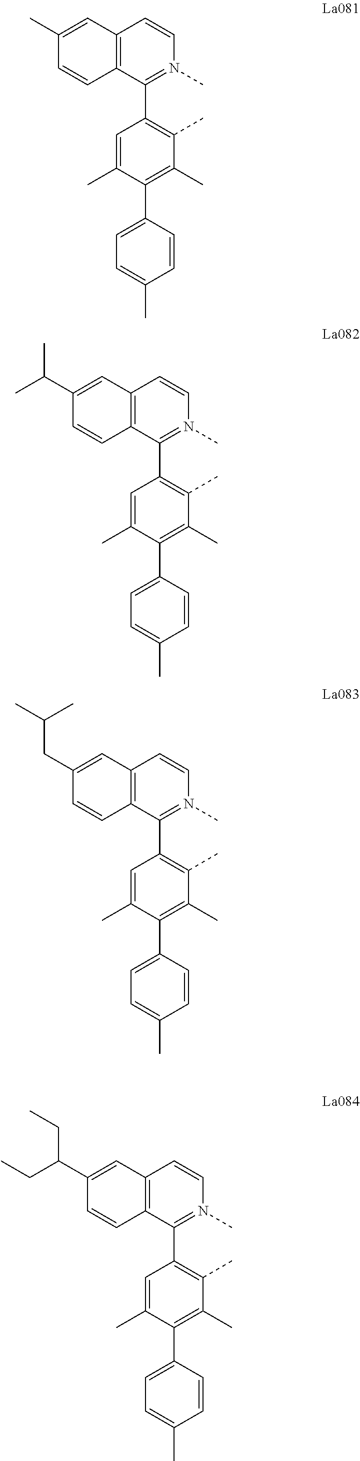

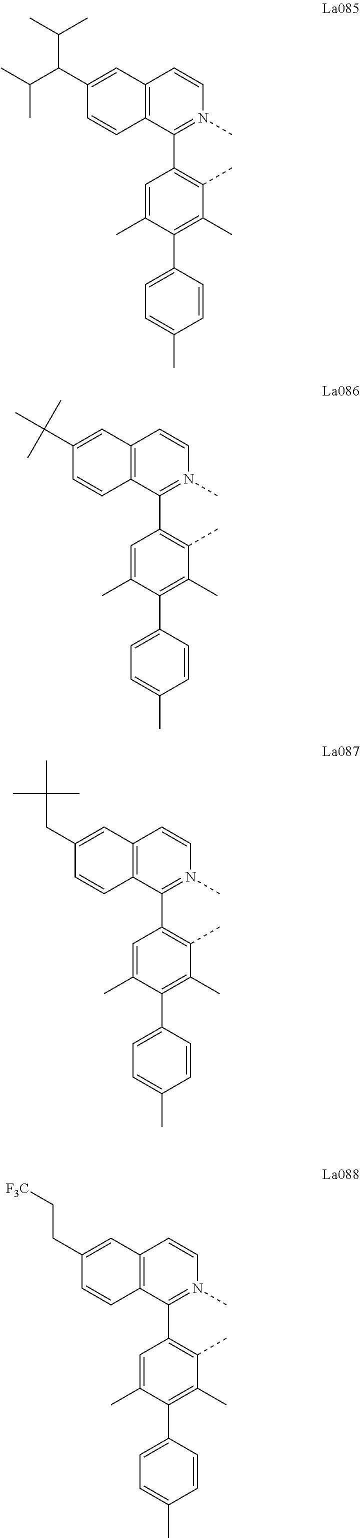

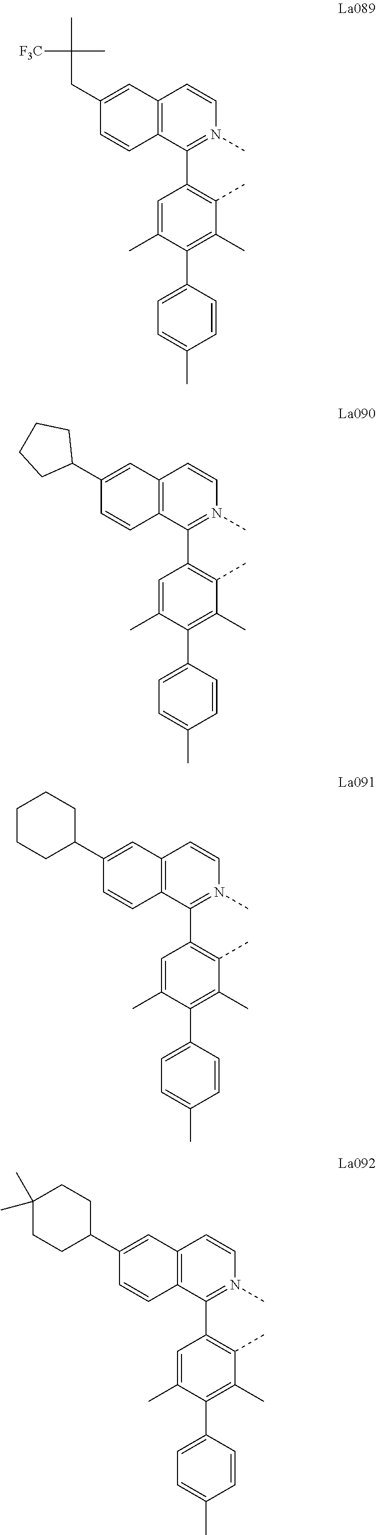

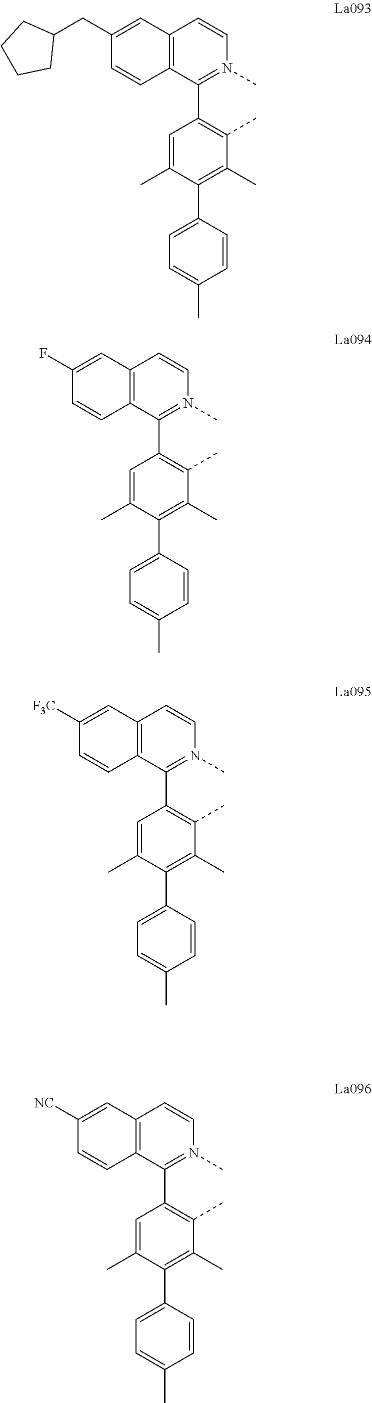

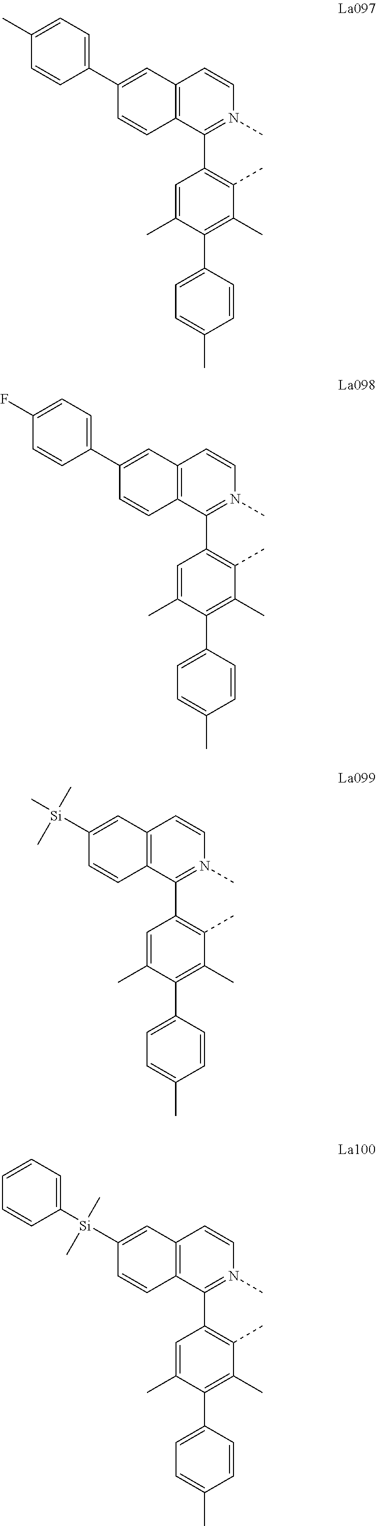

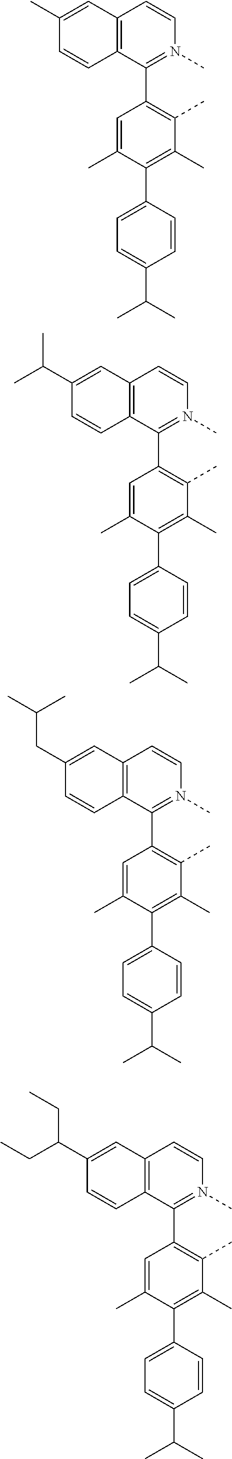

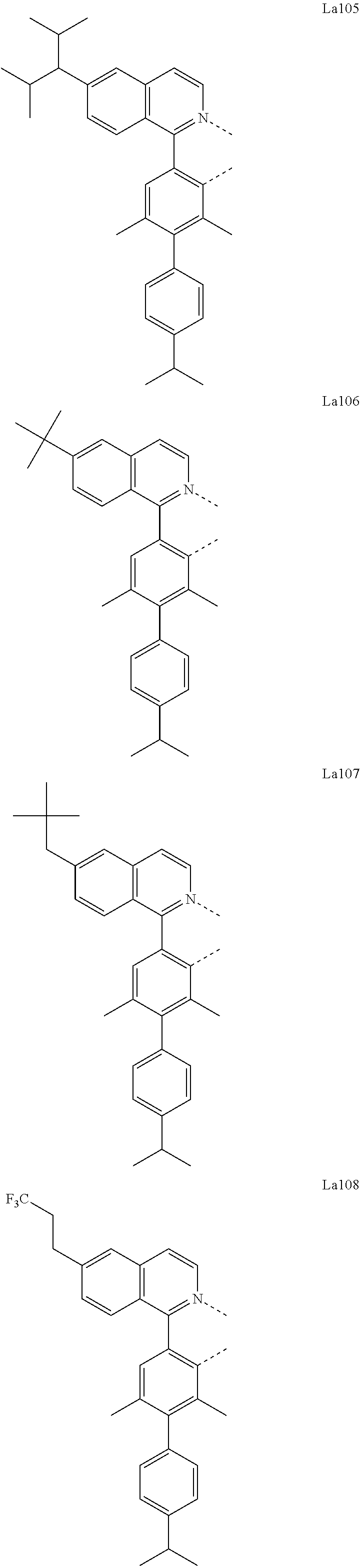

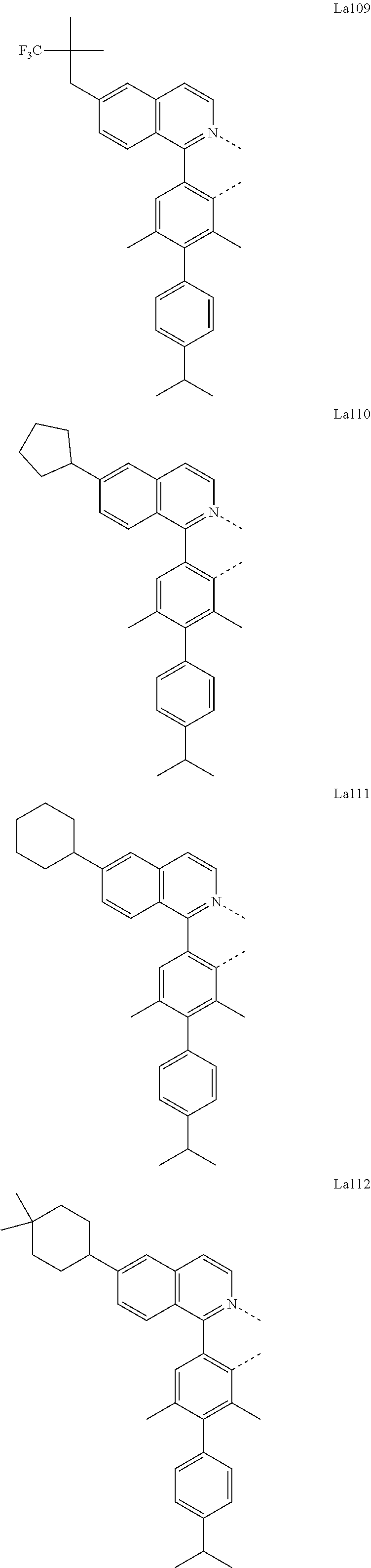

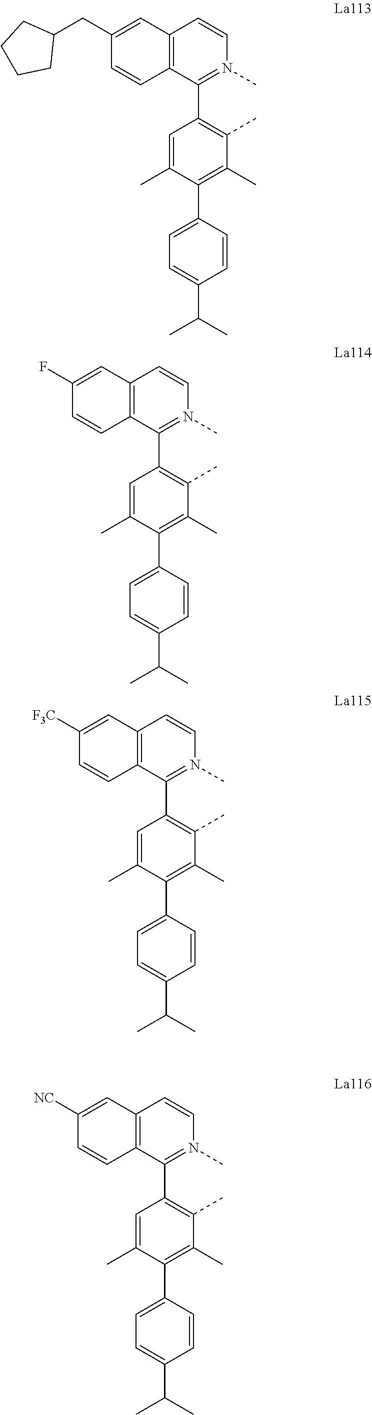

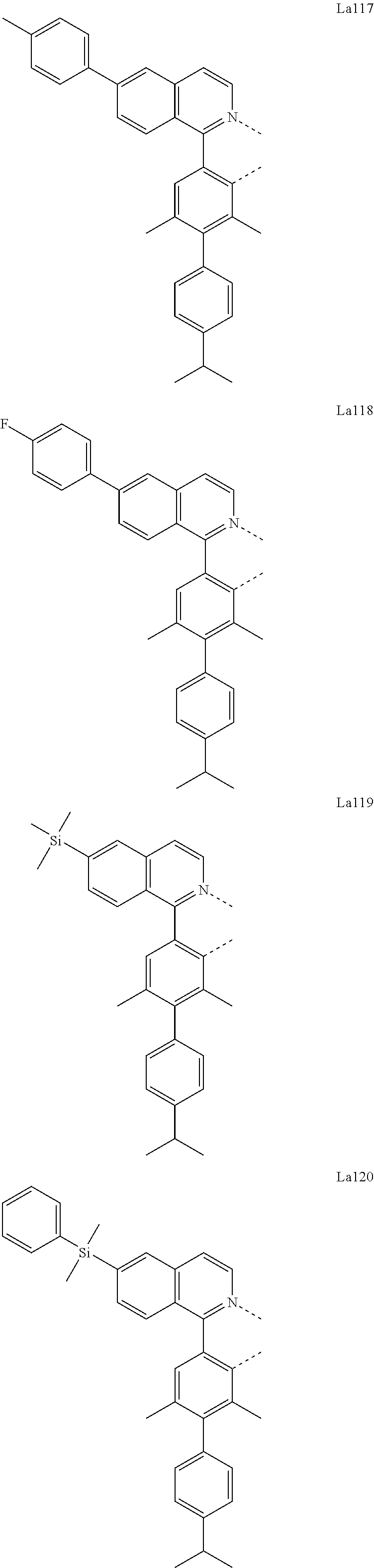

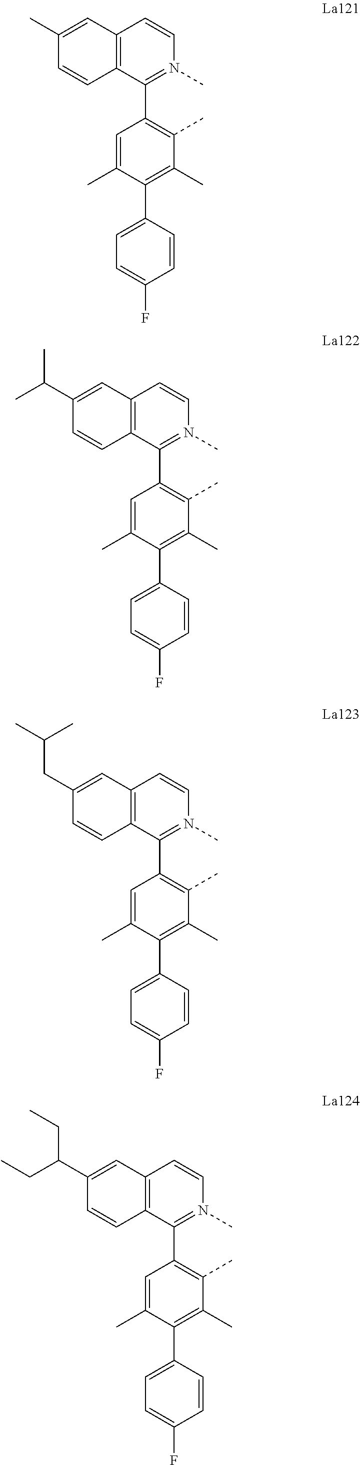

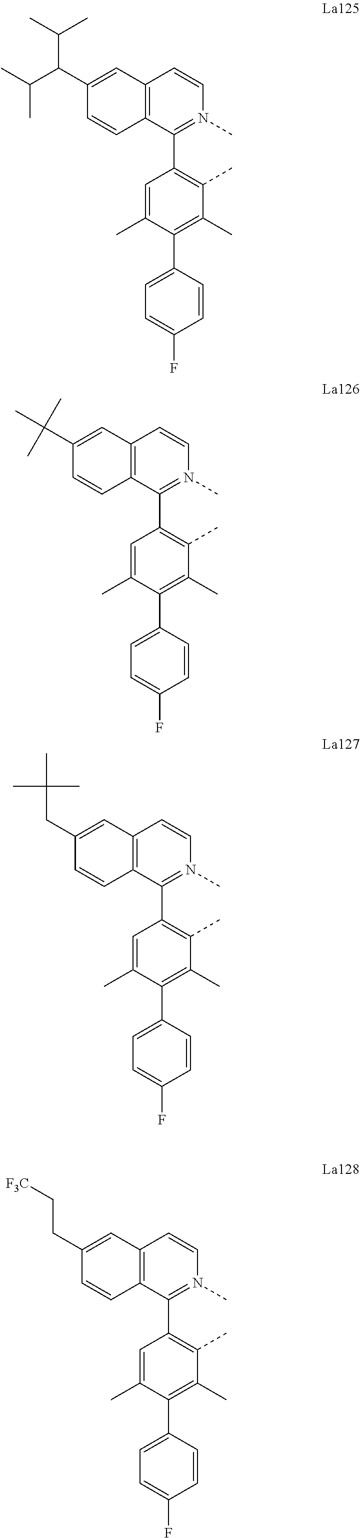

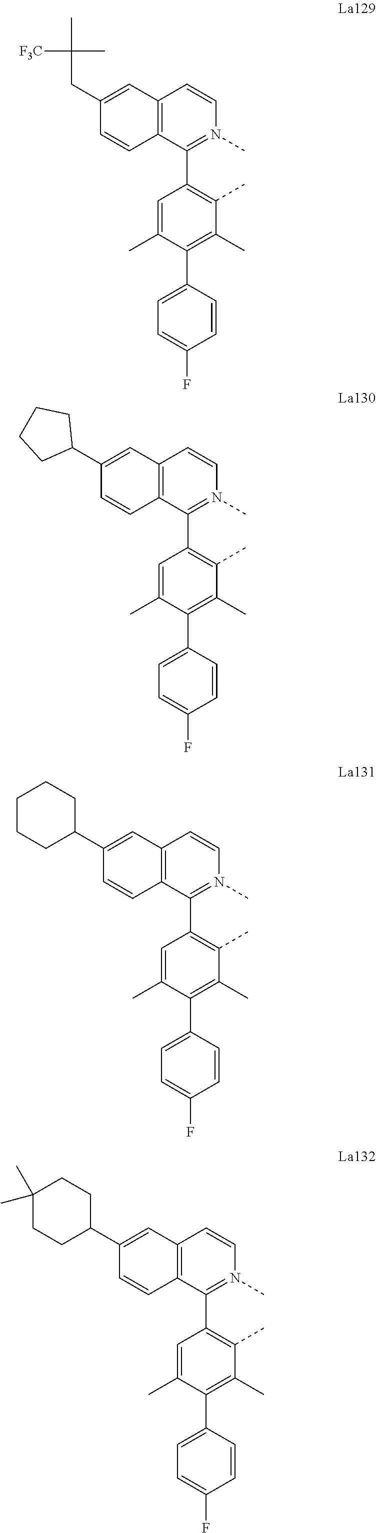

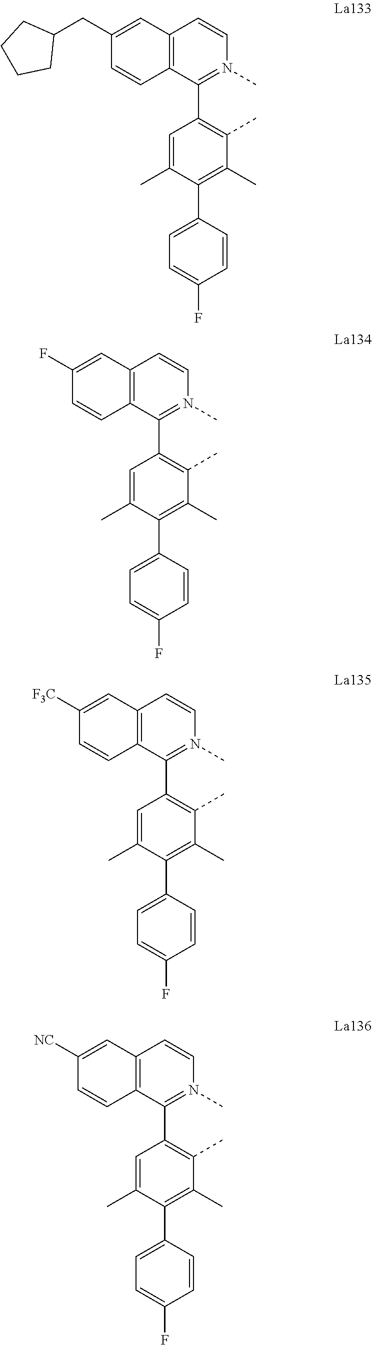

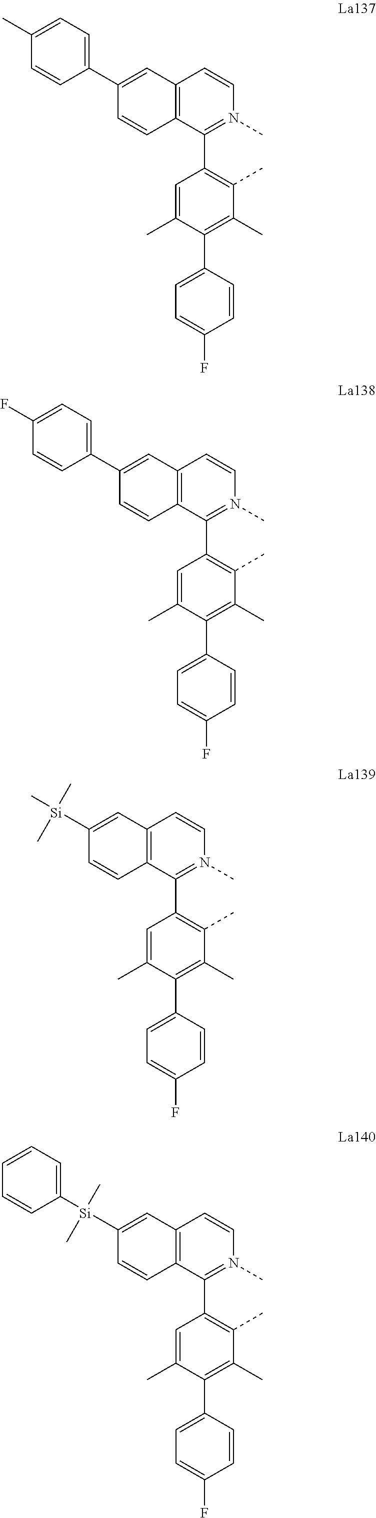

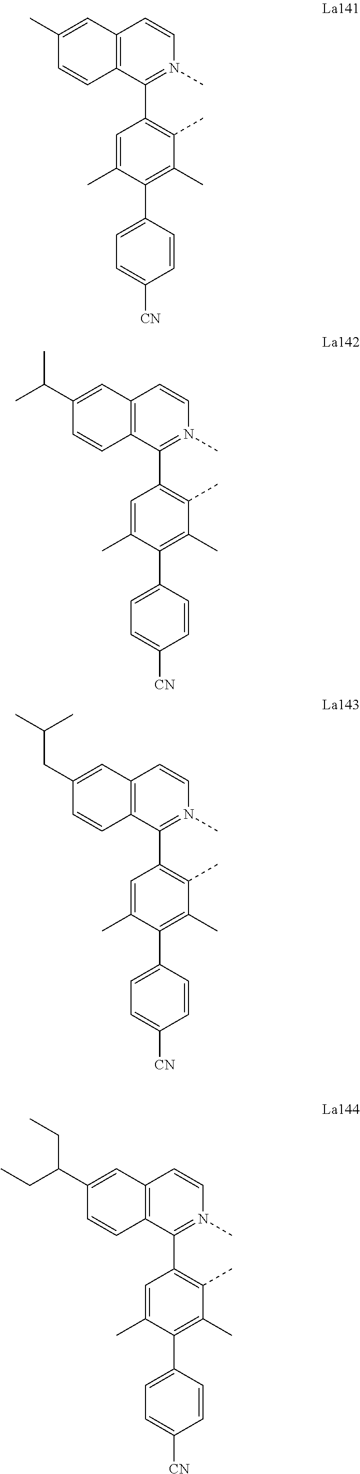

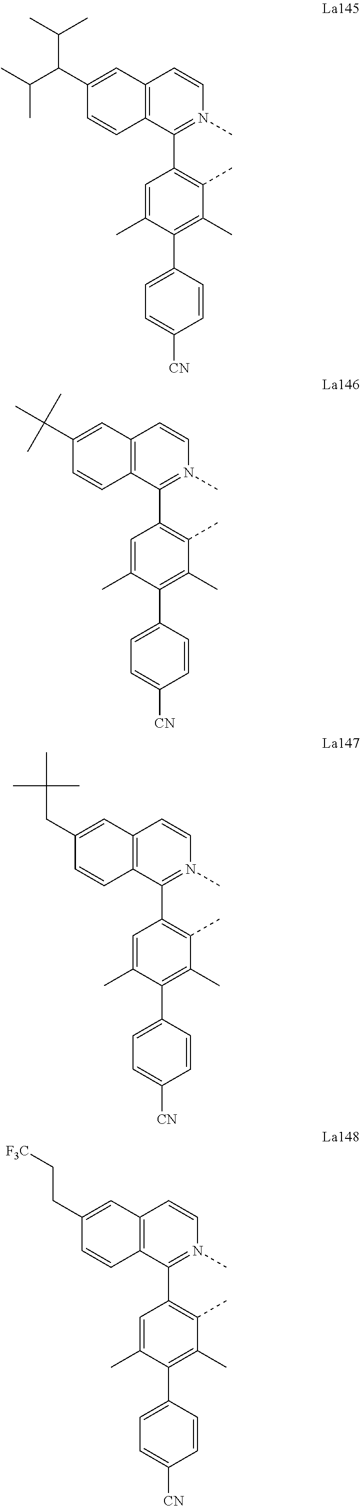

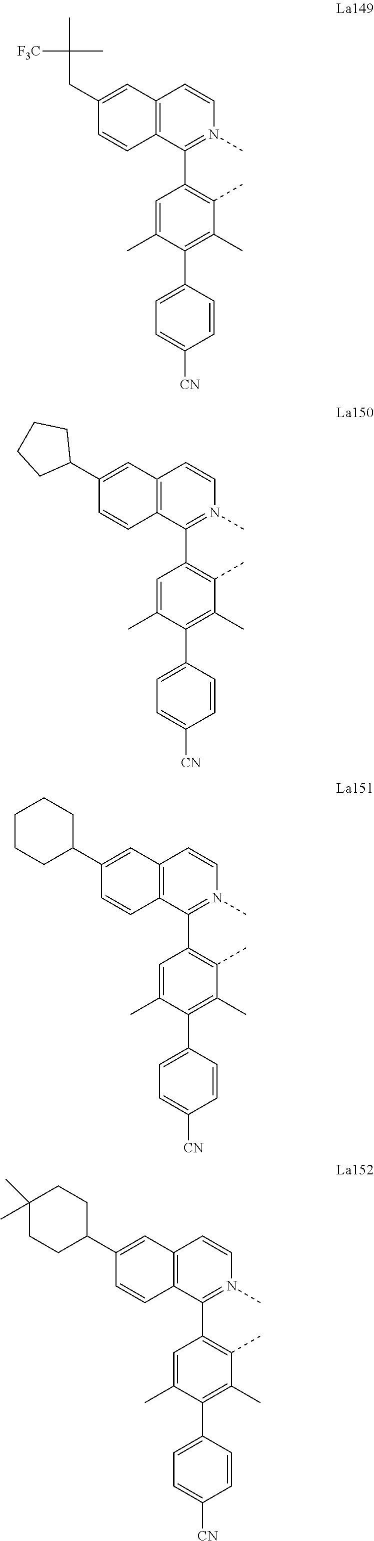

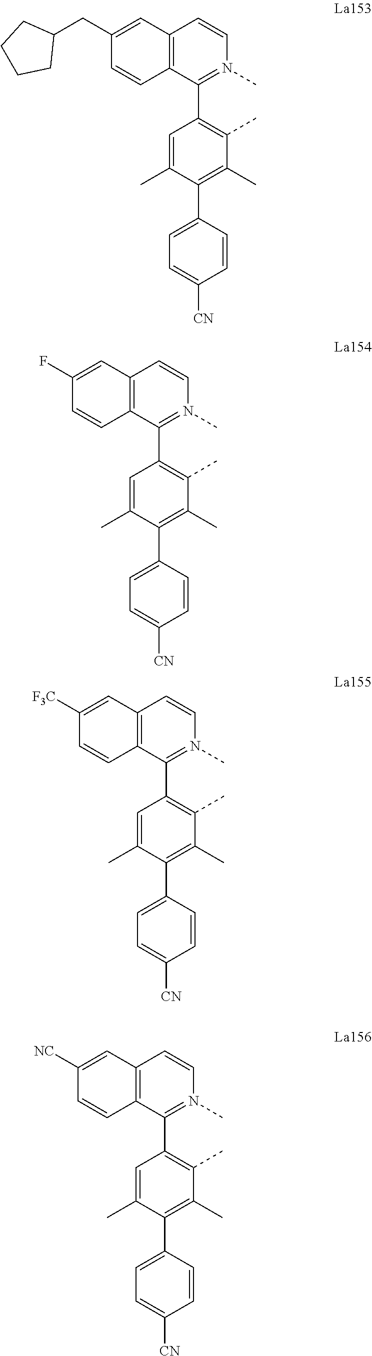

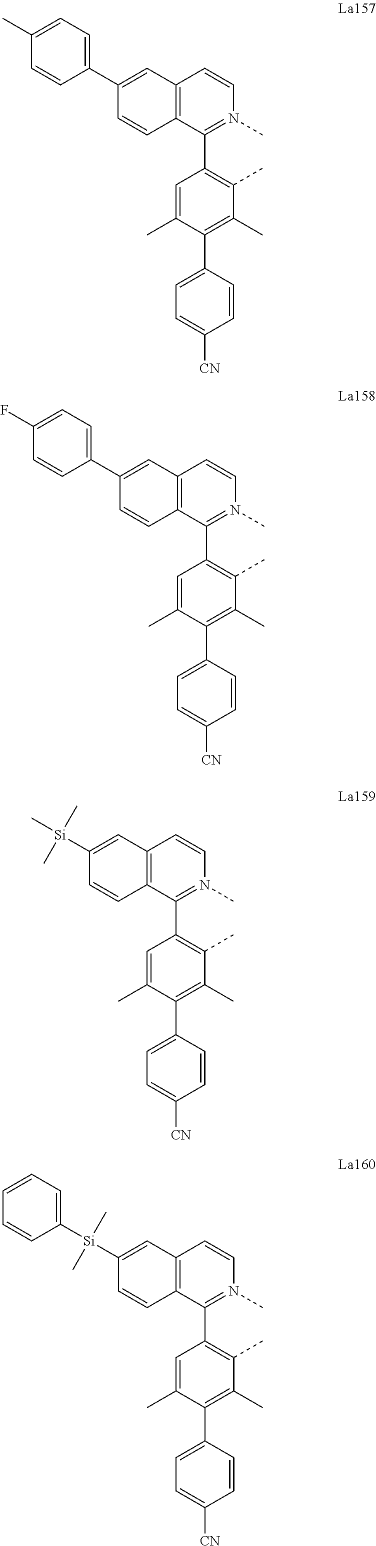

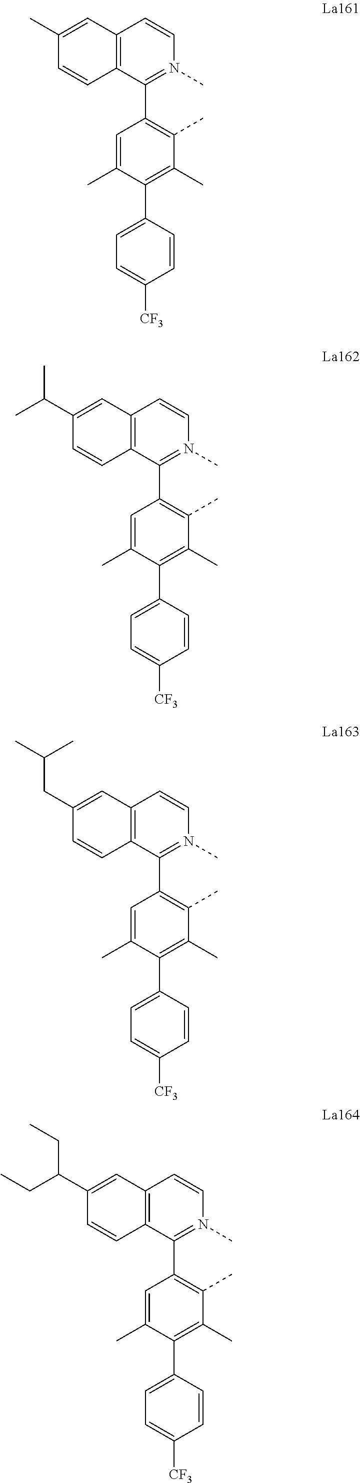

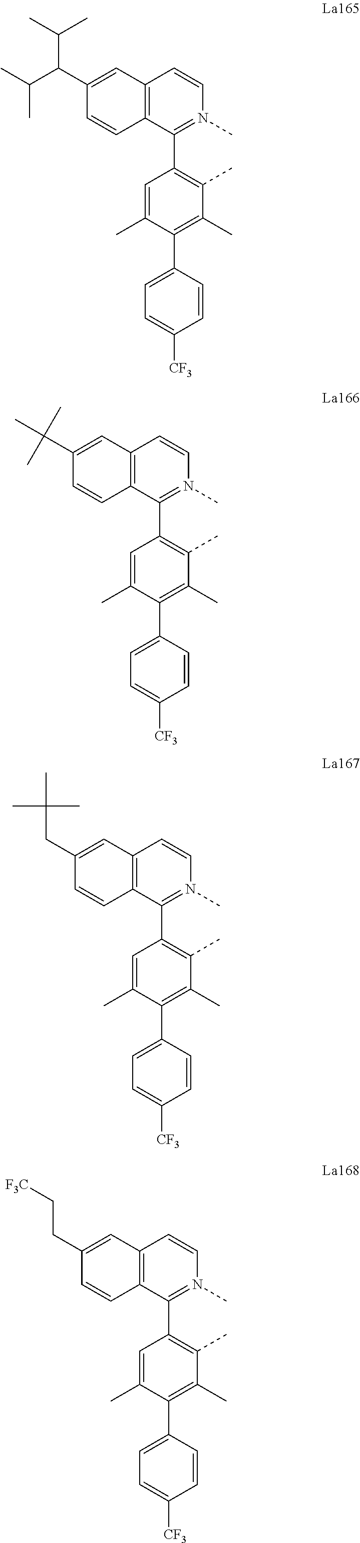

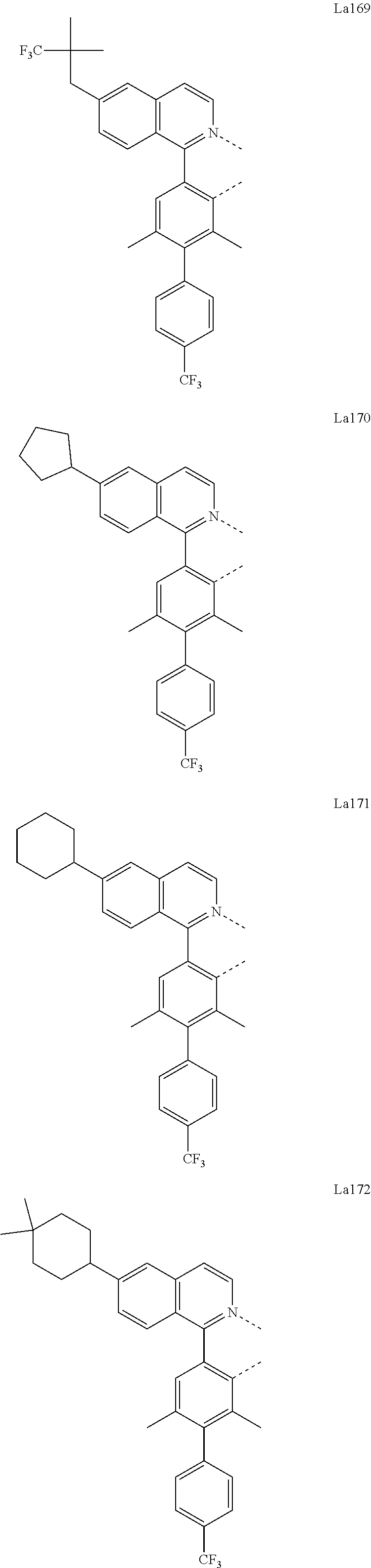

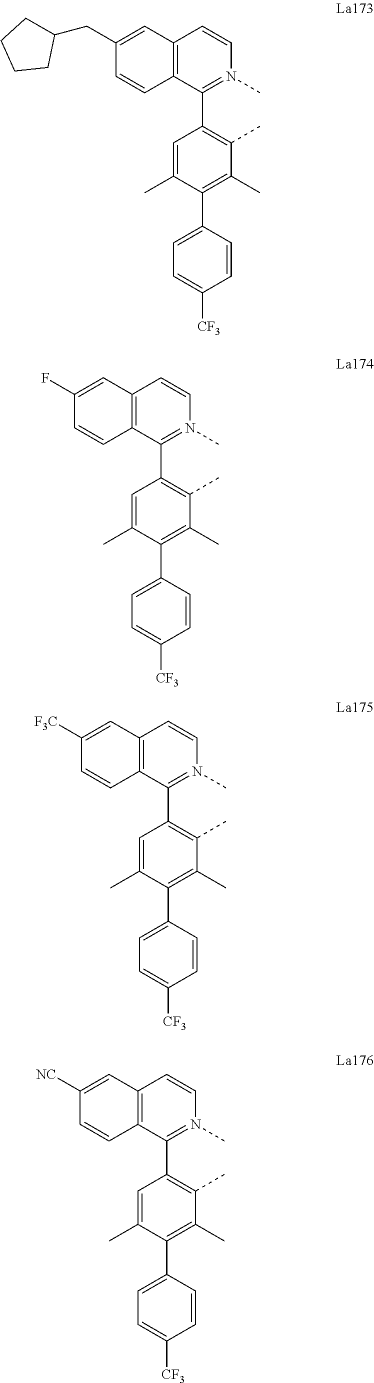

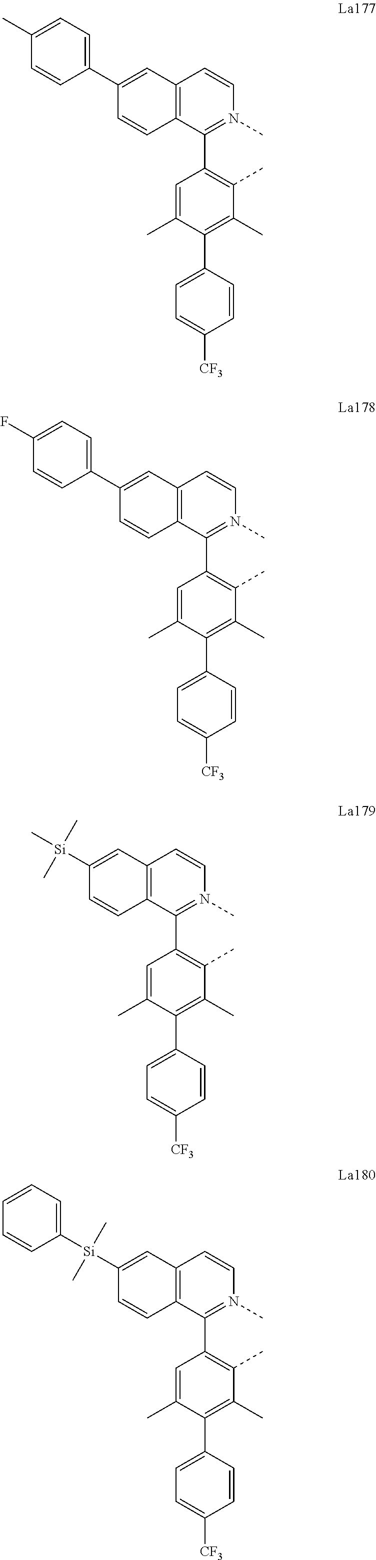

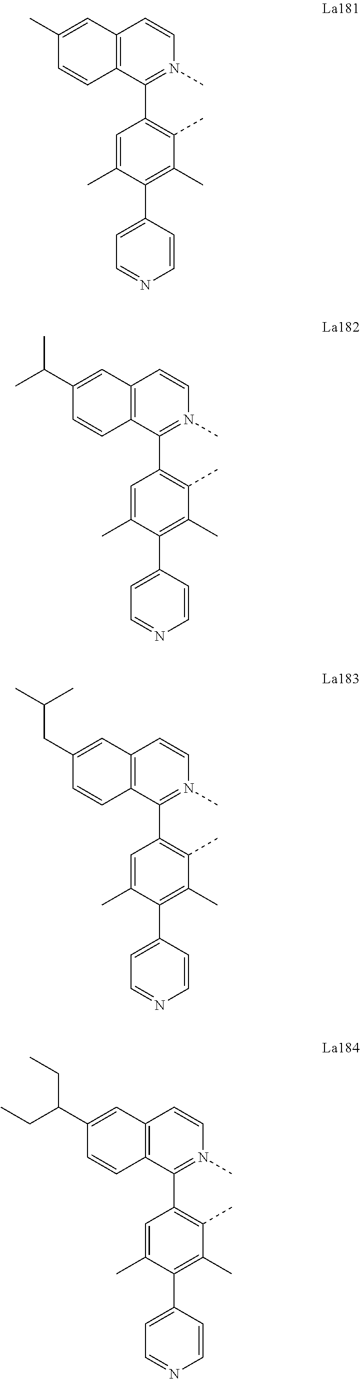

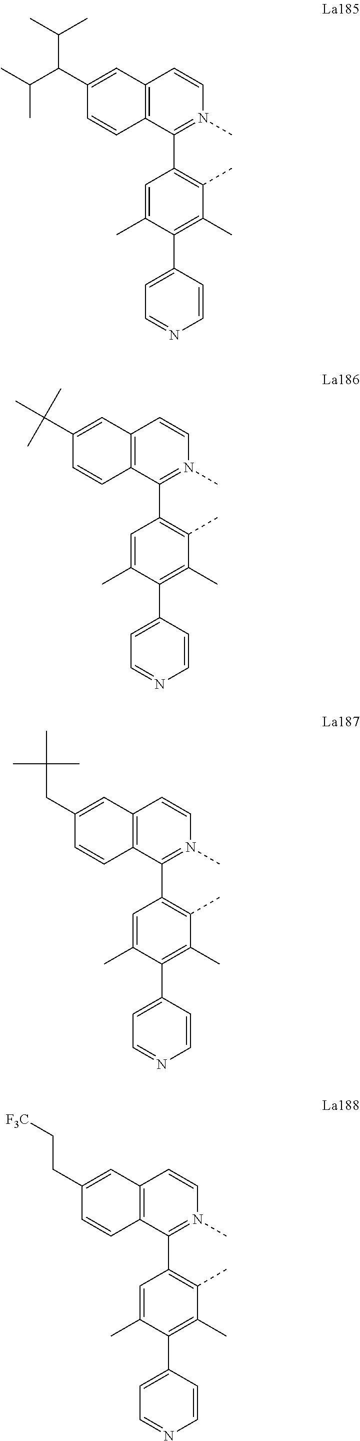

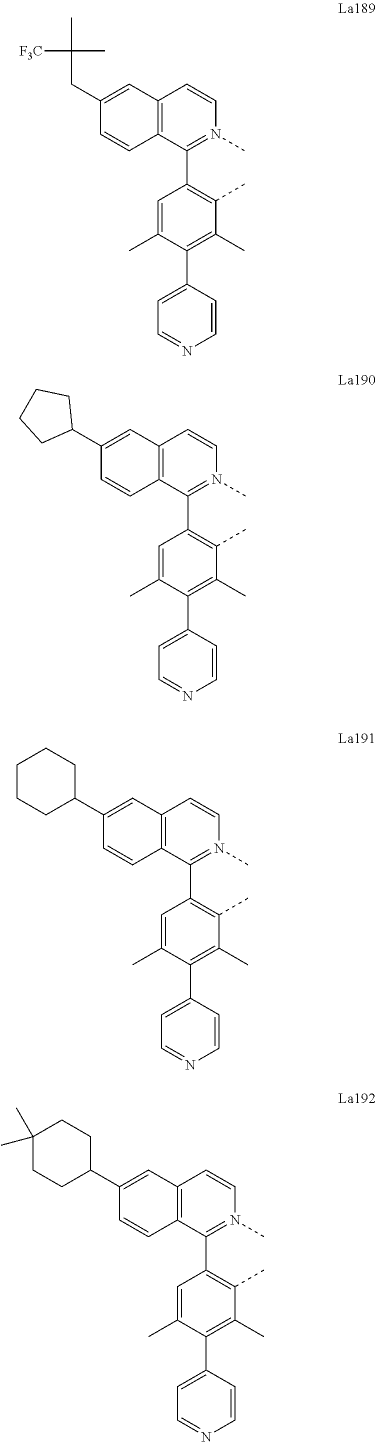

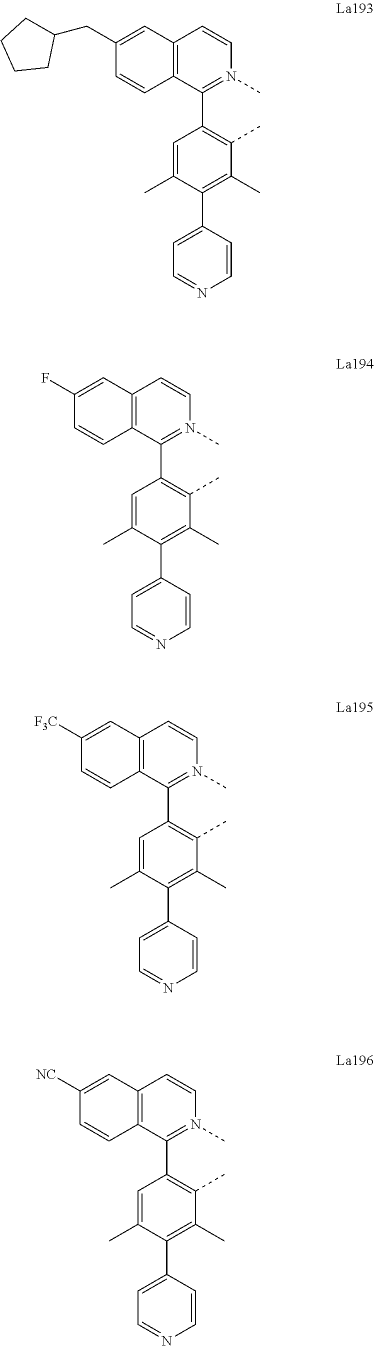

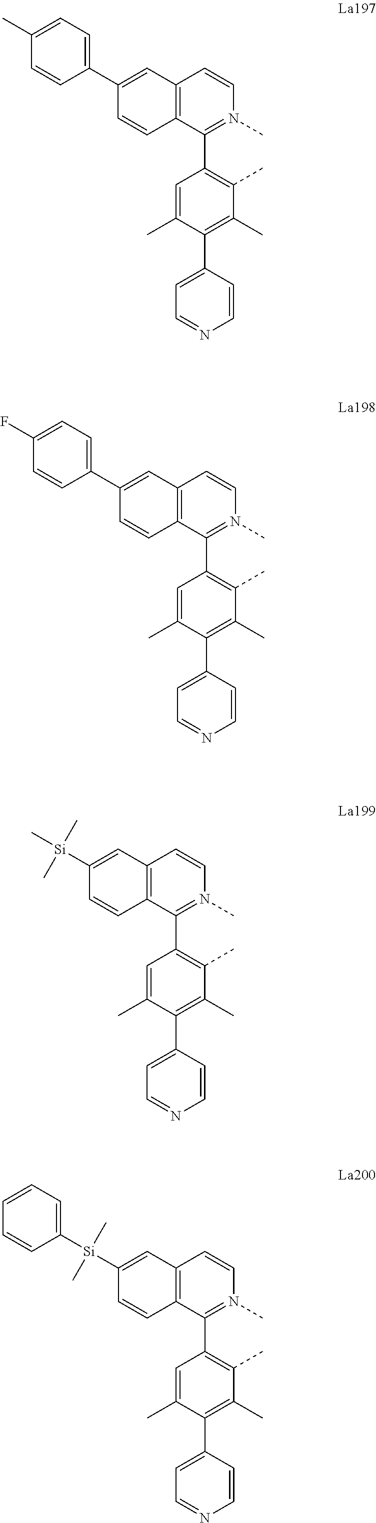

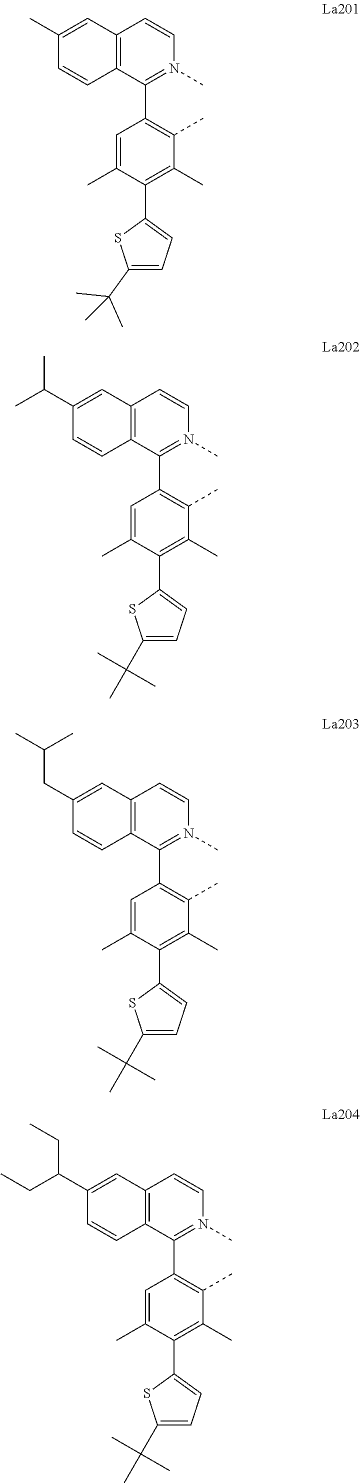

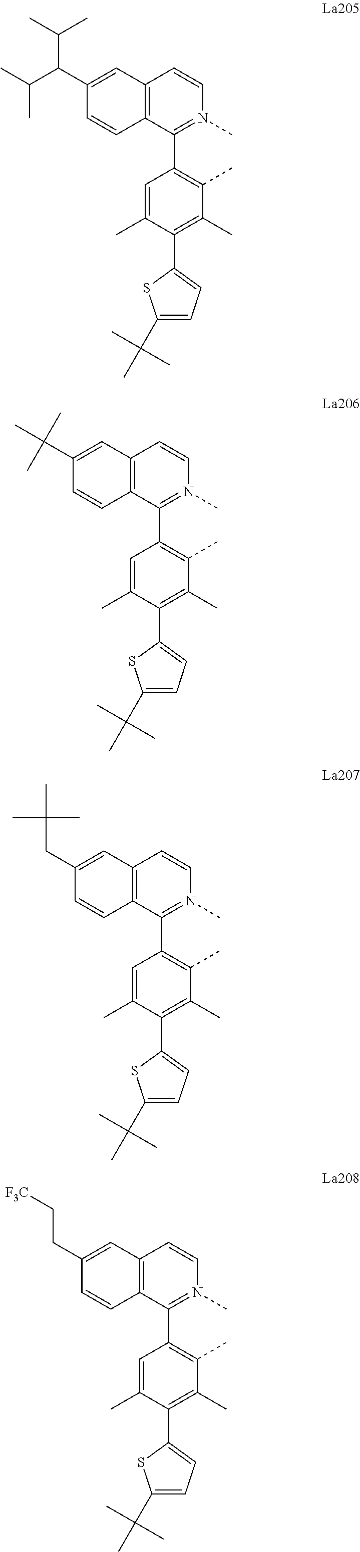

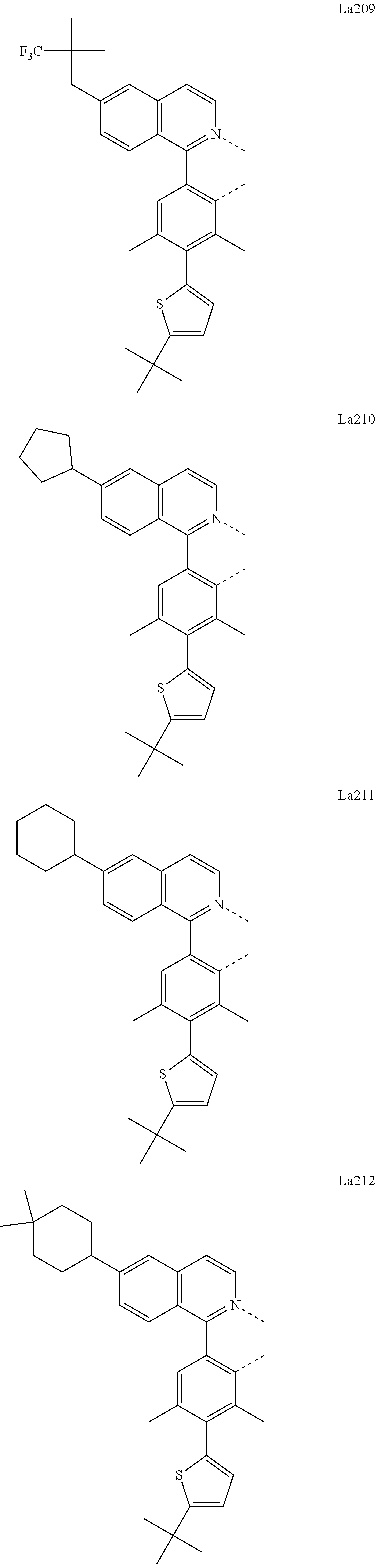

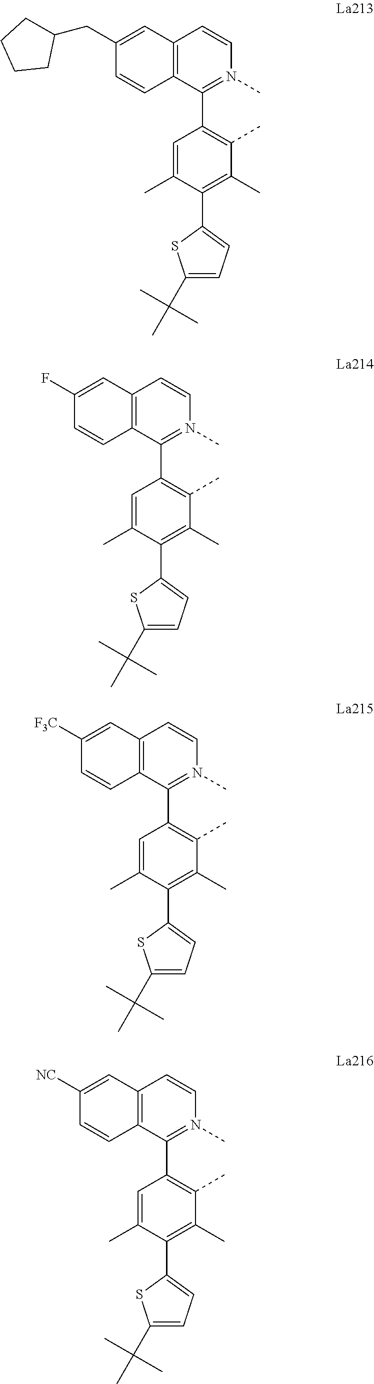

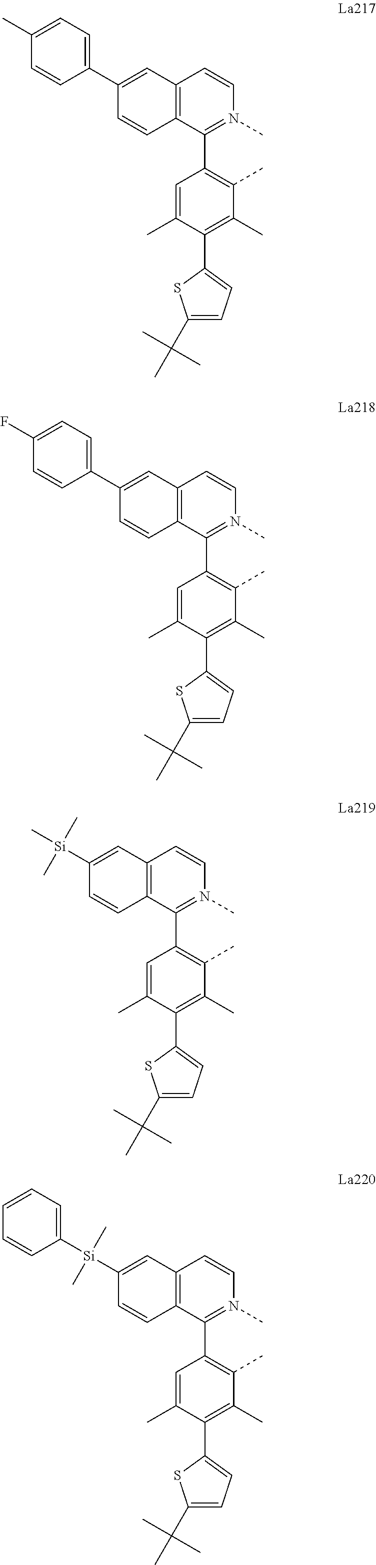

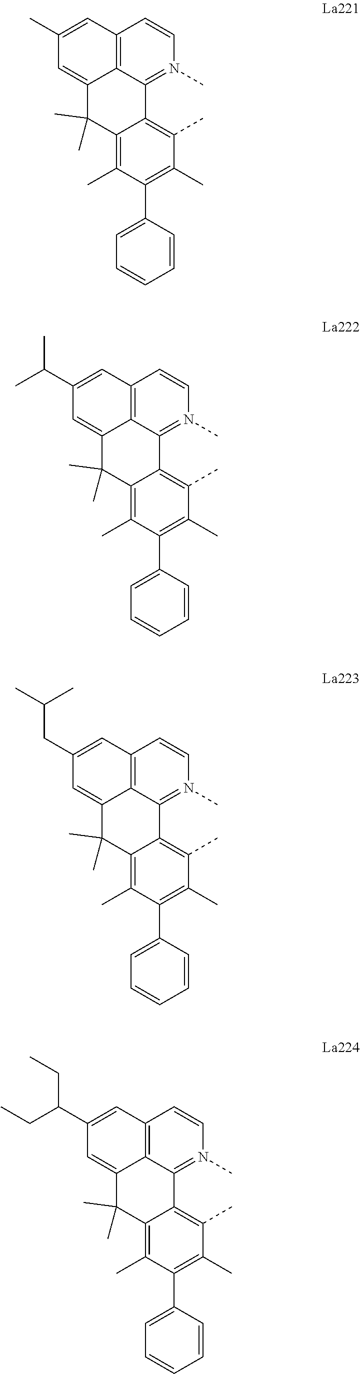

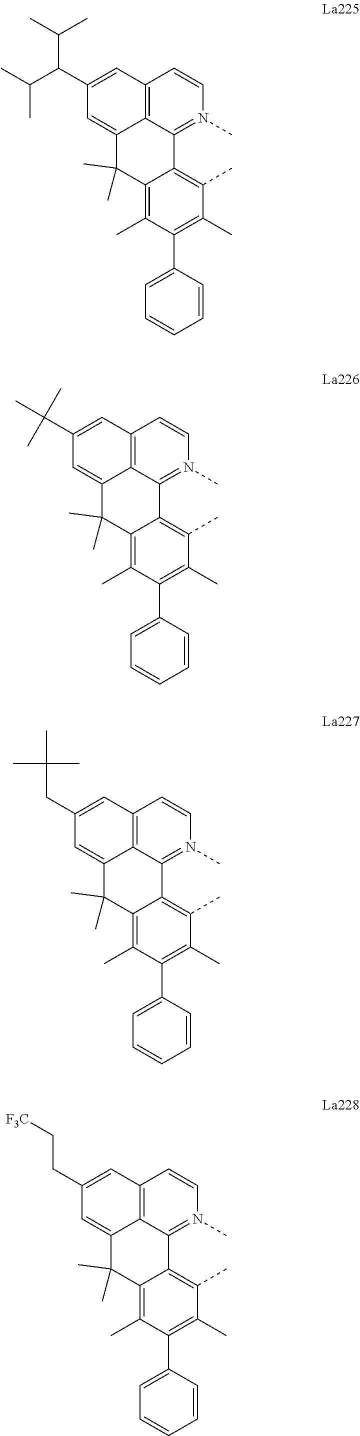

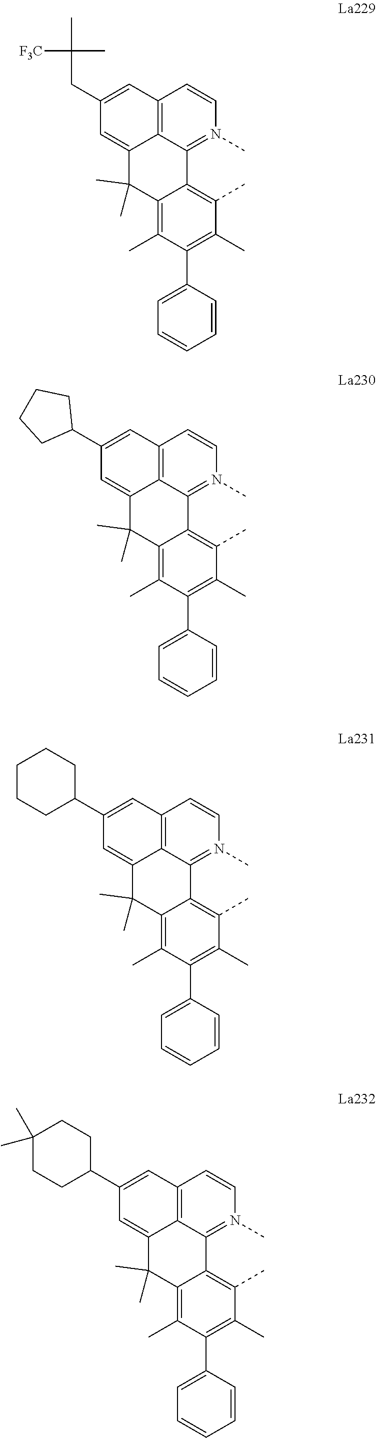

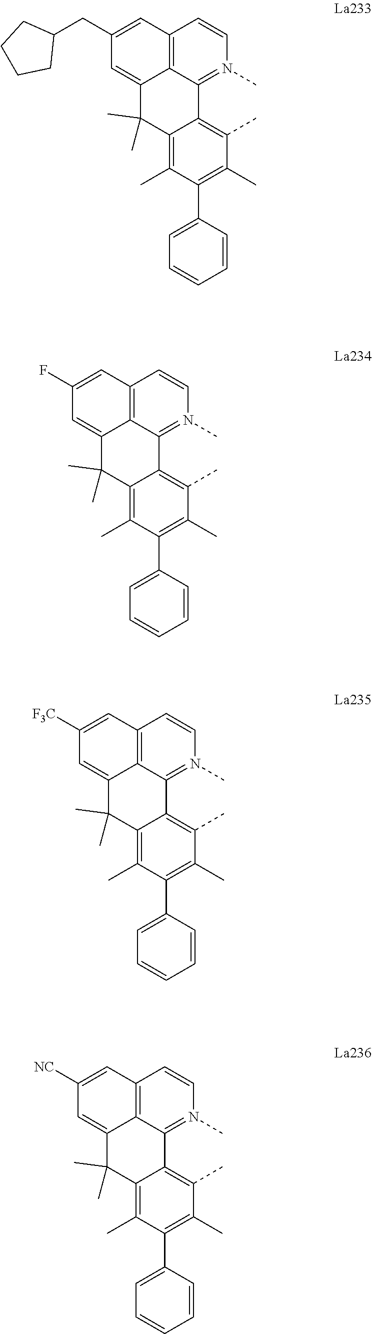

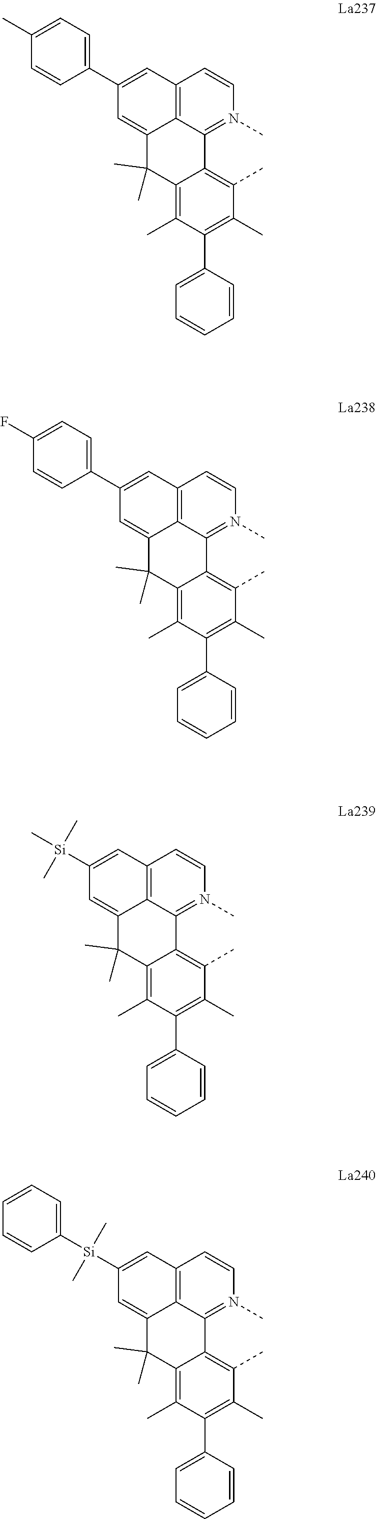

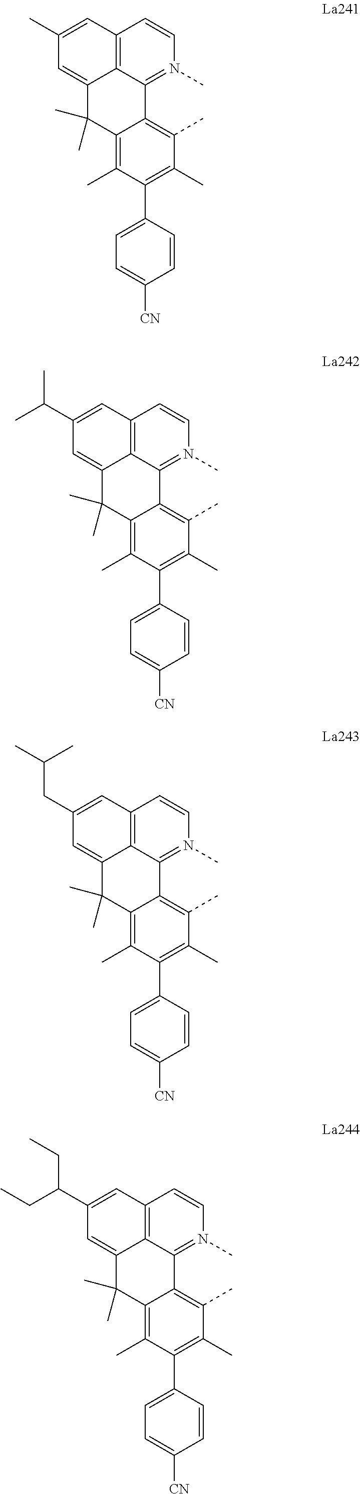

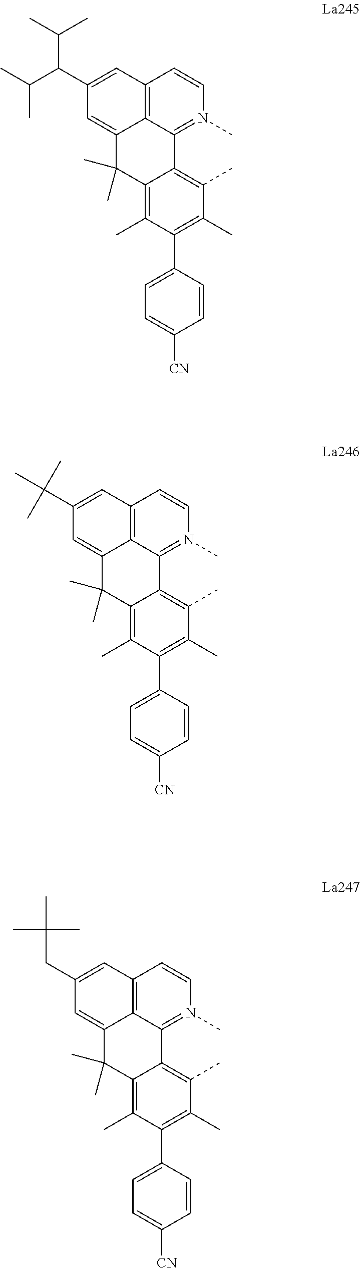

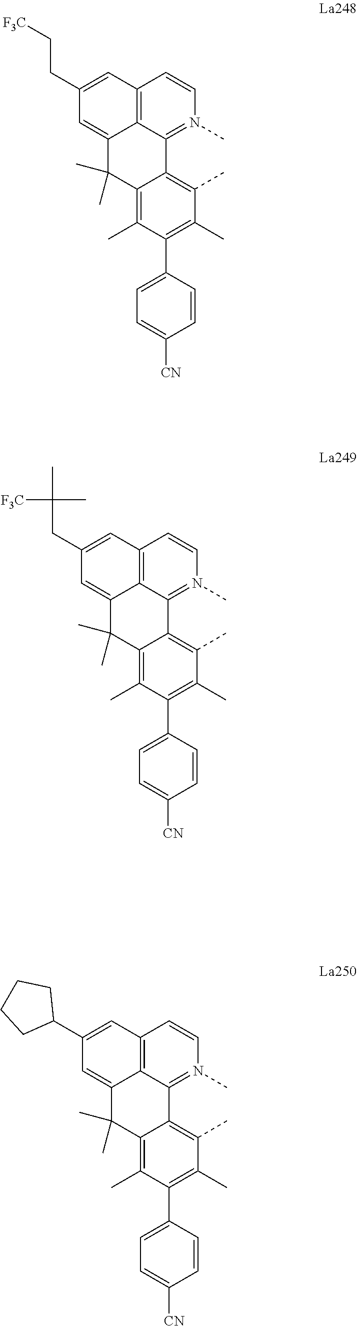

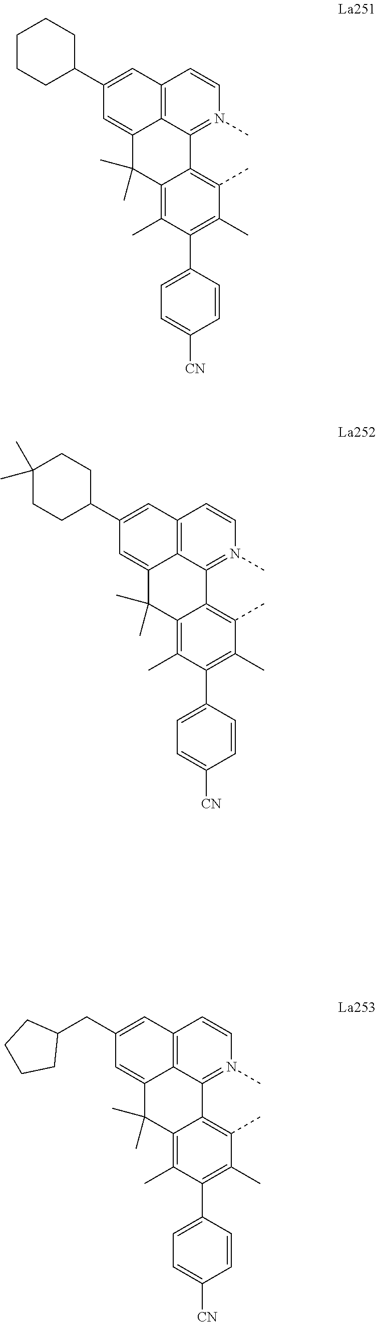

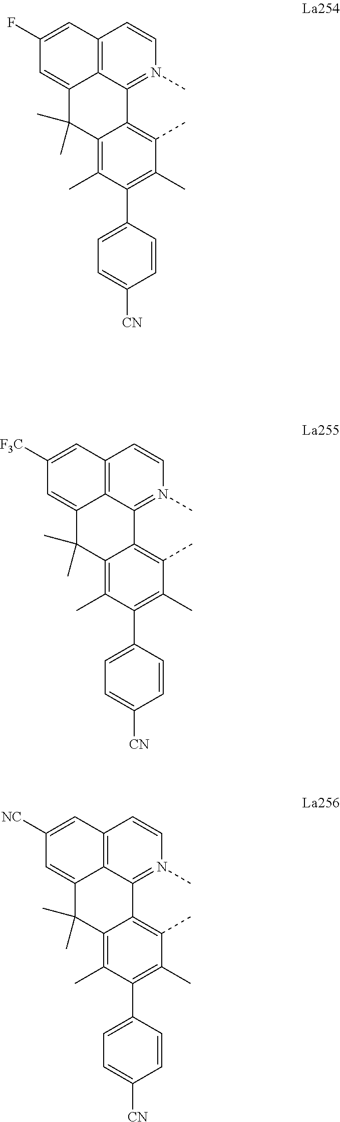

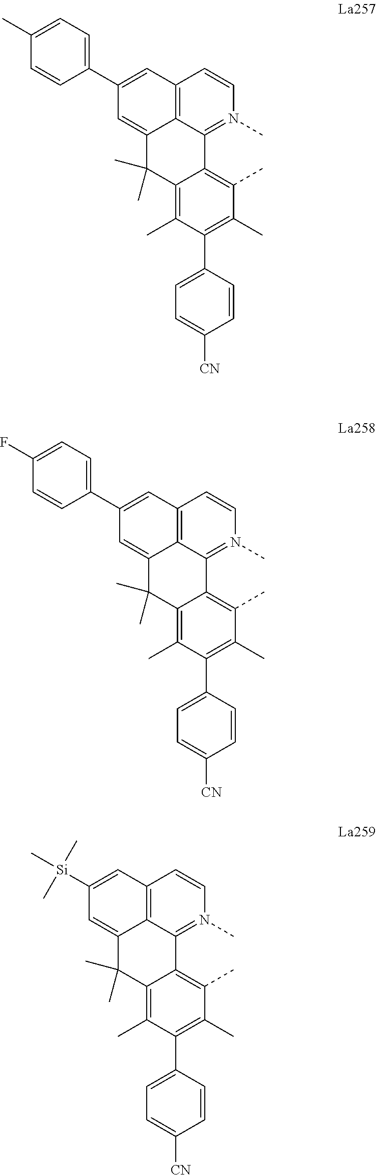

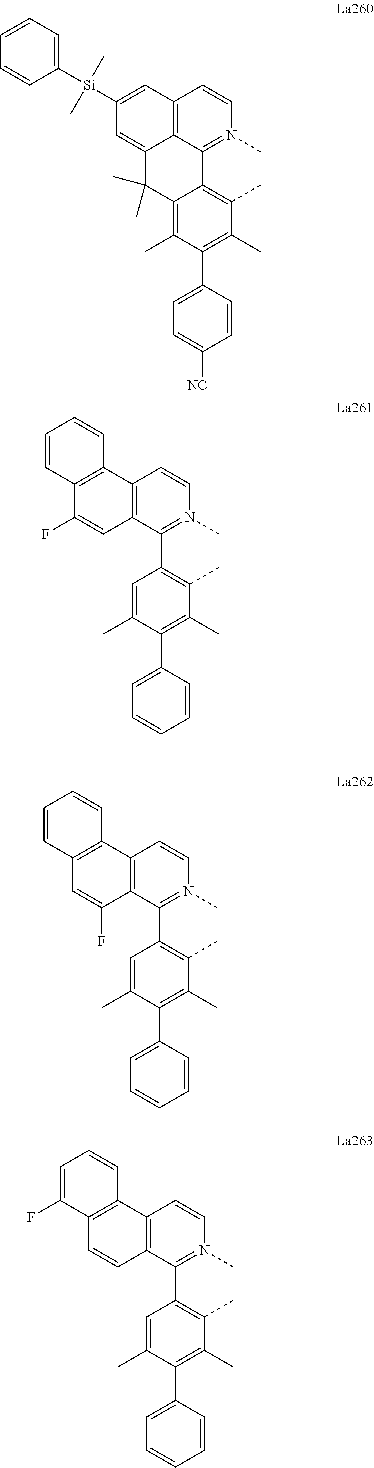

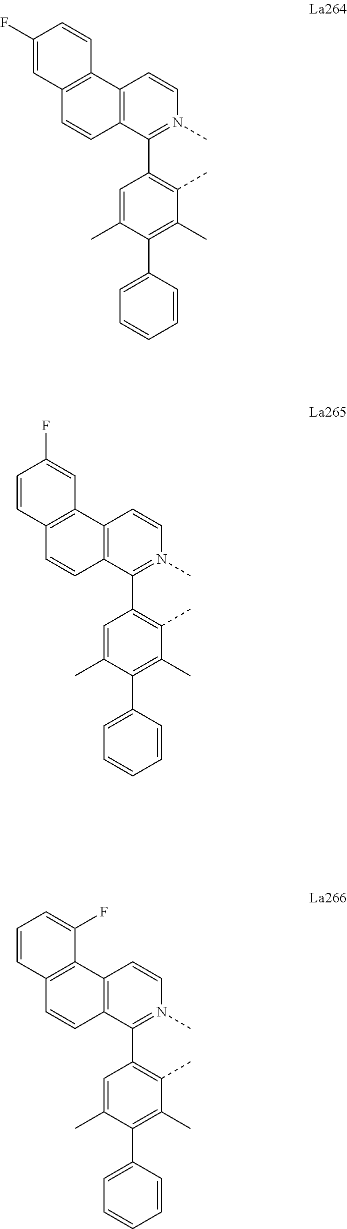

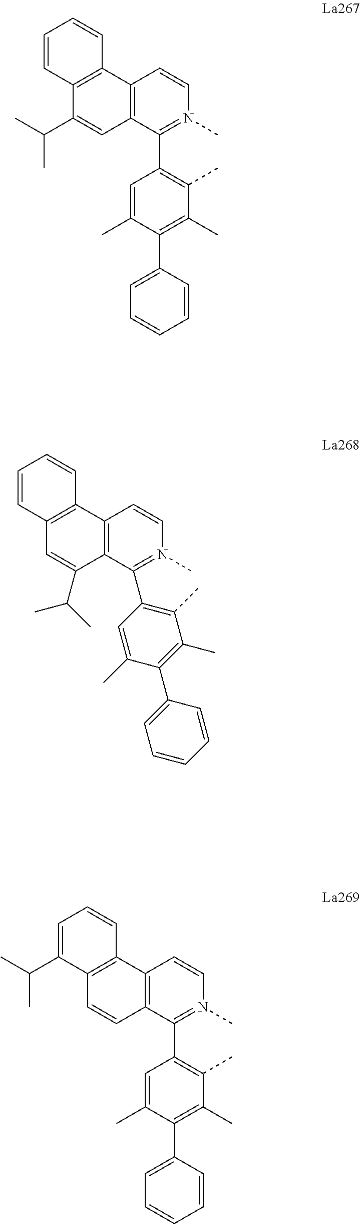

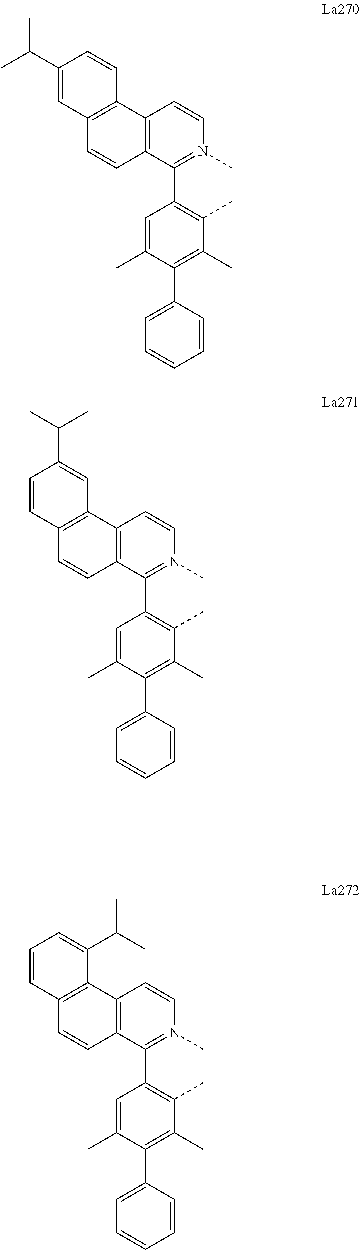

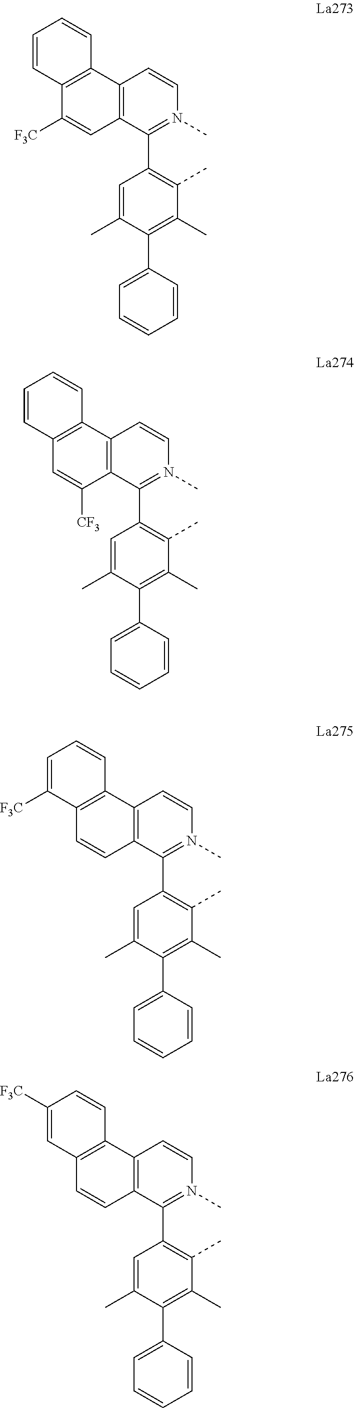

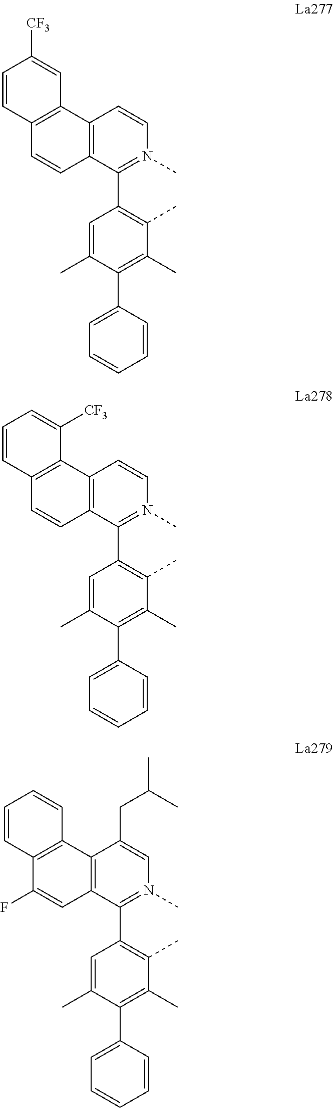

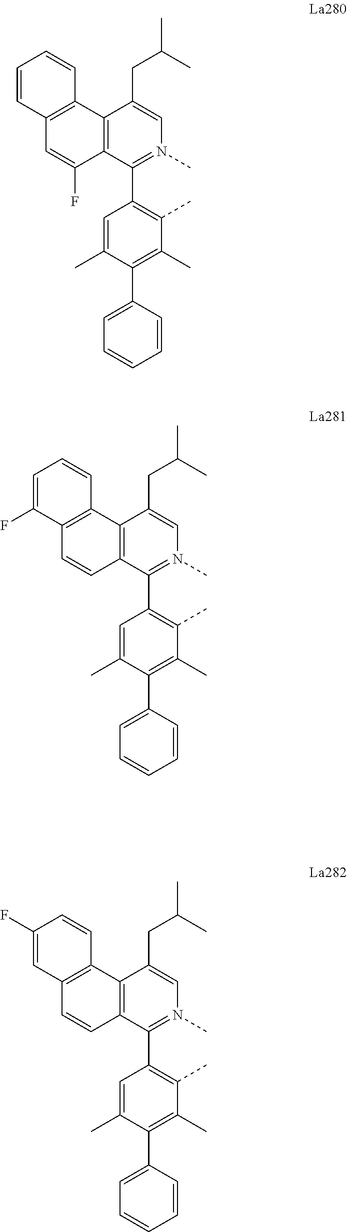

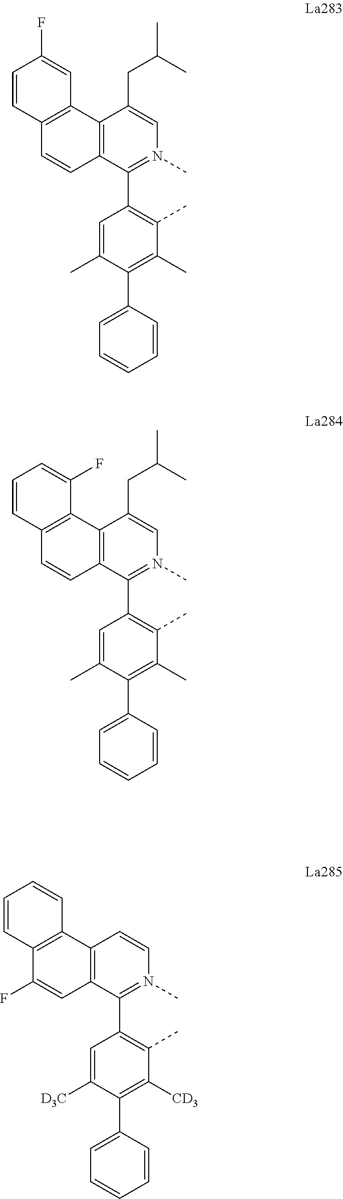

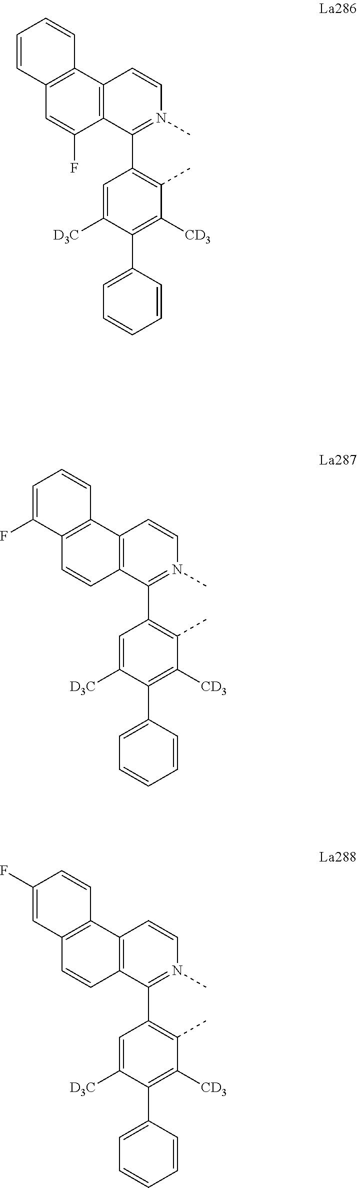

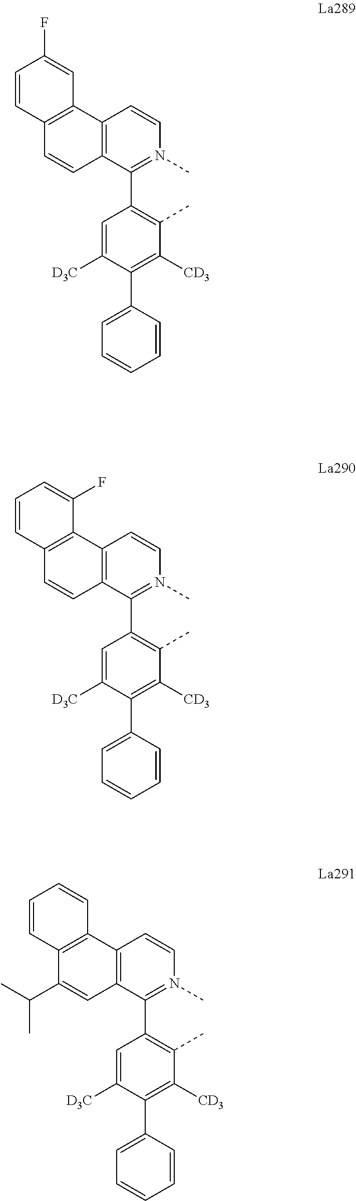

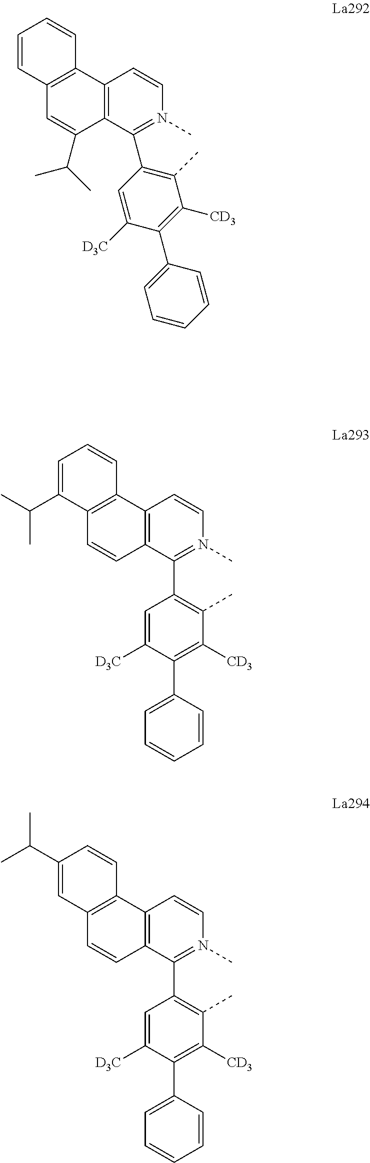

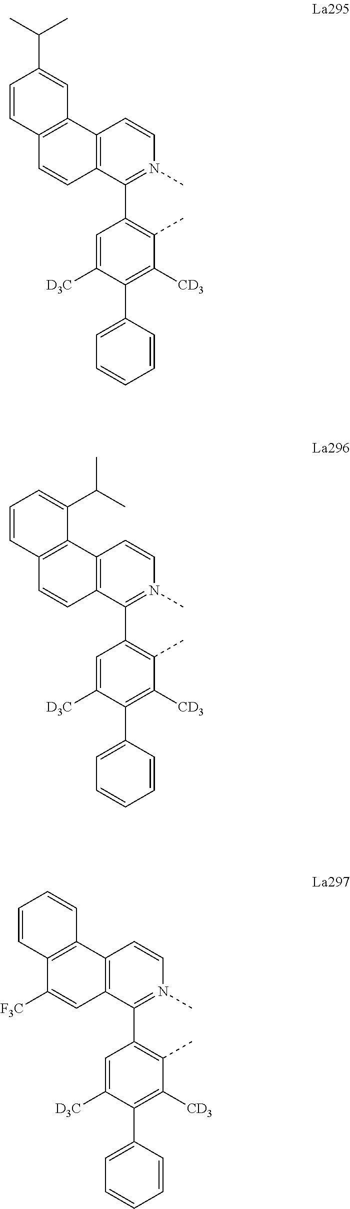

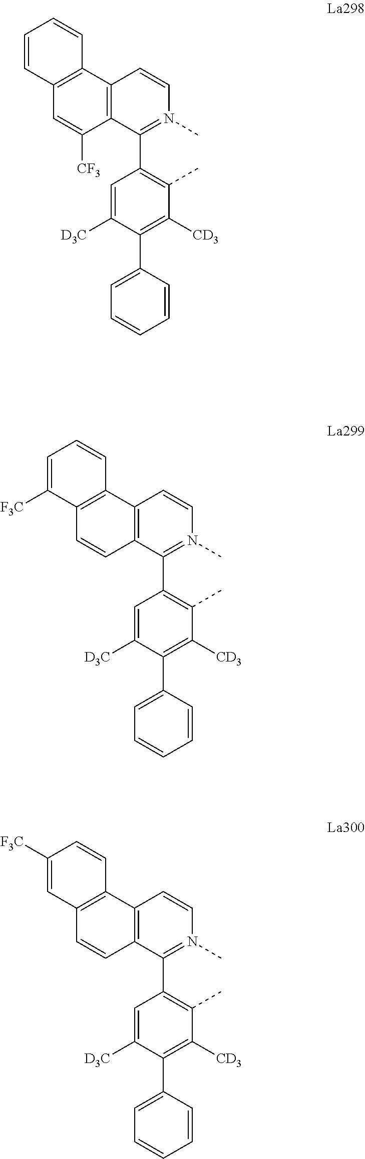

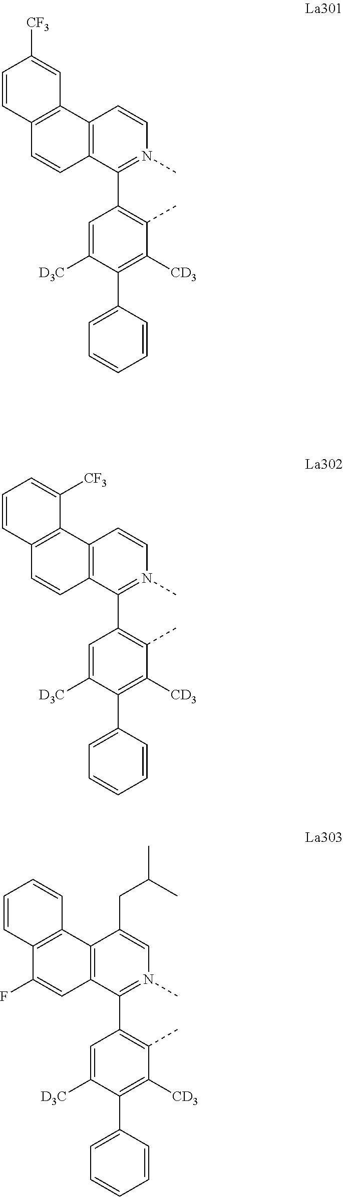

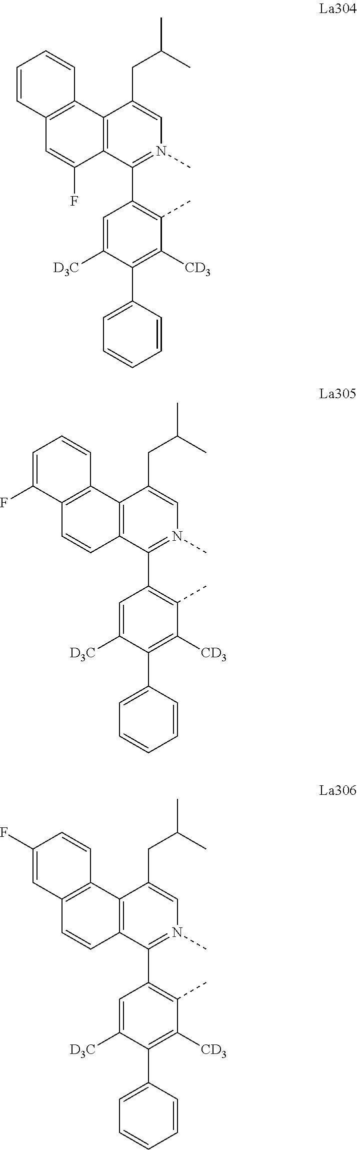

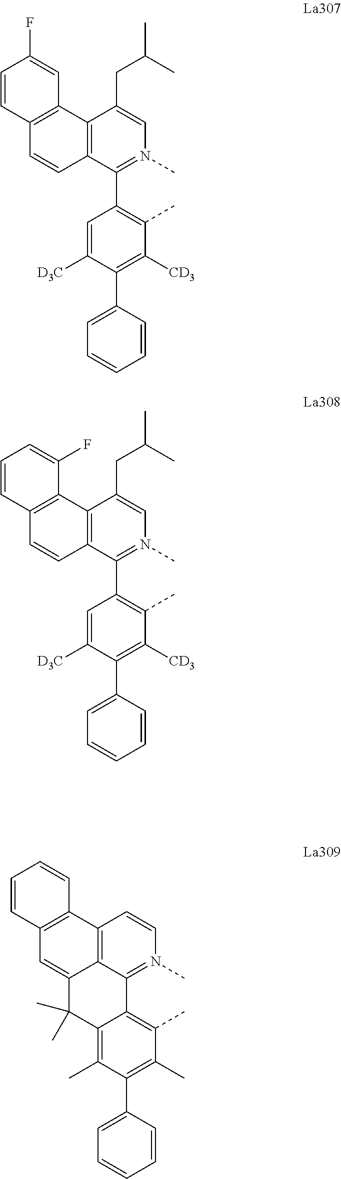

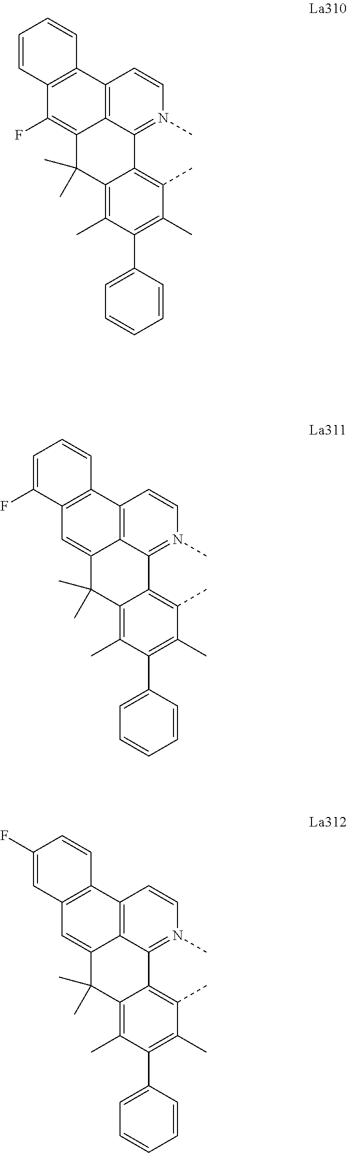

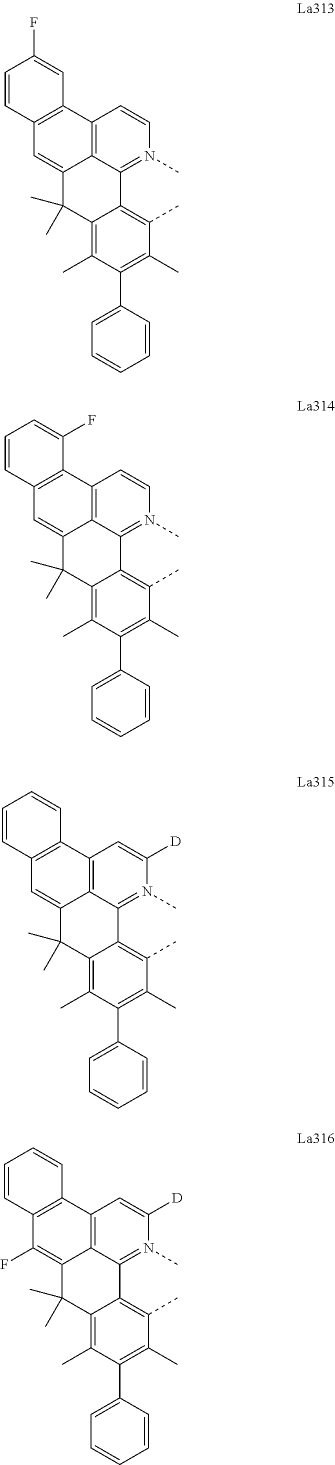

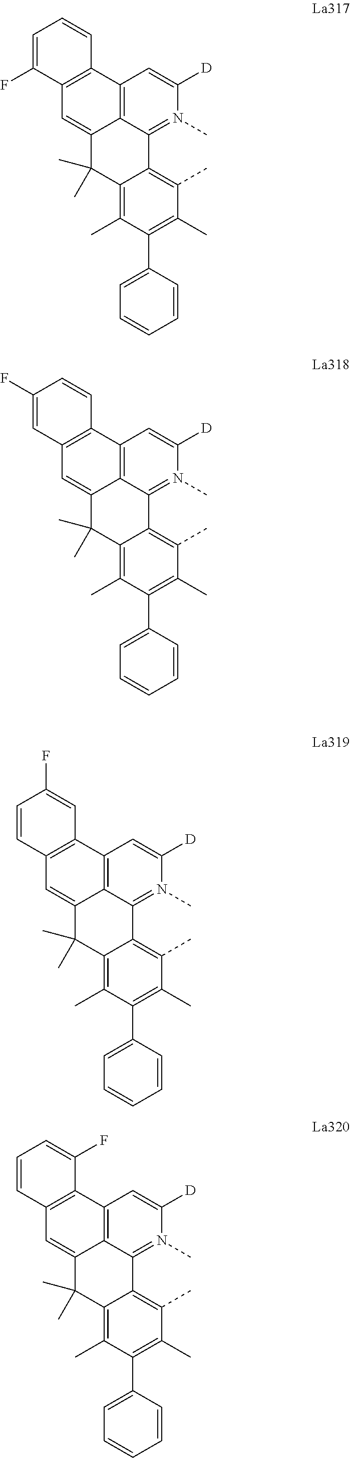

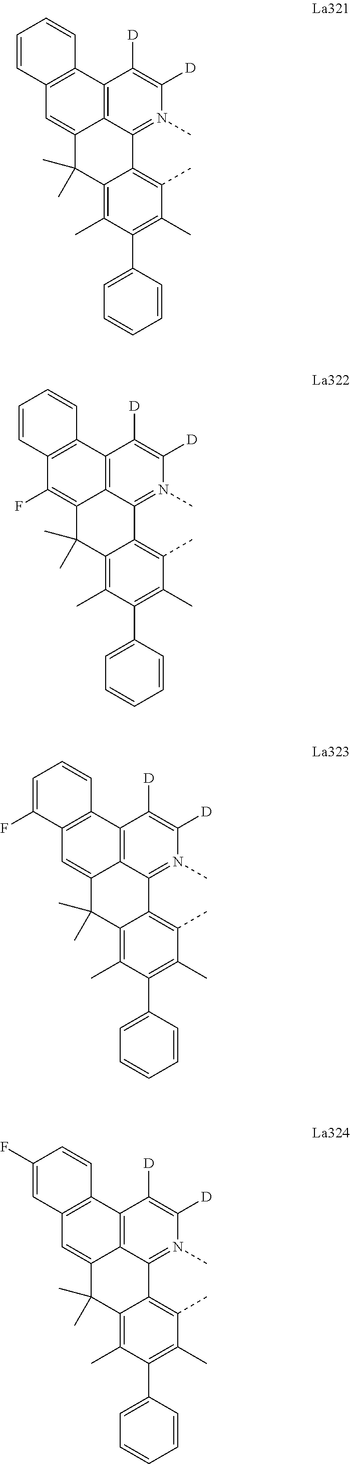

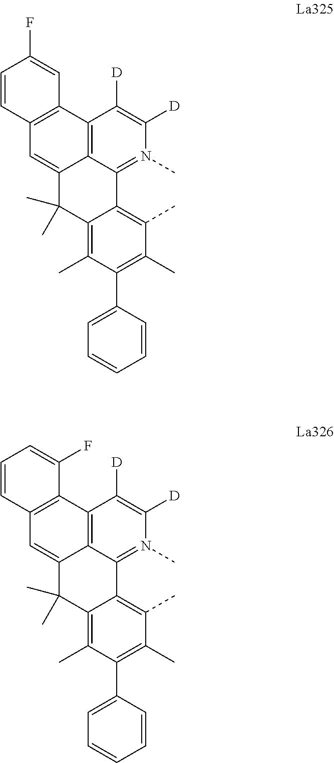

As a preferred metal iridium complex, Lb is one of the following structural formulas, or being correspondingly partially or completely deuterated or fluorinated,

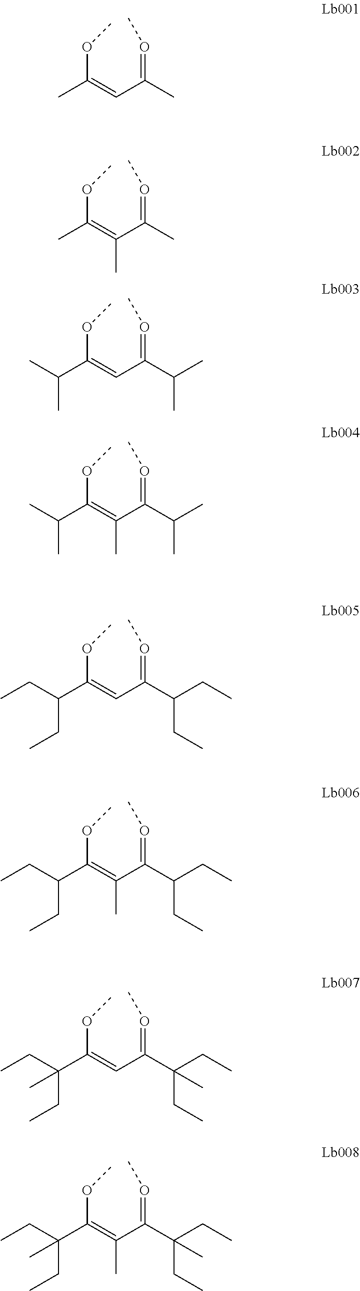

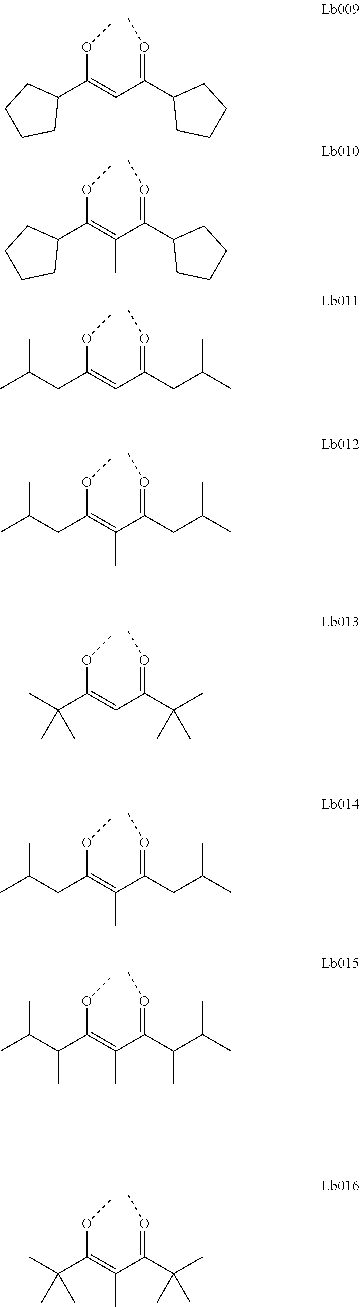

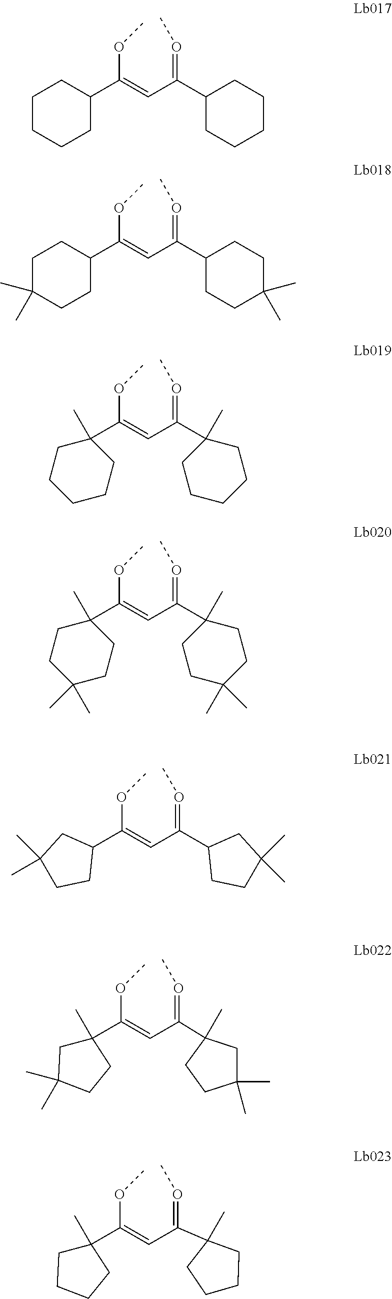

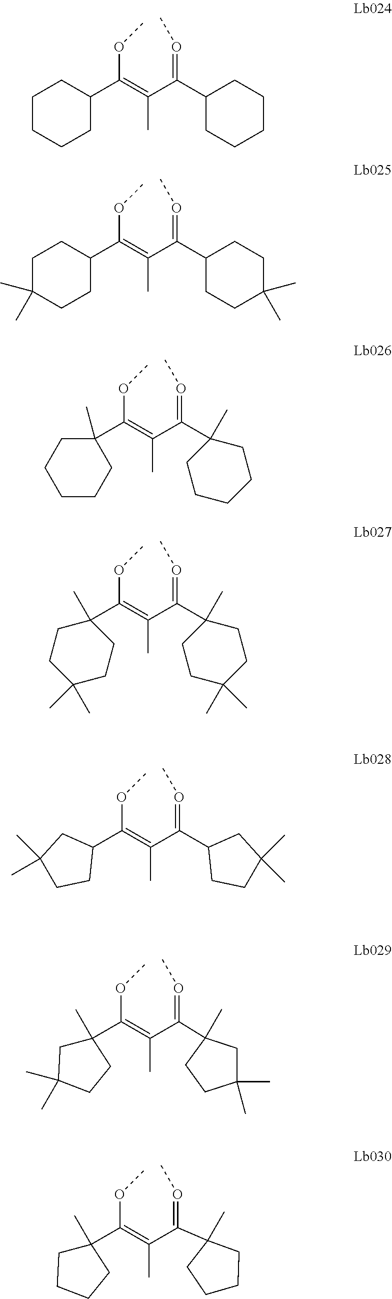

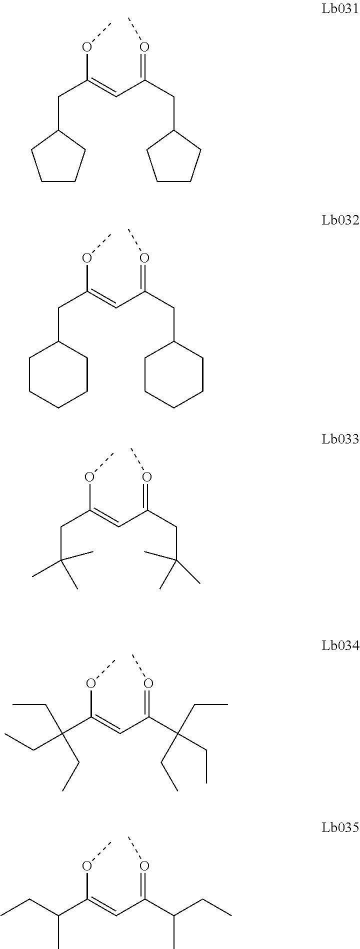

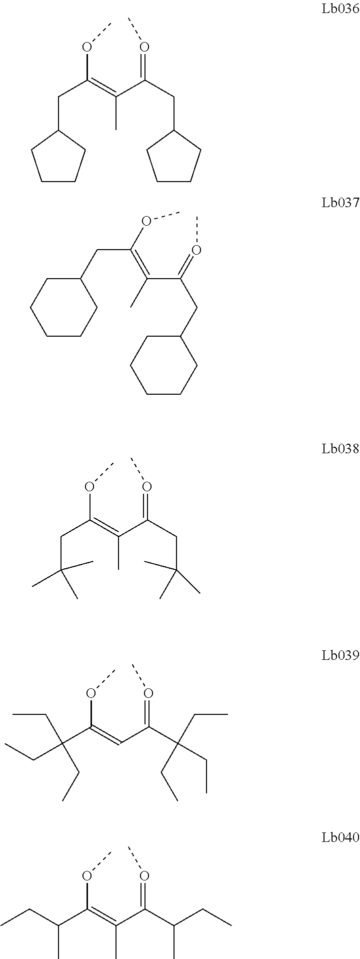

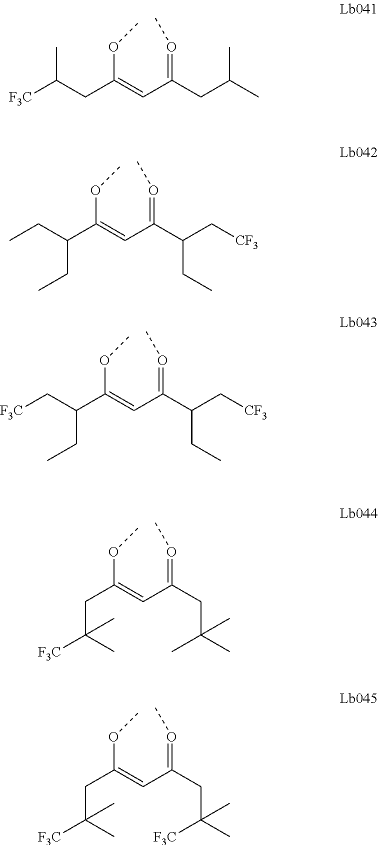

In particular, when part or all of the positions in La and/or Lb and/or Lc are substituted by deuterium, the bond energy of the C-D bond is greater than that of the C—H bond, thereby resulting in improved stability and reduced vibration energy dissipation, which can provide improved device lifetime and slightly improved device efficiency. When part of the positions in La and/or Lb and/or Lc are substituted by F, strong electron-withdrawing properties and special photoelectric physical properties of F atoms can provide an improved device luminescence color and device efficiency and a lower material sublimation temperature.

Another objective of the present disclosure is to provide an electroluminescent device, including a cathode, an anode and an organic layer disposed between the cathode and the anode, where the organic layer includes the above-mentioned organic metallic iridium complex.

The organic layer includes a light-emitting layer, and the metal iridium complex serves as a red light-emitting doping material of the light-emitting layer, or, the organic layer includes a hole injection layer, and the metal iridium complex serves as a hole injection material in the hole injection layer.

A further objective of the present disclosure is to provide a ligand La having a structural formula as follows:

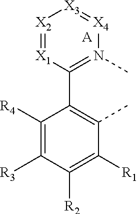

where R₁-R₄ and X₁-X₄ are as defined above.

The material of the present disclosure not only has the advantages of low sublimation temperature, high optical and electrochemical stability, high color saturation, high luminous efficiency, long device lifetime and the like, that can be used in organic light-emitting devices, but also has the potential to be applied to the AMOLED industry especially as red-emitting phosphorescent materials. As a phosphorescent material, the material of the present disclosure can convert triplet excited states into light, and thus can improve the luminous efficiency of the organic electroluminescent devices, thereby reducing energy consumption.

### BRIEF DESCRIPTION OF DRAWINGS

FIG. 1 is a ¹HNMR spectrum of a compound La003 of the present disclosure in a deuterated chloroform solution.

FIG. 2 is a ¹HNMR spectrum of a compound Ir(La003)₂Lb005 of the present disclosure in a deuterated chloroform solution.

FIG. 3 is a ¹HNMR spectrum of a compound La123 of the present disclosure in a deuterated chloroform solution.

FIG. 4 is a ¹HNMR spectrum of a compound Ir(La123)₂Lb005 of the present disclosure in a deuterated chloroform solution.

FIG. 5 is a ¹HNMR spectrum of a compound La143 of the present disclosure in a deuterated chloroform solution.

FIG. 6 is a ¹HNMR spectrum of a compound Ir(La143)₂Lb005 of the present disclosure in a deuterated chloroform solution.

FIG. 7 is a ultraviolet absorption spectrum and emission spectrum of a compound Ir(La003)₂Lb005 of the present disclosure in a dichloromethane solution.

FIG. 8 is a ultraviolet absorption spectrum and emission spectrum of a compound Ir(La123)₂Lb005 of the present disclosure in a dichloromethane solution.

FIG. 9 is a ultraviolet absorption spectrum and emission spectrum of a compound Ir(La143)₂Lb005 of the present disclosure in a dichloromethane solution.

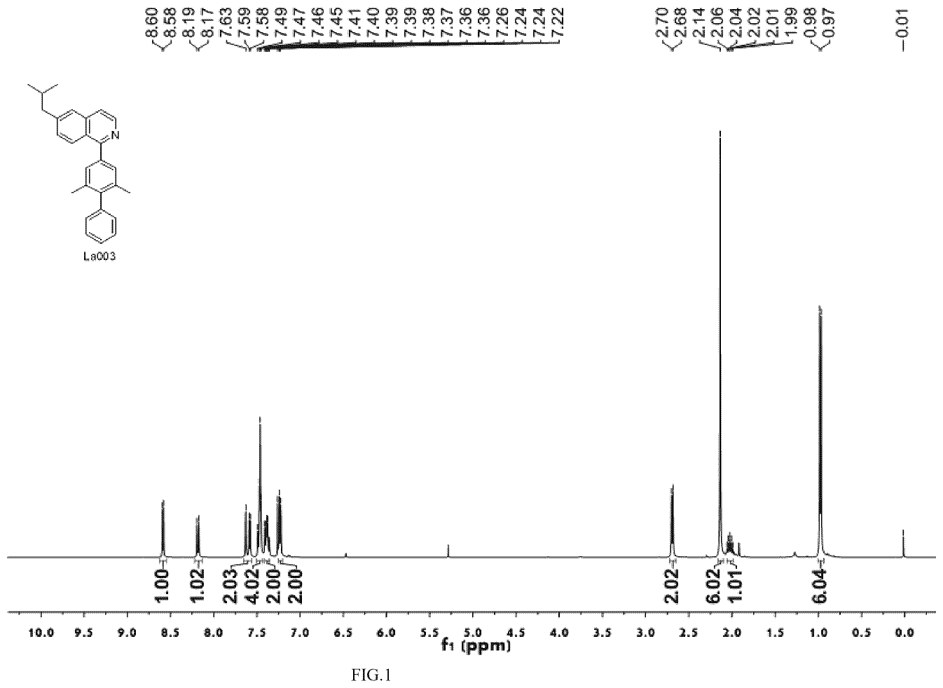

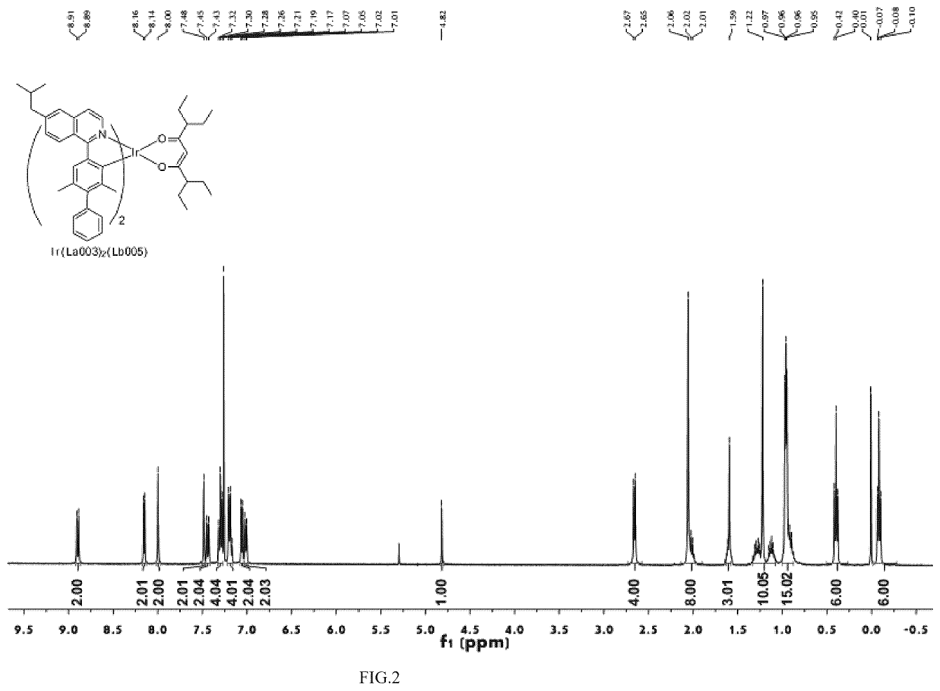

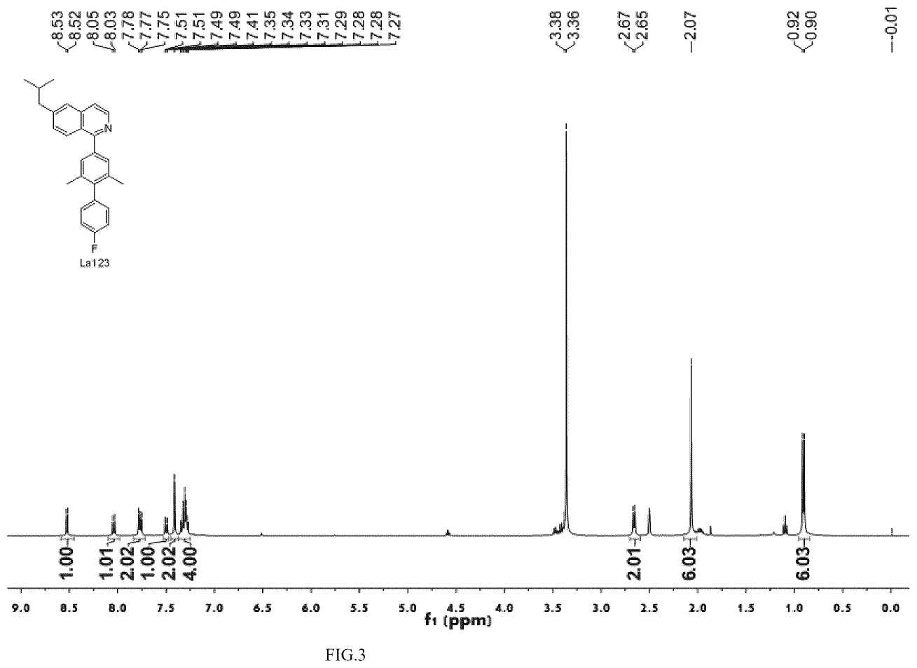

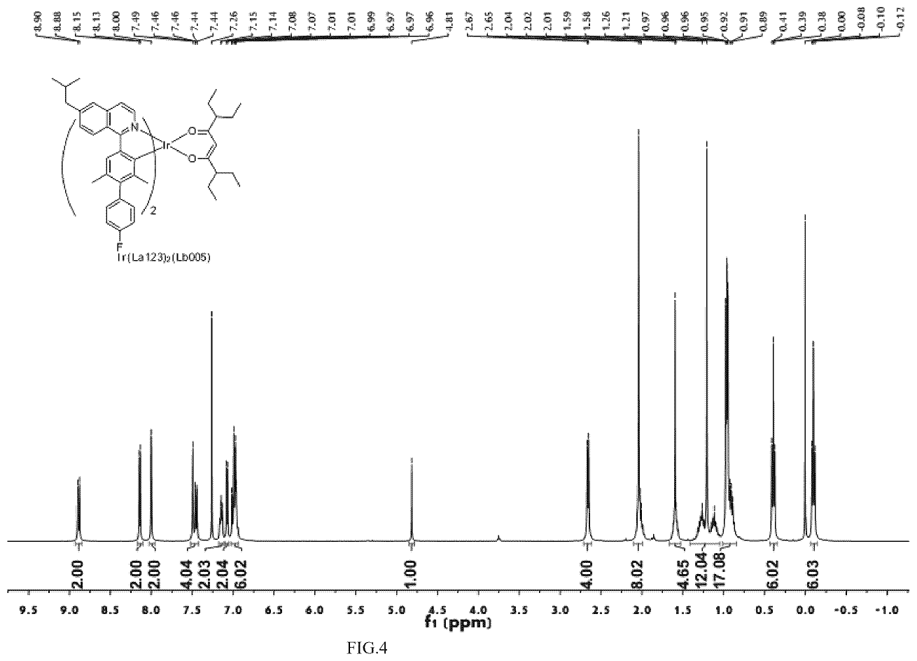

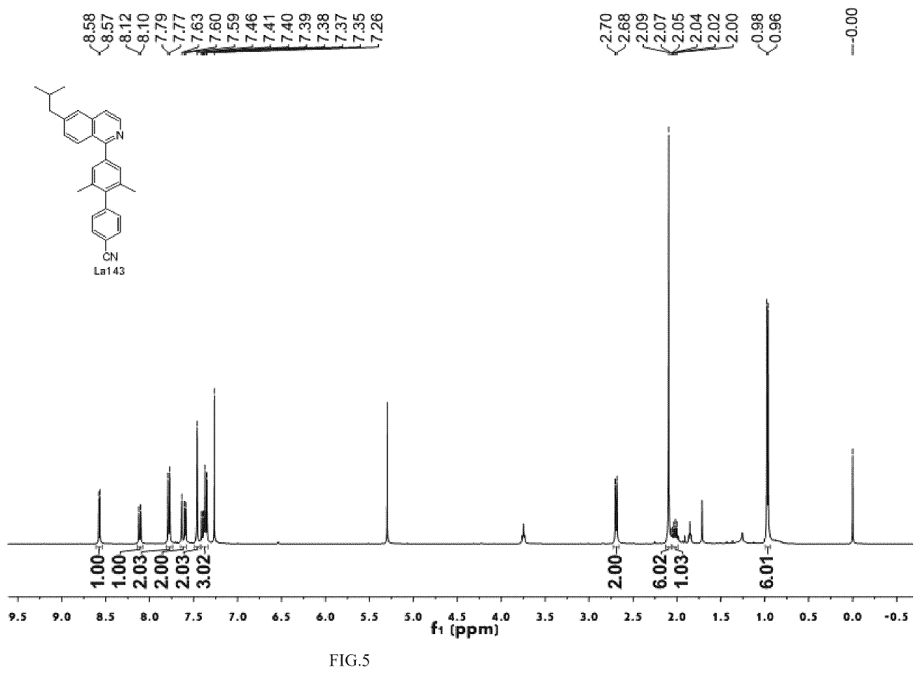

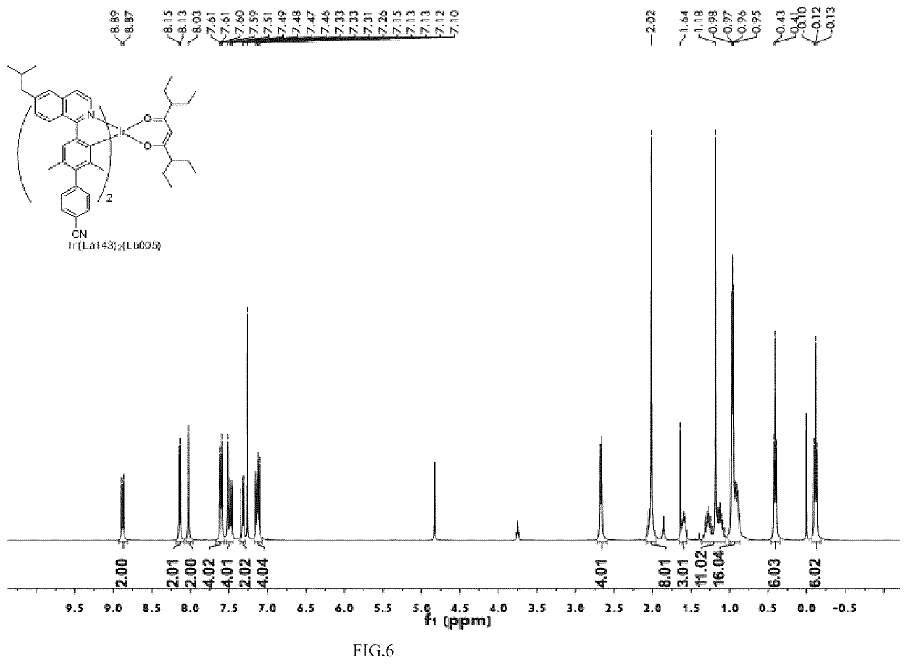

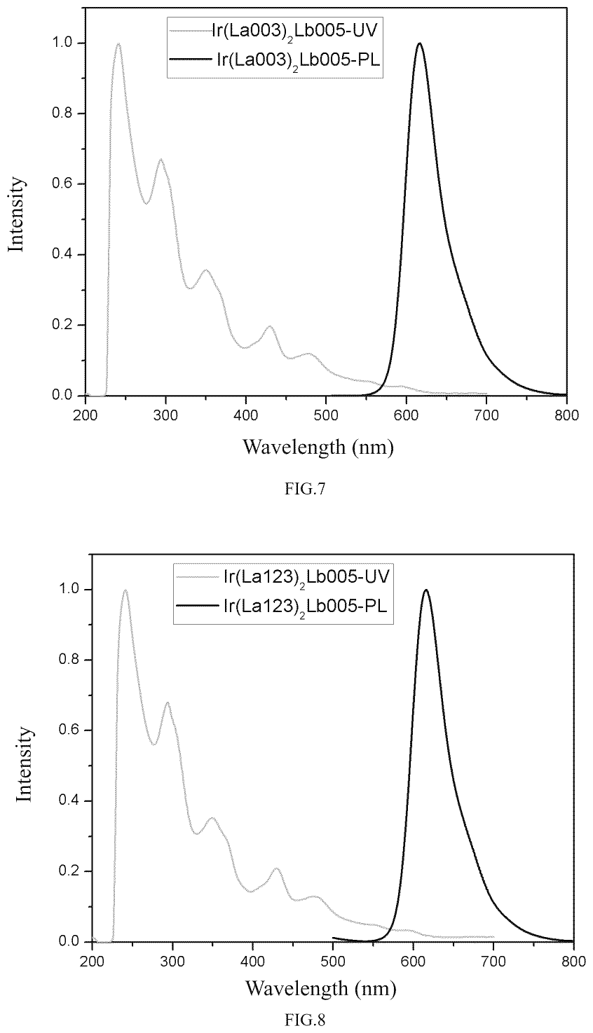

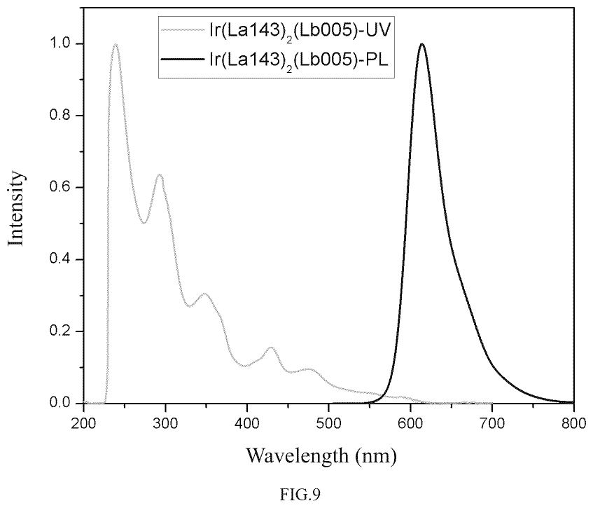

### DETAILED DESCRIPTION

A metal iridium complex of the present disclosure has a general formula Ir(La)(Lb)(Lc), where La is a structure of formula (1,

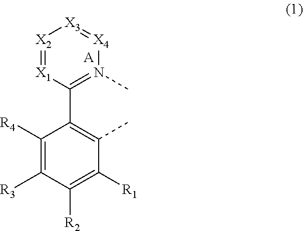

where dotted lines indicate a position connected to a metal Ir;
where X₁-X₄ are independently N or CR₀, and at least two of them are CR₀, and two R₀ are connected to each other to form a five- or six-membered substituted or unsubstituted aromatic ring;
where R₀ and R₄ are independently selected from hydrogen, deuterium, halogen, cyano, a substituted or unsubstituted C1-C10 alkyl group, a substituted or unsubstituted C1-C10 heteroalkyl group, a substituted or unsubstituted C3-C20 cycloalkyl group, a substituted or unsubstituted C3-C20 heterocycloalkyl group, a substituted or unsubstituted C2-C10 alkenyl group, a substituted or unsubstituted C2-C10 alkynyl group, a substituted or unsubstituted C6-C30 aryl group, a substituted or unsubstituted C2-C30 heteroaryl group, a substituted or unsubstituted (tri-C1-C10 alkyl)silyl group, a substituted or unsubstituted (tri-C6-C12 aryl)silyl group, a substituted or unsubstituted (di-C1-C10 alkyl)(mono-C6-C30 aryl)silyl group, a substituted or unsubstituted (mono-C1-C10 alkyl)(di-C6-C30 aryl)silyl group, amino group, thiol group, or hydroxyl group;
where R₁ and R₃ are independently selected from a substituted or unsubstituted C1-C10 alkyl group, a substituted or unsubstituted C1-C10 heteroalkyl group, a substituted or unsubstituted C3-C20 cycloalkyl group, or a substituted or unsubstituted C3-C20 heterocycloalkyl group;
where R₂ is a substituted or unsubstituted C6-C30 aryl group, or a substituted or unsubstituted C2-C30 heteroaryl group;
where the heteroalkyl group, the heterocycloalkyl group and the heteroaryl group contain at least one O, N or S heteroatom;
where the substitution is the substitution by deuterium, F, Cl, Br, a C1-C6 alkyl group, a C3-C6 cycloalkyl group, amine group substituted by a C1-C6 alkyl group, cyano, isocyano group or phosphino, where the substitution is from a monosubstitution to a maximum substitution;
where Lb is a structure of formula (2),

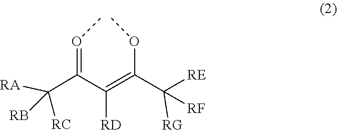

where dotted lines indicate a position connected to the metal Ir;
where RA-RG are independently selected from hydrogen, deuterium, halogen, a substituted or unsubstituted C1-C10 alkyl group, a substituted or unsubstituted C3-C20 cycloalkyl group, a substituted or unsubstituted C1-C10 heteroalkyl group, or a substituted or unsubstituted C3-C20 heterocycloalkyl group, or RA, RB, and RC are connected in pairs to form an aliphatic ring, and RE, RF, and RG are connected in pairs to form an aliphatic ring;
where the heteroalkyl group and the heterocycloalkyl group contain at least one O, N or S heteroatom;
where the substitution is the substitution by deuterium, F, Cl, Br, a C1-C4 alkyl group, a C1-C4 alkoxy group, a C3-C6 cycloalkyl group, amine group substituted by a C1-C4 alkyl group, cyano, nitrile group, isocyano group or phosphino;
where Lc is a monoanionic bidentate ligand, and Lc is different from Lb and not OO-type ligand;
where Lc and La are the same or different, and the difference is that a parent core structure is different, or the parent core structure is the same but substituents are different, or the parent core structure is the same, the substituents are the same, but positions of the substituents are different;
where two or three of La, Lb, and Lc are connected to each other to form a multidentate ligand;
where the cyclic aromatic ring structure can be an aromatic ring structure in which X₁ and X₂ or X₂ and X₃ or X₃ and X₄ each are CR₀ and two R₀ are connected to each other to form a five-membered ring or a six-membered ring, where the five-membered or six-membered aromatic ring structure is fused with a A ring to form a fused ring structure, examples of which include but are not limited to the following structures:

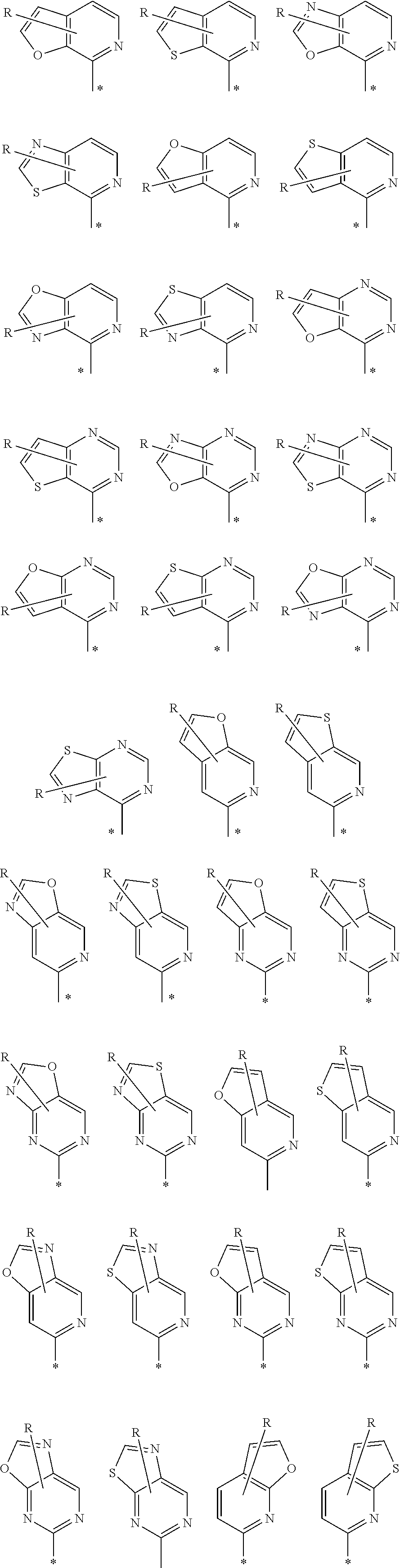

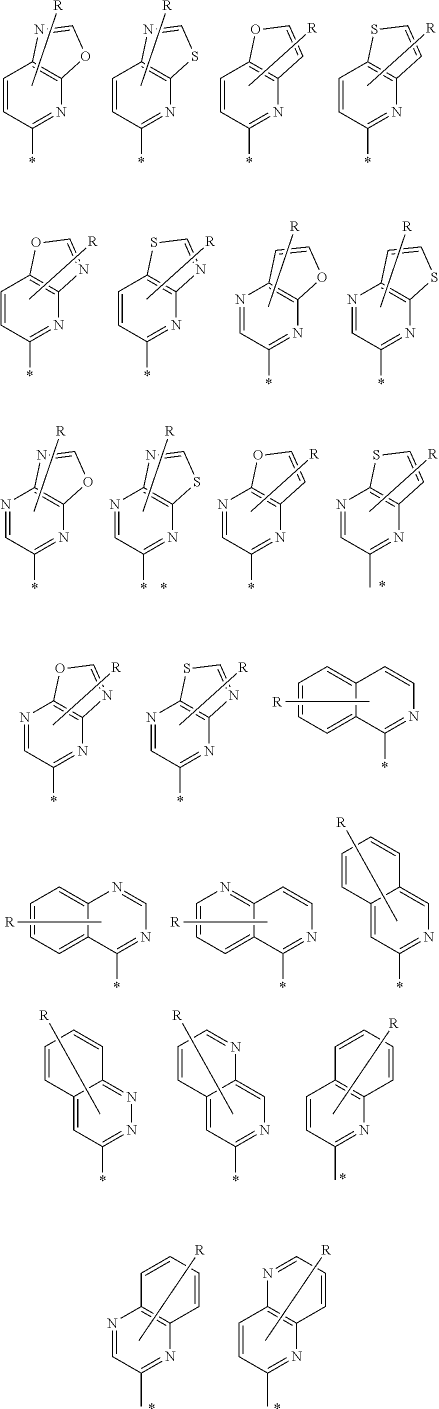

where * represents a position connected to the substituted benzene ring in formula (1);
where R represents no substitution to a maximum possible substitution, and two adjacent R can be connected to each other to form an aliphatic or aromatic ring structure; where no substitution means that the positions that can be connected to carbon are all hydrogen atoms; R is independently selected from deuterium, halogen, cyano, a substituted or unsubstituted C1-C10 alkyl group, a substituted or unsubstituted C1-C10 heteroalkyl group, a substituted or unsubstituted C3-C20 cycloalkyl group, a substituted or unsubstituted C3-C20 heterocycloalkyl group, a substituted or unsubstituted C2-C10 alkenyl group, a substituted or unsubstituted C2-C10 alkynyl group, a substituted or unsubstituted C6-C30 aryl group, a substituted or unsubstituted C2-C30 heteroaryl group, a substituted or unsubstituted (tri-C1-C10 alkyl)silyl group, a substituted or unsubstituted (tri-C6-C12 aryl)silyl group, a substituted or unsubstituted (di-C1-C10 alkyl)(mono-C6-C30 aryl)silyl group, a substituted or unsubstituted (mono-C1-C10 alkyl)(di-C6-C30 aryl)silyl group, amino group, thiol group, or hydroxyl group;
As a preferred metal iridium complex, a structure of formula (3) formed by interconnection between two adjacent R₀ in formula (1) forms a fused ring structure with the A ring:

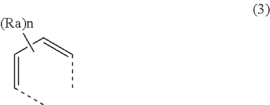

where dotted lines indicate a site connected to the A ring;
where Ra is independently selected from hydrogen, deuterium, halogen, cyano, a substituted or unsubstituted C1-C10 alkyl group, a substituted or unsubstituted C1-C10 heteroalkyl group, a substituted or unsubstituted C3-C20 cycloalkyl group, a substituted or unsubstituted C3-C20 heterocycloalkyl group, a substituted or unsubstituted C2-C10 alkenyl group, a substituted or unsubstituted C2-C10 alkynyl group, a substituted or unsubstituted C6-C30 aryl group, a substituted or unsubstituted C2-C30 heteroaryl group, a substituted or unsubstituted (tri-C1-C10 alkyl)silyl group, a substituted or unsubstituted (tri-C6-C12 aryl)silyl group, a substituted or unsubstituted (di-C1-C10 alkyl)(mono-C6-C30 aryl)silyl group, and a substituted or unsubstituted (mono-C1-C10 alkyl)(di-C6-C30 aryl)silyl group, or two adjacent Ra can be connected to each other to form an alicyclic ring or aromatic ring;
where n is a natural number from 0 to 4; and
the remaining symbol definitions are the same as mentioned above.

As a preferred metal iridium complex, the formula (1) has a structure of formula (4):

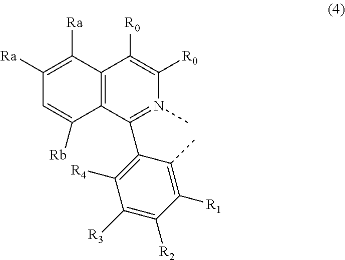

where Rb is H, deuterium, halogen, cyano, a substituted or unsubstituted C1-C10 alkyl, a substituted or unsubstituted C1-C10 heteroalkyl group, a substituted or unsubstituted C3-C20 cycloalkyl group, or a substituted or unsubstituted C3-C20 heterocycloalkyl group;
where at least one of Ra is not H; and
the symbol definition thereof is the same as mentioned above.

Examples of groups of the compounds represented by formula (1) to formula (4) will be defined below.

It should be noted that in the description, the term of “a to b carbon number” in the expression “a substituted or unsubstituted carbon number a to b X group” represents the carbon number of the unsubstituted X group, and does not include the carbon number in the substituent of the substituted X group.

The C1-C10 alkyl group is a linear or branched alkyl group, specifically, it is methyl, ethyl, propyl, isopropyl, n-butyl, isobutyl, sec-butyl, tert-butyl, n-pentyl and its isomers, n-hexyl and its isomers, n-heptyl and its isomers, n-octyl and its isomers, n-nonyl and its isomers, n-decyl and its isomers and the like, preferably methyl, ethyl, propyl, isopropyl, n-butyl, isobutyl, sec-butyl, and tert-butyl, and more preferably propyl, isopropyl, isobutyl, sec-butyl, and tert-butyl.

Examples of the C3-C20 cycloalkyl group include cyclopropyl, cyclobutyl, cyclopentyl, cyclohexyl, 1-adamantyl, 2-adamantyl, 1-norbornyl, 2-norbornyl and the like, preferably cyclopentyl and cyclohexyl.

Examples of the C2-C10 alkenyl group include vinyl, propenyl, allyl, 1-butadienyl, 2-butadienyl, 1-hexatrienyl, 2-hexatrienyl, 3-hexatrienyl and the like, preferably propenyl and allyl.

The C1-C10 heteroalkyl group is linear or branched alkyl, cycloalkyl, etc. containing atoms other than carbon and hydrogen atoms, and examples thereof include thiomethylmethyl, methoxymethyl, ethyloxymethyl, tert-butoxymethyl, N,N-dimethylmethyl, epoxybutyl, epoxypentanyl, epoxyhexyl and the like, preferably methoxymethyl, cyclohexyl.

Specific examples of the aryl group include phenyl, naphthyl, anthracenyl, phenanthrenyl, tetraphenyl, pyrenyl, chrysenyl, benzo[c]phenanthryl, benzo[g]chrysenyl, fluorenyl, benzofluorenyl, dibenzofluorenyl, biphenyl, terphenyl, tetraphenyl, fluoranthene and the like, preferably phenyl and naphthyl.

Specific examples of the heteroaromatic group include pyrrolyl, pyrazinyl, pyridinyl, pyrimidyl, triazinyl, indolyl, isoindolyl, imidazolyl, furanyl, benzofuranly, isobenzofuranyl, dibenzofuranyl, dibenzothiophenyl, azodibenzofuranyl, azodibenzothiophenyl, diazodibenzofuranyl, diazodibenzothiophenyl, quinolyl, isoquinolyl, quinoxalinyl, carbazolyl, phenanthridinyl, acridinyl, phenanthrolinyl, phenazinyl, phenothiazinyl, phenoxazinyl, oxazolinyl, oxadiazolyl, furazanyl, thiophenyl, benzothiophenyl, dihydroacridinyl, azacarbazolyl, diazacarbazolyl, quinazolyl and the like, preferably, pyridinyl, pyrimidyl, triazinyl, dibenzofuranyl, dibenzothiophenyl, azodibenzofuranyl, azodibenzothiophenyl, diazodibenzofuranyl, diazodibenzothiophenyl, carbazolyl, azacarbazolyl, and diazacarbazolyl.

In the description, specific examples of the substituent with a Hammett constant greater than −0.15 include methyl, ethyl, isopropyl, isobutyl, tert-butyl, nitro, cyano, sulfonic acid group, F, Cl, Br, I, trifluoromethyl, trifluoromethylsulfonyl, trifluoromethylsulfinyl, alkynyl, sulfone, sulfoxide, phosphonyl, aldehyde, ketone, ester group, carbonyl, pyrazinyl, pyridinyl, pyrimidyl, triazinyl, quinolyl, isoquinolyl, quinoxalinyl, and the aforementioned alkyl, cycloalkyl, aromatic, and the like.

As a preferred substituent, its Hammett constant is ≥−0.15, particularly preferably ≥0.1, and especially preferably 20.3. Preferred examples are CN and F.

The following embodiments are merely to facilitate understanding of the technical invention and should not be regarded as specific limitations of the present disclosure.

Raw materials and solvents involved in the synthesis of the compounds in the present disclosure are purchased from Alfa, Acros and other suppliers well known to those skilled in the art.

### Synthesis of Compound 2:

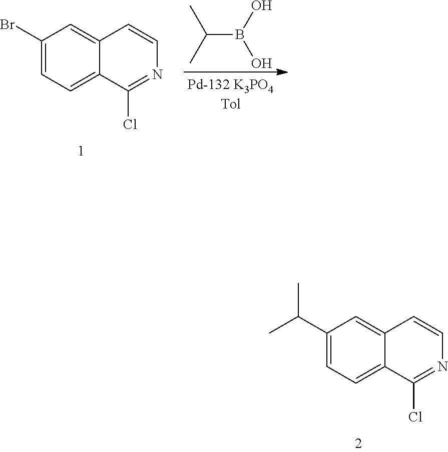

Compound 1 (25.0 g, 103.09 mmol, 1.0 eq), isopropylboronic acid (13.59 g, 154.64 mmol, 1.5 eq), dichloro-di-tert-butyl-(4-dimethylaminophenyl)phosphorus palladium (II) (3.65 g, 5.15 mmol, 0.05 eq), anhydrous potassium phosphate (54.71 g, 257.73 mmol, 2.5 eq), and toluene (375 mL) were added to a 1 L three-necked flask, vacuumed and replaced with nitrogen for 3 times, and stirred at 100° C. for 6 h under nitrogen protection. TLC monitoring showed that compound 1 was reacted completely. The reaction solution was cooled to room temperature, concentrated under reduced pressure to remove organic solvents, extracted in ethyl acetate (180 mL) and deionized water (180 mL), spin-dried and separated by column chromatography (an eluent is that ethyl acetate:n-hexane=1:50). After concentration, a white sugary solid was obtained as compound 2 (12.57 g, yield: 59.3%), mass spectrometry: 206.68 (M+H).

### Synthesis of Ligand La002:

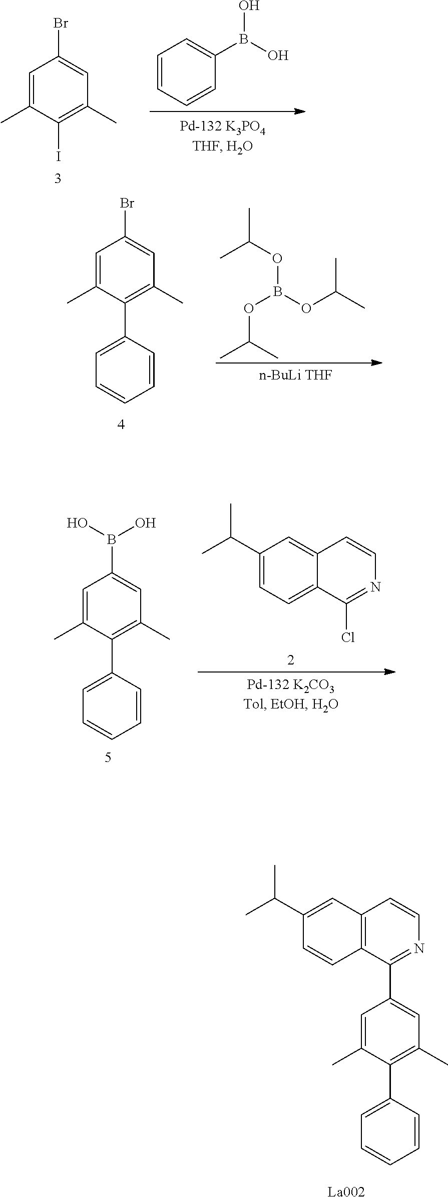

### Synthesis of Compound 4:

Compound 3 (120.0 g, 385.9 mmol, 1.0 eq), phenylboronic acid (50.35 g, 412.92 mmol, 1.07 eq), dichloro-di-tert-butyl-(4-dimethylaminophenyl)phosphorus palladium (11) (2.73 g, 3.86 mmol, 0.01 eq), anhydrous potassium phosphate (163.83 g, 771.81 mmol, 2.0 eq) were added to a 3 L reaction flask, then THF (1.08 L) was added and stirred, and finally H₂O (360 mL) was added. After 3 times of vacuum and nitrogen replacement, the reaction was carried out at 60° C. in an oil bath for 4 h. TLC monitoring showed that compound 3 was reacted almost completely. The reaction solution was cooled to room temperature and separated to collect an organic phase for spin-drying, and the dried organic phase was dissolved in dichloromethane (350 mL), and washed twice with water (150 mL/time). The organic phase was collected and concentrated, and purified by column chromatography (n-hexane elution) to obtain compound 4 (63.90 g, yield: 63.40%), mass spectrometry: 262.16 (M+H).

### Synthesis of Compound 5:

The compound 4 (58.0 g, 222.09 mmol, 1.0 eq) and THF (580 mL) were first added into a 2 L three-necked flask and protected with nitrogen. The reaction was cooled to −78° C. and kept for 5 h after 2.0 M n-butyllithium (133.25 mL, 266.51 mmol, 1.2 eq) was slowly added dropwise, and triisopropyl borate (50.12 g, 266.51 mmol, 1.2 eq) was then slowly added dropwise, and the reaction was gradually returned to room temperature overnight. TLC monitoring showed that the compound 4 was reacted almost completely. 2.5 M dilute hydrochloric acid (300 mL) were further added to adjust the pH to 2-3, and the reaction solution was separated to collect an organic phase to be concentrated to dryness, and then n-hexane (220 mL) was added for beating to obtain a white solid compound 5 (27.93 g, yield: 55.63%), 227.08 (M+H).

### Synthesis of Ligand La002:

Compound 5 (9.10 g, 40.25 mmol, 1.2 eq), compound 2 (6.90 g, 33.54 mmol, 1.0 eq), dichloro-di-tert-butyl-(4-dimethylaminophenyl) phosphorus palladium (II) (2.37 mg, 3.54 mmol, 0.01 eq), potassium carbonate (9.27 g, 67.09 mmol, 2.00 eq), toluene (105 mL), ethanol (35 mL) and deionized water (35 mL) were added to a 500 mL three-necked flask, vacuumed and replaced with nitrogen for 3 times, and stirred for reaction at 70° C. for 1.5 h under nitrogen protection. TLC monitoring showed that compound 2 was reacted completely. The reaction solution was cooled to room temperature and separated to collect an organic layer, and the organic layer was washed twice with water (100 mL/time), concentrated, spin dried and separated by column chromatography (the eluent is ethyl acetate:n-hexane=1:35). After concentration, a white solid was obtained as ligand La002 (9.40 g, yield: 79.69%) after concentration, mass spectrometry: 351.48 (M+H).

### Synthesis of Compound Ir(La002)₂(Lb005):

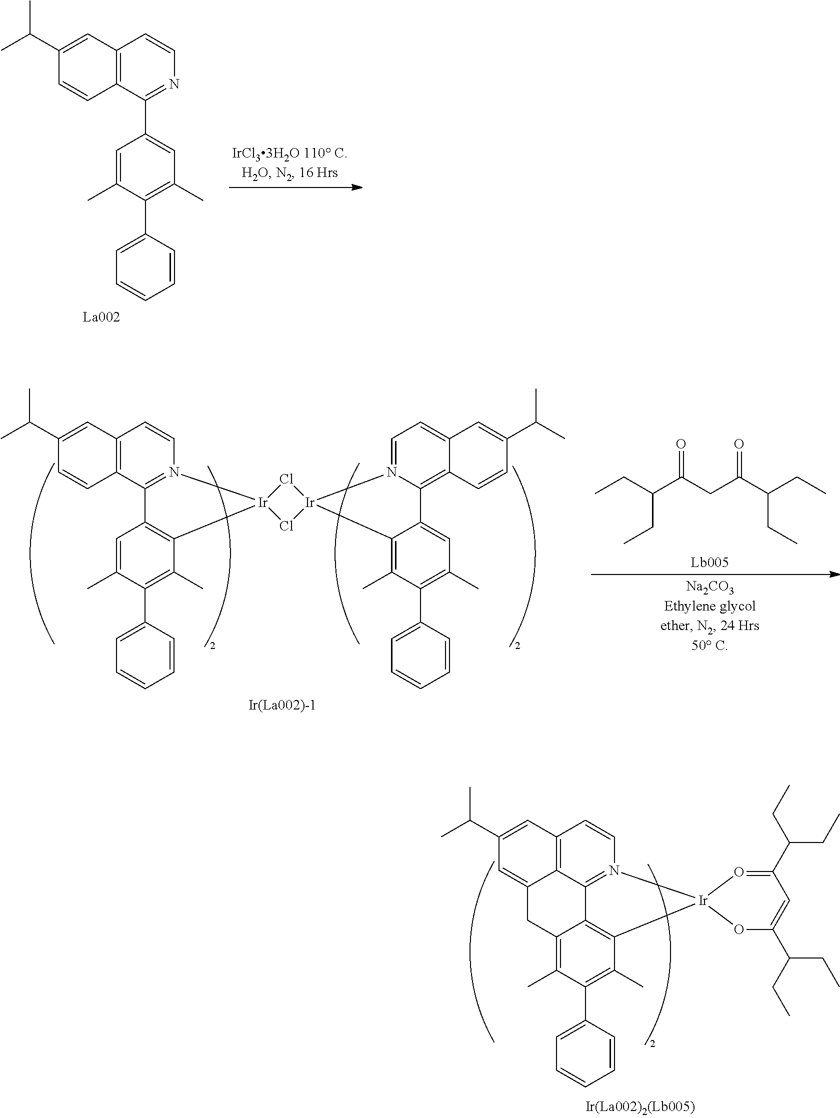

### Synthesis of Compound Ir(La002)-1:

Compound La002 (10.6 g, 29.0 mmol, 3.5 eq) and IrCl₃·3H₂O (2.92 g, 8.29 mmol, 1.0 eq) were placed in a 250 mL single-necked round-bottomed flask, into which ethylene glycol ether (100 mL) and deionized water (30 mL) were added, and the reaction solution was replaced by vacuum for 3 times, and was stirred at 110° C. for 18 h under N₂ protection. After cooling to room temperature, the mixture was added with methanol (100 mL) and stirred to precipitate a solid. The solid was collected by filtration and dried to obtain a dark red oily compound Ir(La002)-1 (6.55 g, 82.6%). The obtained compound was used directly in the next step without further purification.

### Synthesis of Compound Ir(La002)₂(Lb005):

Compound Ir(La002)-1 (6.55 g, 3.55 mmol, 1.0 eq), Lb005 (3.77 g, 17.77 mmol, 5.0 eq), and sodium carbonate (3.77 g, 35.54 mmol, 10.0 eq) was placed in a 250 mL single-necked round-bottomed flask, into which ethylene glycol ether (65 mL) was added. The mixture was replaced by vacuum for 3 times, and stirred at 50° C. for 20 h under N₂ protection. TLC monitoring showed that Ir(La002)-1 was reacted completely. After cooling to room temperature, the mixture was added with methanol (110 mL) for beating at room temperature for 2 h, and filtered by suction to obtain a filter cake. The filter cake was dissolved and clarified with toluene (100 mL), and filtered with silica gel. The filtrate was washed with deionized water (80 mL) for 3 times, and separated to collect an organic phase. The organic phase was concentrated and dried to obtain a dark red solid, which was recrystallized with toluene/n-hexane (product/toluene/n-hexane=1 g/10 mL/20 mL) 3 times and dried to obtain a red solid as compound Ir(La002)₂(Lb005) (3.13 g, yield: 40.32%). 3.13 g of crude Ir(La002)₂(Lb005) was sublimed and purified to obtain pure Ir(La002)₂(Lb005) (1.76 g, yield: 56.23%). Mass Spectrometry: 1105.45 (M+H). ¹HNMR (400 MHz, CDCl₃) δ8.91 (d, J=8.8 Hz, 2H), 8.17 (d, J=6.4 Hz, 2H), 8.04 (s, 2H), 7.47 (s, 2H), 7.43 (d, J=9.5 Hz, 2H), 7.29 (t, J=7.6 Hz, 4H), 7.19 (t, J=7.8 Hz, 4H), 7.08 (d, J=6.4 Hz, 2H), 7.03 (d, J=7.3 Hz, 2H), 4.82 (s, 1H), 2.68 (d, J=7.2 Hz, 4H), 2.14-1.89 (m, 8H), 1.61 (d, J=7.5 Hz, 3H), 1.28-1.06 (m, 6H), 1.00-0.86 (m, 15H), 0.41 (t, J=7.4 Hz, 6H), −0.06 (t, J=7.3 Hz, 6H).

### Synthesis of Ligand La003:

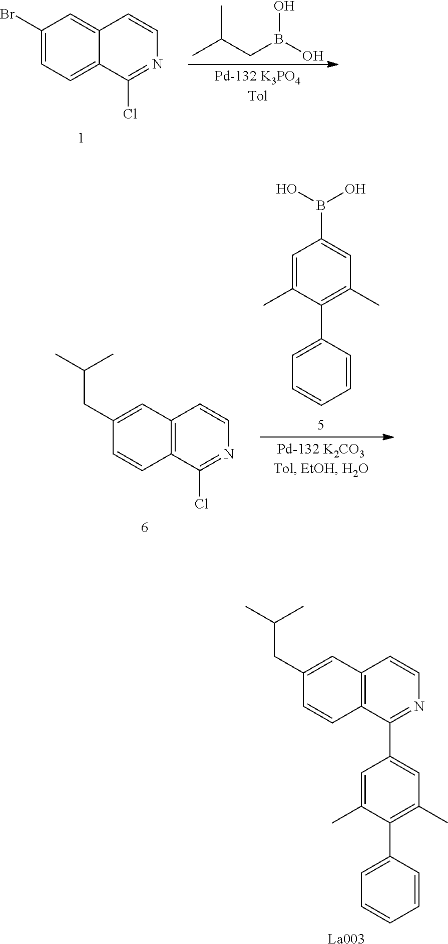

### Synthesis of Compound 6:

Referring to the synthesis and purification method of compound 2, only the corresponding raw materials needed to be changed to obtain a target compound 6, mass spectrometry: 220.71 (M+H).

### Synthesis of Ligand La003:

Referring to the synthesis and purification method of ligand La002, only the corresponding raw materials needed to be changed to obtain a target ligand La003, mass spectrometry: 366.51 (M+H). ¹HNMR (400 MHz, CDCl₃) δ 8.59 (t, J=3.9 Hz, 1H), 8.18 (dd, J=8.7, 2.8 Hz, 1H), 7.66-7.56 (m, 1H), 7.50-7.43 (in, 1H), 7.38 (ddd, J=10.8, 6.7, 1.4 Hz, 1H), 7.24 (dd, J=6.3, 4.9 Hz, 1H), 2.70 (t, J=5.2 Hz, 1H), 2.14 (d, J=2.7 Hz, 1H), 2.05-1.98 (m, 1H), 0.98 (t, J=4.5 Hz, 1H).

### Synthesis of Compound Ir(La003)₂(Lb005):

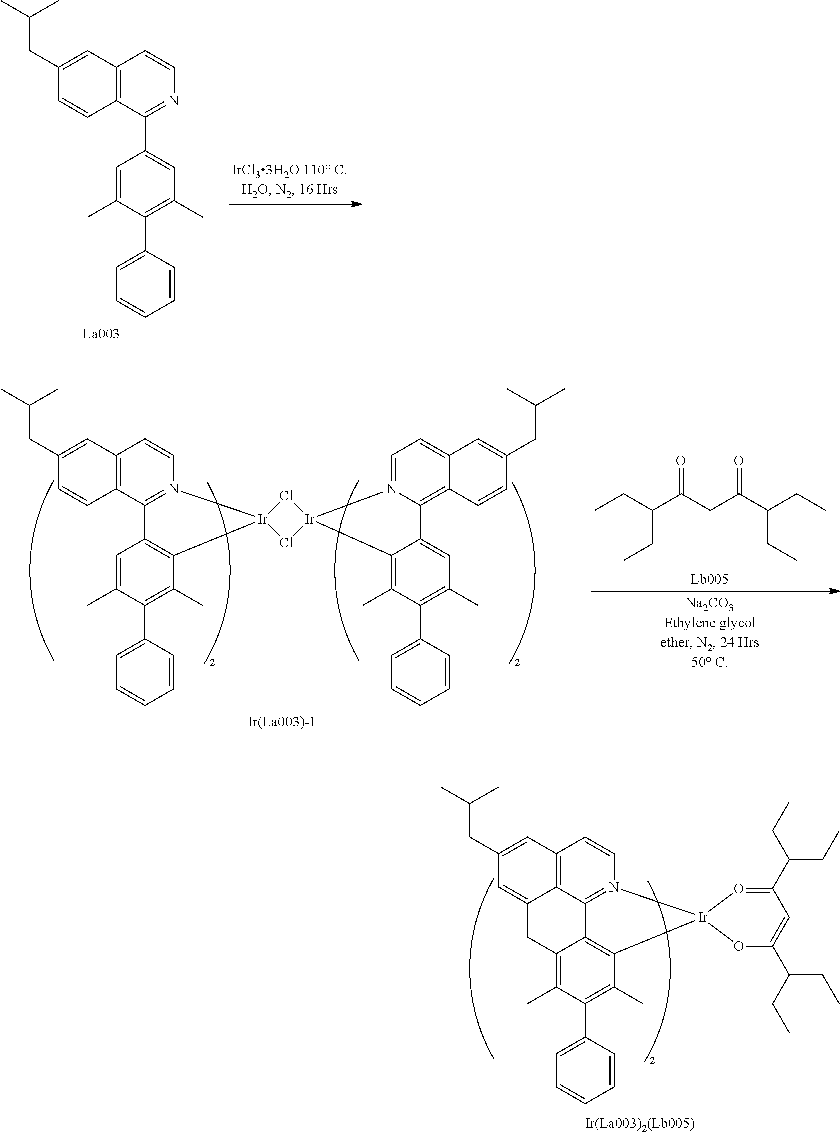

### Synthesis of Compound Ir(La003)-1:

Referring to the synthesis and purification method of compound Ir(La002)-1, only the corresponding raw materials needed to be changed to obtain compound Ir(La003)-1 that is used directly in the next step without purification.

### Synthesis of Compound Ir(La003)₂(Lb005):

Referring to the synthesis and purification method of compound Ir(La002)₂(Lb005), only the corresponding raw materials needed to be changed to obtain a red solid as compound Ir(La003)₂(Lb005) (3.23 g, yield: 43.57%). 3.23 g of crude Ir(La003)₂(Lb005) was sublimed and purified to obtain pure Ir(La003)₂(Lb005) (2.04 g, yield: 63.15%), mass spectrometry: 1133.54 (M+H). ¹HNMR (400 MHz, CDCl₃) δ8.90 (d, J=8.8 Hz, 2H), 8.15 (d, J=6.4 Hz, 2H), 8.00 (s, 2H), 7.48 (s, 2H), 7.44 (d, J=9.5 Hz, 2H), 7.30 (t, J=7.6 Hz, 4H), 7.19 (t, J=7.8 Hz, 4H), 7.06 (d, J=6.4 Hz, 2H), 7.01 (d, J=7.3 Hz, 21H), 4.82 (s, 1H), 2.66 (d, J=7.2 Hz, 4H), 2.14-1.89 (m, 8H), 1.60 (d, J=7.5 Hz, 3H), 1.33-1.08 (in, 10H), 1.00-0.88 (m, 15H), 0.40 (t, J=7.4 Hz, 6H), −0.08 (t, J=7.3 Hz, 6H).

### Synthesis of Compound Ir(La003)₂(Lb031):

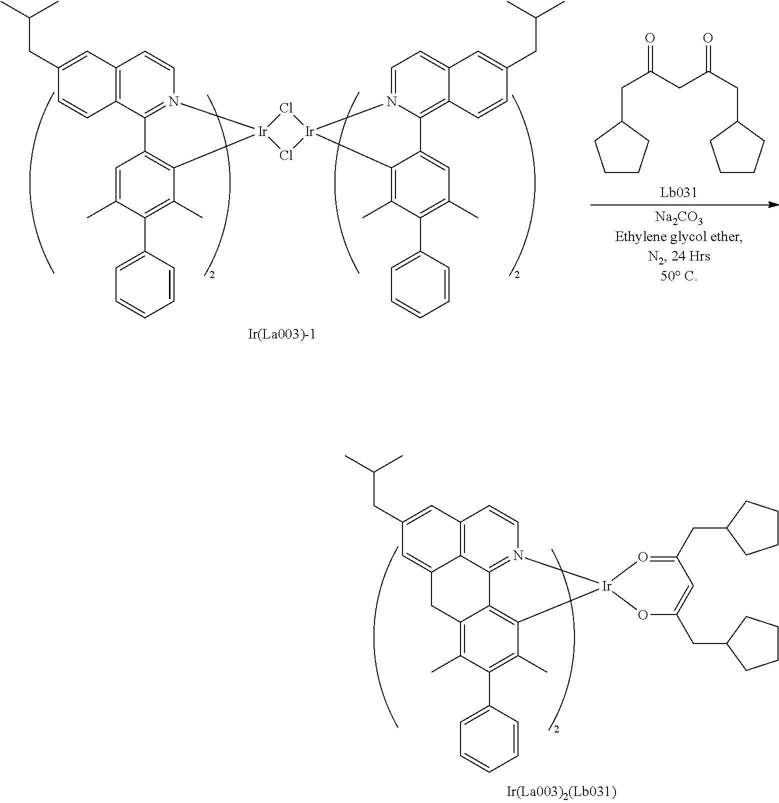

Referring to the synthesis and purification method of compound Ir(La002)₂(Lb005), only the corresponding raw materials needed to be changed to obtain a red solid as Ir(La003)₂(Lb031) (2.87 g, yield: 38.65%). 2.87 g of crude Ir(La003)₂(Lb007) was sublimed and purified to obtain pure Ir(La003)₂(Lb007) (1.77 g, yield: 61.67%), mass spectrometry: 1157.57 (M+H). ¹HNMR (400 MHz, CDCl₃) δ8.88 (d, J=8.7 Hz, 2H), 8.21 (d, J=6.3 Hz, 2H), 8.06 (s, 2H), 7.47 (s, 2H), 7.45 (d, J=9.1 Hz, 2H), 7.32 (t, J=7.4 Hz, 4H), 7.15 (t, J=7.6 Hz, 4H), 7.08 (d, J=6.2 Hz, 2H), 7.02 (d, J=7.4 Hz, 2H), 4.82 (s, 1H), 2.68 (d, J=7.2 Hz, 4H), 2.08-1.91 (m, 6H), 1.72 (d, J=7.6 Hz, 3H), 1.34-1.12 (m, 12H), 1.02-0.89 (nm, 15H), 0.33-0.12 (in, 12H).

### Synthesis of Ligand Lc04:

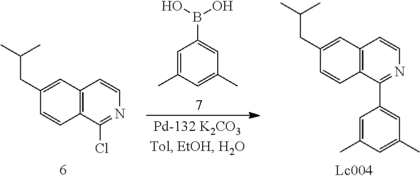

Referring to the synthesis and purification method of ligand La002, only the corresponding raw materials needed to be changed to obtain a target ligand Lc004, mass spectrometry: 290.41 (M+H).

### Synthesis of Compound Ir(La003)(Lb005)(Lc004):

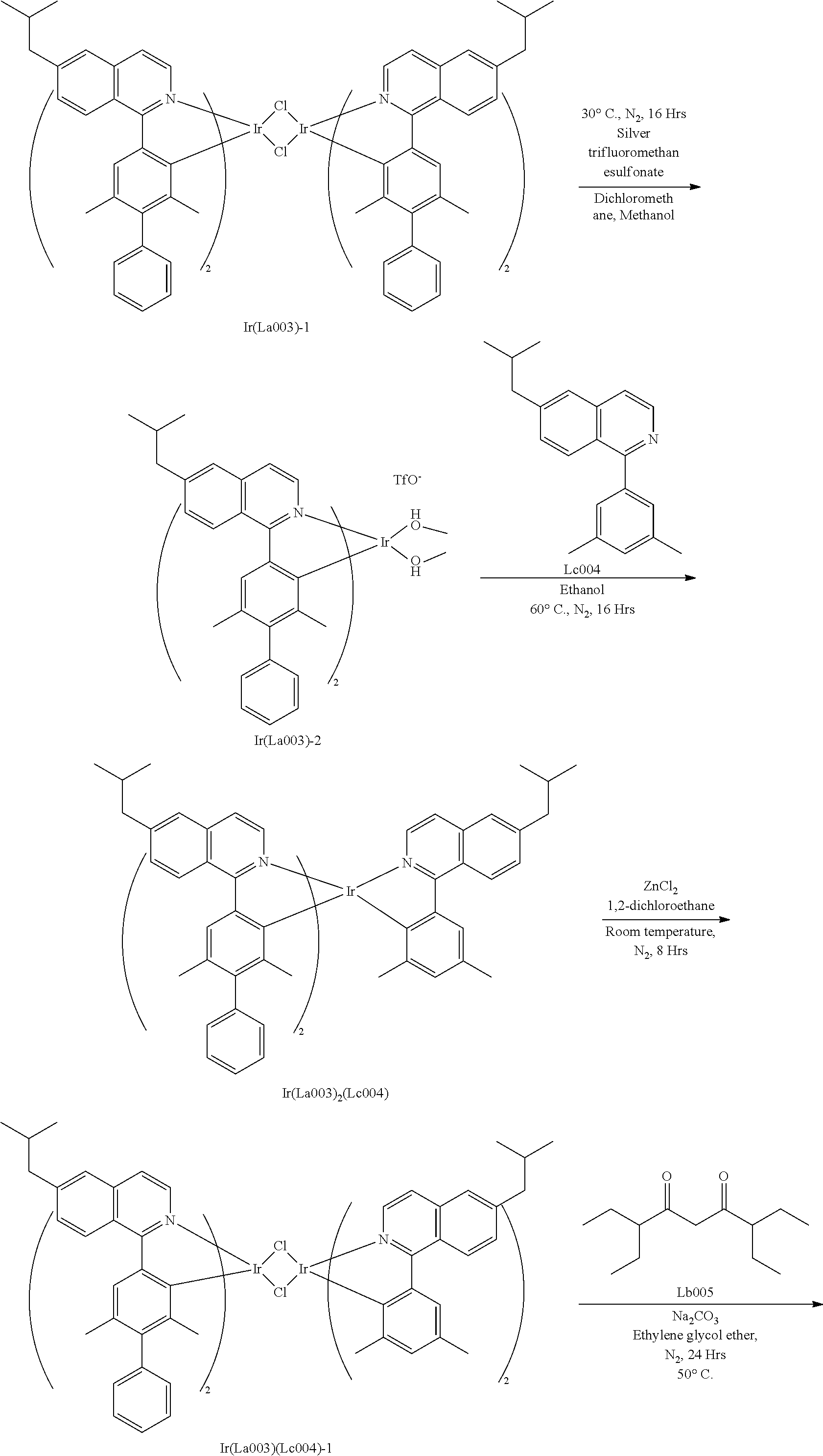

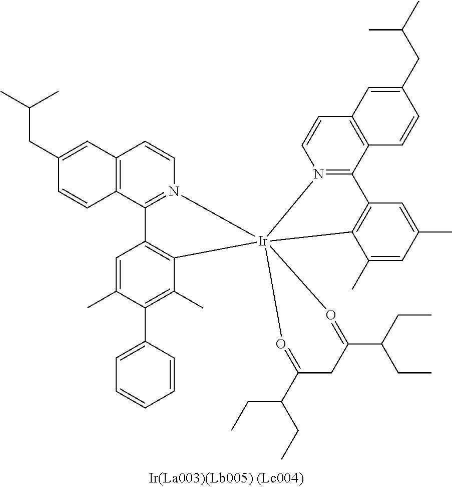

### Synthesis of Compound Ir(La003)-2:

A dimer Ir(La003)-1 (7.85 g, 8.21 mmol, 1.0 eq) and dichloromethane (630 mL) were added to a 3 L three-necked flask, and stirred for dissolving. Silver trifluoromethanesulfonate (4.22 g, 16.41 mmol, 2.0 eq) was dissolved in methanol (253 mL), and then added to a reaction solution in the original flask, and replaced by vacuum for 3 times. The obtained mixture was stirred at room temperature for 16 h under N₂ protection. The reaction solution was then filtered through diatomaceous earth, the filter residue was rinsed with dichloromethane (200 mL), and the filtrate was spin-dried to obtain compound Ir(La003)-2 (7.5 g, 80.62%). The obtained compound was used directly in the next step without purification.

### Synthesis of Compound Ir(La003)₂(Lc004):

Compound Ir(La003)-2 (7.5 g, 6.61 mmol, 1.0 eq) and Lc004 (4.78 g, 16.53 mmol, 2.5 eq) were added into a 250 mL three-necked flask, into which ethanol (113 mL) was added for vacuum replacement 3 times. Under N₂ protection, the obtained mixture was stirred and refluxed for 12 h. After cooling to room temperature, the reaction solution was filtered to collect a solid that was dissolved in dichloromethane (168 mL) and filtered on silica gel to obtain a filter cake. The filter cake was then rinsed with dichloromethane (60 mL) and spin-dried, recrystallized twice with tetrahydrofuran/methanol (product:tetrahydrofuran:methanol=1:5:8), and dried to obtain compound Ir(La003)₂(Lc004) (3.79 g, 47.33%). Mass Spectrometry: 1210.63 (M+H).

### Synthesis of Compound Ir(La003)(Lc004)-1:

Compound Ir(La003)₂(Lc004) (5.25 g, 4.34 mmol, 1.0 eq) and zinc chloride (29.58 g, 217.01 mmol, 50 eq) were placed into a 1 L single-necked flask, into which 1,2 dichloroethane (350 mL) was added for vacuum replacement 3 times. Under N₂ protection, the obtained mixture was stirred and refluxed for 18 h. The TLC spot plate monitoring showed that the raw material Ir(La003)₂(Lc004) was reacted almost completely. After cooling to room temperature, the mixture was washed with deionized water 3 times (120 mL/time) to obtain a filtrate that was spin-dried to obtain compound Ir(La003)(Lc004)-1 (3.22 g, 84.32%). The obtained compound was used directly in the next step without purification.

### Synthesis of Compound Ir(La003)(Lb005)(Lc004):

The compound Ir(La003)(Lc004)-1 (4.32 g, 2.45 mmol, 1.0 eq), Lb005 (2.6 g, 12.26 mmol, 5.0 eq) and sodium carbonate (2.6 g, 24.53 mmol, 10.0 eq) were placed in a 250 mL single-necked round-bottomed flask, into which ethylene glycol ether (65 mL) was added for vacuum replacement 3 times.

The obtained mixture was stirred at 50° C. for 24 h under N₂ protection. TLC monitoring showed that the Ir(La003)(Lc004)-1 was reacted completely. After cooling to room temperature, the mixture was added with 106 mL of methanol for beating at room temperature for 2 h, and filtered by suction to obtain a filter cake. The filter cake was dissolved with dichloromethane (90 mL) and filtered with silica gel, and then rinsed with dichloromethane (50 mL) to collect the filtrate that was washed with deionized water for 3 times (50 mL/time), separated to collect an organic phase. The organic phase was concentrated and dried to obtain a dark red solid, which was recrystallized with tetrahydrofuran/methanol (product:tetrahydrofuran:methanol=1:5:7) for 3 times to obtain a red solid as compound Ir(La003)(Lb005)(Lc004) (1.22 g, yield: 46.87%). 1.22 g of crude Ir(La003)(Lb005)(Lc004) were sublimated and purified to obtain a sublimated pure Ir(La003)(Lb005)(Lc004) (0.78 g, yield: 63.93%). Mass Spectrometry: 1058.45 (M+H). ¹H NMR (400 MHz, CDCl₃) δ8.91 (s, 1H), 8.87 (s, 1H), 8.26 (s, 1H), 8.11 (s, 1H), 7.51 (s, 1H), 7.37 (m, 4H), 7.16 (m, 4H), 7.11 (d, J=7.2 Hz, 2H), 7.04 (d, J=6.6 Hz, 2H), 6.97 (d, J=7.6 Hz, 2H), 4.83 (s, 1H), 2.62 (d, J=6.8 Hz, 2H), 2.58 (d, J=7.4 Hz, 2H), 2.11-1.96 (m, 6H), 1.76 (m, 6H), 1.37-1.18 (m, 12H), 0.96-0.81 (m, 14H), 0.21-0.08 (m, 10H).

### Synthesis of Ligand Lc025:

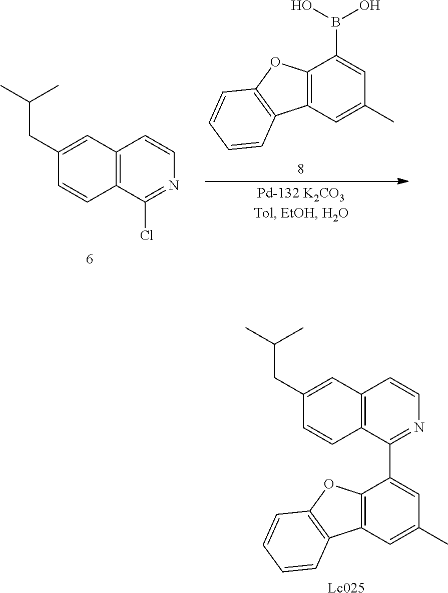

Referring to the synthesis and purification method of ligand La002, only the corresponding raw materials needed to be changed to obtain a target ligand Lc025, mass spectrometry: 366.47 (M+H).

### Synthesis of Compound Ir(La003)(Lb005)(Lc025):

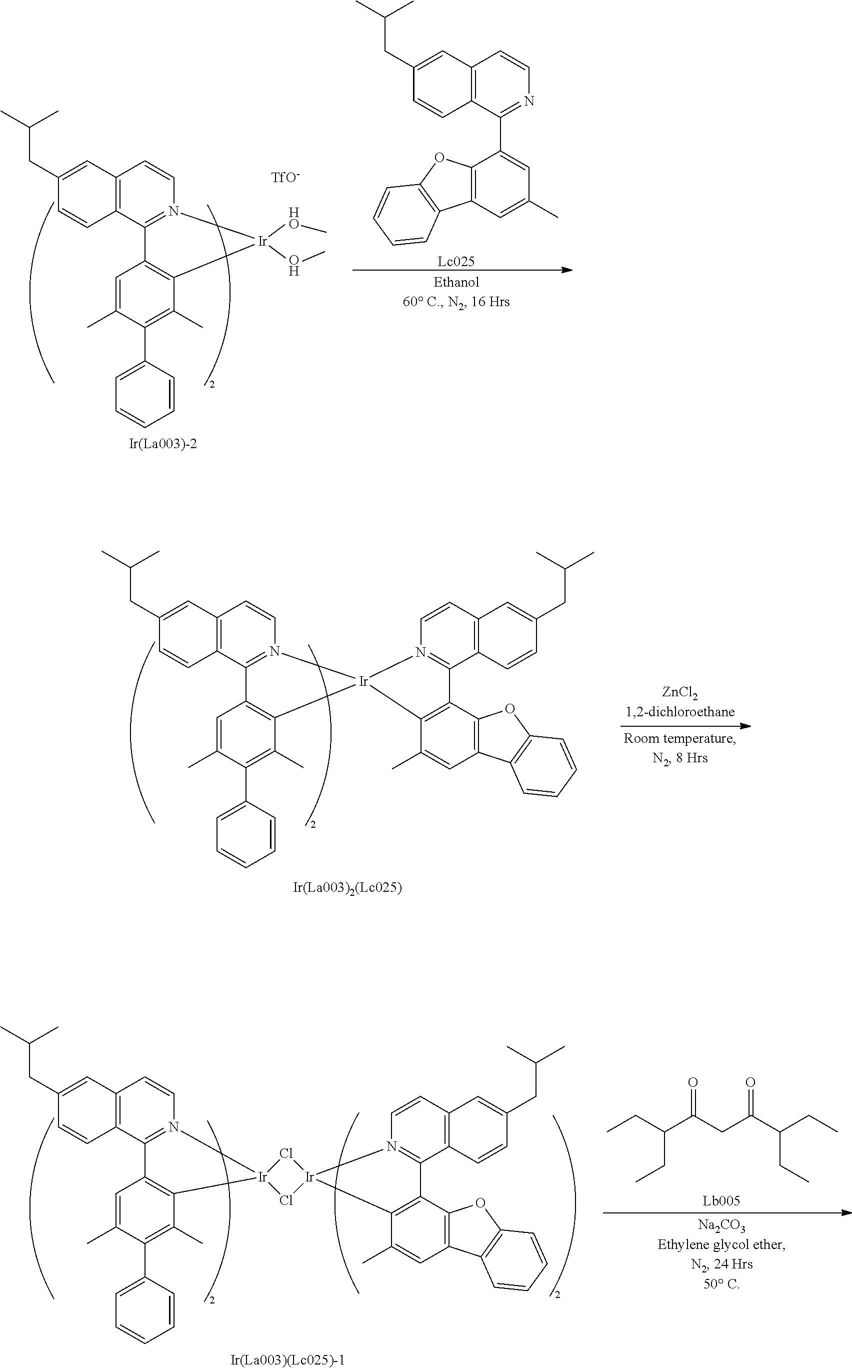

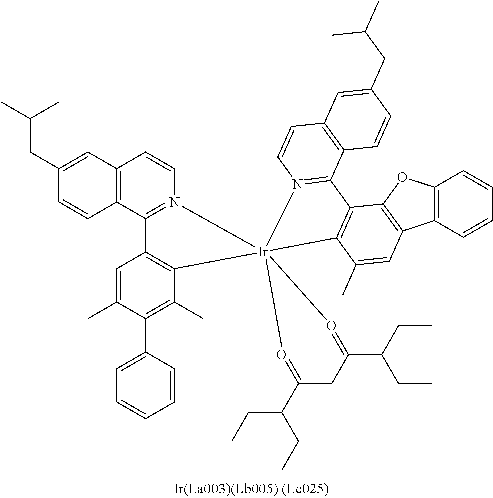

### Synthesis of Compound Ir(La003)₂(Lc025):

Referring to the synthesis and purification method of compound Ir(La003)₂(Lc004), only the corresponding raw materials needed to be changed to obtain a target compound Ir(La003)₂(Lc025), mass spectrometry-1286.68 (M+H).

### Synthesis of Compound Ir(La003)(Lc025)-1:

Referring to the synthesis and purification method of the ligand Ir(La003)(Lc004)-1, only the corresponding raw materials needed to be changed to obtain a target compound Ir(La003)(Lc025)-1 that was used directly in the next step without purification.

### Synthesis of Compound Ir(La003)(Lb005)(Lc025):

Referring to the synthesis and purification method of compound Ir(La003)(Lb005)(Lc004), only the corresponding raw materials needed to be changed to obtain a red solid as compound Ir(La003)(Lb005)(Lc025) (1.54 g, yield: 41.21%). 1.54 g of crude Ir(La003)(Lb005)(Lc25) were sublimated and purified to obtain a sublimated pure Ir(La003)(Lb005)(Lc025) (0.87 g, yield: 56.49%), mass spectrometry: 1134.51 (M+H). ¹H NMR (400 MHz, CDCl₃) δ8.90 (s, 1H), 8.86 (s, 1H), 8.19 (s, 1H), 8.16 (s, 1H), 7.44-7.38 (m, 6H), 7.15-7.09 (m, 5H), 7.04-6.97 (d, J=7.6 Hz, 6H), 4.81 (s, 1H), 2.63 (d, J=7.0 Hz, 2H), 2.59 (d, J=7.2 Hz, 2H), 2.21-2.04 (m, 6H), 1.76 (m, 6H), 1.37-1.18 (m, 12H), 0.96-0.81 (m, 12H), 0.21-0.14 (in, 9H).

### Synthesis of Ligan Lc026:

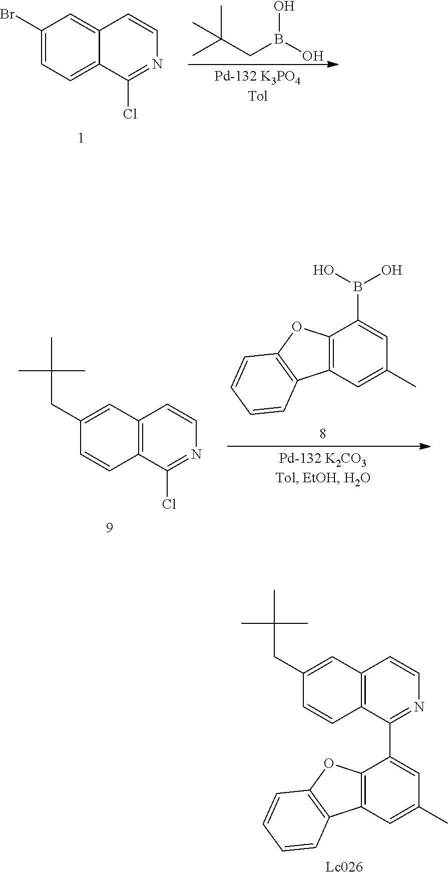

### Synthesis of Compound 9:

Referring to the synthesis and purification method of compound 2, only the corresponding raw materials needed to be changed to obtain a target compound 9, mass spectrometry: 234.74 (M+H).

### Synthesis of Ligand Lc026:

Referring to the synthesis and purification method of ligand La002, only the corresponding raw materials needed to be changed to obtain a target ligand Lc026, mass spectrometry: 380.49 (M+H).

### Synthesis of Compound Ir(La003)(Lb005)(Lc026):

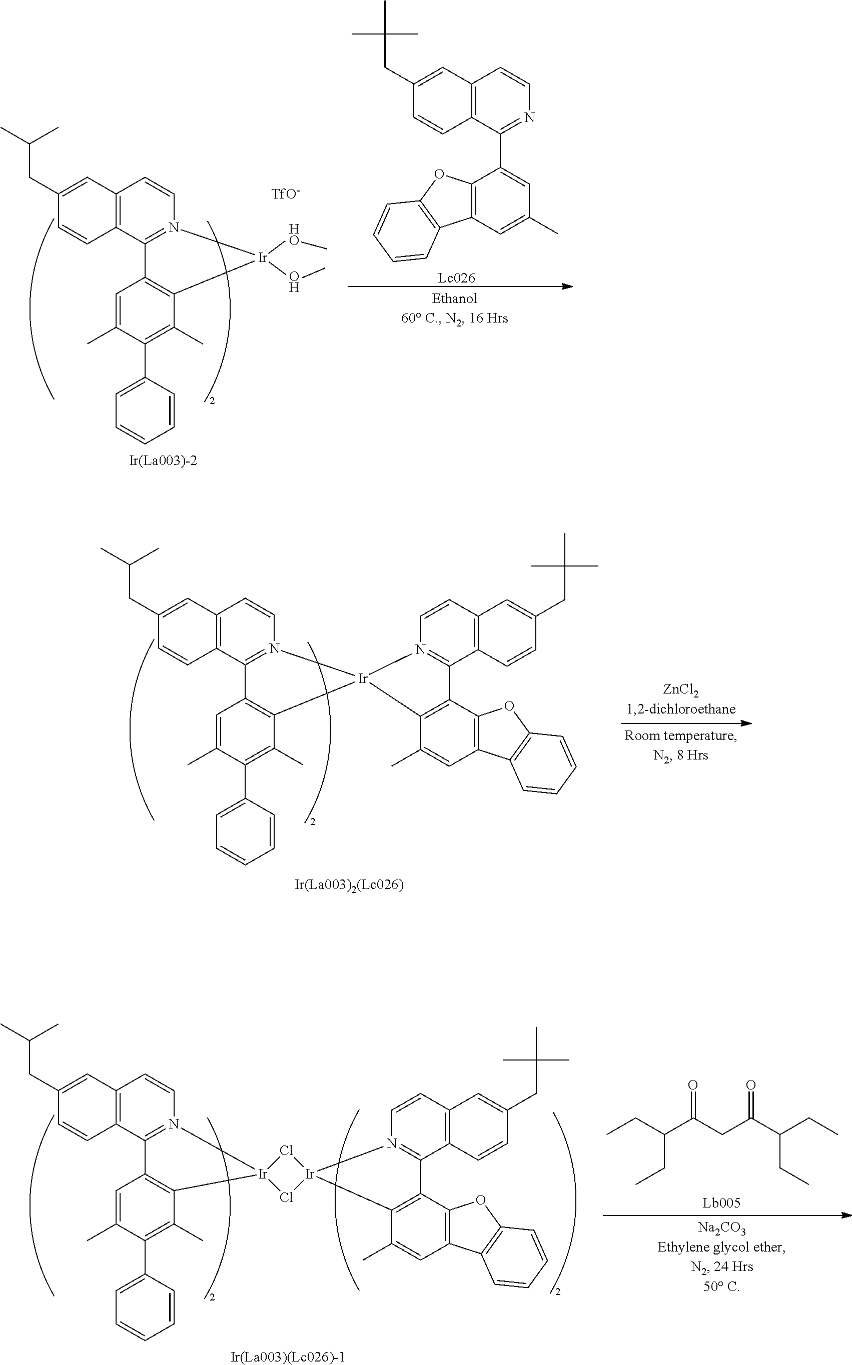

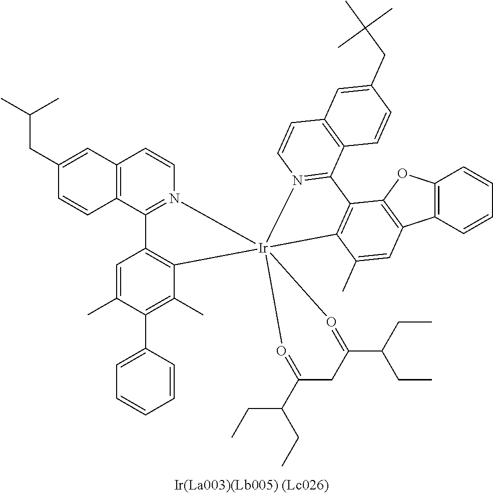

### Synthesis of Compound Ir(La003)₂(Lc026):

Referring to the synthesis and purification method of compound Ir(La003)₂(Lc004), only the corresponding raw materials needed to be changed to obtain a target compound Ir(La003)₂(Lc026), mass spectrometry: 1230.71 (M+H).

### Synthesis of Compound Ir(La003)(Lc026)-1:

Referring to the synthesis and purification method of the ligand Ir(La003)(Lc004)-1, only the corresponding raw materials needed to be changed to obtain a target compound Ir(La003)(Lc026)-1 that was used directly in the next step without purification.

### Synthesis of Compound Ir(La003)(Lb005)(Lc026):

Referring to the synthesis and purification method of compound Ir(La003)(Lb005)(Lc004), only the corresponding raw materials needed to be changed to obtain a red solid as compound Ir(La003)(Lb005)(Lc026) (1.75 g, yield: 42.35%). 1.75 g of crude Ir(La003)(Lb005)(Lc026) were sublimated and purified to obtain a sublimated pure Ir(La003)(Lb005)(Lc026)(1.02 g, yield: 58.28%), mass spectrometry: 1148.53 (M+H). ¹H NMR (400 MHz, CDCl₃) δ8.89 (s, 1H), 8.85 (s, 1H), 8.18 (s, 1H), 8.15 (s, 1H), 7.46-7.39 (m, 6H), 7.18-7.12 (m, 5H), 7.07-6.98 (d, J=7.4 Hz, 6H), 4.81 (s, 1H), 2.62 (d, J=7.6 Hz, 2H), 2.58 (d, J=7.4 Hz, 2H), 2.21-2.04 (m, 6H), 1.76 (m, 6H), 1.37-1.18 (m, 12H), 0.96-0.81 (m, 10H), 0.43-0.38 (m, 6H), 0.16-0.11 (m, 10H).

### Synthesis of Ligand La004:

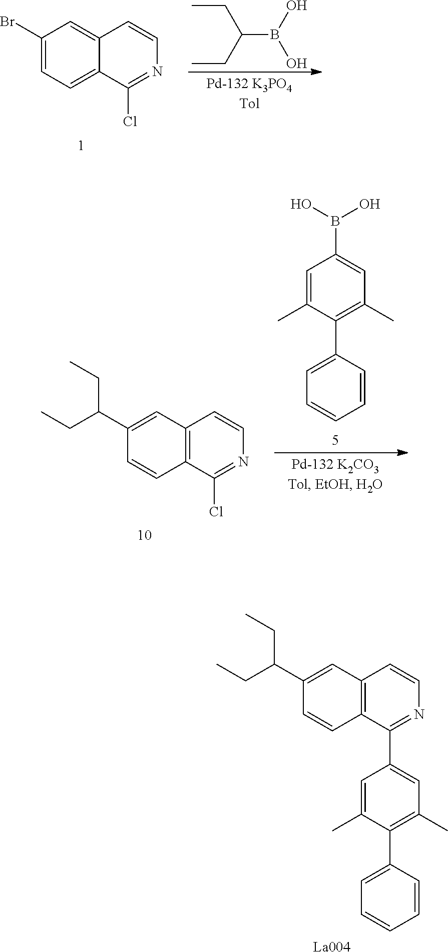

### Synthesis of Compound 10:

Referring to the synthesis and purification method of compound 2, only the corresponding raw materials needed to be changed to obtain a target compound 10, mass spectrometry: 234.74 (M+H).

### Synthesis of Ligand La004:

Referring to the synthesis and purification method of ligand La002, only the corresponding raw materials needed to be changed to obtain a target ligand La004, mass spectrometry: 380.54 (M+H).

### Synthesis of Compound Ir(La004)₂(Lb005):

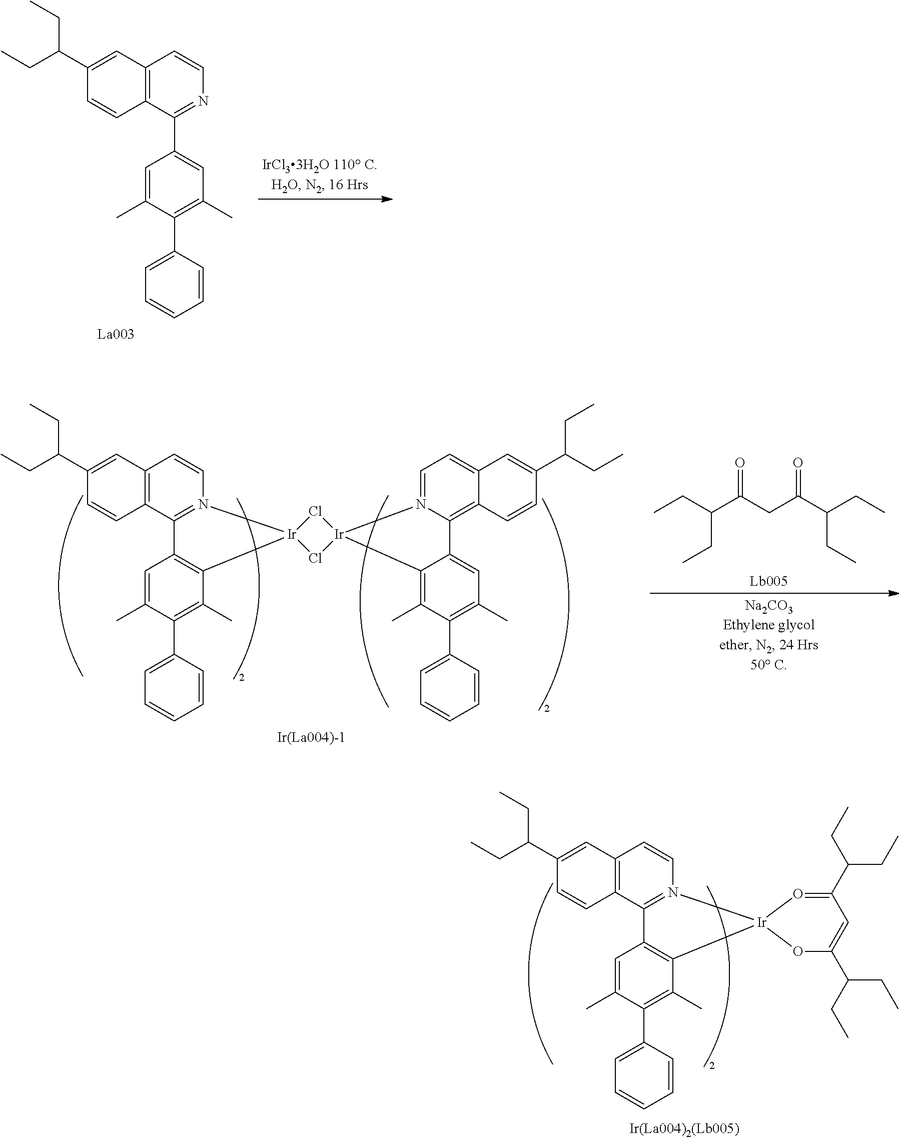

### Synthesis of Compound Ir(La004)-1:

Referring to the synthesis and purification method of compound Ir(La002)-1, only the corresponding raw materials needed to be changed to obtain a compound Ir(La004)-1 that was used directly in the next step without purification.

### Synthesis of Compound Ir(La004)₂(Lb005):

Referring to the synthesis and purification method of compound Ir(La002)₂(Lb005), only the corresponding raw materials needed to be changed to obtain a red solid as compound Ir(La004)₂(Lb005) (2.64 g, yield: 46.37%). 2.64 g of crude Ir(La004)₂(Lb005) was sublimated and purified to obtain a sublimated pure Ir(La004)₂(Lb005) (1.74 g, yield: 65.9/a), mass spectrometry: 1161.59 (M+H). ¹HNMR (400 MHz, CDCl₃) δ8.91 (d, J=8.6 Hz, 2H), 8.17 (d, J=6.6 Hz, 2H), 8.03 (s, 2H), 7.46 (s, 2H), 7.42 (d, J=9.3 Hz, 2H), 7.32 (t, J=7.9 Hz, 4H), 7.21 (t, J=7.5 Hz, 4H), 7.08 (d, J=6.4 Hz, 2H), 7.02 (d, J=7.1 Hz, 2H), 4.82 (s, 1H), 2.66 (d, J=7.4 Hz, 4H), 2.15-1.91 (m, 8H), 1.61 (d, J=7.5 Hz, 3H), 1.33-1.08 (m, 10H), 1.00-0.88 (m, 15H), 0.67-0.58 (m, 4H), 0.41 (t, J=7.4 Hz, 6H), −0.08 (t, J=7.1 Hz, 6H).

### Synthesis of Ligand La005:

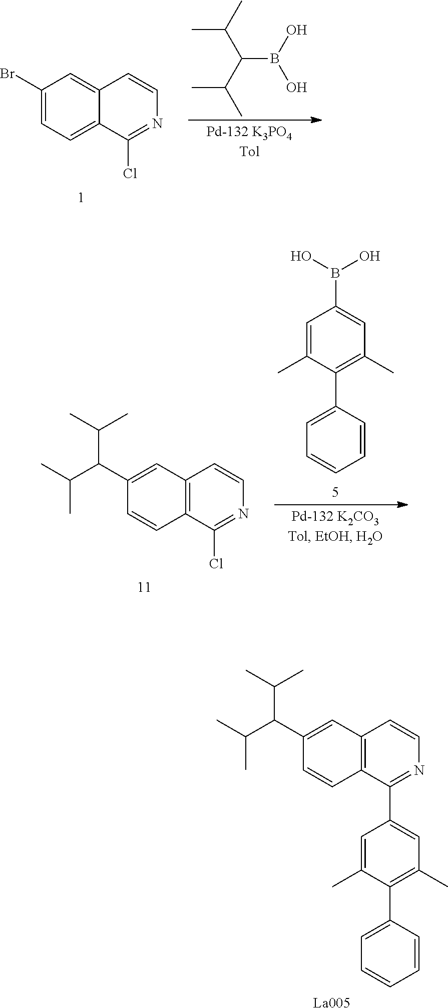

### Synthesis of Compound 11:

Referring to the synthesis and purification method of compound 2, only the corresponding raw materials needed to be changed to obtain a target compound 11, mass spectrometry: 261.79 (M+H).

### Synthesis of Ligand La005:

Referring to the synthesis and purification method of ligand La002, only the corresponding raw materials needed to be changed to obtain the target ligand La005. Mass spectrometry: 408.59 (M+H).

Synthesis of compound Ir(La005)₂(Lb005):

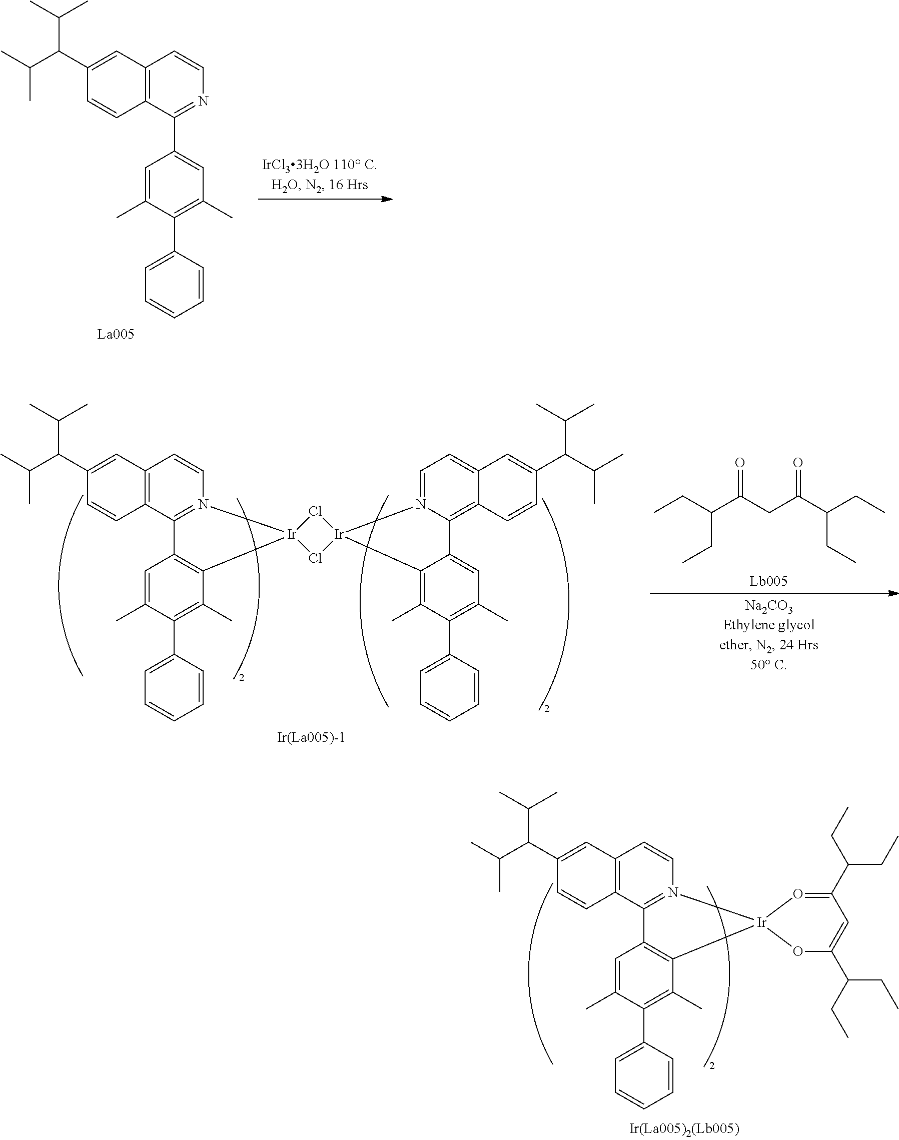

### Synthesis of Compound Ir(La005)-1:

Referring to the synthesis and purification method of compound Ir(La002)-1, only the corresponding raw materials needed to be changed to obtain compound Ir(La005)-1 that was used directly in the next step without purification.

### Synthesis of Compound Ir(La005)₂(Lb005):

Referring to the synthesis and purification method of compound Ir(La002)₂(Lb005), only the corresponding raw materials needed to be changed to obtain a red solid as compound Ir(La005)₂(Lb005) (2.74 g, yield: 44.21%). 2.74 g of crude Ir(La005)₂(Lb005) was sublimated and purified to obtain a sublimated pure Ir(La005)₂(Lb005) (1.68 g, yield: 61.31%), mass spectrometry: 1217.7 (M+H). ¹HNMR (400 MHz, CDCl₃) δ8.90 (d, J=8.7 Hz, 2H), 8.16 (d, J=6.5 Hz, 2H), 8.01 (s, 2H), 7.47 (s, 2H), 7.41 (d, J=9.1 Hz, 2H), 7.30 (t, J=7.8 Hz, 4H), 7.22 (t, J=7.5 Hz, 4H), 7.11 (d, J=6.4 Hz, 2H), 7.04 (d, J=7.1 Hz, 2H), 4.82 (s, 1H), 2.65 (d, J=7.2 Hz, 4H), 2.15-1.91 (m, 8H), 1.61 (d, J=7.5 Hz, 3H), 1.42-1.36 (m, 6H), 1.23-1.07 (m, 12H), 0.92-0.84 (m, 16H), 0.41-0.36 (m, 10H), 0.15-0.08 (m, 6H).

### Synthesis of Ligand La007:

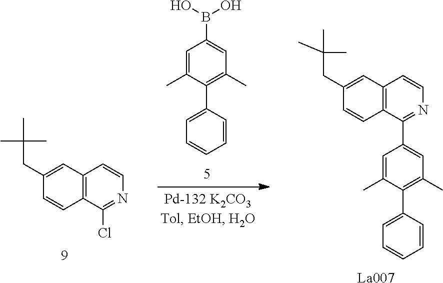

### Synthesis of Ligand La007:

Referring to the synthesis and purification method of ligand La002, only the corresponding raw materials needed to be changed to obtain a target ligand La007, mass spectrometry: 380.54 (M+H).

### Synthesis of Compound Ir(La007)₂(Lb005):

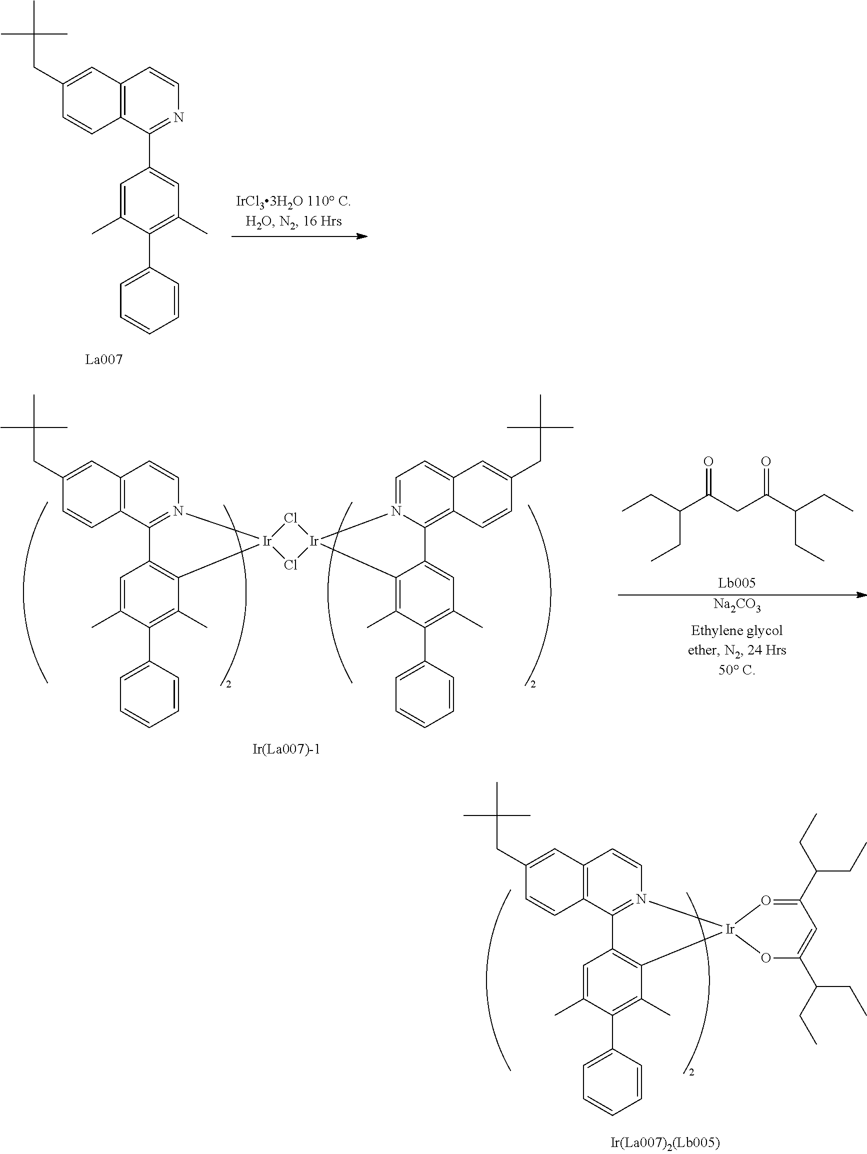

### Synthesis of Compound Ir(La007)-1:

Referring to the synthesis and purification method of compound Ir(La002)-1, only the corresponding raw materials needed to be changed to obtained compound Ir(La007)-1 that was used directly in the next step without purification.

### Synthesis of Compound Ir(La007)₂(Lb005):

Referring to the synthesis and purification method of compound Ir(La002)₂(Lb005), only the corresponding raw materials needed to be changed to obtain a red solid as compound Ir(La007)₂(Lb005) (2.74 g, yield: 44.21%). 2.74 g of crude Ir(La007)₂(Lb005) was sublimated and purified to obtain a sublimated pure Ir(La007)₂(Lb005) (1.68 g, yield: 61.31%), mass spectrometry: 1161.59 (M+H). ¹HNMR (400 MHz, CDCl₃) δ8.89 (d, J=8.7 Hz, 2H), 8.19 (d, J=6.6 Hz, 2H), 8.04 (s, 2H), 7.48 (s, 2H), 7.43 (d, J=9.1 Hz, 2H), 7.33 (t, J=8.2 Hz, 4H), 7.24 (t, J=7.5 Hz, 4H), 7.08 (d, J=6.4 Hz, 2H), 7.02 (d, J=7.1 Hz, 2H), 4.82 (s, 1H), 2.52 (d, J=7.4 Hz, 4H), 2.15-1.91 (m, 8H), 1.61 (d, J=7.5 Hz, 3H), 1.33-1.08 (m, 10H), 0.96-0.86 (m, 15H), 0.48-0.35 (m, 10H), −0.08 (t, J=7.1 Hz, 6H).

### Synthesis of Ligand La010:

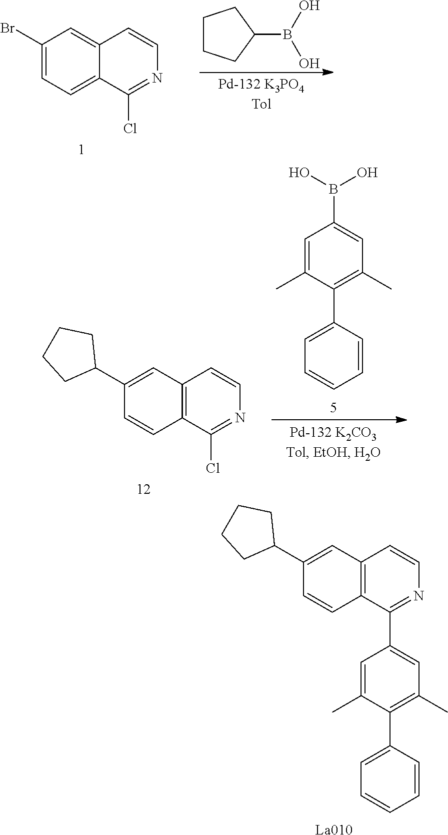

### Synthesis of Compound 12:

Referring to the synthesis and purification method of compound 2, only the corresponding raw materials needed to be changed to obtain a target compound 12, mass spectrometry: 232.72 (M+H).

### Synthesis of Ligand La010:

Referring to the synthesis and purification method of ligand La002, only the corresponding raw materials needed to be changed to obtain a target ligand La010, mass spectrometry: 378.52 (M+H).

### Synthesis of Compound Ir(La010)₂(Lb005):

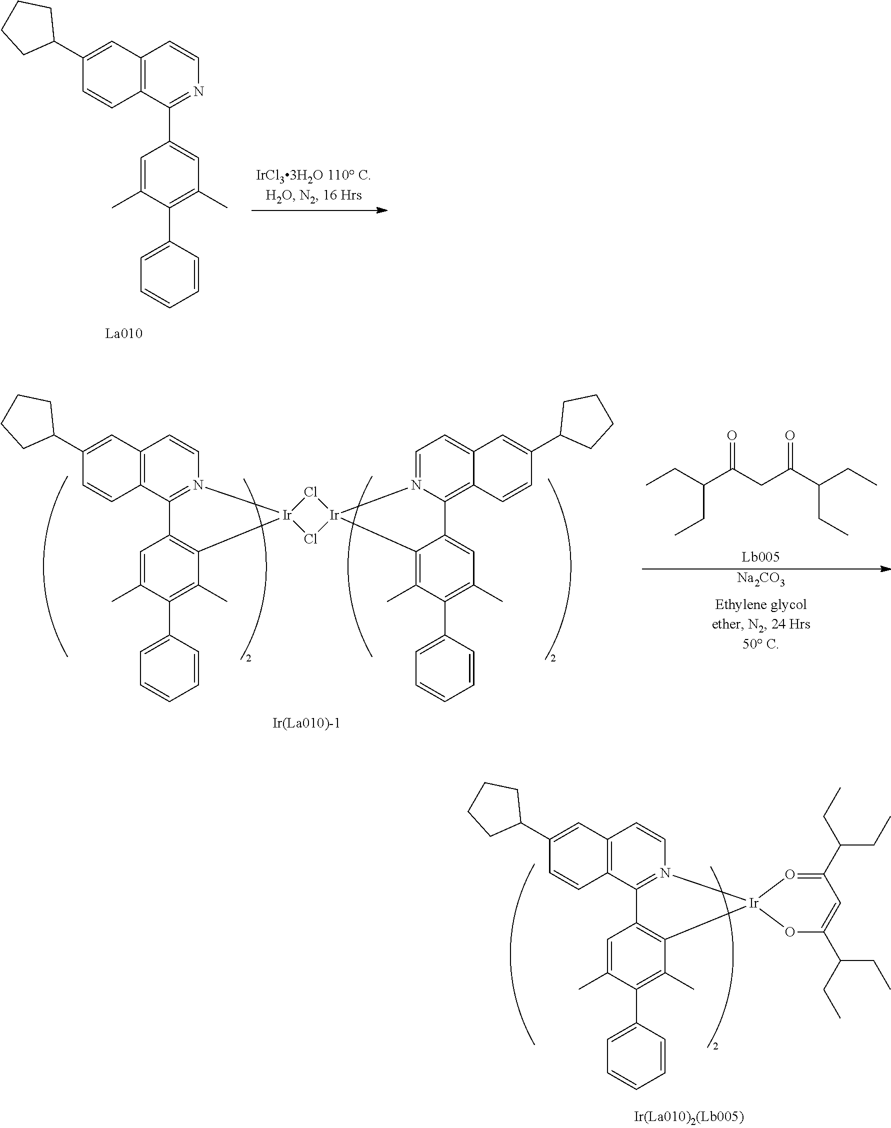

### Synthesis of Compound Ir(La010)-1:

Referring to the synthesis and purification method of compound Ir(La002)-1, only the corresponding raw materials needed to be changed to obtain compound Ir(La010)-1 that was used directly in the next step without purification.

### Synthesis of Compound Ir(La010)(Lb005):

Referring to the synthesis and purification method of compound Ir(La002)₂(Lb005), only the corresponding raw materials needed to be changed to obtain a red solid as compound Ir(La010)₂(Lb005) (2.74 g, yield: 44.21%). 2.74 g of crude Ir(La010)₂(Lb005) was sublimated and purified to obtain a sublimated pure Ir(La010)₂(Lb005) (1.68 g, yield: 61.31%), mass spectrometry: 1157.56 (M+H). ¹HNMR (400 MHz, CDCl₃) δ8.92 (d, J=8.8 Hz, 2H), 8.21 (d, J=6.8 Hz, 2H), 8.08 (s, 2H), 7.51 (s, 2H), 7.45 (d, J=9.1 Hz, 2H), 7.34 (t, J=8.2 Hz, 4H), 7.22 (t, J=7.5 Hz, 4H), 7.11 (d, J=6.4 Hz, 2H), 7.03 (d, J=7.1 Hz, 2H), 4.82 (s, 1H), 2.51 (d, J=7.4 Hz, 4H), 2.14-2.03 (m, 8H), 1.63 (d, J=7.5 Hz, 3H), 1.27-1.12 (m, 10H), 0.96-0.86 (m, 15H), 0.48-0.35 (m, 6H), −0.08 (t, J=7.1 Hz, 6H).

### Synthesis of Ligand La017:

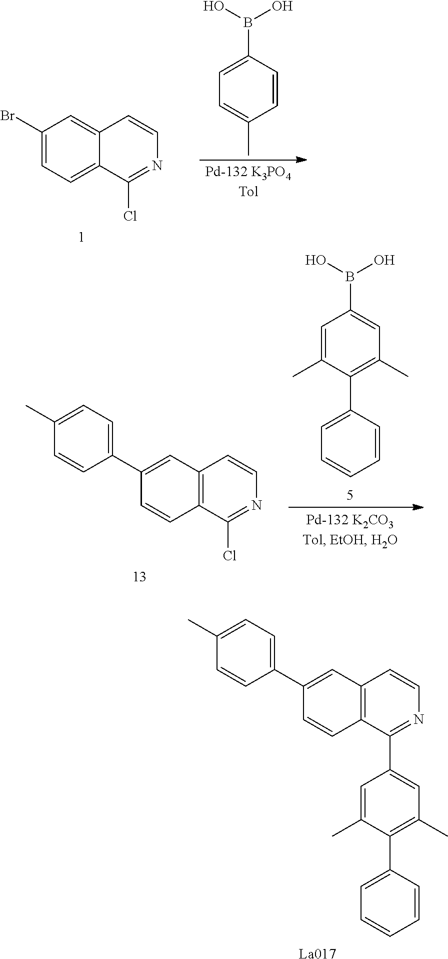

### Synthesis of Compound 13:

Referring to the synthesis and purification method of compound 2, only the corresponding raw materials needed to be changed to obtain a target compound 13, mass spectrometry: 254.73 (M+H).

### Synthesis of Ligand La017:

Referring to the synthesis and purification method of ligand La002, only the corresponding raw materials needed to be changed to obtain a target ligand La017, mass spectrometry: 400.53 (M+H).

### Synthesis of Compound Ir La017 Lb005):

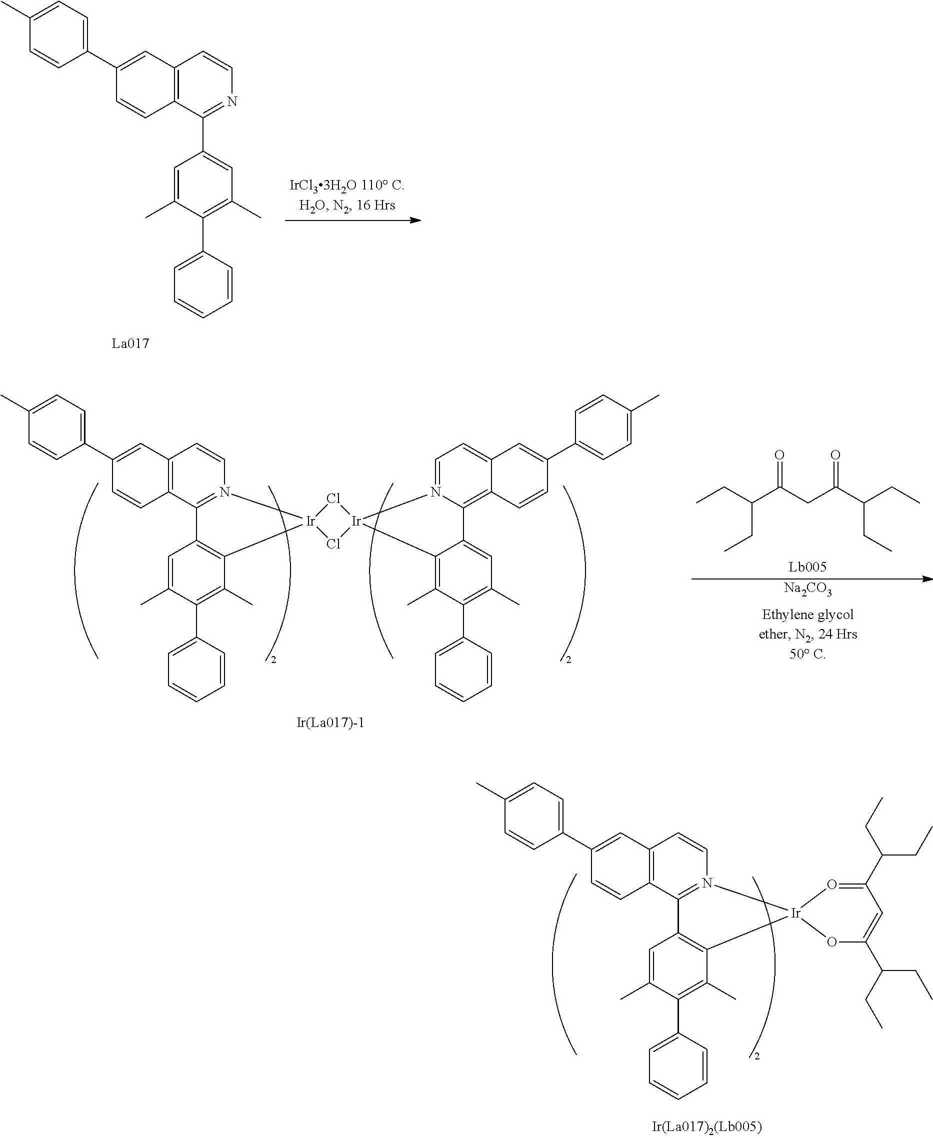

### Synthesis of Compound Ir(La017)-1:

Referring to the synthesis and purification method of compound Ir(La002)-1, only the corresponding raw materials needed to be changed to obtain compound Ir(La017)-1 that was used directly in the next step without purification.

### Synthesis of Compound Ir(La017)₂(Lb005):

Referring to the synthesis and purification method of compound Ir(La002)₂(Lb005), only the corresponding raw materials needed to be changed to obtain a red solid as compound Ir(La017)₂(Lb005) (2.21 g, yield: 39.21%). 2.21 g of crude Ir(La017)₂(Lb005) was sublimated and purified to obtain a sublimated pure Ir(La017)₂(Lb005) (1.24 g, yield: 56.10%), mass spectrometry: 1201.57 (M+H). ¹HNMR (400 MHz, CDCl₃) δ8.92 (d, J=8.8 Hz, 2H), 8.25 (d, J=6.6 Hz, 2H), 8.04 (s, 2H), 7.48 (s, 2H), 7.63-7.56 (m, 6H), 7.36-7.28 (m, 8H), 7.16 (t, J=7.8 Hz, 4H), 7.06 (d, J=6.4 Hz, 2H), 7.04 (d, J=7.3 Hz, 2H) 0.4.82 (s, 1H), 1.91 (m, 8H), 1.62 (s, 6H), 1.24-1.12 (m, 8H), 0.96-0.86 (m, 6H), 0.48-0.35 (m, 6H), −0.08 (t, J=7.2 Hz, 6H).

### Synthesis of Ligand La023:

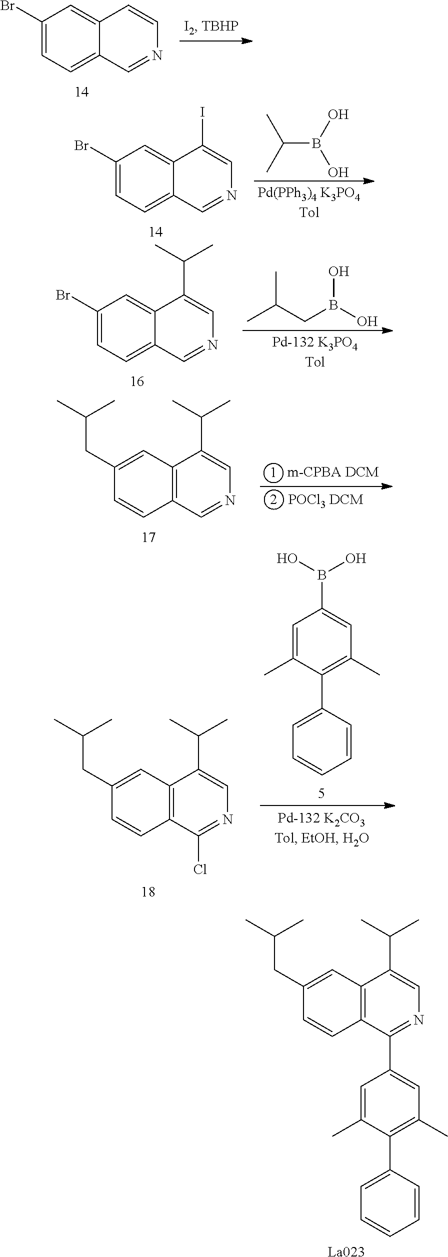

### Synthesis of Compound 15:

Compound 14 (25.0 g, 120.16 mmol, 1.0 eq), iodine (36.6 g, 144.19 mmol, 1.2 eq), 70% tert-butyl hydroperoxide (123.76 g, 0.96 mol, 8.0 eq) and tetrahydrofuran (150 mL) were added to a 500 mL reaction bottle, and the reaction was carried out in an oil bath at 80° C. for 6 h after vacuum and nitrogen replacement 3 times. TLC monitoring showed that compound 14 was reacted completely. The reaction solvent was cooled to room temperature and spin-dried, added with a saturated sodium thiosulfate solution (200 mL) and ethyl acetate (150 mL), stirred and washed, and separated to collect an organic phase that was washed once with water (150 mL). The collected organic phase was concentrated and purified by column chromatography (eluted with n-hexane:ethyl acetate=15:1) to obtain compound 15 (25.81 g, yield: 64.32%), mass spectrometry: 334.95 (M+H).

### Synthesis of Compound 16:

Referring to the synthesis and purification method of compound 2, only the corresponding raw materials and catalysts needed to be changed to obtain a target compound 16, mass spectrometry: 334.95 (M+H).

### Synthesis of Compound 17:

Referring to the synthesis and purification method of compound 2, only the corresponding raw materials needed to be changed to obtain a target compound 17, mass spectrometry: 251.13 (M+H).

### Synthesis of Compound 18:

Compound 17 (14.5 g, 63.78 mmol, 1.0 eq) and dichloromethane (145 mL) were into a 500 mL reaction bottle for reaction and cooled to 0° C., and the reaction solution was returned to room temperature overnight after addition of m-chloroperoxybenzoic acid (33.02 g, 191.34 mmol, 3.0 eq) in batches. TLC monitoring showed that compound 17 was reacted almost completely. The reaction solution was filtered directly to collect a filtrate that was spin-dried, and the next reaction was carried out directly. The dry nitrogen oxide was added to methylene chloride (50 mL) for reaction, the reaction solution was cooled to 0° C., phosphorus oxychloride (50 mL) was slowly added to the reaction solution, and the reaction solution was heated to 45° C. for reaction for 2 h. The reaction solution was slowly dropped into 250 mL of ice water at room temperature to quench, then saturated sodium carbonate solution was added to adjust the pH to 8-9, ethyl acetate (150 mL/time) was added for extraction 3 times to obtain an organic phase. The organic phase was concentrated to dryness, and purified by column chromatography (hexane:ethyl acetate=100:1) to obtain compound 18 (6.78 g, yield: 40.62%), mass spectrometry: 262.79 (M+H).

### Synthesis of Ligand La023:

Referring to the synthesis and purification method of ligand La002, only the corresponding raw materials needed to be changed to obtain a target ligand La023, mass spectrometry: 408.59 (M+H).

### Synthesis of Compound Ir(La023)₂(Lb005):

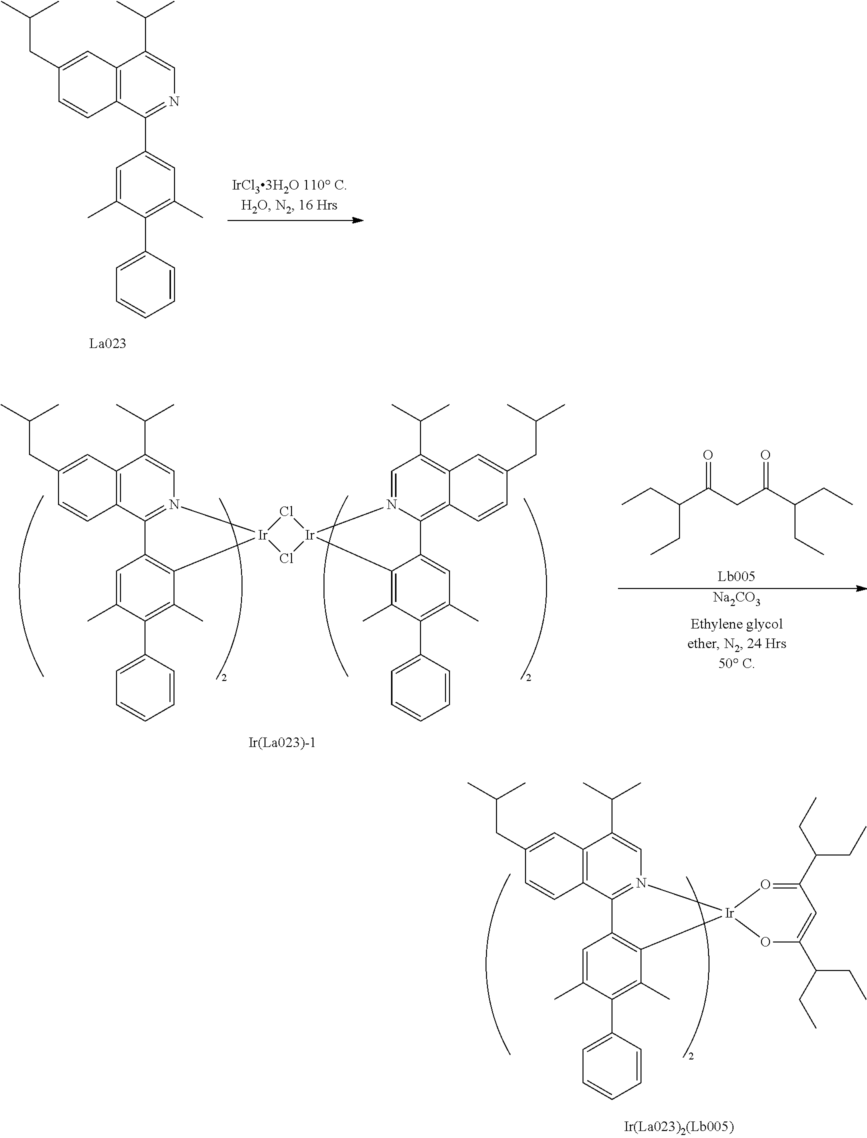

### Synthesis of Compound Ir(La023)-1:

Referring to the synthesis and purification method of compound Ir(La002)-1, only the corresponding raw materials needed to be changed to obtain a compound Ir(La023)-1 that was used directly in the next step without purification.

### Synthesis of Compound Ir(La023)₂(Lb005):

Referring to the synthesis and purification method of compound Ir(La002)₂(Lb005), only the corresponding raw materials needed to be changed to obtain a red solid as compound Ir(La023)₂(Lb005) (2.45 g, yield: 38.62%). 2.45 g of crude Ir(La023)₂(Lb005) was sublimated and purified to obtain a sublimated pure Ir(La023)₂(Lb005) (1.59 g, yield: 64.89%), mass spectrometry: 1217.7 (M+H). ¹HNMR (400 MHz, CDCl₃) δ8.92 (s, J=8.8 Hz, 2H), 8.21 (d, J=6.8 Hz, 2H), 8.08 (s, 2H), 7.51 (s, 2H), 7.34 (t, J=8.2 Hz, 4H), 7.22 (t, J=7.5 Hz, 4H), 7.11 (d, J=6.4 Hz, 2H), 7.03 (d, J=7.1 Hz, 2H), 4.82 (s, 1H), 2.52 (d, J=7.4 Hz, 4H), 2.43 (d, J=6.8 Hz, 2H), 2.32 (d, J=7.0 Hz, 2H), 1.96-1.85 (m, 12H), 1.71 (m, 6H), 1.22-1.08 (m, 9H), 0.92-0.84 (m, 16H), 0.48-0.35 (m, 9H), −0.08 (t, J=7.1 Hz, 6H).

### Synthesis of Ligand La043:

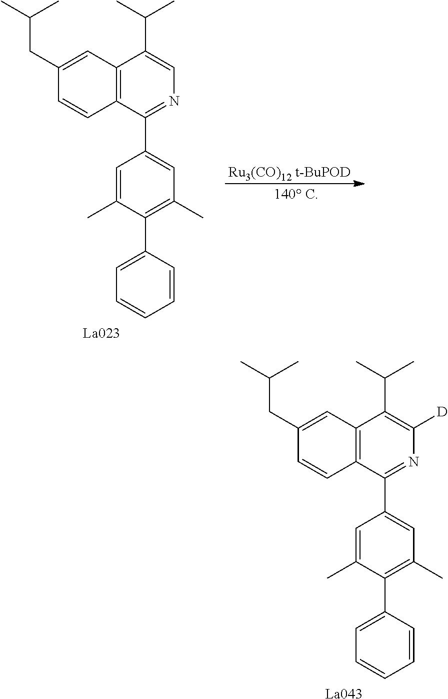 [SMILES: Cc1cc(-c2ncc(C(C)C)c3cc(CC(C)C)ccc23)cc(C)c1-c1ccccc1 ; "Ru3(CO)12 t-BuPOD" ; "La023" ; [2H]c1nc(-c2cc(C)c(-c3ccccc3)c(C)c2)c2ccc(CC(C)C)cc2c1C(C)C ; "La043"]

Ligand La023 (14.3 g, 30.18 mmol, 1.0 eq), triruthenium dodecacarbonyl (1.93 g, 3.02 mmol, 0.1 eq), and deuterated tert-butanol (75 mL) were added into a 250 mL reaction flask for reaction in an oil bath at 75° C. for 6 h after 3 times of vacuum and nitrogen replacement. The reaction solution was cooled to room temperature, and the reaction solvent was spin-dried, then methylene chloride (80 mL) was added to dissolve and filter to collect an organic phase to be spin-dried. The reaction was carried out once again according to the original feeding method. After the reaction was completed, the solvent was spined off and purified by column chromatography (eluted with n-hexane:ethyl acetate=30:1) to obtain a ligand La043 (6.28 g, yield: 50.9%), mass spectrometry: 409.6 (M+H).

### Synthesis of Compound Ir(La043)₂(Lb005):

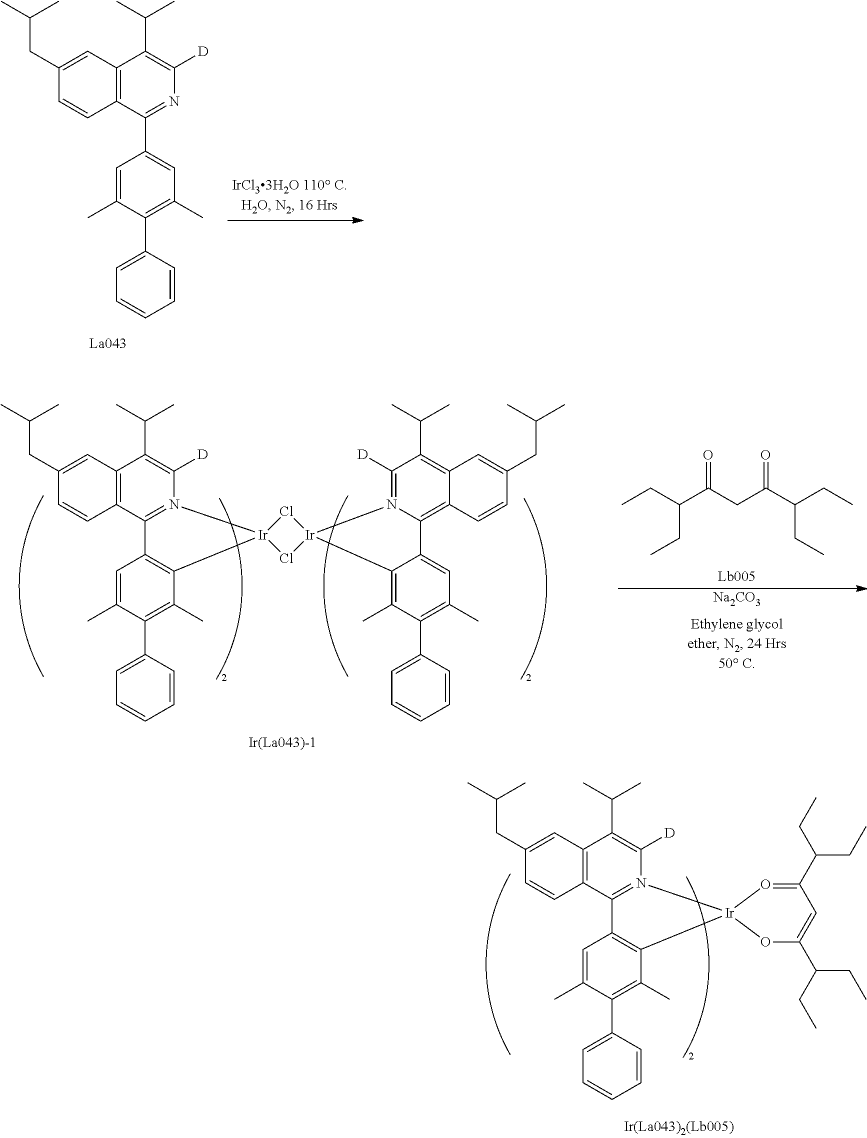

### Synthesis of Compound Ir(La043)-1:

Referring to the synthesis and purification method of compound Ir(La002)-1, only the corresponding raw materials needed to be changed to obtain compound Ir(La043)-1 that was used directly in the next step without purification.

### Synthesis of Compound Ir(La043)₂(Lb005):

Referring to the synthesis and purification method of compound Ir(La002)₂(Lb005), only the corresponding raw materials needed to be changed to obtain a red solid as compound Ir(La043)₂(Lb005) (1.68 g, yield: 37.62%). 1.68 g of crude Ir(La043)₂(Lb005) was sublimated and purified to obtain a sublimated pure Ir(La043)₂(Lb005) (0.93 g, yield: 55.35%), mass spectrometry: 1219.71 (M+H). ¹HNMR (400 MHz, CDCl₃) 8.56 (d, J=6.8 Hz, 2H), 8.08 (s, 2H), 7.51 (s, 2H), 7.34 (t, J=8.2 Hz, 4H), 7.22 (t, J=7.5 Hz, 4H), 7.11 (d, J=6.4 Hz, 2H), 7.03 (d, J=7.1 Hz, 2H), 4.81 (s, 1H), 2.50 (d, J=7.4 Hz, 4H), 2.42 (d, J=6.8 Hz, 2H), 2.31 (d, J=7.0 Hz, 2H), 1.95-1.84 (m, 12H), 1.72 (m, 6H), 1.22-1.08 (m, 9H), 0.92-0.84 (m, 16H), 0.48-0.35 (m, 9H), −0.08 (t, J=7.1 Hz, 6H).

### Synthesis of Ligand La083:

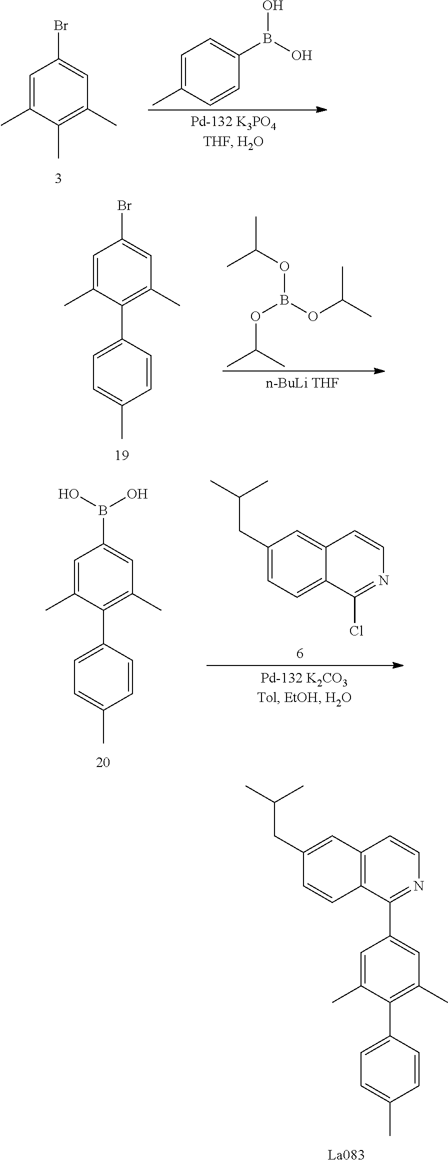

### Synthesis of Compound 19:

Referring to the synthesis and purification method of compound 4, only the corresponding raw materials needed to be changed to obtain a target compound 19, mass spectrometry: 276.18 (M+H).

### Synthesis of Compound 20:

Referring to the synthesis and purification method of compound 5, only the corresponding raw materials needed to be changed to obtain a target compound 20, mass spectrometry: 241.44 (M+H).

### Synthesis of Ligand La083:

Referring to the synthesis and purification method of ligand La002, only the corresponding raw materials needed to be changed to obtain a target ligand La083, mass spectrometry: 380.54 (M+H).

### Synthesis of Compound Ir(La083)₂(Lb005):

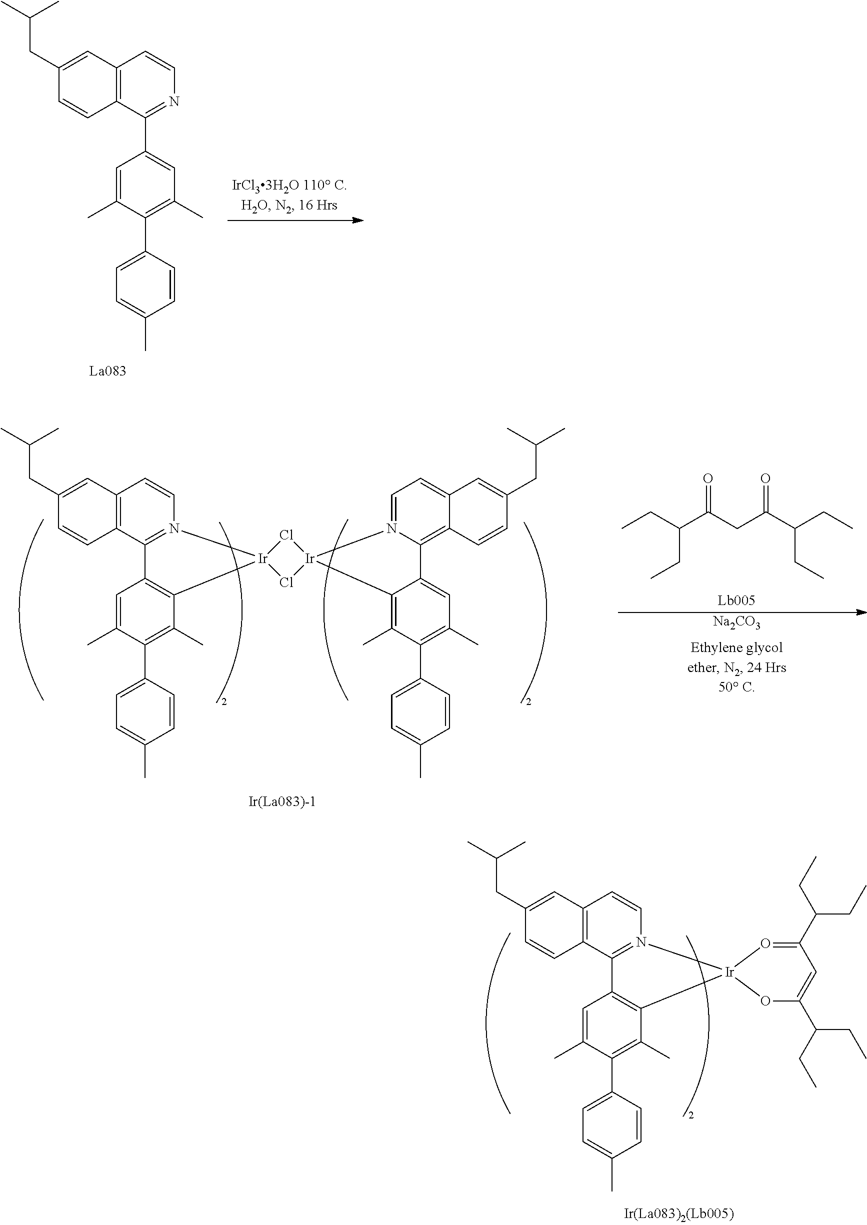

### Synthesis of Compound Ir(La083)-1:

Referring to the synthesis and purification method of compound Ir(La002)-1, only the corresponding raw materials needed to be changed to obtain compound Ir(La083)-1 that was used directly in the next step without purification.

### Synthesis of Compound Ir(La083)₂(Lb005):

Referring to the synthesis and purification method of compound Ir(La002)₂(Lb005), only the corresponding raw materials needed to be changed to obtain a red solid as compound Ir(La083)₂(Lb005) (1.85 g, yield: 39.67%). 1.85 g of crude Ir(La083)₂(Lb005) was sublimated and purified to obtain a sublimated pure Ir(La083)₂(Lb005) (1.14 g. yield: 61.62%), mass spectrometry: 1161.59 (M+H). ¹HNMR (400 MHz, CDCl₃) δ8.91 (d, J=8.9 Hz, 2H), 8.16 (d, J=6.6 Hz, 2H), 8.02 (s, 2H), 7.46 (s, 2H), 7.46 (d, J=9.6 Hz, 2H), 7.30 (t, J=7.8 Hz, 4H), 7.19 (t, J=7.6 Hz, 4H), 7.01 (d, J=7.6 Hz, 2H), 4.82 (s, 1H), 2.66 (di, J=7.4 Hz, 4H), 2.14-1.89 (m, 8H), 1.62 (di, J=7.5 Hz, 3H), 1.2-1.08 (m, 10H), 1.02-0.89 (m, 15H), 0.41 (t, J=7.4 Hz, 6H), −0.07 (t, J=7.3 Hz, 6H).

### Synthesis of Ligand La107:

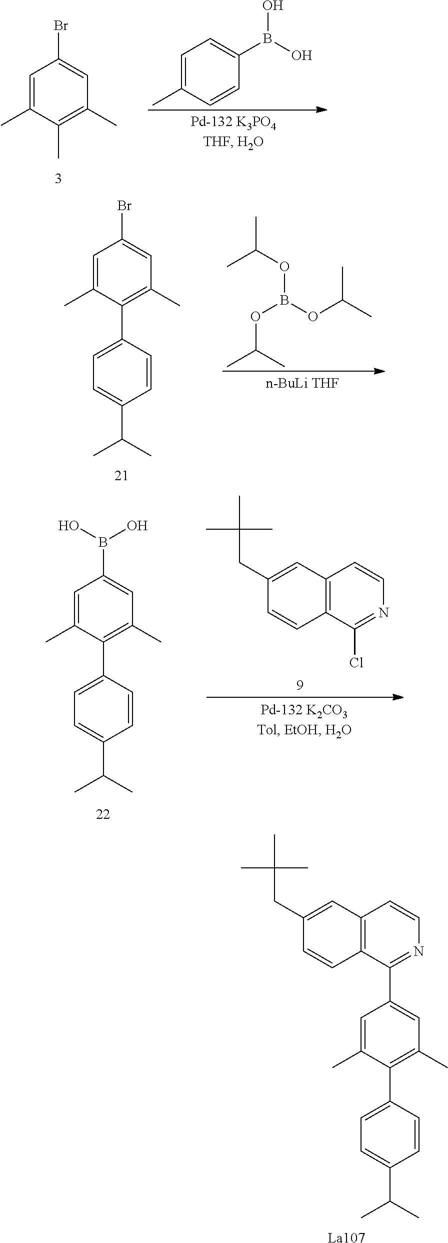

### Synthesis of Compound 21:

Referring to the synthesis and purification method of compound 4, only the corresponding raw materials needed to be changed to obtain a target compound 21, mass spectrometry: 304.24 (M+H).

### Synthesis of Compound 22:

Referring to the synthesis and purification method of compound 5, only the corresponding raw materials needed to be changed to obtain a target compound 22, mass spectrometry: 269.16 (M+H).

### Synthesis of Ligand La1107:

Referring to the synthesis and purification method of ligand La002, only the corresponding raw materials needed to be changed to obtain a target ligand La107, mass spectrometry: 422.62 (M+H).

### Synthesis of Compound Ir(La107)₂(Lb005):

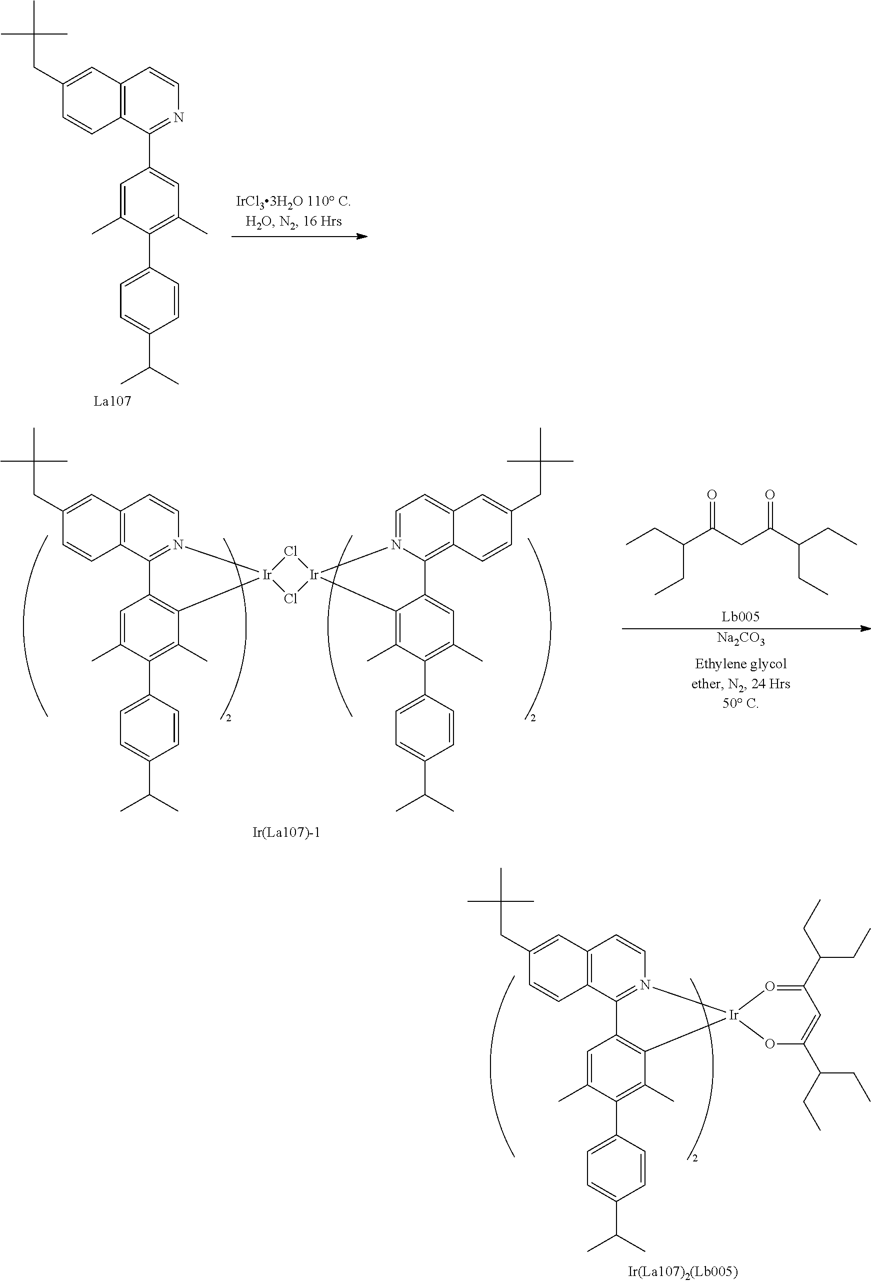

### Synthesis of Compound Ir(La107)-1:

Referring to the synthesis and purification method of compound Ir(La002)-1, only the corresponding raw materials needed to be changed to obtain compound Ir(La107)-1 that was used directly in the next step without purification.

### Synthesis of Compound Ir(La107)₂(Lb005):

Referring to the synthesis and purification method of compound Ir(La002)₂(Lb005), only the corresponding raw materials needed to be changed to obtain to obtain a red solid compound Ir(La107)₂(Lb005) (2.04 g, yield: 42.4%). 2.04 g of crude Ir(La107)₂(Lb005) were sublimated and purified to obtain a sublimated pure Ir(La107)₂(Lb005) (1.21 g, yield: 59.31;%), mass spectrometry: 1245.7.5 (M+H). ¹HNMR (400 MHz, CDCl₃) δ8.91 (d, J=8.9 Hz, 2H), 8.16 (di, J=6.6 Hz, 2H), 8.02 (s, 2H), 7.46 (s, 2H), 7.46 (d, J=9.6 Hz, 2H), 7.30 (t, J=7.8 Hz, 4H), 7.19 (t, J=7.6 Hz, 4H), 7.01 (d, J=7.6 Hz, 2H), 4.82 (s, 1H) 2.66 (d, J=7.4 Hz, 4H), 2.23 (m, 2H), 2.14-1.89 (m, 8H), 1.62 (m, 12H), 1.2-1.08 (in, 12H), 1.02-0.89 (m, 16H), 0.41 (t, J=7.4 Hz, 6H), −0.07 (in, 10H).

### Synthesis of Ligand La123:

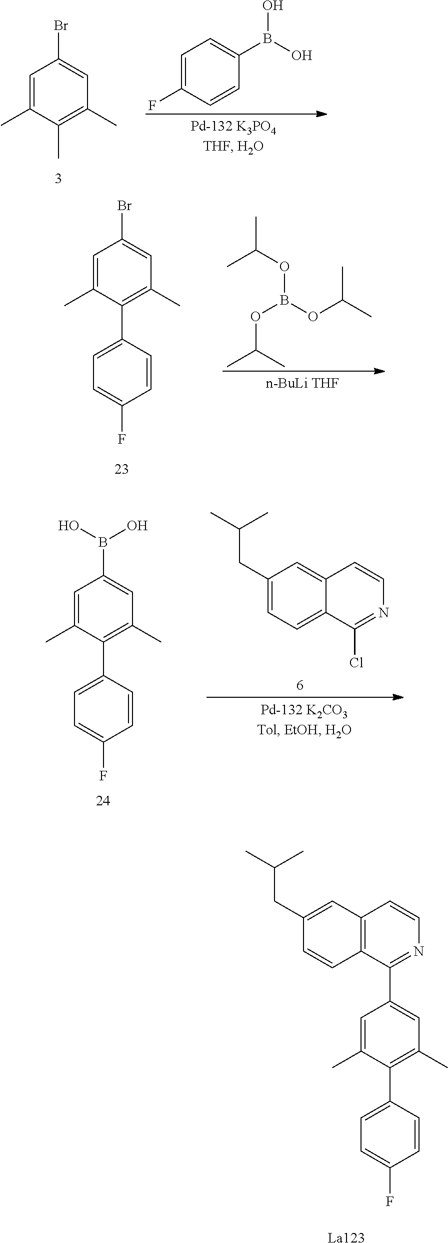

### Synthesis of Compound 23:

Referring to the synthesis and purification method of compound 4, only the corresponding raw materials needed to be changed to obtain a target compound 23, mass spectrometry: 280.15 (M+H).

### Synthesis of Compound 24:

Referring to the synthesis and purification method of compound 5, only the corresponding raw materials needed to be changed to obtain a target compound 24, mass spectrometry: 245.07 (M+H).

### Synthesis of Ligand La123:

Referring to the synthesis and purification method of ligand La002, only the corresponding raw materials needed to be changed to obtain a target ligand La123, mass spectrometry: 384.5 (M+H). ¹HNMR (400 MHz, DMSO) δ 8.52 (d, J=5.6 Hz, 1H), 8.04 (d, J=8.7 Hz, 1H), 7.84-7.71 (m, 2H), 7.50 (dd, J=8.7, 1.4 Hz, 1H), 7.41 (s, 2H), 7.37-7.25 (m, 4H), 2.66 (d, J=7.1 Hz, 2H), 2.07 (s, 6H), 0.91 (d, J=6.6 Hz, 6H).

### Synthesis of Compound Ir(La123)₂(Lb005):

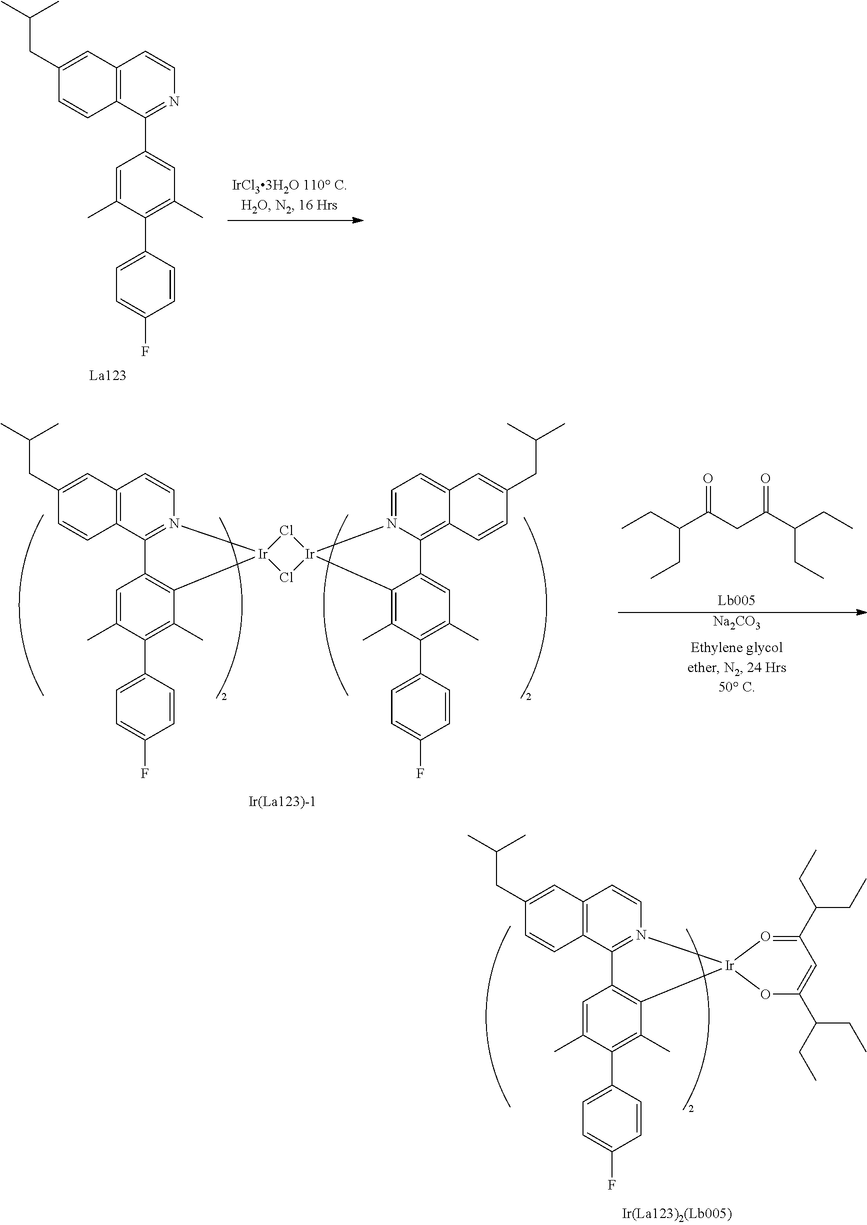

### Synthesis of Compound Ir(La123)-1:

Referring to the synthesis and purification method of compound Ir(La002)-1, only the corresponding raw materials needed to be changed to obtain compound Ir(La123)-1 that was used directly in the next step without purification.

### Synthesis of Compound Ir(La123)₂(Lb005):

Referring to the synthesis and purification method of compound Ir(La002)₂(Lb005), only the corresponding raw materials needed to be changed to obtain a red solid as compound Ir(La123)₂(Lb005) (1.87 g, yield: 39.64%). 1.87 g of crude Ir(La123)₂(Lb005) was sublimated and purified to obtain a sublimated pure Ir(La123)₂(Lb005) (0.98 g, yield: 52.4%), mass spectrometry: 1169.52 (M+H). ¹HNMR (400 MHz, CDCl₃) δ 8.89 (d, J=8.8 Hz, 2H), 8.14 (d, J=6.4 Hz, 2H), 8.00 (s, 2H), 7.52-7.42 (m, 4H), 7.17-7.12 (m, 2H), 7.07 (d, J=6.4 Hz, 2H), 7.02-6.94 (m, 6H), 4.81 (s, 1H), 2.66 (d, J=7.2 Hz, 4H), 2.10-1.99 (m, 8H), 1.59 (d, J=5.0 Hz, 5H), 1.41-1.05 (m, 12H), 1.01-0.84 (m, 17H), 0.39 (t, J=7.4 Hz, 6H), −0.10 (t, J=7.3 Hz, 6H).

### Synthesis of Ligand La127:

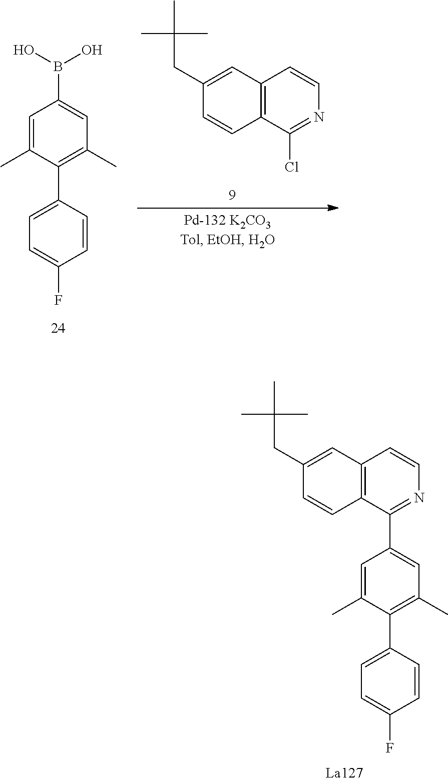

Referring to the synthesis and purification method of ligand La002, only the corresponding raw materials needed to be changed to obtain a target ligand La127, mass spectrometry: 398.53 (M+H).

### Synthesis of Compound Ir(La127)₂(Lb005):

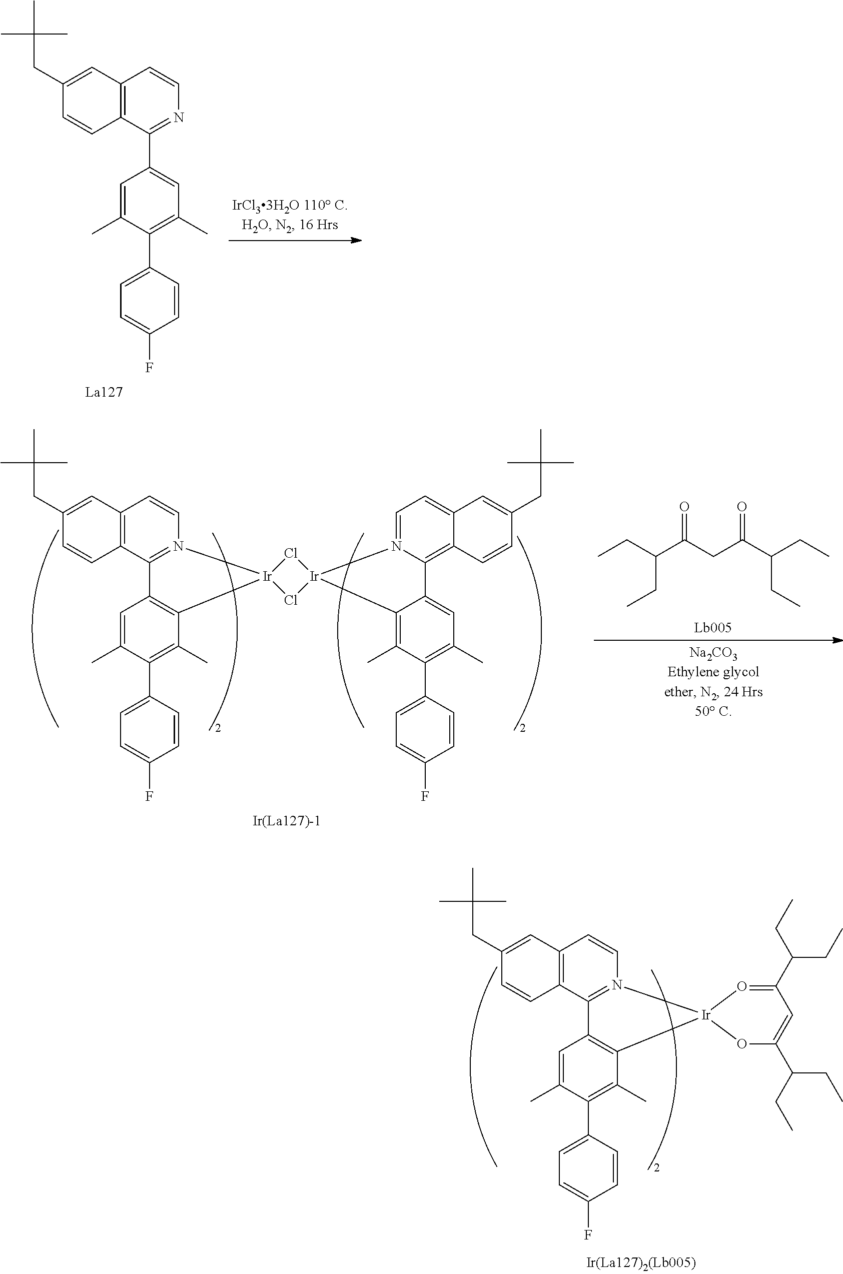

### Synthesis of Compound Ir(La127)-1:

Referring to the synthesis and purification method of compound Ir(La002)-1, only the corresponding raw materials needed to be changed to obtain compound Ir(La127)-1 that was used directly in the next step without purification.

### Synthesis of Compound Ir(La127)₂(Lb005):

Referring to the synthesis and purification method of compound Ir(La002)₂(Lb005), only the corresponding raw materials needed to be changed to obtain a red solid as compound Ir(La127)₂(Lb005) (1.63 g, yield: 38.5%). 1.63 g of crude Ir(La127)₂(Lb005) were sublimated and purified to obtain a sublimated pure Ir(La127)₂(Lb005) (0.92 g, yield: 56.44%), mass spectrometry: 1197.59 (M+H). ¹HNMR (400 MHz, CDCl₃) δ 8.90 (d, J=8.8 Hz, 2H), 8.15 (d, J=6.4 Hz, 2H), 8.02 (s, 2H), 7.52-7.42 (m, 4H), 7.16-7.12 (m, 2H), 7.06 (d, J=6.4 Hz, 2H), 7.02-6.94 (m, 6H), 4.82 (s, 1H), 2.68 (d, J=7.2 Hz, 4H), 2.13-2.01 (m, 8H), 1.61 (d, J=5.0 Hz, 5H), 1.39-1.04 (m, 14H), 1.01-0.84 (m, 17H), 0.41 (t, J=7.4 Hz, 8H), −0.12 (t, J=7.3 Hz, 6H).

### Synthesis of Ligand La143:

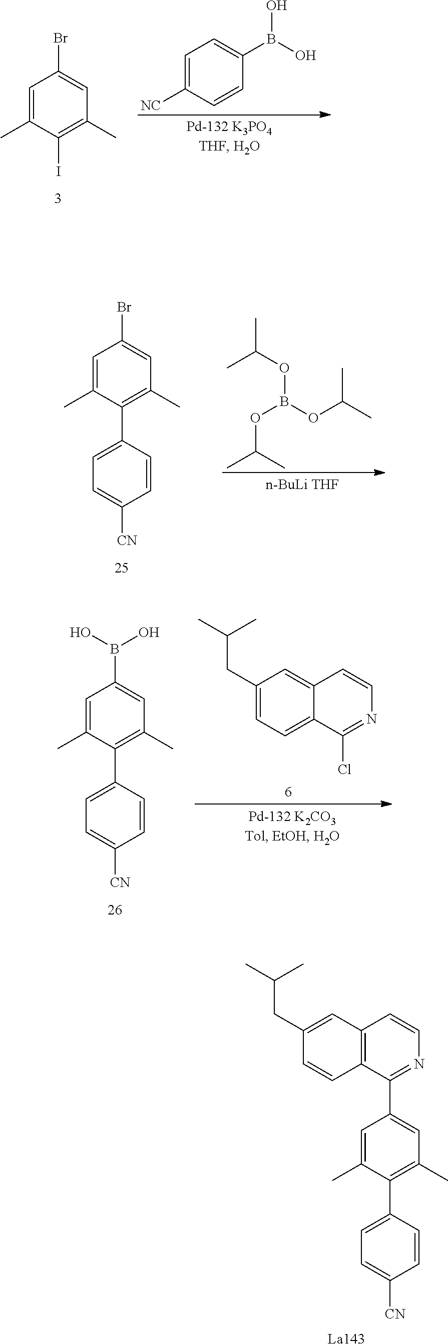

### Synthesis of Compound 25:

Referring to the synthesis and purification method of compound 4, only the corresponding raw materials needed to be changed to obtain a target compound 25, mass spectrometry: 287.14 (M+H).

### Synthesis of Compound 26:

Referring to the synthesis and purification method of compound 5, only the corresponding raw materials needed to be changed to obtain a target compound 26, mass spectrometry: 252.09 (M+H).

### Synthesis of Ligand La143:

Referring to the synthesis and purification method of ligand La002, only the corresponding raw materials needed to be changed to obtain a target ligand La143, mass spectrometry: 391.52 (M+H). ¹HNMR (400 MHz, CDCl₃) δ 8.58 (dd, J=6.9, 4.7 Hz, 1H), 8.12 (t, J=6.0 Hz, 1H), 7.79 (t, J=5.9 Hz, 1H), 7.65-7.58 (m, 1H), 7.47 (d, J=4.2 Hz, 1H), 7.41-7.34 (m, 1H), 2.70 (t, J=5.7 Hz, 1H), 2.10 (d, J=3.9 Hz, 1H), 2.01 (dd, J=12.7, 5.9 Hz, 1H), 0.97 (t, J=5.1 Hz, 1H).

### Synthesis of Compound Ir(La143)₂(Lb005)

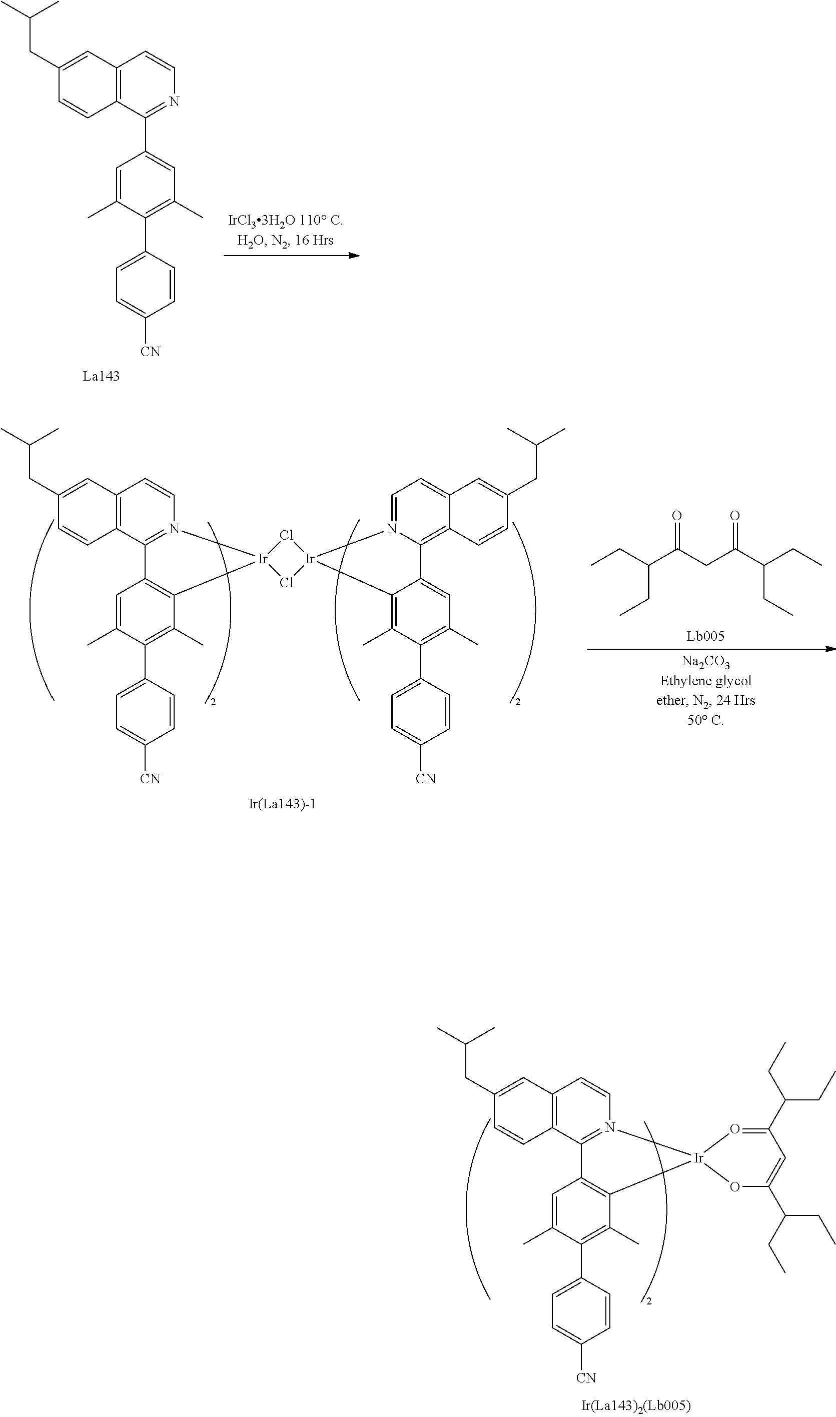

### Synthesis of Compound Ir(La143)-1:

Referring to the synthesis and purification method of compound Ir(La002)-1, only the corresponding raw materials needed to be changed to obtain a compound Ir(La143)-1 that was used directly in the next step without purification.

### Synthesis of Compound Ir(La143)₂(Lb005):

Referring to the synthesis and purification method of compound Ir(La002)₂(Lb005), only the corresponding raw materials needed to be changed to obtain a red solid as compound Ir(La143)₂(Lb005) (1.77 g, yield: 39.62%). 1.77 g of crude Ir(La143)₂(Lb005) were sublimated and purified to obtain a sublimated pure Ir(La143)₂(Lb005) (1.04 g, yield: 58.75%), mass spectrometry: 1183.56 (M+H). ¹HNMR (400 MHz, CDCl₃) δ 8.88 (d, J=8.8 Hz, 2H), 8.14 (d, J=6.4 Hz, 2H), 8.03 (s, 2H), 7.60 (dd, J=5.3, 2.0 Hz, 4H), 7.55-7.43 (m, 4H), 7.37-7.29 (m, 2H), 7.19-7.05 (m, 4H), 4.83 (s, 1H), 2.67 (d, J=7.1 Hz, 4H), 2.11-1.96 (m, 8H), 1.69-1.52 (m, 3H), 1.36-1.03 (m, 1H), 1.03-0.78 (m, 17H), 0.41 (t, J=7.4 Hz, 6H), −0.12 (t, J=7.3 Hz, 6H).

### Synthesis of Ligand La147:

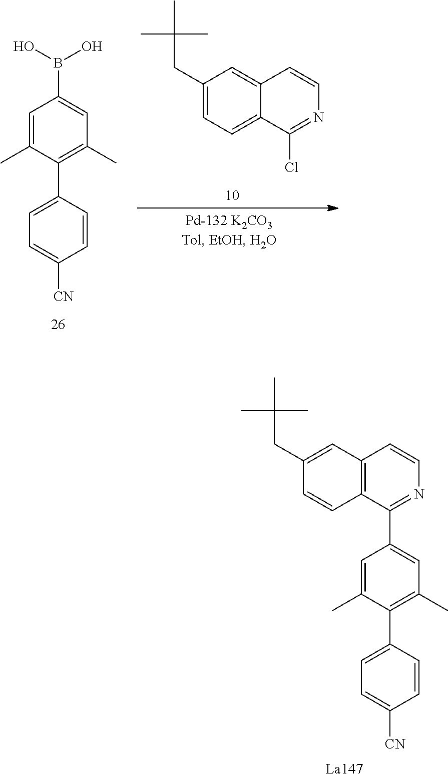

Referring to the synthesis and purification method of ligand La002, only the corresponding raw materials needed to be changed to obtain a target ligand La147, mass spectrometry: 405.55 (M+H).

### Synthesis of Compound Ir(La147)₂(Lb005):

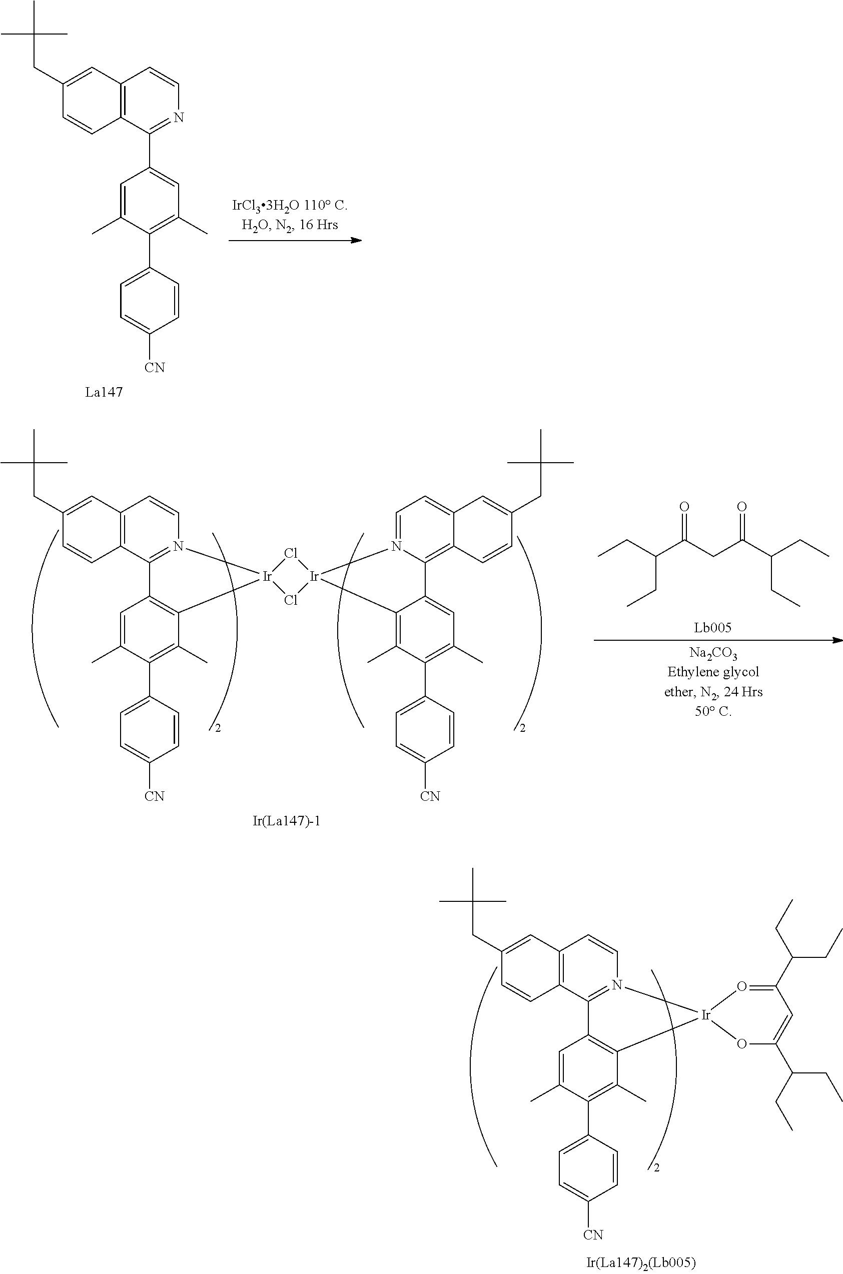

### Synthesis of Compound Ir(La147)-1:

Referring to the synthesis and purification method of compound Ir(La002)-1, only the corresponding raw materials need to be changed to obtained compound Ir(La147)-1 that was used directly in the next step without purification.

### Synthesis of Compound Ir(La147)₂(Lb005):

Referring to the synthesis and purification method of compound Ir(La002)₂(Lb005), only the corresponding raw materials need to be changed to obtained a red solid as compound Ir(La147)₂(Lb005) (1.94 g, yield: 43.6%). 1.94 g of crude Ir(La147)₂(Lb005) were sublimated and purified to obtain a sublimated pure Ir(La147)₂(Lb005) (1.21 g, yield: 62.37%), mass spectrometry: 1211.61 (M+H). ¹HNMR (400 MHz, CDCl₃) δ 8.88 (d, J=8.8 Hz, 2H), 8.14 (d, J=6.4 Hz, 2H), 8.03 (s, 2H), 7.60 (dd, J=5.3, 2.0 Hz, 4H), 7.55-7.43 (m, 4H), 7.37-7.29 (m, 2H), 7.19-7.05 (m, 4H), 4.83 (s, 1H), 2.68 (d, J=7.2 Hz, 4H), 2.13-2.01 (m, 8H), 1.61 (d, J=5.0 Hz, 5H), 1.39-1.04 (m, 14H), 1.01-0.84 (m, 17H), 0.41 (t, J=7.4 Hz, 8H), −0.12 (t, J=7.3 Hz, 6H).

### Synthesis of Ligand La203:

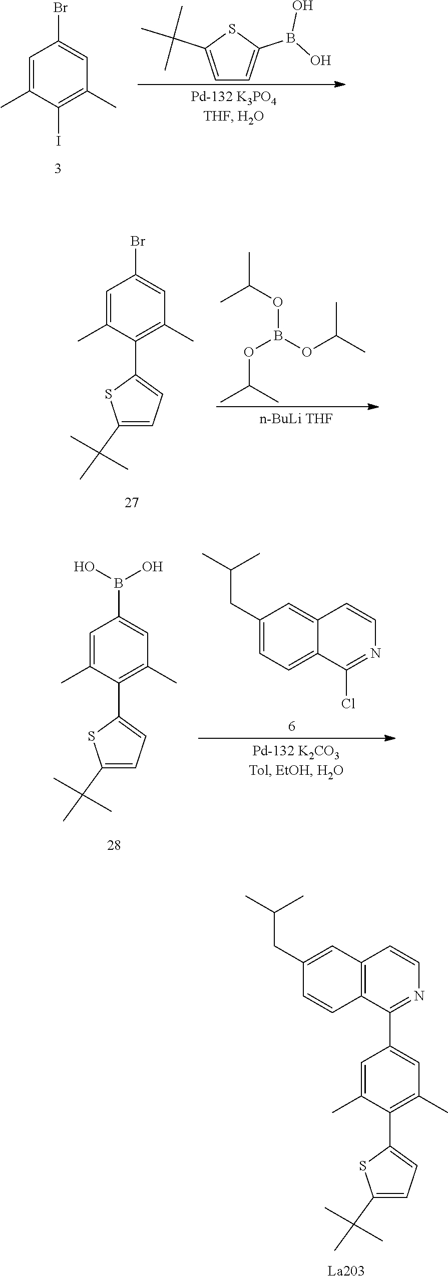

### Synthesis of Compound 27:

Referring to the synthesis and purification method of compound 4, only the corresponding raw materials needed to be changed to obtain a target compound 27, mass spectrometry: 324.29 (M+H).

### Synthesis of Compound 28:

Referring to the synthesis and purification method of compound 5, only the corresponding raw materials needed to be changed to obtain a target compound 28, mass spectrometry: 289.21 (M+H).

### Synthesis of Ligand La203:

Referring to the synthesis and purification method of ligand La002, only the corresponding raw materials needed to be changed to obtain a target ligand La203, mass spectrometry: 428.64 (M+H).

### Synthesis of Compound Ir(La203)₂(Lb005):

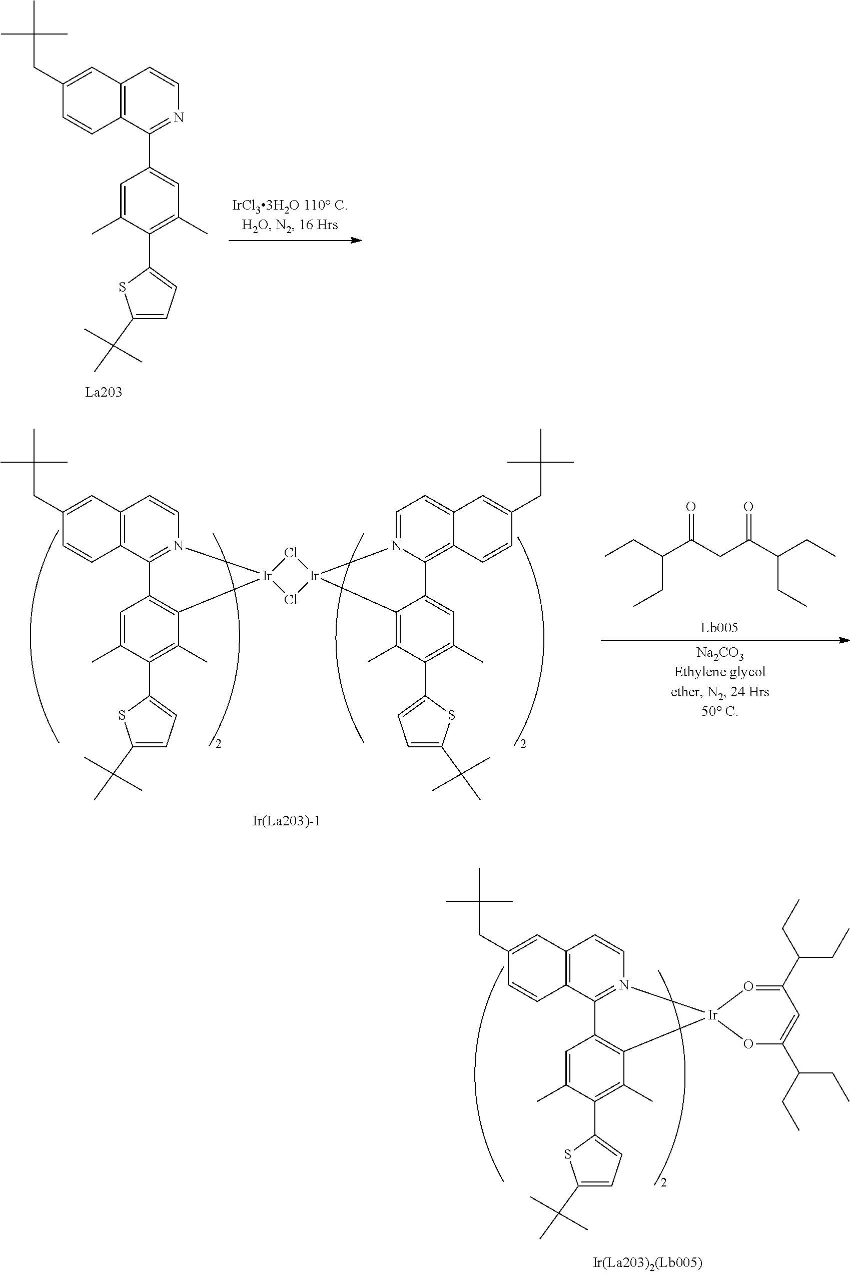

### Synthesis of Compound Ir(La203)-1:

Referring to the synthesis and purification method of compound Ir(La002)-1, only the corresponding raw materials needed to be changed to obtain compound Ir(La203)-1 that was used directly in the next step without purification.

### Synthesis of Compound Ir(La203)₂(Lb005):

Referring to the synthesis and purification method of compound Ir(La002)₂(Lb005), only the corresponding raw materials needed to be changed to a red solid as compound Ir(La203)₂(Lb005) (1.52 g, yield: 35.64%). 1.52 g of crude Ir(La203)₂(Lb005) were sublimated and purified to obtain a sublimated pure Ir(La203)₂(Lb005) (0.86 g, yield: 56.57%), mass spectrometry: 1257.81 (M+H). ¹HNMR (400 MHz, CDCl₃) δ 8.89 (d, J=8.8 Hz, 2H), 8.16 (d, J=6.4 Hz, 2H), 8.05 (s, 2H), 7.60 (d, 2H), 7.62-7.46 (m, 4H), 7.37 (d, J=6.8 Hz, 2H), 6.81 (d, J=6.4 Hz, 2H), 4.83 (s, 1H), 2.68 (d, J=7.2 Hz, 4H), 1.85-1.61 (m, 12H), 1.39-1.04 (m, 14H), 1.01-0.84 (m, 16H), 0.76-054 (m, 10H), 0.42 (t, J=7.4 Hz, 8H), −0.08 (t, J=7.3 Hz, 6H).

### Synthesis of Ligand La223:

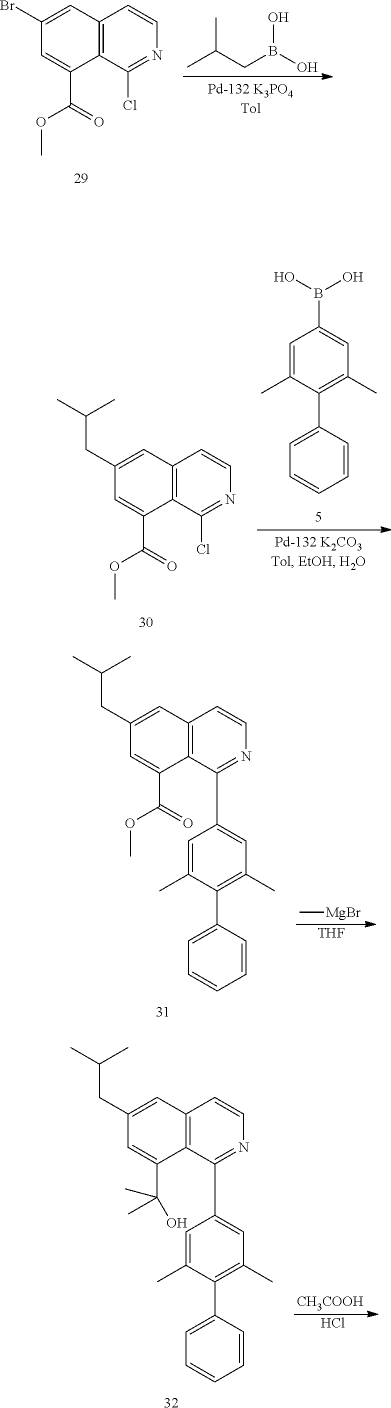

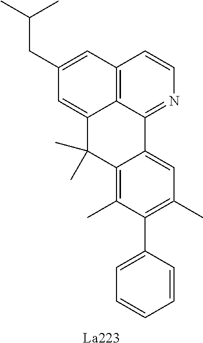 [SMILES: Cc1cc2c(c(C)c1-c1ccccc1)C(C)(C)c1cc(CC(C)C)cc3ccnc-2c13 ; "La223"]

### Synthesis of Compound 30:

Referring to the synthesis and purification method of compound 4, only the corresponding raw materials needed to be changed to obtain a target compound 30, mass spectrometry: 278.75 (M+H).

### Synthesis of Compound 31:

Referring to the synthesis and purification method of compound 5, only the corresponding raw materials needed to be changed to obtain a target compound 31, mass spectrometry: 424.55 (M+H).

### Synthesis of Compound 32:

Compound 31 (12.5 g, 29.51 mmol, 1.0 eq) and anhydrous tetrahydrofuran (125 mL) were added into a 250 mL three-necked reaction flask, and cooled to −10° C. after 3 times of vacuum and nitrogen replacement, and the reaction solution was stirred for 1 h after 2M methylmagnesium bromide (7.74 g, 64.93 mmol, 2.2 eq) were slowly dropped into the reaction solution stir for 1 h. The reaction solution was returned to room temperature for reaction for 3 h. TLC monitoring showed that compound 31 was reacted almost completely. 2M dilute hydrochloric acid (60 mL) were added with stirring to quench the reaction, and the obtained solution was separated to collect an organic phase. The organic phase was then extracted and washed twice with water (80 mL/time), and the resultant solution was separated and spin-dried to obtain the organic phase, which was purified by column chromatography purification (eluted with n-hexane:ethyl acetate=30:1) to obtain compound 32 (9.61 g, yield: 76.9%), mass spectrometry: 424.59 (M+H).

### Synthesis of Ligand La223:

Compound 32 (9.5 g, 22.43 mmol, 1.0 eq), acetic acid (100 mL), and dilute hydrochloric acid (10 mL) were added into a 250 mL three-necked reaction flask, and was heated to 100° C. for 4 h after 3 times of vacuum and nitrogen replacement. TLC monitoring showed compound 32 was reacted almost completely. Deionized water (150 mL) was added with stirring to quench the reaction, and the organic phase was then extracted with ethyl acetate (150 mL) and washed twice with water (80 mL/time), and the resultant solution was separated and spin-dried for column chromatography purification (eluted with n-hexane:ethyl acetate=40:1) to obtain ligand La223 (7.85 g, yield: 86.32%), mass spectrometry: 406.57 (M+H).

### Synthesis of Compound Ir(La223)₂(Lb005):

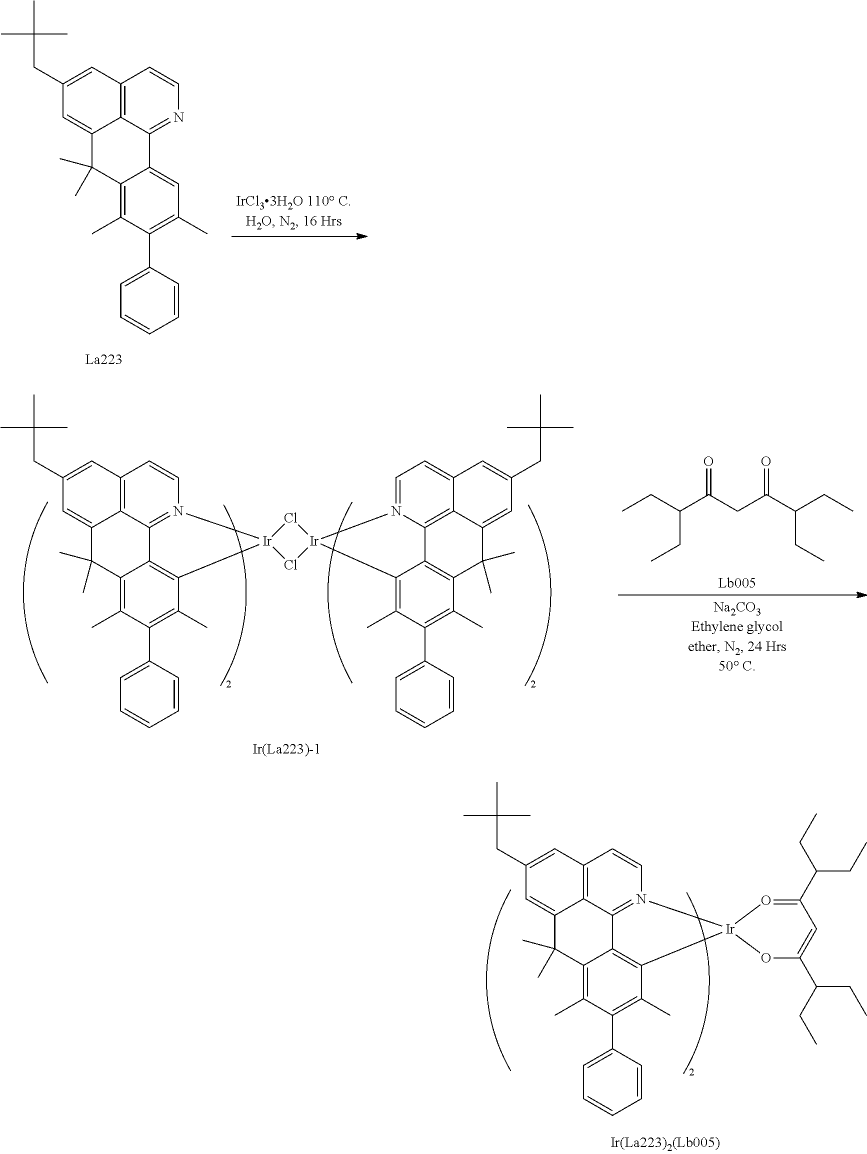

### Synthesis of Compound Ir(La223)-1:

Referring to the synthesis and purification method of compound Ir(La002)-1, only the corresponding raw materials needed to be changed to obtain compound Ir(La223)-1 that was used directly in the next step without purification.

### Synthesis of Compound Ir(La223)₂(Lb005):

Referring to the synthesis and purification method of compound Ir(La002)₂(Lb005), only the corresponding raw materials needed to be changed to obtain a red solid as compound Ir(La223)₂(Lb005) (1.71 g, yield: 44.8%). 1.71 g of crude Ir(La223)₂(Lb005) were sublimated and purified to obtain a sublimated pure Ir(La223)₂(Lb005) (1.04 g, yield: 60.81%), mass spectrometry: 1213.67 (M+H). ¹HNMR (400 MHz, CDCl₃) δ8.90 (d, J=8.8 Hz, 2H), 8.03 (s, 2H), 7.49 (s, 2H), 7.43 (d, J=9.5 Hz, 2H), 7.32 (t, J=7.6 Hz, 4H), 7.23 (t, J=7.8 Hz, 2H), 7.04 (d, J=6.4 Hz, 4H), 4.83 (s, 1H), 2.62 (d, J=7.2 Hz, 4H), 2.12-1.88 (m, 8H), 1.61 (d, J=7.5 Hz, 3H), 1.32-1.11 (m, 10H), 1.00-0.88 (m, 15H), 0.43 (t, J=7.4 Hz, 6H), −0.12 (t, J=7.3 Hz, 6H).

### Synthesis of Ligand La261:

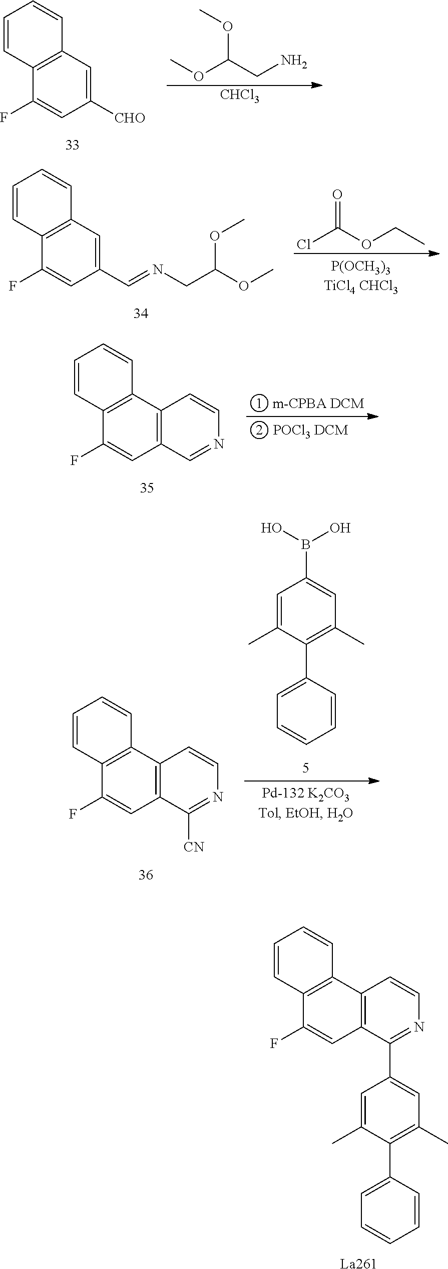

### Synthesis of Compound 34:

Compound 33 (13.5 g, 77.51 mmol, 1.0 eq) and chloroform (100 mL) into a 250 mL three-necked flask, into which aminoacetaldehyde dimethyl acetal (12.22 g, 116.26 mmol, 1.5 eq) was slowly added. After 1 h, TLC monitoring showed that compound 33 was reacted almost completely. The obtained mixture was spun-dried to remove the solvent. The resultant crude compound 34 was then subjected to the next reaction without further purification.

### Synthesis of Compound 35:

Compound 34 (28.6 g, 109.46 mmol, 1.0 eq) and chloroform (150 mL) into a 500 mL three-necked flask, into which ethyl chloroformate (11.88 g, 109.46 mmol, 1.0 eq) and trimethyl phosphite (16.3 g, 131.35 mmol, 1.2 eq) were respectively slowly added dropwise at a temperature of 0° C. After the reaction was stirred at room temperature for 16 h, 1.0 M titanium tetrachloride (83.05 g, 437.83 mmol, 4.0 eq) was slowly added dropwise at 0° C. into the reaction solution. The resulting mixture was then heated at reflux for about 16 h. TLC monitoring showed that compound 34 was reacted almost completely. The reaction was cooled to room temperature, and then ice water (200 mL) was added. The resulting solution was separated to collected an organic phase, and an aqueous layer was extracted with dichloromethane (100 mL). The organic phase was combined with the addition of tartrate solution (250 mL) to form a mixture, and the mixture was neutralized with saturated NaHCO₃ solution, and the resulting solution was separated to collect the organic phase, which was spin-dried to remove the solvent and purified by column chromatography (eluted with n-hexane:ethyl acetate=25:1) to obtain compound 35 (8.33 g, yield: 38.6%), mass spectrometry: 198.21 (M+H).

### Synthesis of Compound 36:

Referring to the synthesis and purification method of compound 18, only the corresponding raw materials needed to be changed to obtain a target compound 36, mass spectrometry: 232.65 (M+H).

### Synthesis of Ligand La261:

Referring to the synthesis and purification method of ligand La002, only the corresponding raw materials needed to be changed to obtain a target ligand La261, mass spectrometry: 378.45 (M+H).

### Synthesis of Compound Ir(La261)₂(Lb005):

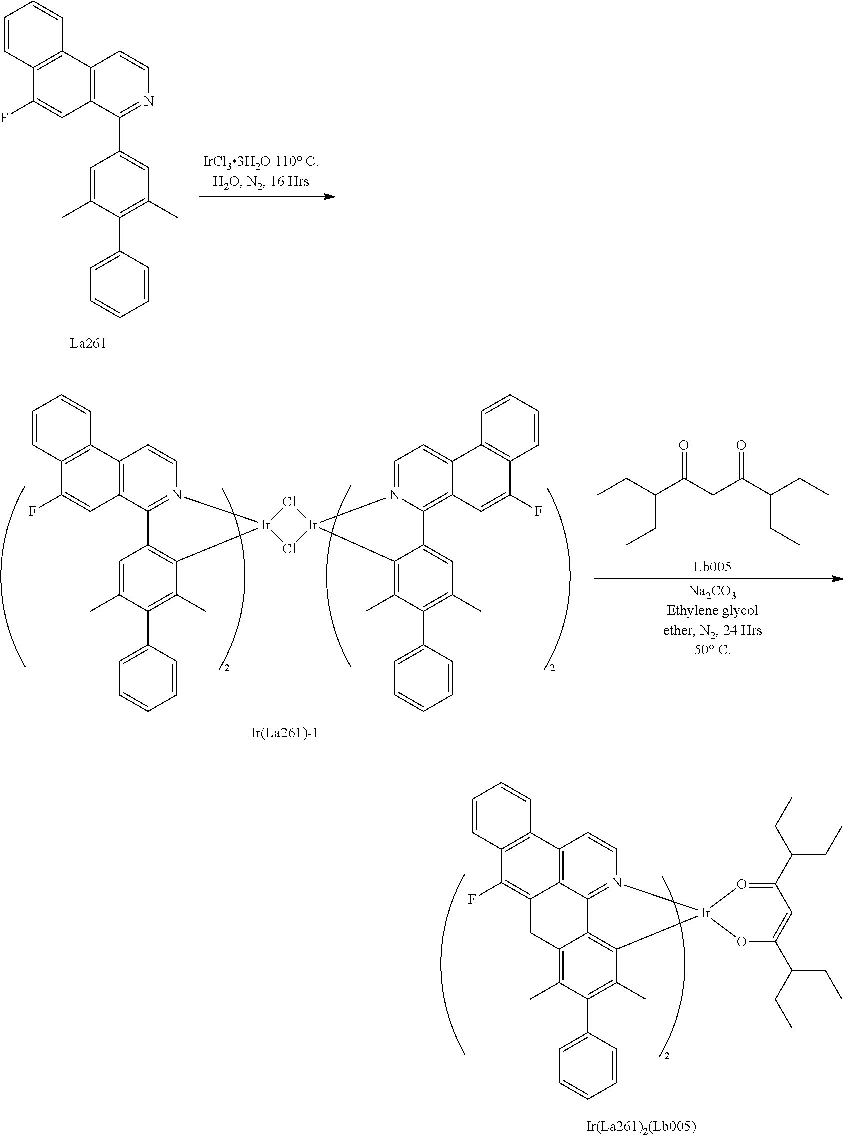

### Synthesis of Compound Ir(La261)-1:

Referring to the synthesis and purification method of compound Ir(La002)-1, only the corresponding raw materials needed to be changed to obtain compound Ir(La261)-1 that was used directly in the next step without purification.

### Synthesis of Compound Ir(La261)₂(Lb005):

Referring to the synthesis and purification method of compound Ir(La002)₂(Lb005), only the corresponding raw materials needed to be changed to obtain a red solid as compound Ir(La261)₂(Lb005) (1.82 g, yield: 36.35%). 1.82 g of crude Ir(La261)₂(Lb005) was sublimated and purified to obtain a sublimated pure Ir(La261)₂(Lb005) (0.96 g, yield: 52.74%), mass spectrometry: 1157.43 (M+H). ¹HNMR (400 MHz, CDCl₃) δ8.96 (d, J=7.8 Hz, 2H), 68.88 (d, J=8.6 Hz, 2H), 68.42 (m, 4H), 8.02 (s, 2H), 7.56 (m, 4H), 7.39 (m, 4H), 7.18 (t, J=7.8 Hz, 4H), 7.04 (d, J=6.4 Hz, 4H), 4.83 (s, 1H), 2.03-1.86 (m, 8H), 1.32-1.11 (m, 14H), 1.00-0.88 (m, 6H), 0.43 (t, J=7.4 Hz, 6H).

### Synthesis of Ligand La261:

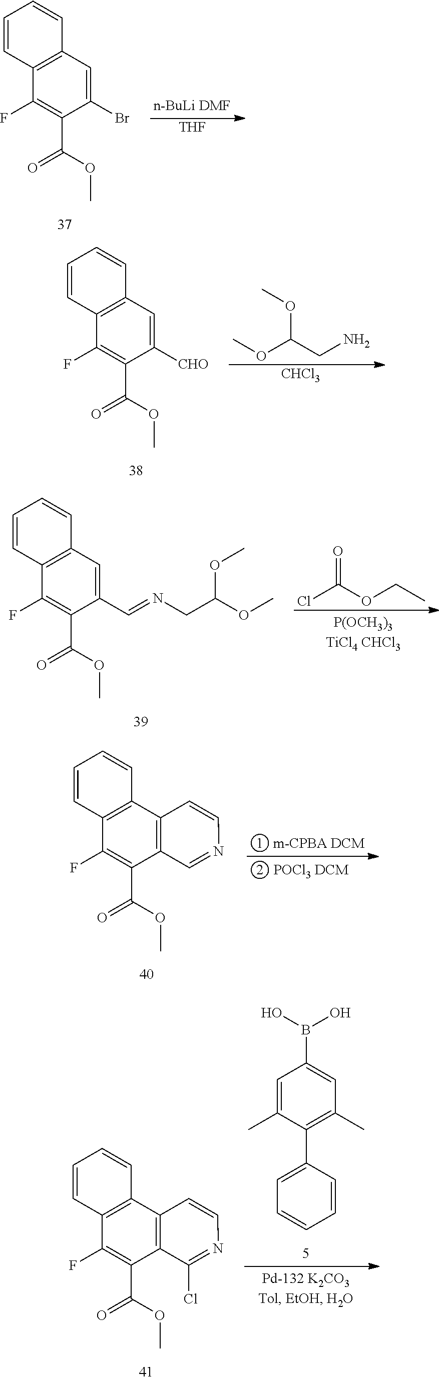

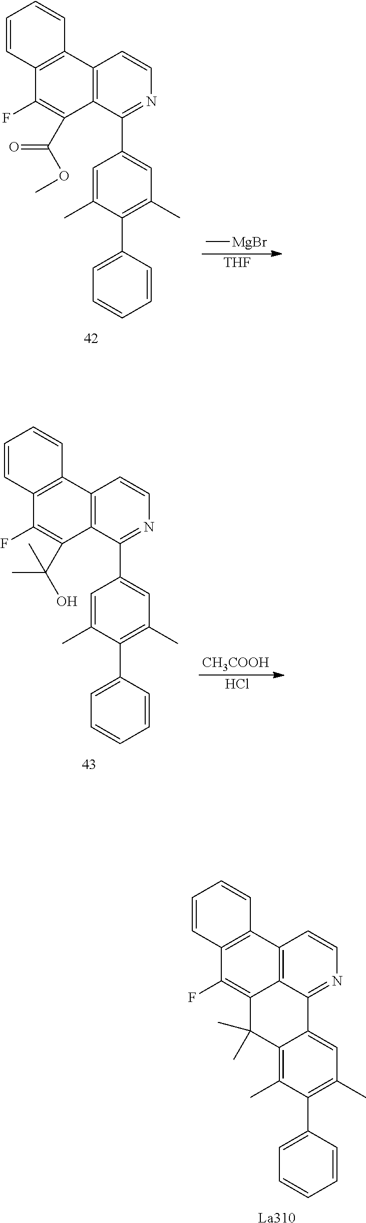

### Synthesis of Compound 38:

Compound 37 (23.5 g, 83.01 mmol, 1.0 eq) and anhydrous tetrahydrofuran (188 mL) were added into a 500 mL three-necked reaction flask, and cooled to −78° C. after three times of vacuum and nitrogen replacement. The reaction solution was stirred for 1 h after 2M n-butyllithium (49.81 mL, 99.61 mmol, 1.2 eq) were slowly added into the reaction solution. Then, after anhydrous DMF (12.14 g, 166.02 mmol, 2.0 eq) was slowly added, the solution was stirred for 0.5 h and returned to room temperature for reaction for 2 h. TLC monitoring showed that compound 37 was reacted almost completely. 2M dilute hydrochloric acid (80 mL) were added for stirring to quench the reaction, and the solution was separated to collect an organic phase. The organic phase was then extracted and washed twice with water (100 mL/time), and separated and spin-dried for column chromatography purification (eluted with n-hexane:ethyl acetate=15:1) to obtain compound 38 (10.77 g, yield: 55.87%), mass spectrometry: 233.21 (M+H).

### Synthesis of Compound 39:

Referring to the synthesis and purification method of compound 34, only the corresponding raw materials needed to be changed to obtain a target compound 39, mass spectrometry: 320.33 (M+H).

### Synthesis of Compound 40:

Referring to the synthesis and purification method of compound 35, only the corresponding raw materials needed to be changed to obtain a target compound 40, mass spectrometry: 256.24 (M+H).

### Synthesis of Compound 41:

Referring to the synthesis and purification method of compound 18, only the corresponding raw materials needed to be changed to obtain a target compound 41, mass spectrometry: 290.69 (M+H).

### Synthesis of Compound 42:

Referring to the synthesis and purification method of ligand La002, only the corresponding raw materials needed to be changed to obtain a target compound 42, mass spectrometry: 436.49 (M+H).

### Synthesis of Compound 43:

Referring to the synthesis and purification method of compound 32, only the corresponding raw materials needed to be changed to obtain a target compound 43, mass spectrometry: 436.53 (M+H).

### Synthesis of Ligand La261:

Referring to the synthesis and purification method of ligand La223, only the corresponding raw materials needed to be changed to obtain a target ligand La261, mass spectrometry: 418.52 (M+H).

### Synthesis of Compound Ir(La310)₂(Lb005):

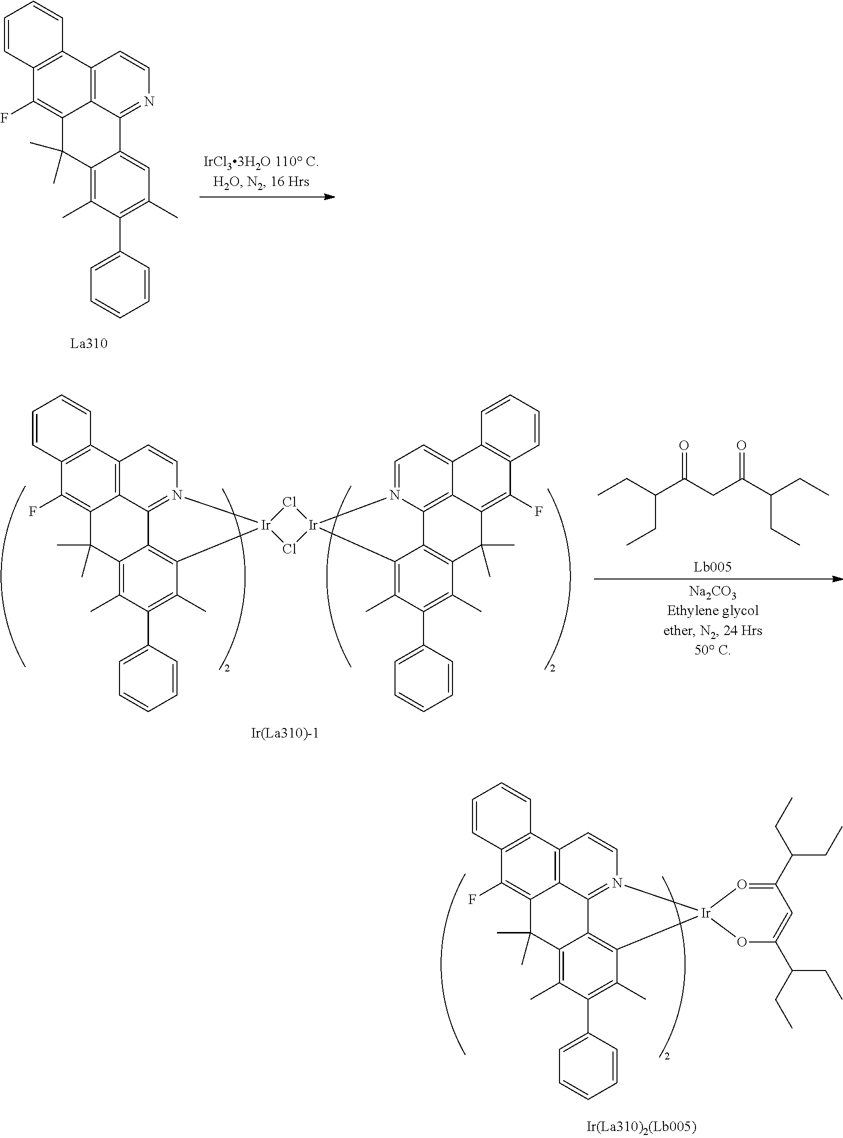

### Synthesis of Compound Ir(La310)-1:

Referring to the synthesis and purification method of compound Ir(La002)-1, only the corresponding raw materials needed to be changed to obtain compound Ir(La310)-1 that was used directly in the next step without purification.

### Synthesis of Compound Ir(La310)₂(Lb005):

Referring to the synthesis and purification method of compound Ir(La002)₂(Lb005), only the corresponding raw materials needed to be changed to obtain a red solid as compound Ir(La310)₂(Lb005) (1.47 g, yield: 36.35%). 1.47 g of crude Ir(La310)₂(Lb005) were sublimated and purified to obtain a sublimated pure Ir(La310)₂(Lb005) (0.84 g, yield: 57.14%), mass spectrometry: 1237.55 (M+H). ¹HNMR (400 MHz, CDCl₃) δ8.96 (d, J=7.8 Hz, 2H), 88.88 (d, J=8.6 Hz, 2H), δ8.34 (m, 2H), 8.06 (s, 2H), 7.42 (m, 4H), 7.32 (m, 2H), 7.16 (t, J=7.8 Hz, 4H), 7.05 (d, J=6.4 Hz, 4H), 4.83 (s, 1H), 2.03-1.86 (m, 8H), 1.29-1.08 (m, 14H), 1.00-0.88 (m, 6H), 0.76-0.58 (m, 12H), 0.26 (t, J=7.4 Hz, 6H).

The corresponding materials were selected, and similar methods could be used to synthesize and sublimate other compounds.

### Application Examples: Fabrication of an Organic Electroluminescent Devices

A 50 mm*50 mm*1.0 mm glass substrate with an ITO (100 nm) anode electrode was ultrasonically cleaned in ethanol for 10 min, dried at 150° C. and then treated with N₂ Plasma for 30 min. The washed glass substrate was installed on a substrate holder of a vacuum evaporation device. Firstly, compounds HTM1 and P-dopant (a ratio of 97%:3%) were evaporated in a co-evaporation mode by covering the electrode on a surface with anode electrode wires to form a thin film with a thickness of 10 nm, and then a layer of HTM1 was evaporated thereon to form a thin film with a thickness of about 60 nm, and a layer of HTM2 was further evaporated on the HTM1 thin film to form a film with a thickness of 10 nm, and then, host material 1, host material 2 and a doping compound were evaporated on the layer of the HTM2 thin film in the co-evaporation mode (a ratio of 48.5%: 48.5%: 3%, Comparative compound X, the compound of the present disclosure) to form a thin film with a thickness of 40 nm. ETL:LiQ (35 nm, 50%:50%) were evaporated on the light-emitting layer in the co-evaporation mode, followed by a layer of Yb (1 nm) being evaporated on the electron transport layer material, and finally a layer of metal Ag (15 nm) was evaporated as the electrode.

| | | | | | An electron |
|---|---|---|---|---|---|
| Example | HIL | HTL | EBL | An emission layer | transport layer |
| A1 | HTM1:NDP-9 | HTM1 | HTM2 | H1:H2:Ir(La002)₂(Lb005) | ETL:QQ |
| A2 | HTM1:NDP-9 | HTM1 | HTM2 | H1:H2:Ir(La003)₂(Lb005) | ETL:QQ |
| A3 | HTM1:NDP-9 | HTM1 | HTM2 | H1:H2:Ir(La004)₂(Lb005) | ETL:QQ |
| A4 | HTM1:NDP-9 | HTM1 | HTM2 | H1:H2:Ir(La005)₂(Lb005) | ETL:QQ |
| A5 | HTM1:NDP-9 | HTM1 | HTM2 | H1:H2:Ir(La007)₂(Lb005) | ETL:QQ |
| A6 | HTM1:NDP-9 | HTM1 | HTM2 | H1:H2:Ir(La010)₂(Lb005) | ETL:QQ |
| A7 | HTM1:NDP-9 | HTM1 | HTM2 | H1:H2:Ir(La017)₂(Lb005) | ETL:QQ |
| A8 | HTM1:NDP-9 | HTM1 | HTM2 | H1:H2:Ir(La023)₂(Lb005) | ETL:QQ |
| A9 | HTM1:NDP-9 | HTM1 | HTM2 | H1:H2:Ir(La043)₂(Lb005) | ETL:QQ |
| A10 | HTM1:NDP-9 | HTM1 | HTM2 | H1:H2:Ir(La083)₂(Lb005) | ETL:QQ |
| A11 | HTM1:NDP-9 | HTM1 | HTM2 | H1:H2:Ir(La107)₂(Lb005) | ETL:QQ |
| A12 | HTM1:NDP-9 | HTM1 | HTM2 | H1:H2:Ir(La123)₂(Lb005) | ETL:QQ |
| A13 | HTM1:NDP-9 | HTM1 | HTM2 | H1:H2:Ir(La127)₂(Lb005) | ETL:QQ |
| A14 | HTM1:NDP-9 | HTM1 | HTM2 | H1:H2:Ir(La143)₂(Lb005) | ETL:QQ |
| A15 | HTM1:NDP-9 | HTM1 | HTM2 | H1:H2:Ir(La147)₂(Lb005) | ETL:QQ |
| A16 | HTM1:NDP-9 | HTM1 | HTM2 | H1:H2:Ir(La203)₂(Lb005) | ETL:QQ |
| A17 | HTM1:NDP-9 | HTM1 | HTM2 | H1:H2:Ir(La223)₂(Lb005) | ETL:QQ |
| A18 | HTM1:NDP-9 | HTM1 | HTM2 | H1:H2:Ir(La261)₂(Lb005) | ETL:QQ |
| A18 | HTM1:NDP-9 | HTM1 | HTM2 | H1:H2:Ir(La310)₂(Lb005) | ETL:QQ |
| Comparative | HTM1:NDP-9 | HTM1 | HTM2 | H1:H2:Comparative | ETL:QQ |
| example 1 | | | | compound 1 | |
| Comparative | HTM1:NDP-9 | HTM1 | HTM2 | H1:H2:Comparative | ETL:QQ |
| example 2 | | | | compound 2 | |
| Comparative | HTM1:NDP-9 | HTM1 | HTM2 | H1:H2:Comparative | ETL:QQ |
| example 3 | | | | compound 3 | |
| Comparative | HTM1:NDP-9 | HTM1 | HTM2 | H1:H2:Comparative | ETL:QQ |
| example 4 | | | | compound 4 | |
| Comparative | HTM1:NDP-9 | HTM1 | HTM2 | H1:H2:Comparative | ETL:QQ |
| example 5 | | | | compound 5 | |
| Comparative | HTM1:NDP-9 | HTM1 | HTM2 | H1:H2:Comparative | ETL:QQ |
| example 6 | | | | compound 6 | |

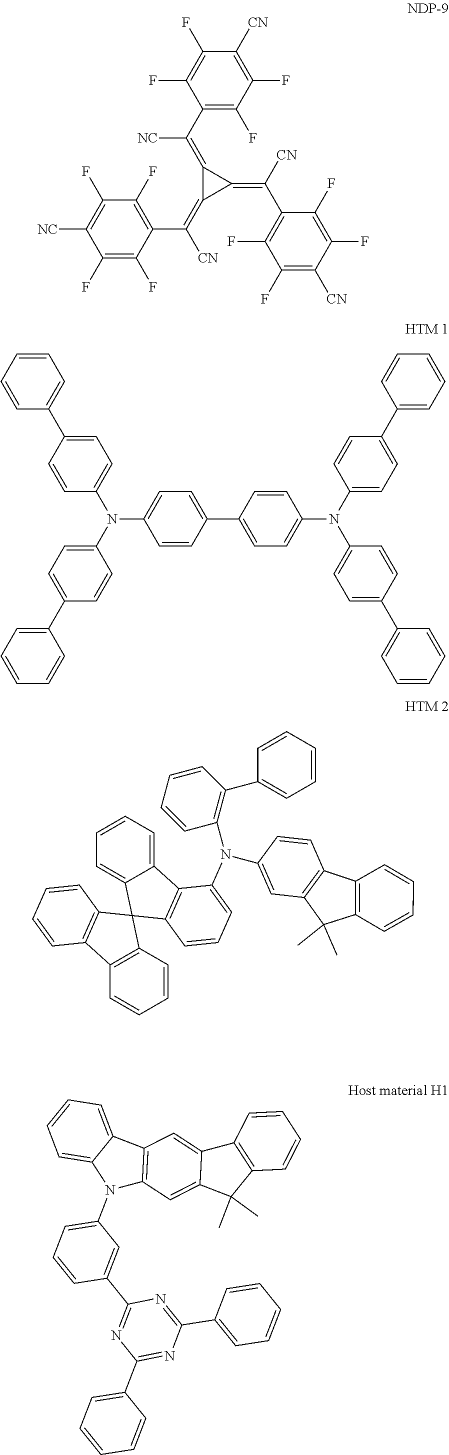 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; CC1(C)c2ccccc2-c2ccc(N(c3ccccc3-c3ccccc3)c3cccc4c3-c3ccccc3C43c4ccccc4-c4ccccc43)cc21 ; CC1(C)c2ccccc2-c2cc3c4ccccc4n(-c4cccc(-c5nc(-c6ccccc6)nc(-c6ccccc6)n5)c4)c3cc21]

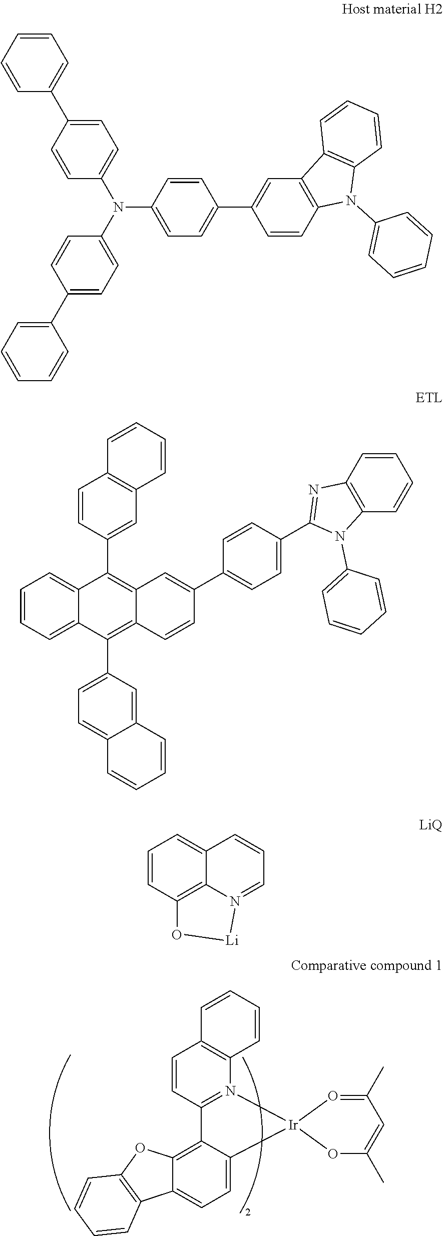

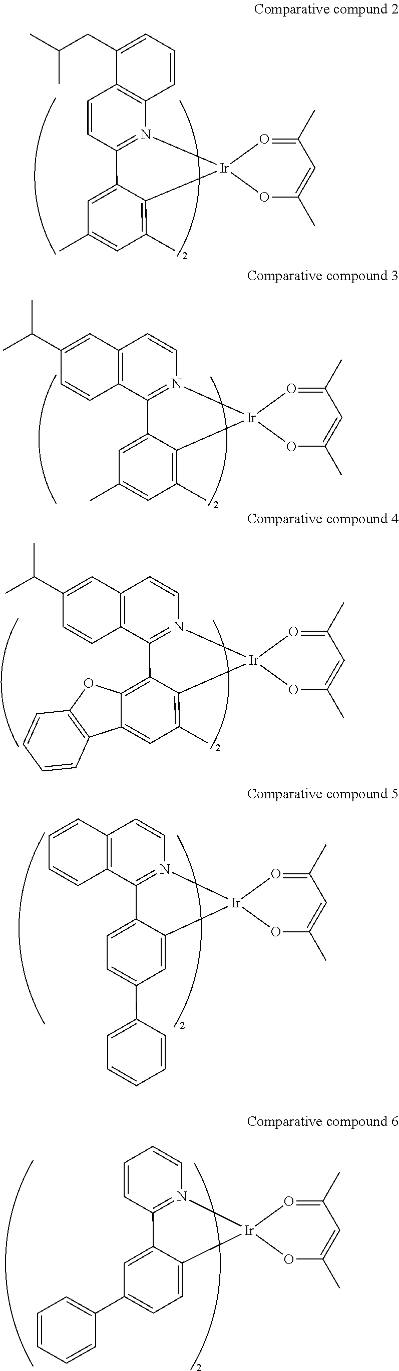

Evaluation: The above devices were tested for device performance. In each example and comparative example, a constant current power supply (Keithley 2400) was used, a fixed current density was used to flow through the light-emitting elements, and the luminescence spectrum was measured using a spectroradiometer (CS 2000). In addition, the voltage value and the time when the test brightness was 95% of the initial brightness (LT95) were measured simultaneously. The results were provided follows: the current efficiency and device lifetime were calculated based on the value of Comparative Compound 4 as 100%.

| | Starting | | | |
|---|---|---|---|---|
| | voltage | Current | Peak | LT95@ |
| | @20 mA/cm² | efficiency | wavelength/ | 5000 |
| | V | @20 mA/cm² | nm | nits |
| Example A1 | 3.98 | 117 | 618 | 136 |
| Example A2 | 3.97 | 124 | 619 | 141 |
| Example A3 | 3.95 | 123 | 619 | 163 |
| Example A4 | 4.03 | 127 | 618 | 133 |
| Example A5 | 4.06 | 132 | 620 | 125 |
| Example A6 | 4.01 | 136 | 619 | 138 |
| Example A7 | 4.12 | 122 | 624 | 134 |
| Example A8 | 4.07 | 136 | 620 | 143 |
| Example A9 | 4.02 | 137 | 620 | 151 |
| Example A10 | 3.93 | 124 | 620 | 131 |
| Example A11 | 4.02 | 135 | 619 | 142 |
| Example A12 | 3.90 | 122 | 617 | 139 |
| Example A13 | 3.92 | 128 | 618 | 128 |
| Example A14 | 3.88 | 132 | 617 | 139 |
| Example A15 | 3.90 | 127 | 618 | 134 |
| Example A16 | 4.03 | 135 | 619 | 124 |
| Example A17 | 4.05 | 141 | 619 | 163 |
| Example A18 | 4.01 | 138 | 620 | 151 |
| Example A18 | 4.04 | 139 | 619 | 143 |
| Comparative | 4.87 | 75 | 610 | 82 |
| example 1 | | | | |
| Comparative | 4.91 | 72 | 608 | 89 |
| example 2 | | | | |
| Comparative | 4.78 | 82 | 622 | 81 |
| example 3 | | | | |
| Comparative | 4.88 | 100 | 616 | 100 |
| example 4 | | | | |
| Comparative | 5.01 | 84 | 624 | 73 |
| example 5 | | | | |
| Comparative | 4.92 | 79 | 618 | 77 |
| example 6 | | | | |

From the comparison of the data in the above table, it can be seen that the organic electroluminescent device in which the compounds of the present disclosure were used dopants exhibited better performance in terms of driving voltage, luminous efficiency and device lifetime than the comparative compounds in devices with the same color scale.

Sublimation temperature comparison: the sublimation temperature was defined as a temperature at which the evaporation rate was 1 angstrom per second at a vacuum degree of 10⁻⁷ Torr. The test results are as follows:

| Material | Sublimation temperature |
|---|---|
| Ir(La003)₂(Lb005) | 269 |
| Ir(La007)₂(Lb005) | 271 |
| Ir(La083)₂(Lb005) | 272 |
| Ir(La123)₂(Lb005) | 267 |
| Ir(La143)₂(Lb005) | 273 |
| Ir(La203)₂(Lb005) | 272 |
| Ir(La223)₂(Lb005) | 275 |
| Comparative compound 1 | 280 |
| Comparative compound 2 | 288 |
| Comparative compound 3 | 276 |
| Comparative compound 4 | 268 |
| Comparative compound 5 | 287 |
| Comparative compound 5 | 279 |

It can be seen from the comparison of the data in the above table that the metallic iridium complex of the present disclosure had a lower sublimation temperature, which was beneficial to industrial application.

Through the special combination of substituents, the present disclosure unexpectedly provides low driving voltage, a better device luminous efficiency and an improved lifetime compared to the existing technology, and provides low sublimation temperature and high saturated red-emitting. The above results show that the compounds of the present disclosure have the advantages of lower sublimation temperature, good optical and electrochemical stability, high color saturation, high luminous efficiency, long device lifetime, and the like, can be used in organic light-emitting devices, and especially as a red luminous dopant, and has the possibility of being applied to the OLED industry.

1. A metal iridium complex having a general formula Ir(La)(Lb)(Lc), wherein La is a structure of formula (1), 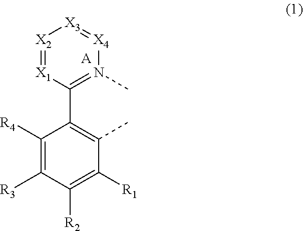
wherein dotted lines indicate a position connected to a metal Ir;
wherein X₁-X₄ are independently N or CR₀, and at least two of the X₁-X₄ are CR₀, and two R₀ are connected to each other to form a five- or six-membered substituted or unsubstituted aromatic or heteroaromatic ring;
wherein R₀ and R₄ are independently selected from hydrogen, deuterium, halogen, cyano, a substituted or unsubstituted C1-C10 alkyl group, a substituted or unsubstituted C1-C10 heteroalkyl group, a substituted or unsubstituted C3-C20 cycloalkyl group, a substituted or unsubstituted C3-C20 heterocycloalkyl group, a substituted or unsubstituted C2-C10 alkenyl group, a substituted or unsubstituted C2-C10 alkynyl group, a substituted or unsubstituted C6-C30 aryl group, a substituted or unsubstituted C2-C30 heteroaryl group, a substituted or unsubstituted (tri-C1-C10 alkyl)silyl group, a substituted or unsubstituted (tri-C6-C12 aryl)silyl group, a substituted or unsubstituted (di-C1-C10 alkyl)(mono-C6-C30 aryl)silyl group, a substituted or unsubstituted (mono-C1-C10 alkyl)(di-C6-C30 aryl)silyl group, amino group, thiol group, or hydroxyl group;
wherein R₁ and R₃ are independently selected from a substituted or unsubstituted C1-C10 alkyl group, a substituted or unsubstituted C1-C10 heteroalkyl group, a substituted or unsubstituted C3-C20 cycloalkyl group, or a substituted or unsubstituted C3-C20 heterocycloalkyl group;
wherein R₂ is a substituted or unsubstituted C6-C30 aryl group, or a substituted or unsubstituted C2-C30 heteroaryl group;
wherein Lb is a structure of formula (2), 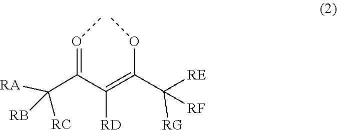
wherein dotted lines indicate a position connected to the metal Ir;
wherein RA-RG are independently selected from hydrogen, deuterium, halogen, a substituted or unsubstituted C1-C10 alkyl group, a substituted or unsubstituted C3-C20 cycloalkyl group, a substituted or unsubstituted C1-C10 heteroalkyl group, or a substituted or unsubstituted C3-C20 heterocycloalkyl group, or RA, RB, and RC are connected in pairs to form an aliphatic ring, and RE, RF, and RG are connected in pairs to form an aliphatic ring;
wherein the heteroaromatic ring, the heteroalkyl group, the heterocycloalkyl group and the heteroaryl group contain at least one 0, N or S heteroatom;
wherein the substitution is a substitution by deuterium, F, Cl, Br, C1-C6 alkyl group, C1-C6 alkoxy group, C3-C6 cycloalkyl group, amino group substituted by C1-C6 alkyl group, cyano, isocyano group or phosphino, wherein the substitution is from a monosubstitution to a maximum substitution;
wherein Lc is a monoanionic bidentate ligand; Lc is different from Lb and not OO -type ligand;
wherein Lc and La are the same or different, and the difference is that a parent core structure is different; or substituents are different; or positions of the substituents are different;
or, two or three of La, Lb, and Lc are connected to each other to form a multidentate ligand.

2. The metal iridium complex according to claim 1, wherein the aromatic ring structure or the heteroaromatic ring structure is an aromatic ring or heteroaromatic ring in which X₁ and X₂ are CR₀, and two R₀ are connected to each other to form a five-membered ring or a six-membered ring; or the aromatic ring structure or the heteroaromatic ring structure is an aromatic ring or heteroaromatic ring in which X₂ and X₃ are CR₀, and two R₀ are connected to each other to form a five-membered ring or a six-membered ring: the aromatic ring structure or the heteroaromatic ring structure is an aromatic ring or heteroaromatic ring in which X₃ and X₄ each CR₀, and two R₀ are connected to each other to form a five-membered ring or a six-membered ring;
wherein the aromatic ring structure or the heteroaromatic ring structure of the five-membered ring or six-membered ring is fused with an A ring to form a fused ring structure, and the fused ring structure is represented by one of the following formulas: 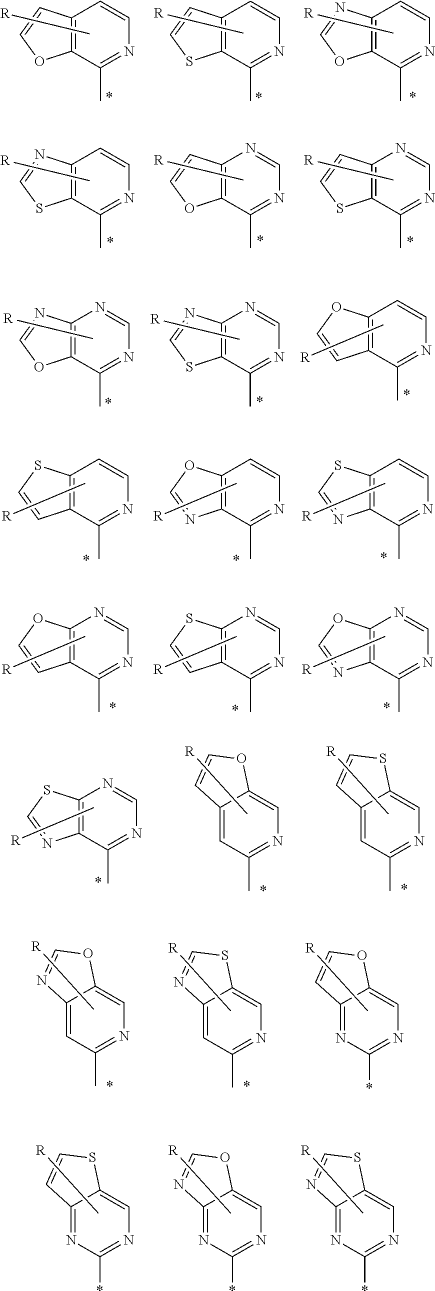 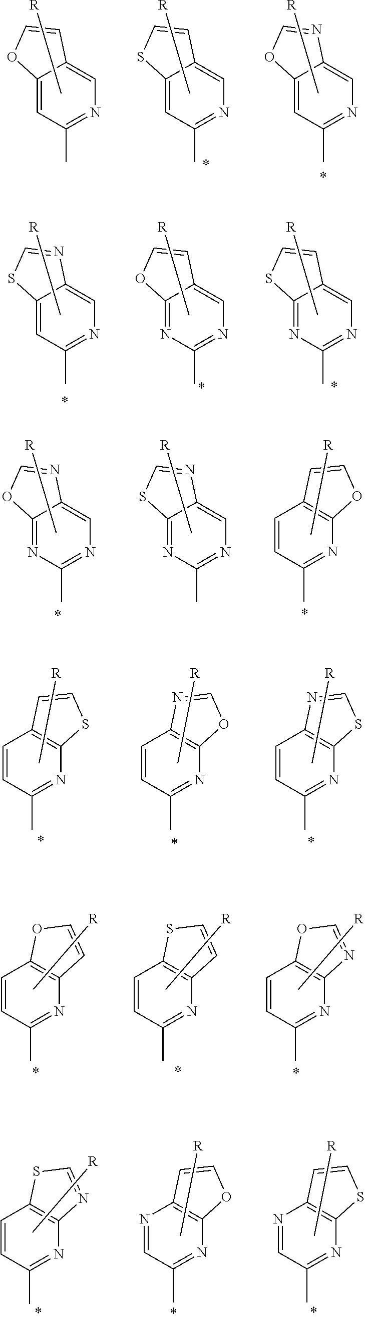 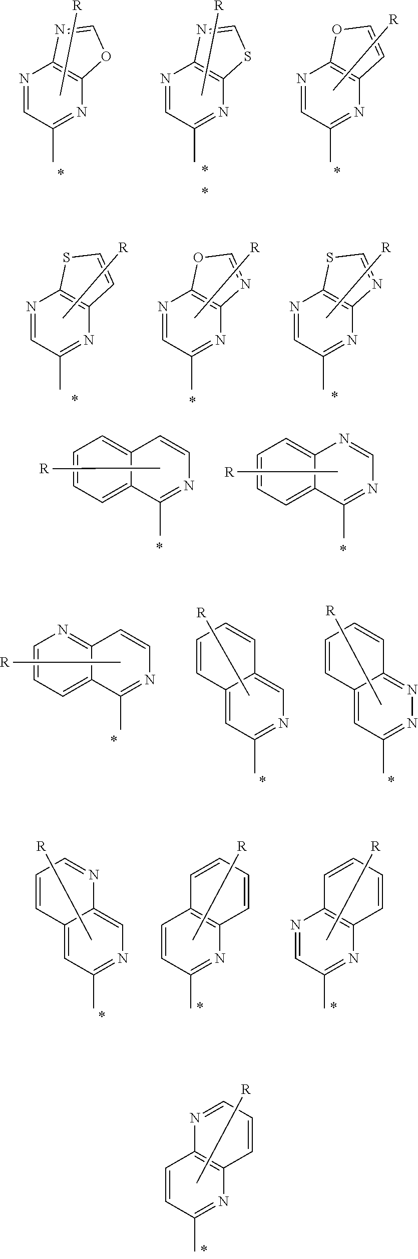
wherein * represents a position connected to a R₁-R₄-substituted benzene ring in formula (1);
wherein R represents no substitution to a maximum possible substitution, or two adjacent R are connected to each other to form an aliphatic or aromatic ring structure, and wherein no substitution means that the positions connected to carbon are all hydrogen atoms, and R is independently selected from deuterium, halogen, cyano, a substituted or unsubstituted C1-C10 alkyl group, a substituted or unsubstituted C1-C10 heteroalkyl group, a substituted or unsubstituted C3-C20 cycloalkyl group, a substituted or unsubstituted C3-C20 heterocycloalkyl group, a substituted or unsubstituted C2-C10 alkenyl group, a substituted or unsubstituted C2-C10 alkynyl group, a substituted or unsubstituted C6-C30 aryl group, a substituted or unsubstituted C2-C30 heteroaryl group, a substituted or unsubstituted (tri-C1-C10 alkyl)silyl group, a substituted or unsubstituted (tri-C6-C12 aryl)silyl group, a substituted or unsubstituted (di-C1-C10 alkyl)(mono-C6-C30 aryl)silyl group, a substituted or unsubstituted (mono-C1-C10 alkyl)(di-C6-C30 aryl)silyl group, amino group, thiol group, or hydroxyl group.

3. The metal iridium complex according to claim 2, wherein a structure of formula (3) is formed by interconnection between two adjacent R₀ in formula (1); 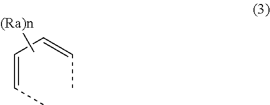
wherein dotted lines indicate a site connected to the A ring, the structure of formula (3) is formed a fused ring structure with the A ring;
wherein Ra is selected from hydrogen, deuterium, halogen, cyano, a substituted or unsubstituted C1-C10 alkyl group, a substituted or unsubstituted C1-C10 heteroalkyl group, a substituted or unsubstituted C3-C20 cycloalkyl group, a substituted or unsubstituted C3-C20 heterocycloalkyl group, a substituted or unsubstituted C2-C10 alkenyl group, a substituted or unsubstituted C2-C10 alkynyl group, a substituted or unsubstituted C6-C30 aryl group, a substituted or unsubstituted C2-C30 heteroaryl group, a substituted or unsubstituted (tri-C1-C10 alkyl)silyl group, a substituted or unsubstituted (tri-C6-C12 aryl)silyl group, a substituted or unsubstituted (di-C1-C10 alkyl)(mono-C6-C30 aryl)silyl group, or a substituted or unsubstituted (mono-C1-C10 alkyl)(di-C6-C30 aryl)silyl group, or two adjacent Ra are connected to each other to form an alicyclic ring or aromatic ring;
wherein n is a natural number from 0 to 4.

4. The metal iridium complex according to claim 3, wherein formula (1) has a structure of formula (4): 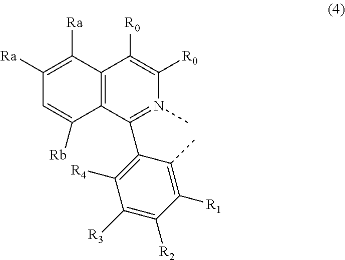
wherein Rb is H, deuterium, halogen, cyano, a substituted or unsubstituted C1-C10 alkyl group, a substituted or unsubstituted C1-C10 heteroalkyl group, a substituted or unsubstituted C3-C20 cycloalkyl group, or a substituted or unsubstituted C3-C20 heterocycloalkyl group; and
wherein at least one of Ra is not H.

5. The metal iridium complex according to claim 4, having a structure of formula (5): 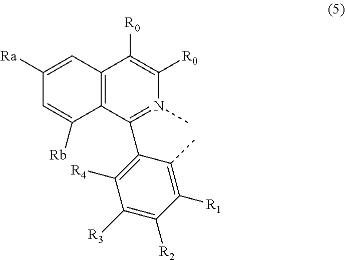
wherein Ra is selected from a substituted or unsubstituted C1-C10 alkyl group, a substituted or unsubstituted C1-C10 heteroalkyl group, a substituted or unsubstituted C3-C20 cycloalkyl group, a substituted or unsubstituted C3-C20 heterocycloalkyl group, a substituted or unsubstituted C2-C10 alkenyl group, a substituted or unsubstituted C2-C10 alkynyl group, a substituted or unsubstituted C6-C30 aryl group, or a substituted or unsubstituted C2-C30 heteroaryl group; and wherein R₂ is a substituted or unsubstituted C6-C12 aryl group, or a substituted or unsubstituted C4-C12 heteroaryl group.

6. The metal iridium complex according to claim 5, wherein R₂ is a substituted or unsubstituted phenyl group, or a substituted or unsubstituted C4 heteroaryl group.

7. The metal iridium complex according to claim 6, wherein the substitution in R₂ is the substitution by a group containing deuterium; F; CN; C1-C6 alkyl; a C3-C6 cycloalkyl group containing deuterium, F, CN or C1-C6 alkyl; or a deuterium or F substituted C1-C6 alkyl.

8. The metal iridium complex according to claim 7, wherein R₁ and R₃ are a deuterium or F substituted or unsubstituted C1-C6 alkyl group, or a deuterium or F substituted or unsubstituted C3-C6 cycloalkyl group.

9. The metal iridium complex according to claim 8, wherein R₄ is hydrogen, deuterium, F, CN, or a substituted or unsubstituted C1-C4 alkyl group.

10. The metal iridium complex according to claim 3, wherein R₄ and one of Ra are connected to each other to form a structure of formula (6): 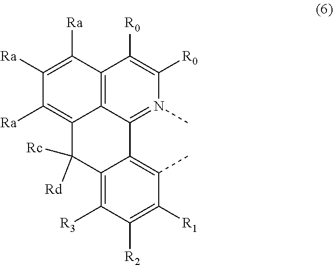
wherein Rc and Rd are H, deuterium, F, CN, or a substituted or unsubstituted C1-C4 alkyl group, wherein the substitution is the substitution by deuterium, F or a C1-C4 alkyl group; and at least one of Ra is not H.

11. The metal iridium complex according to claim 10, having s structure of formula (7): 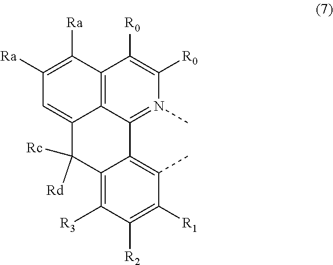
wherein at least one of Ra is not H.

12. The metal iridium complex according to claim 1, wherein Lc and La are different.

13. The metal iridium complex according to claim 12, wherein Lc is a structure of formula (8), 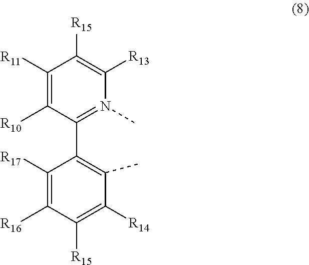
wherein dotted lines indicate a position connected to the metal Ir;
wherein R₁₀-R₁₃ are independently selected from hydrogen, deuterium, halogen, cyano, hydroxyl group, amino group, amine group, a substituted or unsubstituted C1-C10 alkyl group, a substituted or unsubstituted C1-C10 heteroalkyl group, a substituted or unsubstituted C3-C20 cycloalkyl group, a substituted or unsubstituted C2-C10 alkenyl group, a substituted or unsubstituted C2-C10 alkynyl group, a substituted or unsubstituted C6-C18 aryl group, a substituted or unsubstituted C2-C17 heteroaryl group, a substituted or unsubstituted (tri-C1-C10 alkyl)silyl group, a substituted or unsubstituted (tri-C6-C12 aryl)silyl group, a substituted or unsubstituted (di-C1-C10 alkyl)(mono-C6-C30 aryl)silyl group, or a substituted or unsubstituted (mono-C1-C10 alkyl)(di-C6-C30 aryl)silyl group; or
at least one group of two adjacent groups in R₁₀-R₁₃ forms an aromatic ring as represented by the formula (9); 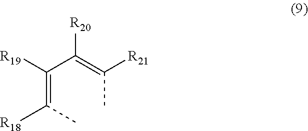
wherein dotted lines in the formula (9) indicate a position connected to a pyridine ring;
wherein R₁₈-R₂₁ are independently selected from hydrogen, deuterium, halogen, cyano, a substituted or unsubstituted C1-C10 alkyl group, a substituted or unsubstituted C1-C10 heteroalkyl group, a substituted or unsubstituted C3-C20 cycloalkyl group, a substituted or unsubstituted C2-C10 alkenyl group, a substituted or unsubstituted C2-C10 alkynyl group, a substituted or unsubstituted C6-C18 aryl group, a substituted or unsubstituted C2-C17 heteroaryl group, a substituted or unsubstituted (tri-C1-C10 alkyl)silyl group, a substituted or unsubstituted (tri-C6-C12 aryl)silyl group, a substituted or unsubstituted (di-C1-C10 alkyl)(mono-C6-C30 aryl)silyl group, and a substituted or an unsubstituted (mono-C1-C10 alkyl)(di-C6-C30 aryl)silyl group, or two adjacent groups of R₁₈-R₂₁ are connected to each other to form an alicyclic ring or aromatic ring;
wherein R₁₄-R₁₇ are independently selected from hydrogen, deuterium, halogen, cyano, hydroxyl group, amino group, amine group, a substituted or unsubstituted C1-C10 alkyl group, a substituted or unsubstituted C1-C10 heteroalkyl group, a substituted or unsubstituted C3-C20 cycloalkyl group, a substituted or unsubstituted C2-C10 alkenyl group, a substituted or unsubstituted C2-C10 alkynyl group, a substituted or unsubstituted C6-C18 aryl group, a substituted or unsubstituted C2-C17 heteroaryl group, a substituted or unsubstituted (tri-C1-C10 alkyl)silyl group, a substituted or unsubstituted (tri-C6-C12 aryl)silyl group, a substituted or unsubstituted (di-C1-C10 alkyl)(mono-C6-C30 aryl)silyl group, or a substituted or unsubstituted (mono-C1-C10 alkyl)(di-C6-C30 aryl)silyl group; and at least two of R₁₄-R₁₇ are not hydrogen;
wherein the heteroalkyl group and the heteroaryl group contain at least one O, N or S heteroatom; and
wherein the substitution is the substitution by deuterium, F, Cl, Br, a C1-C6 alkyl group, a C3-C6 cycloalkyl group, amino group substituted by a C1-C6 alkyl group, cyano, isocyano group or phosphino; and wherein the substitution is a monosubstitution to a maximum substitution.

14. The metal iridium complex according to claim 13, wherein Lc is one of the following structural formulas, or is one of the following structural formulas in which hydrogen is partially or completely replaced by deuterium or fluorine,

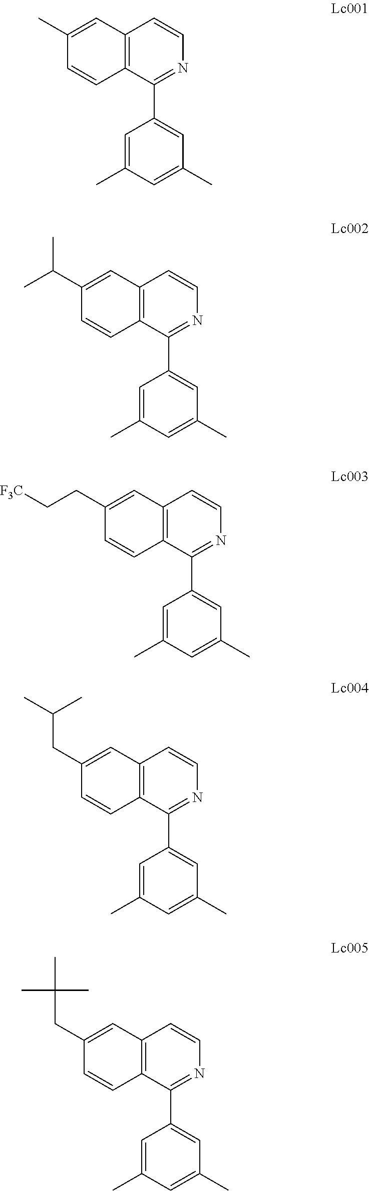

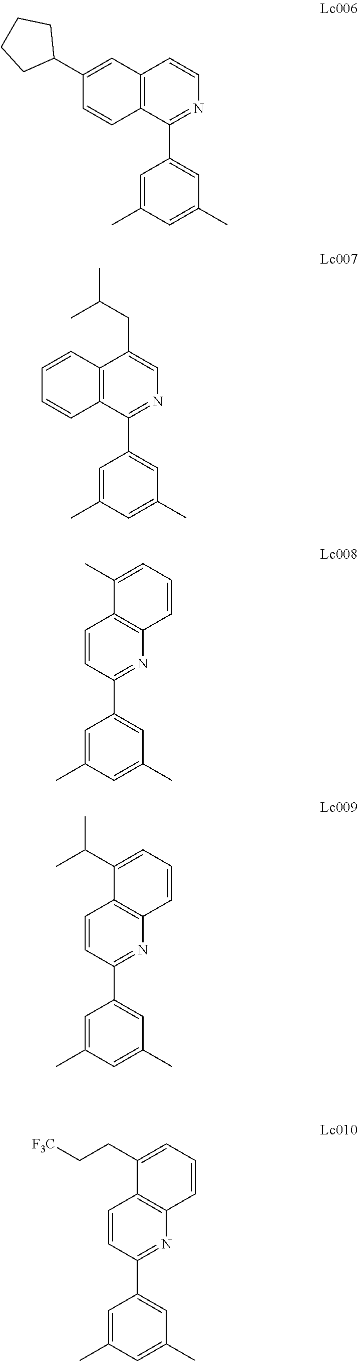

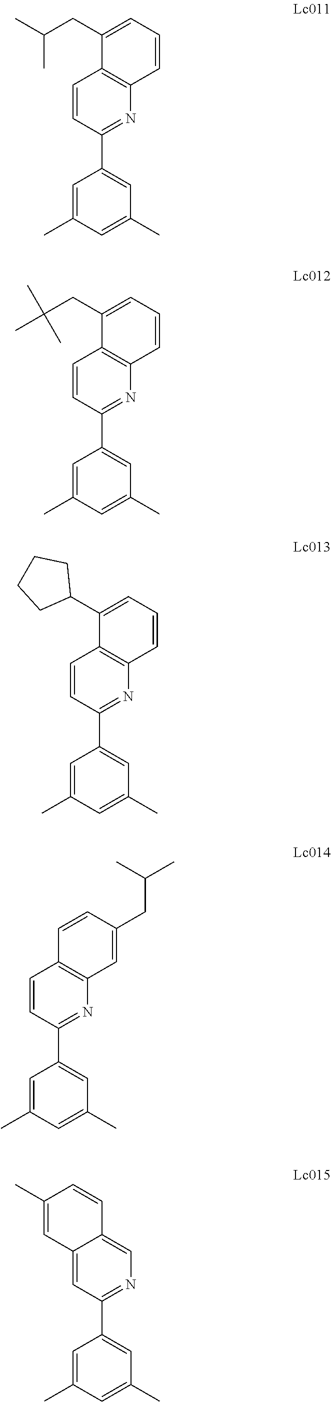

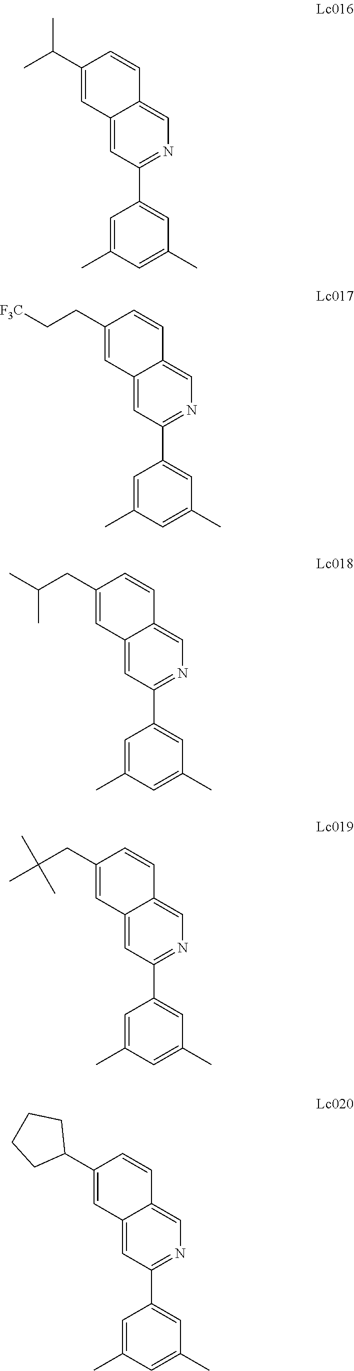

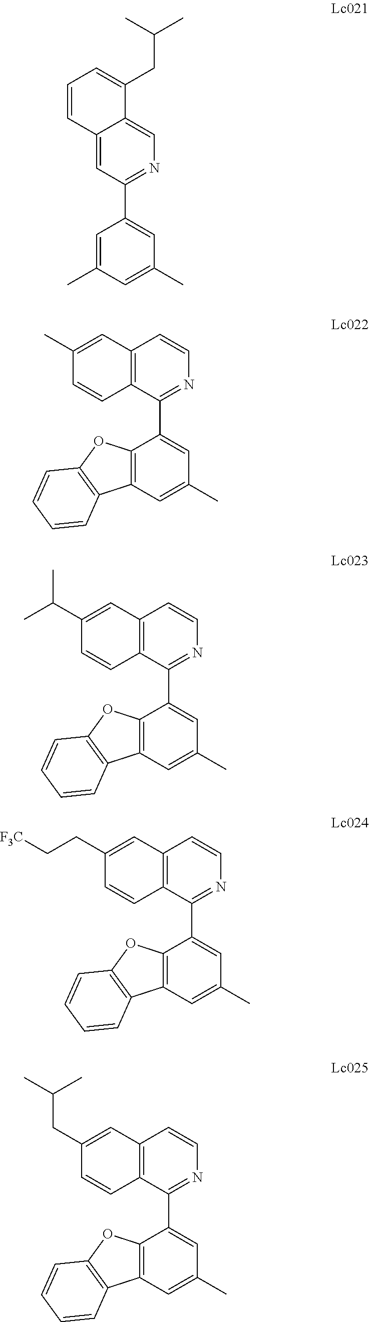

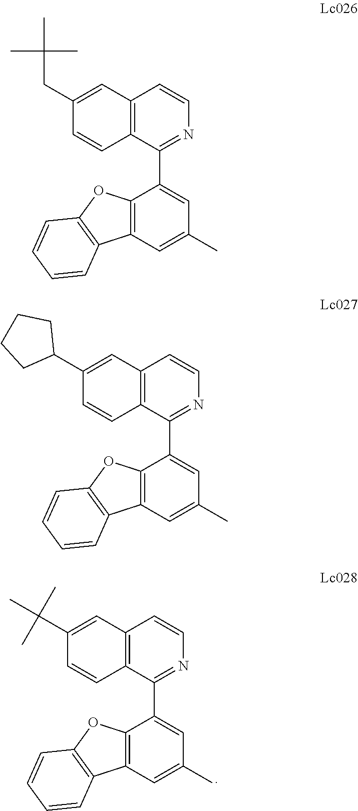

15. The metal iridium complex according to claim 3, wherein La is one of the following structural formulas, or is one of the following structural formulas in which hydrogen is partially or completely replaced by deuterium or fluorine,

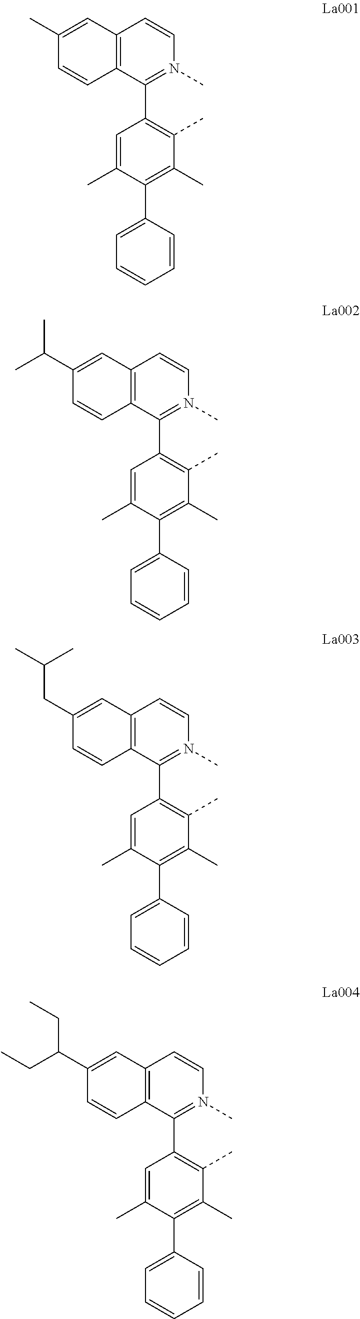

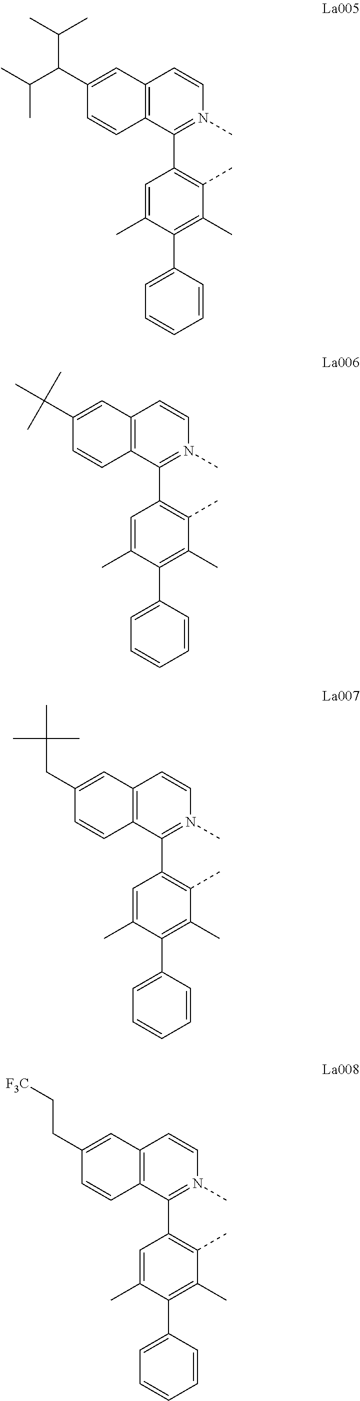

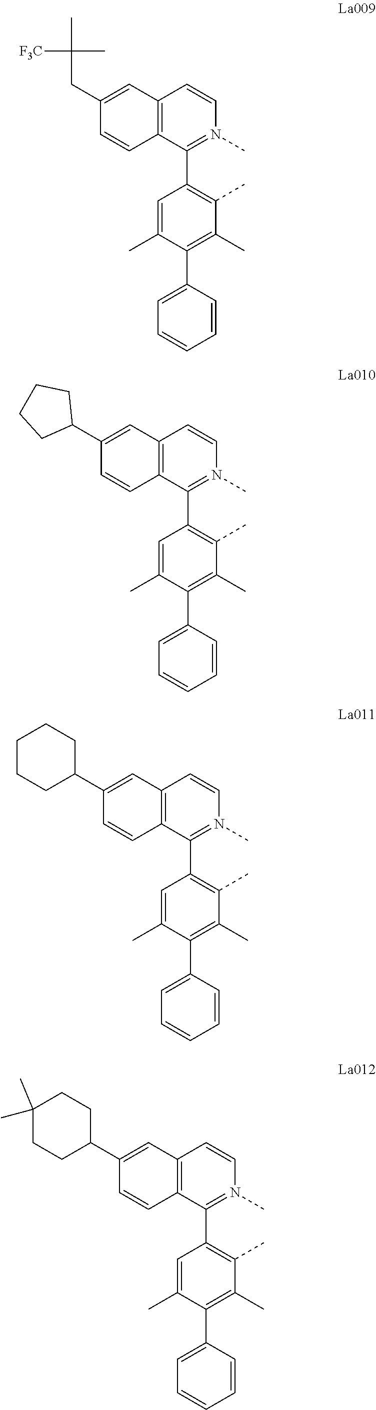

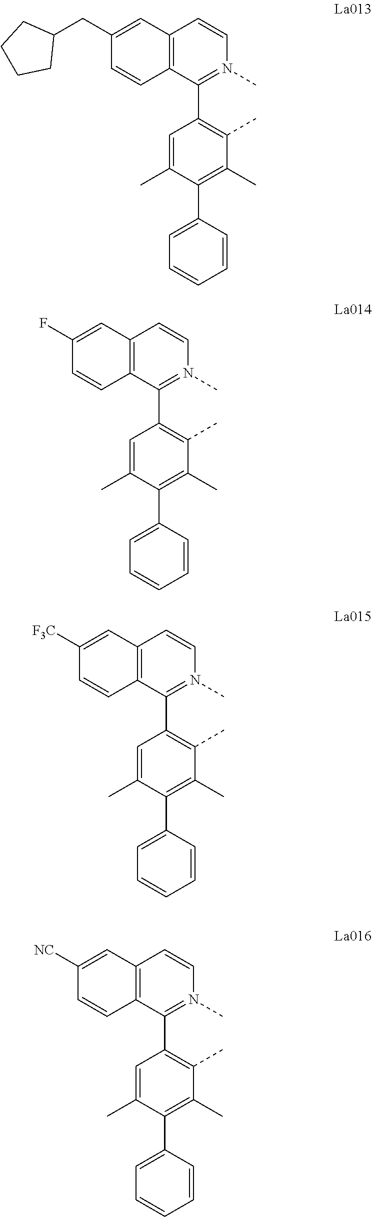

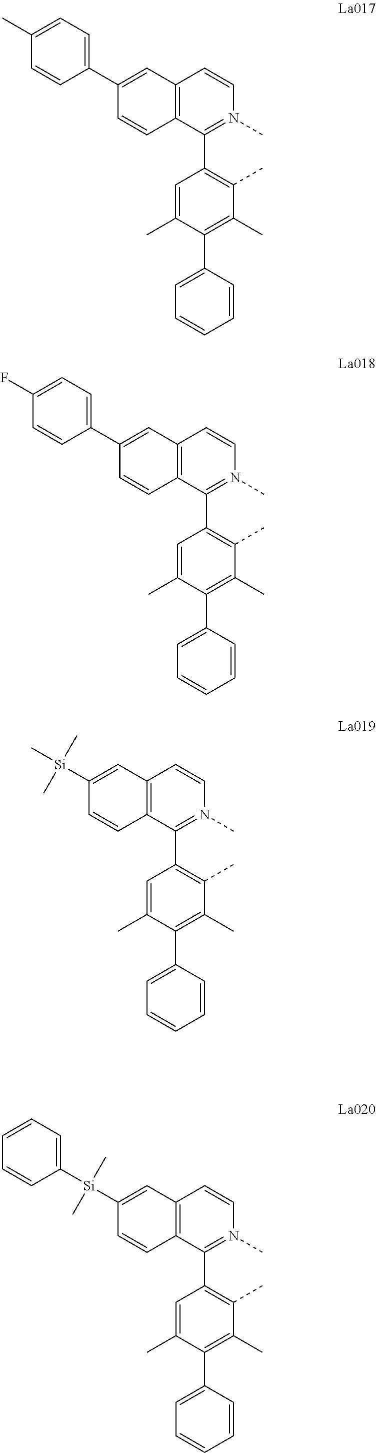

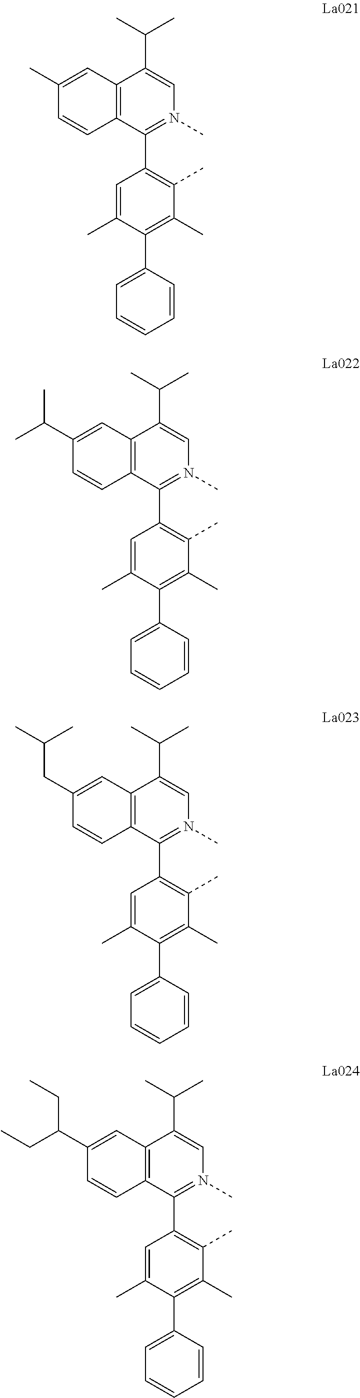

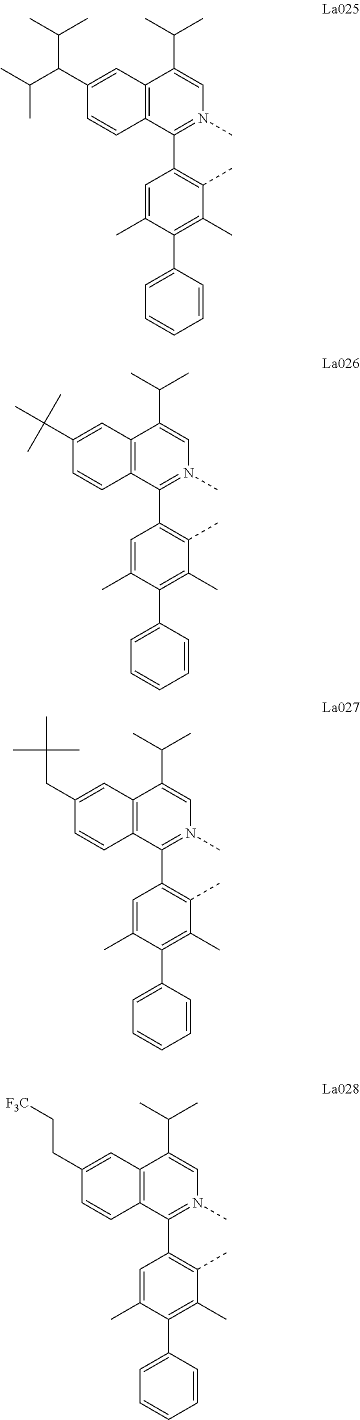

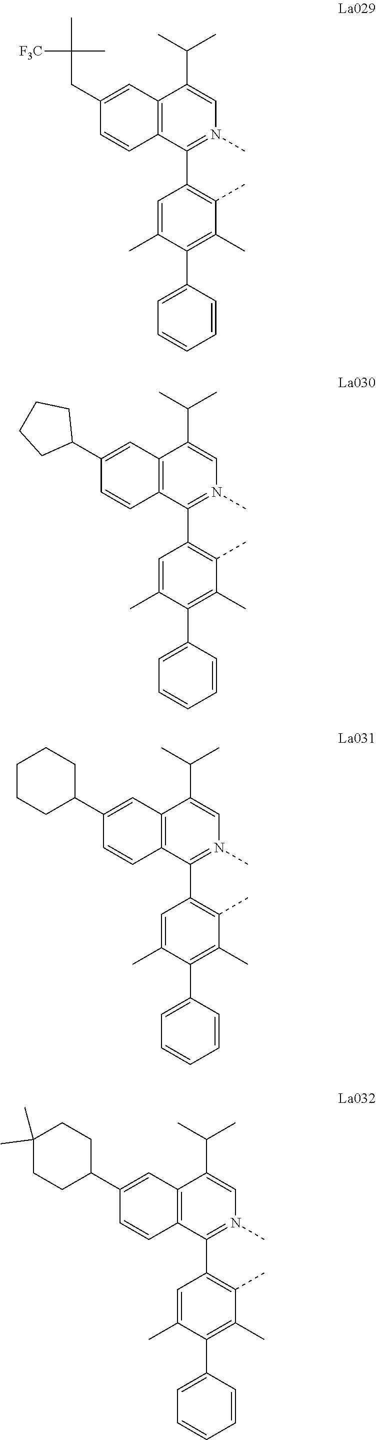

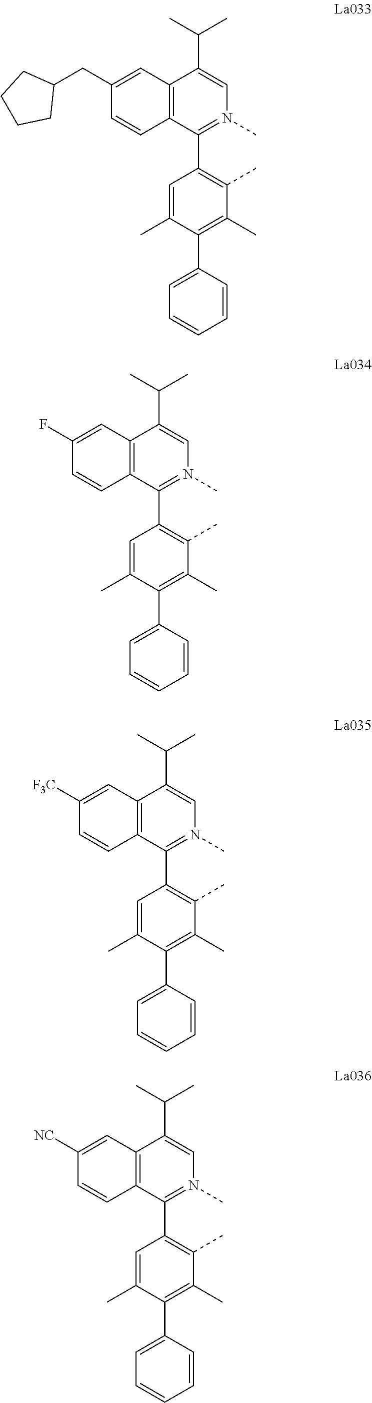

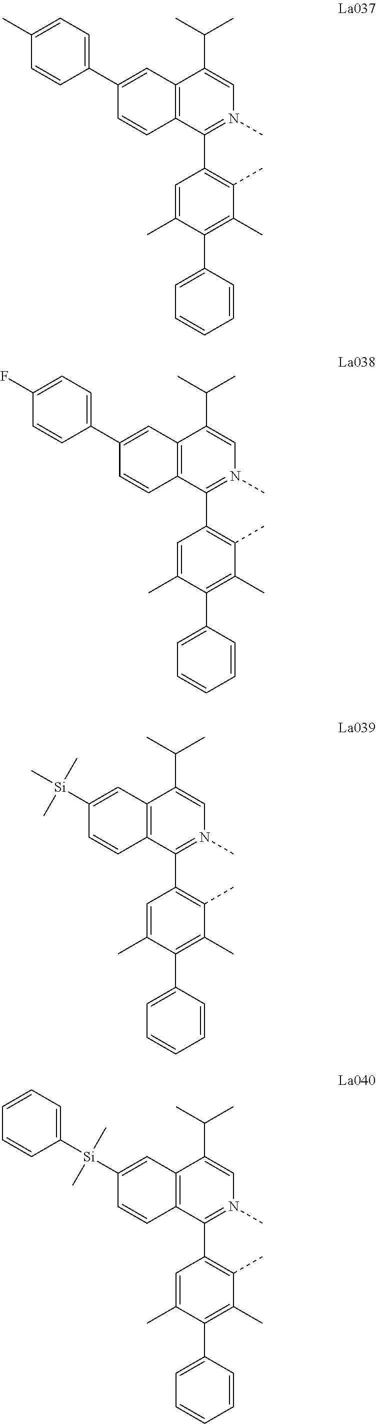

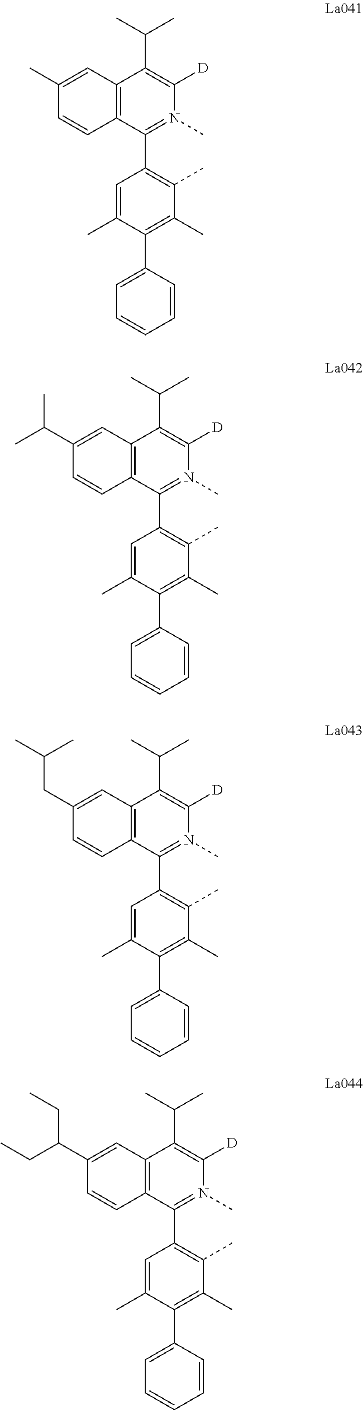

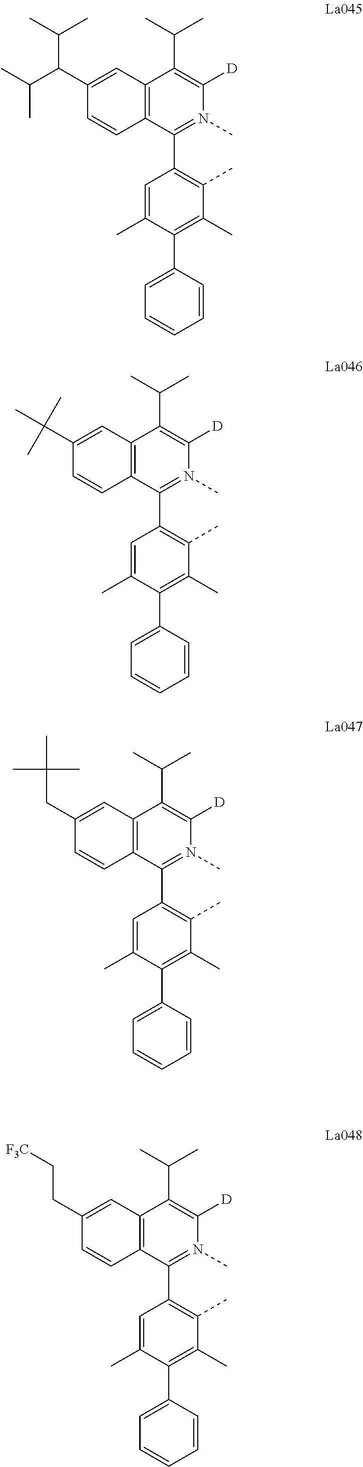

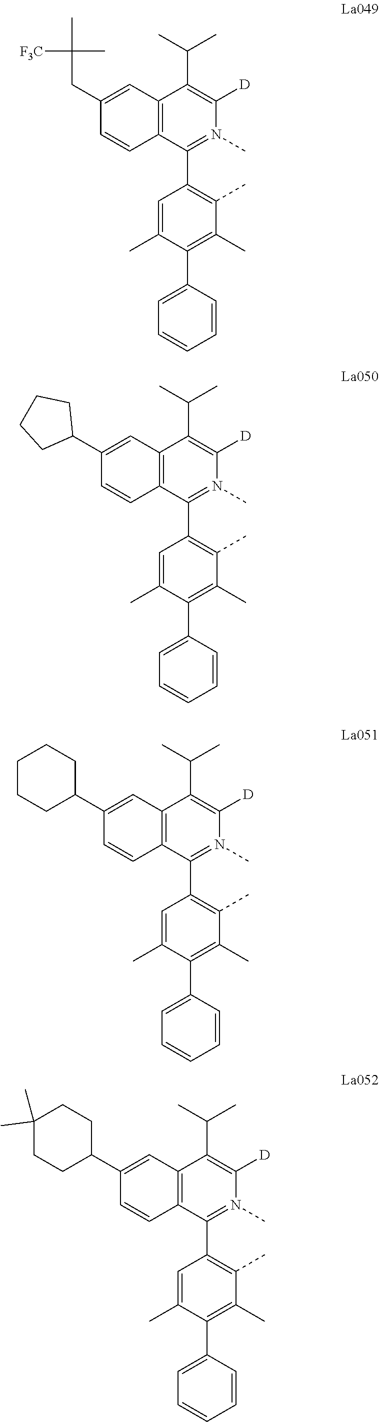

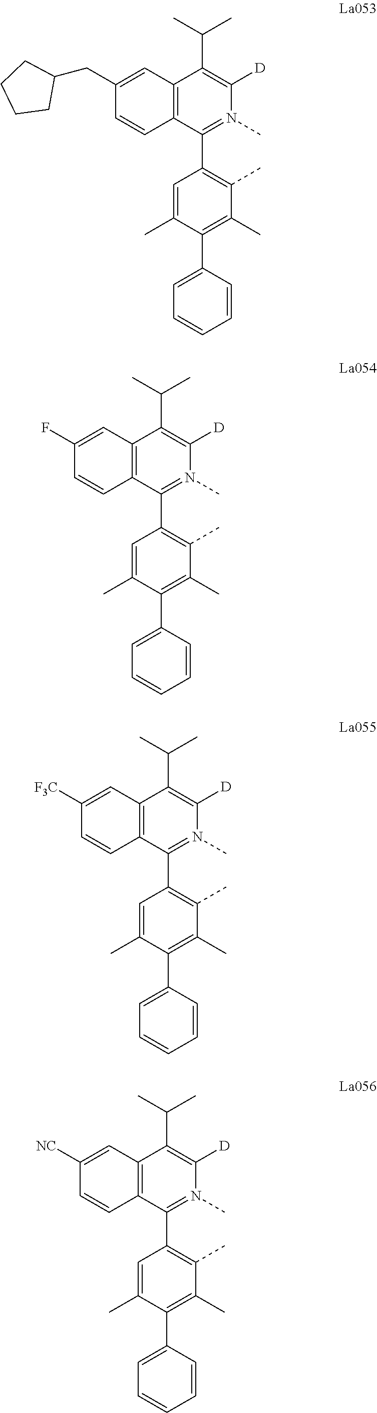

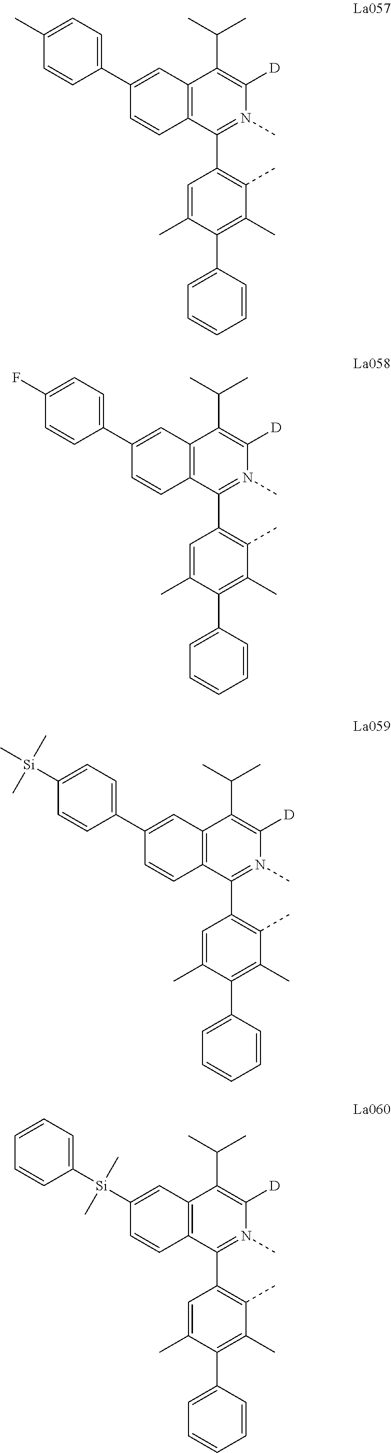

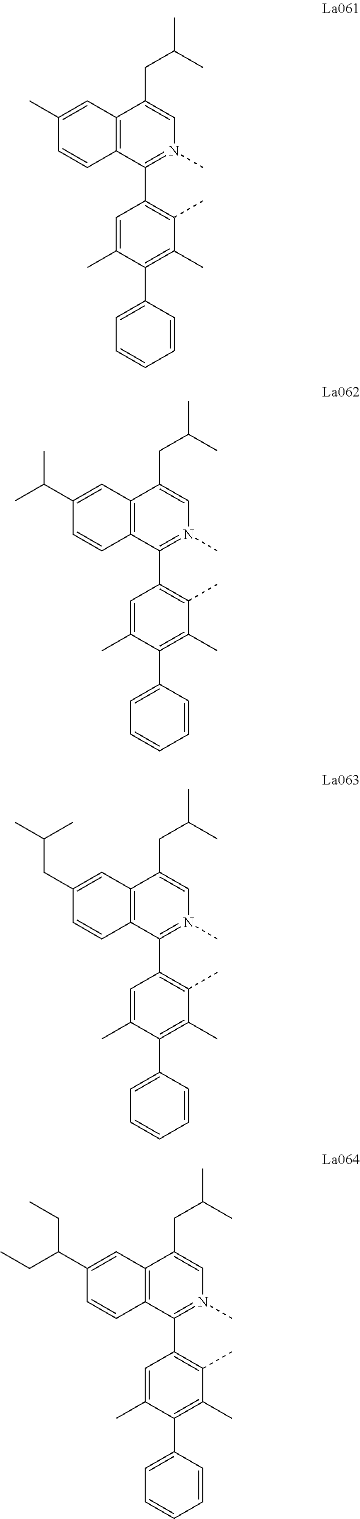

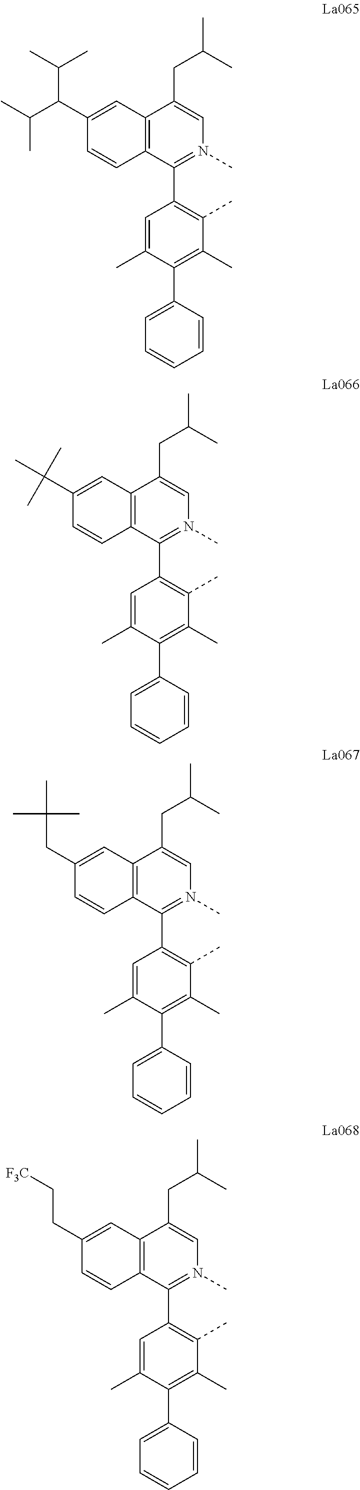

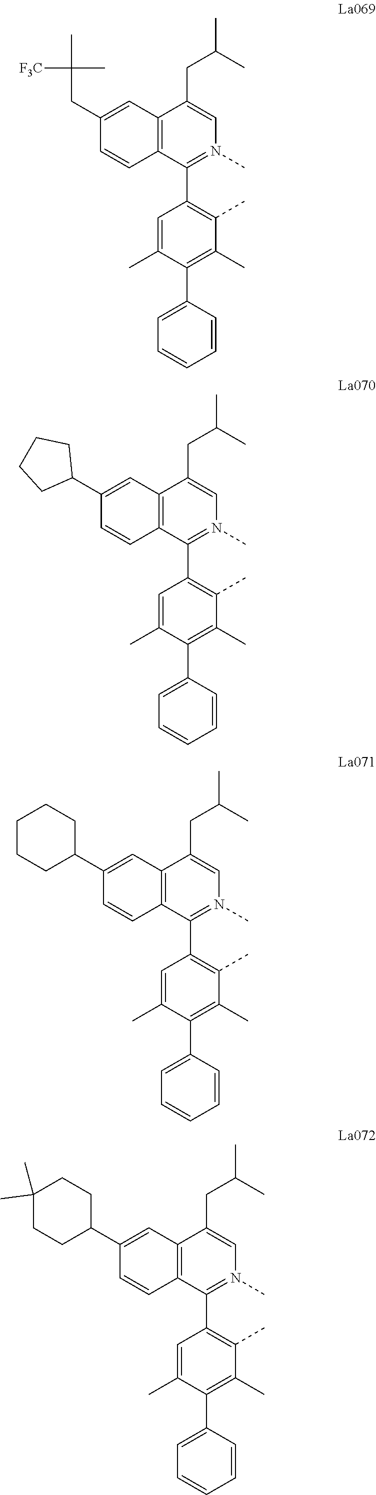

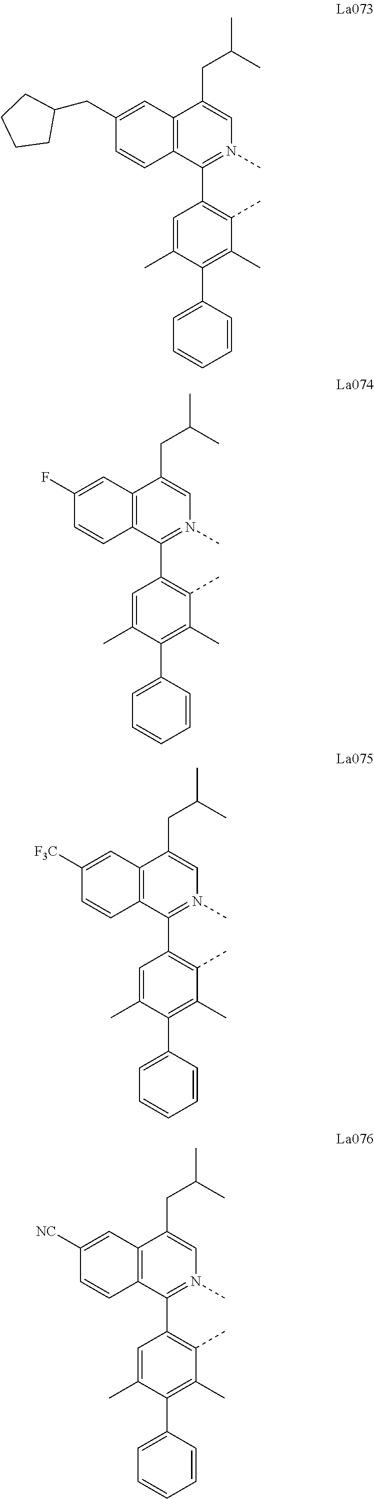

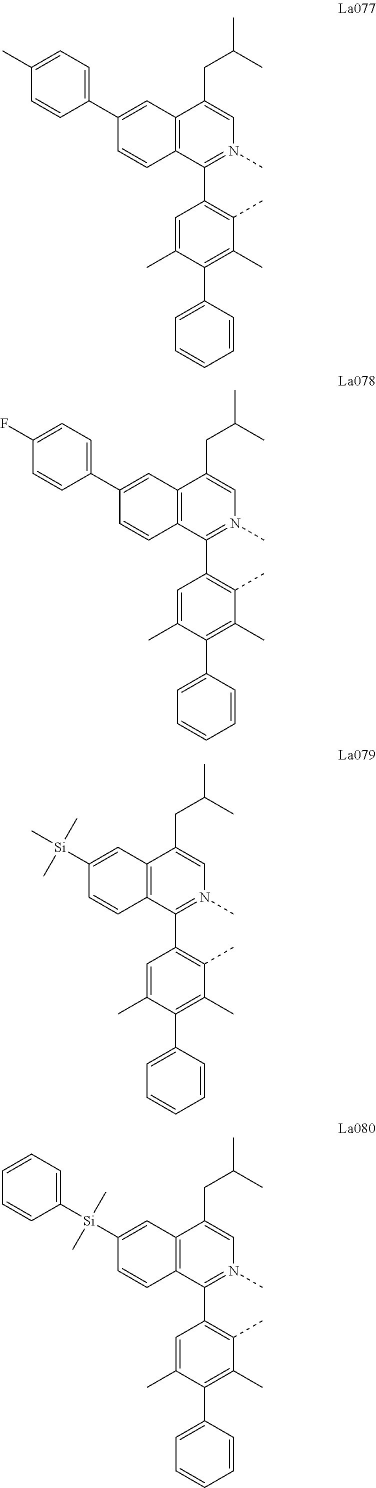

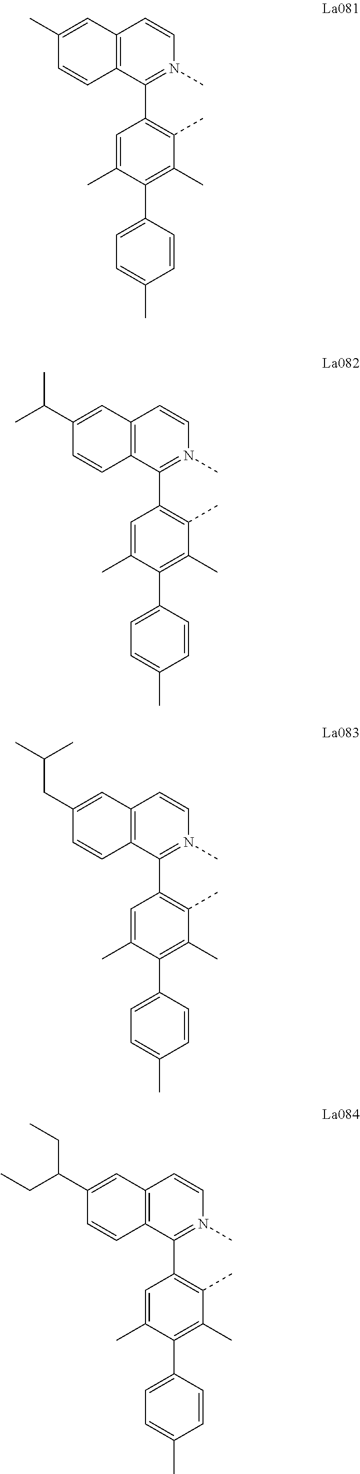

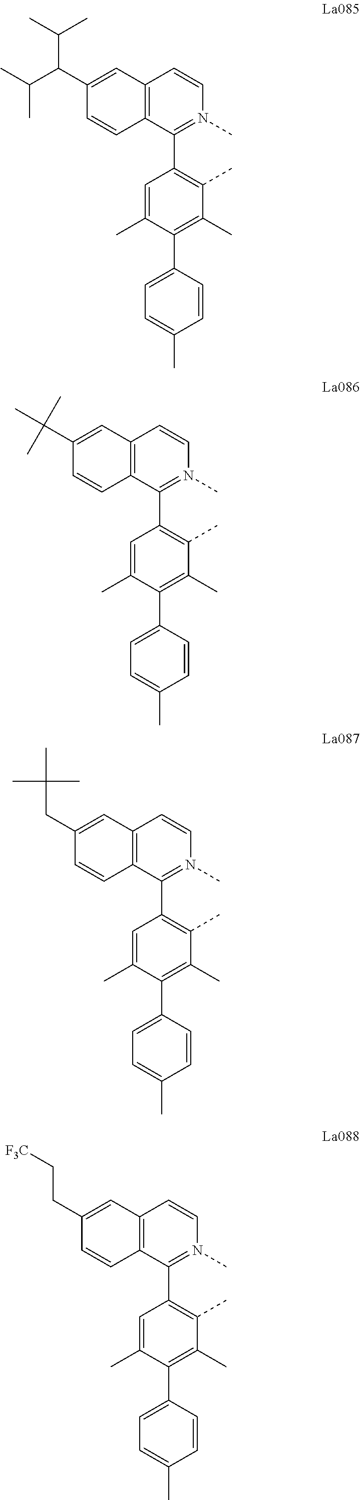

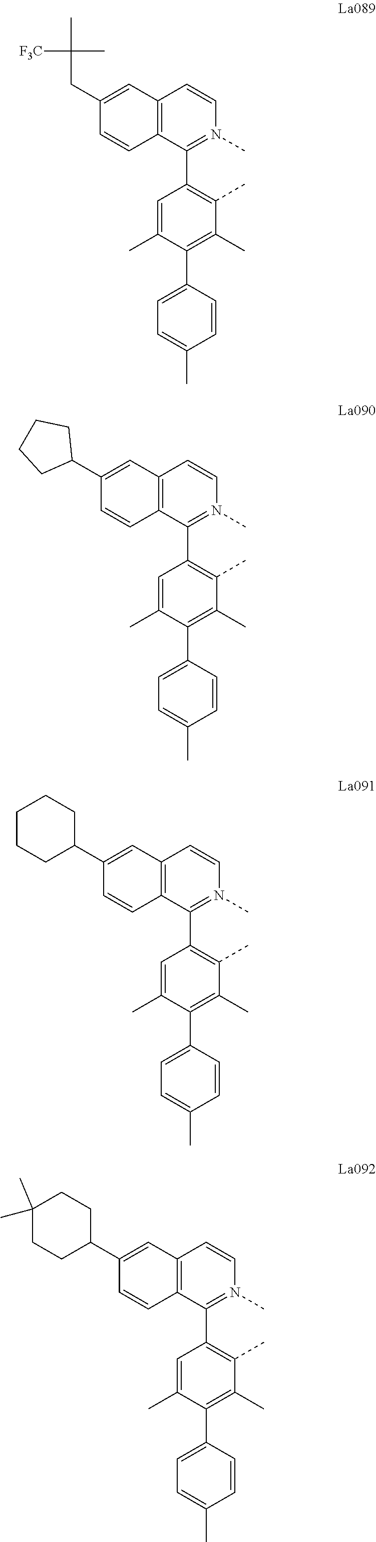

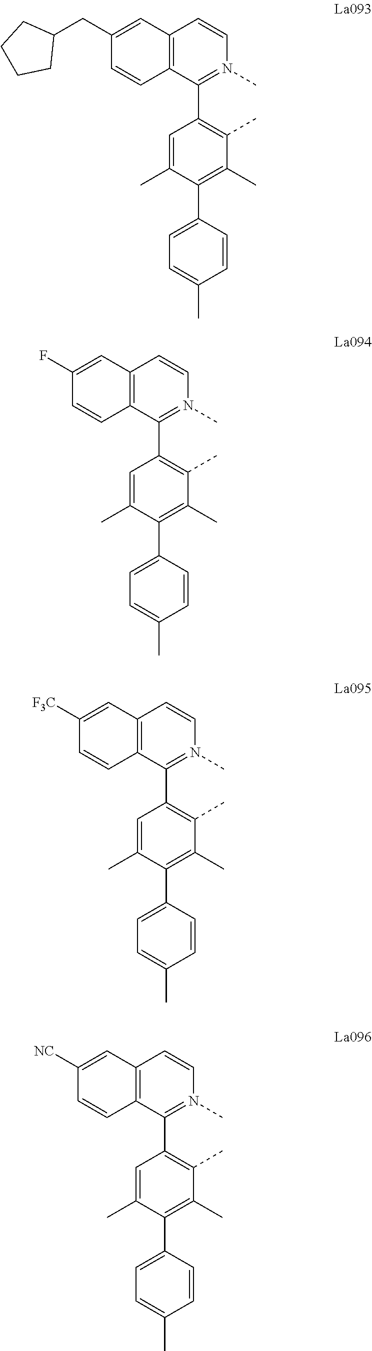

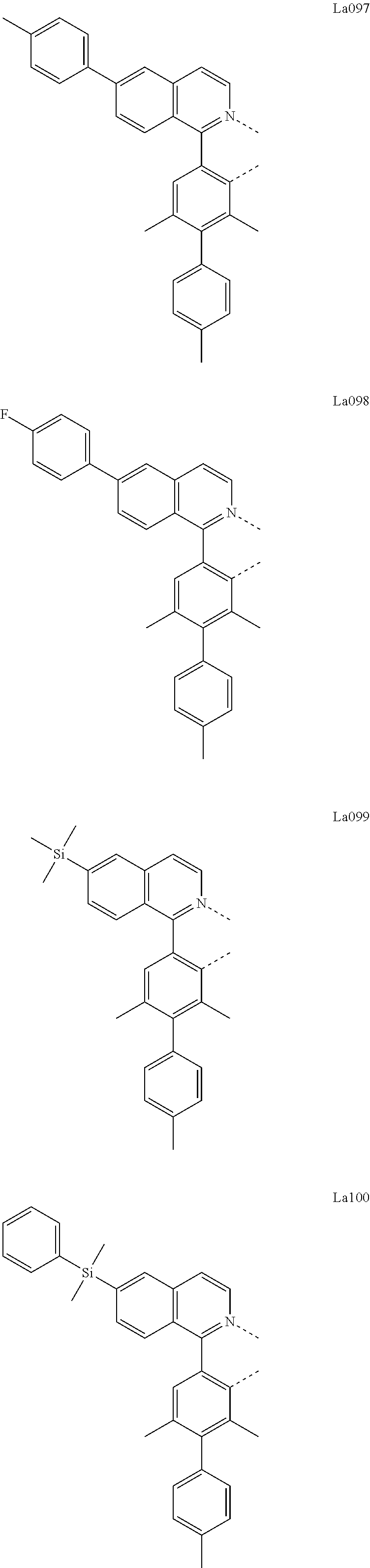

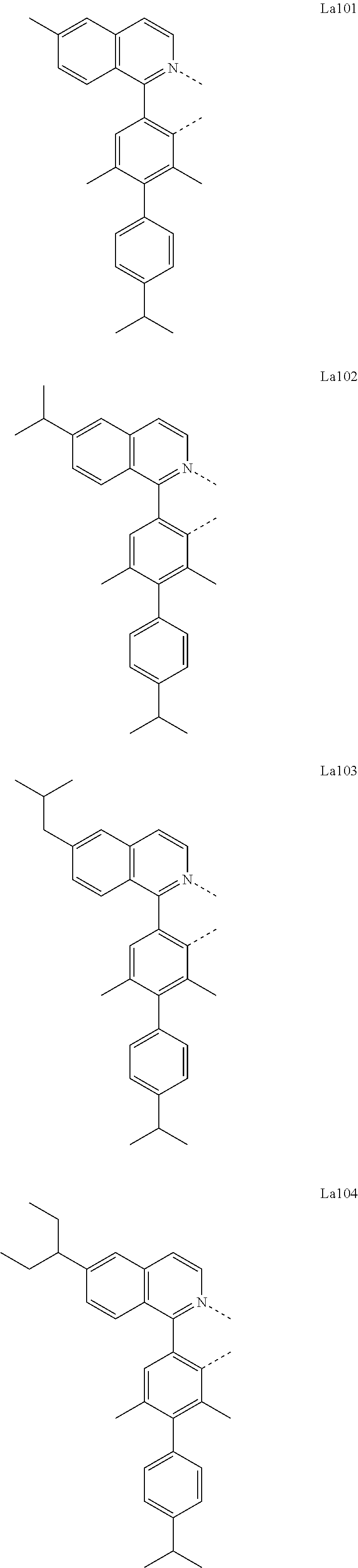

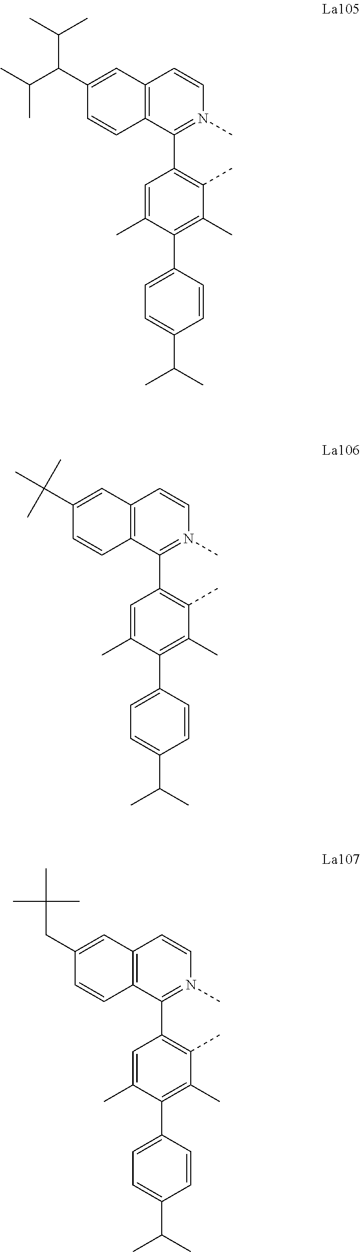

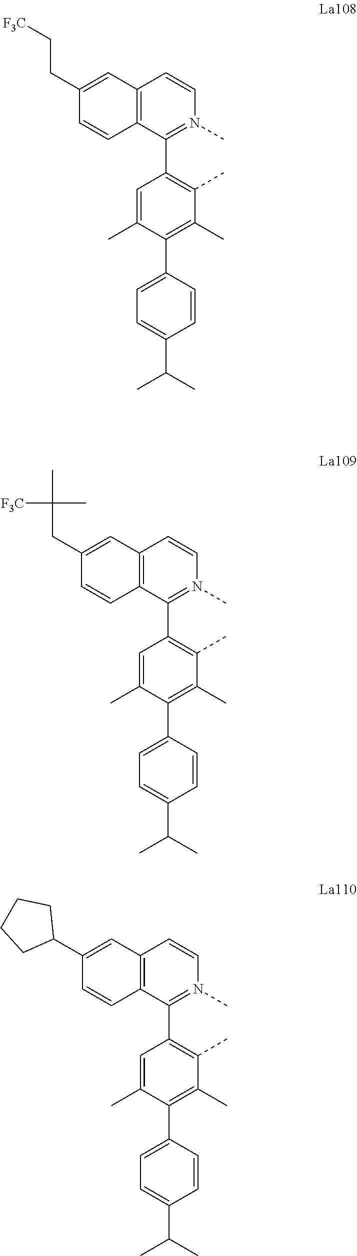

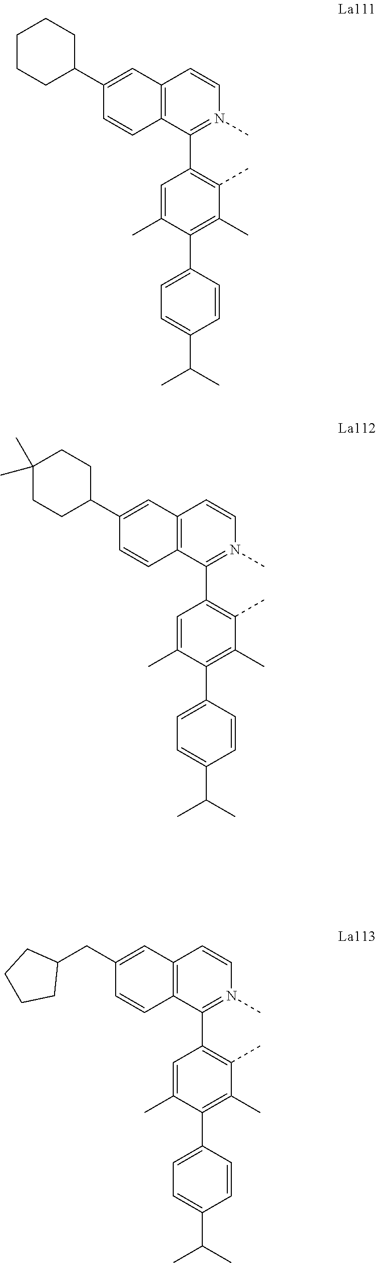

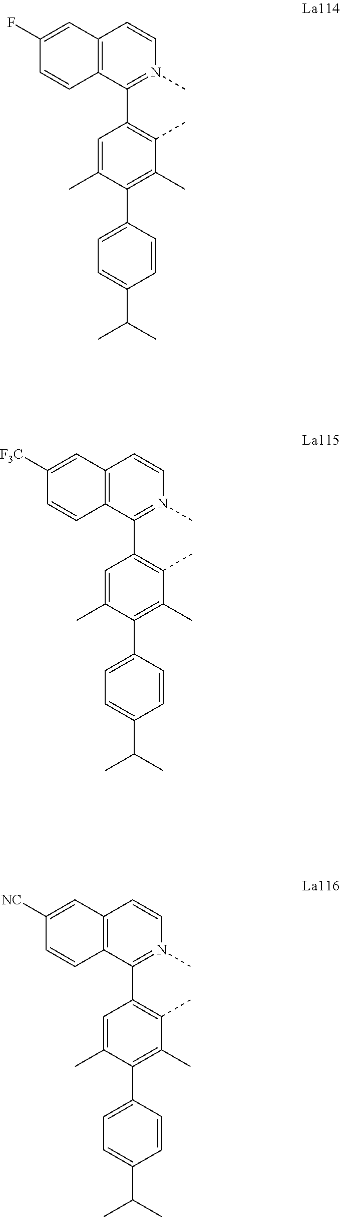

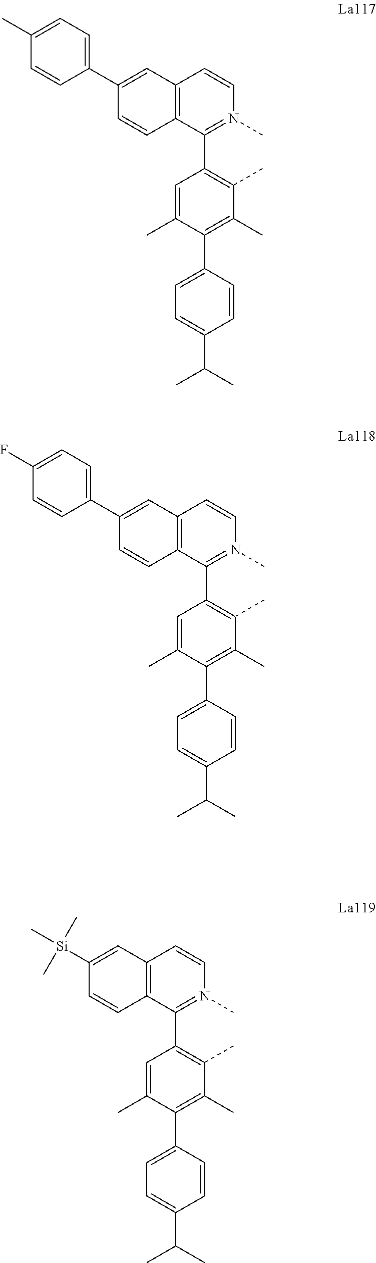

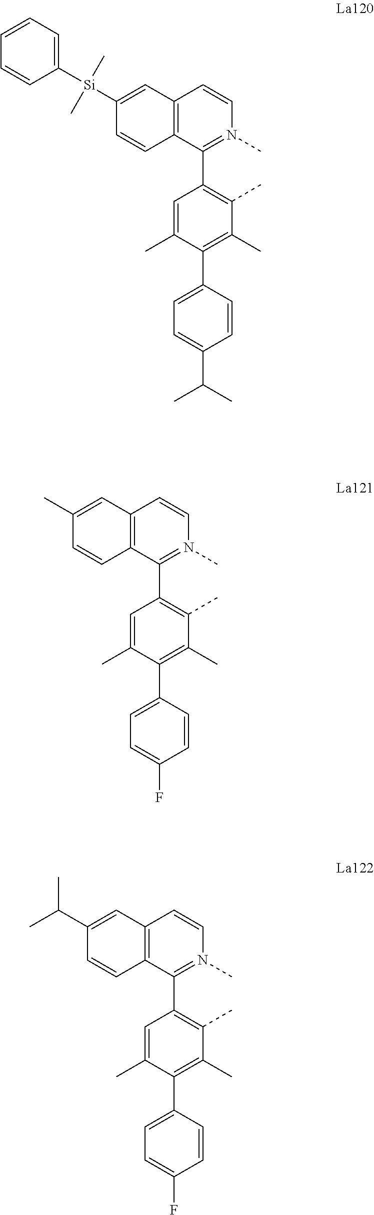

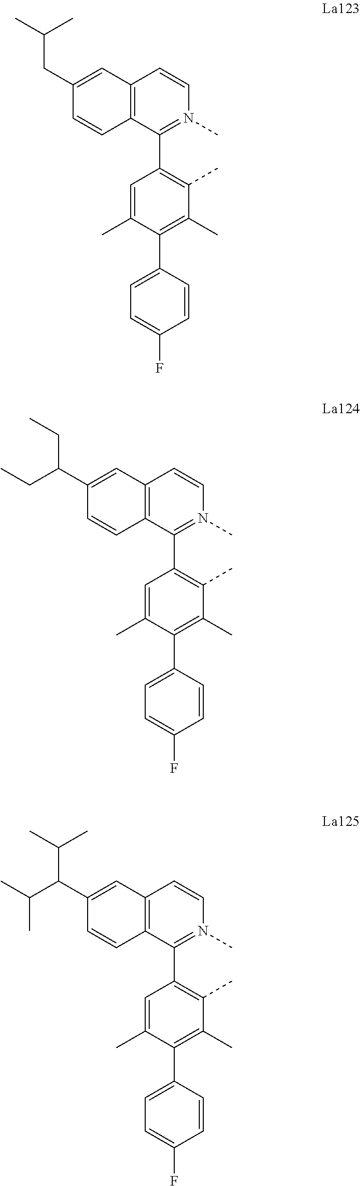

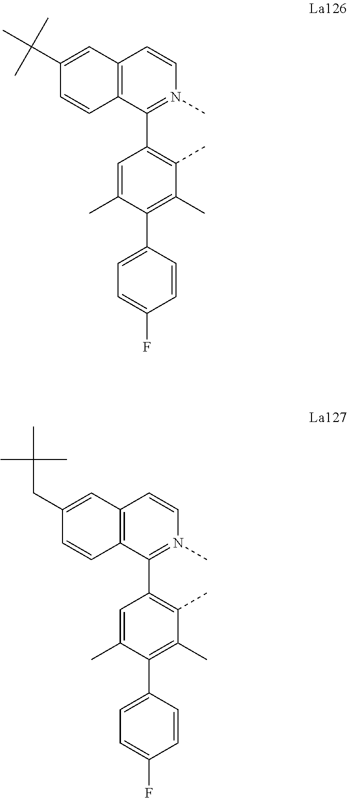

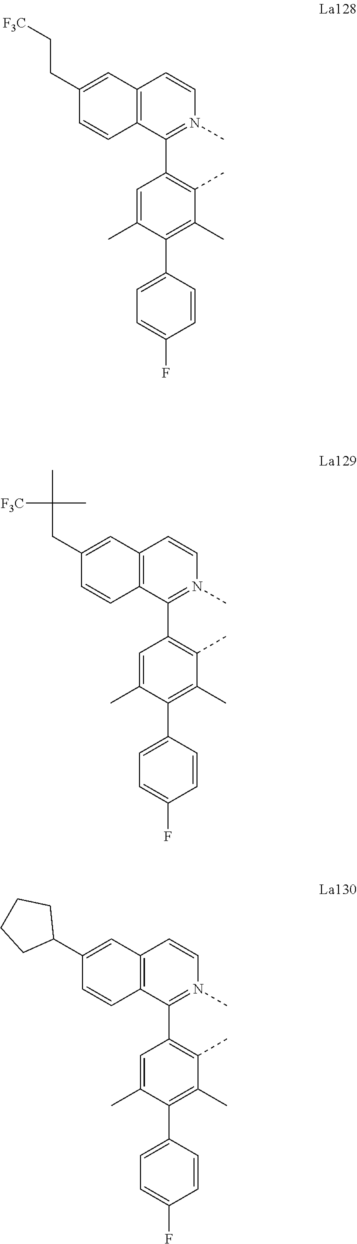

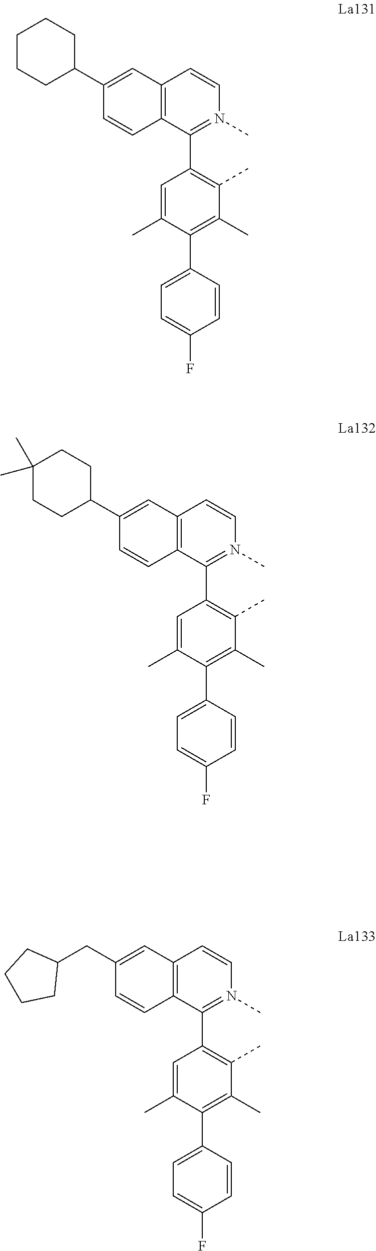

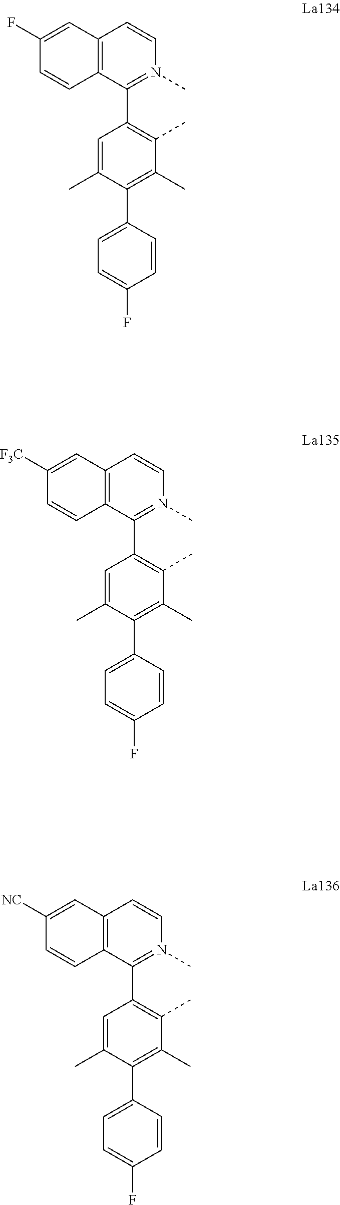

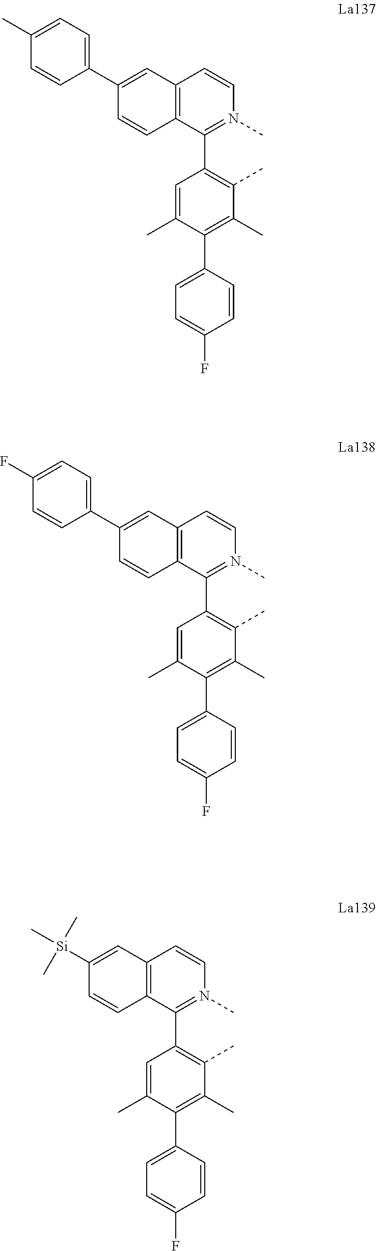

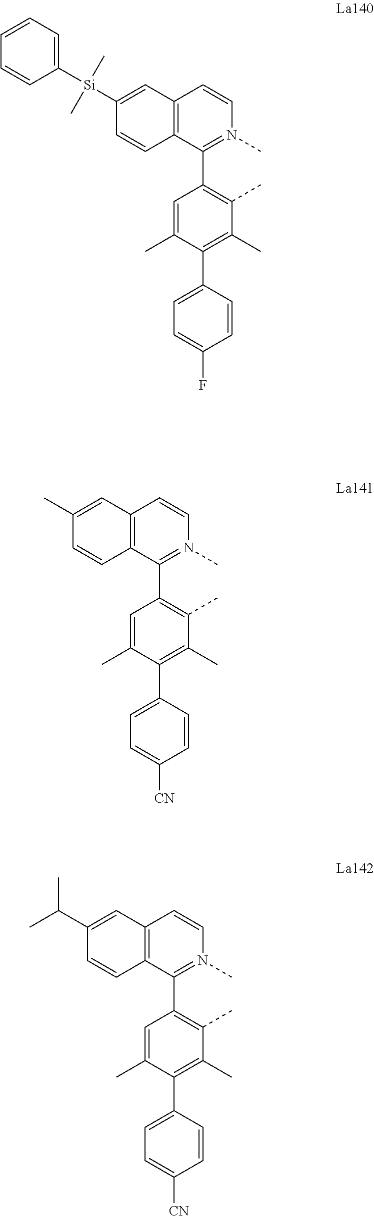

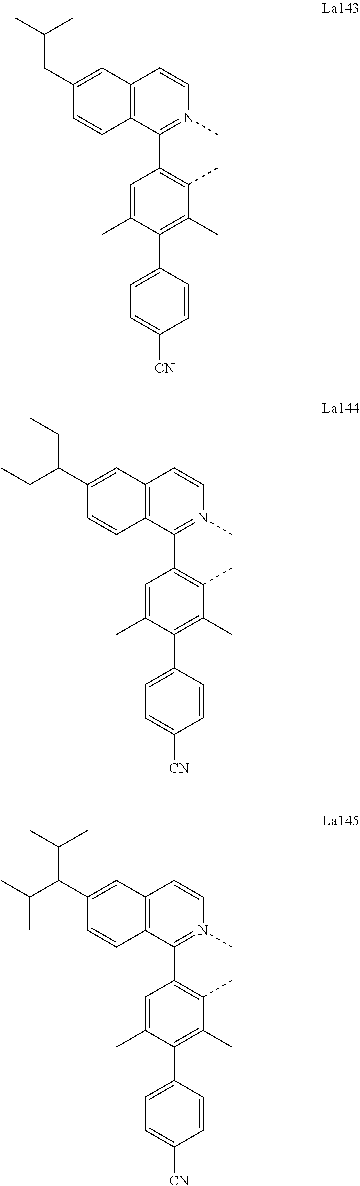

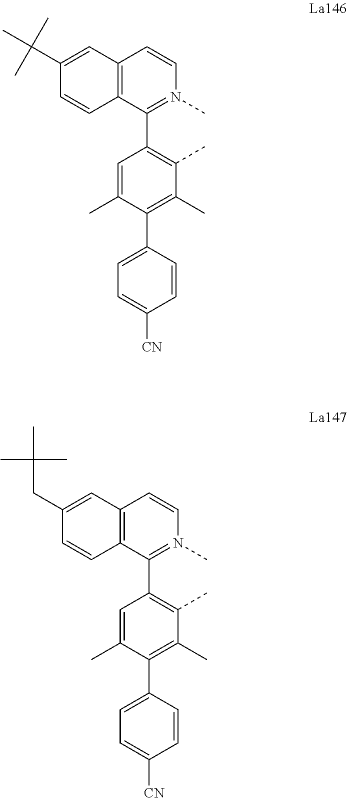

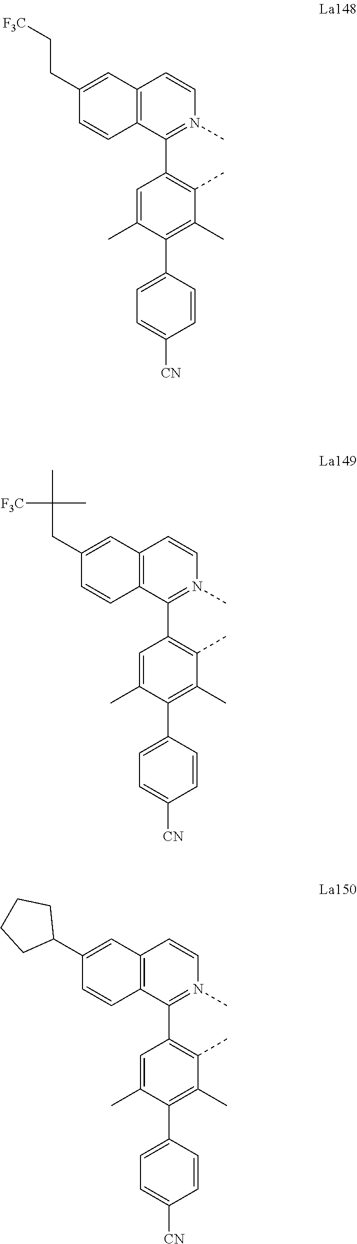

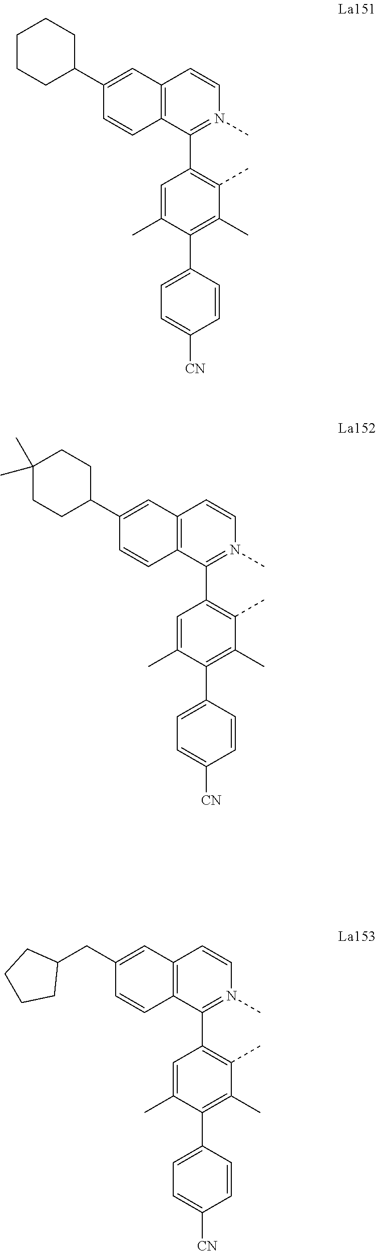

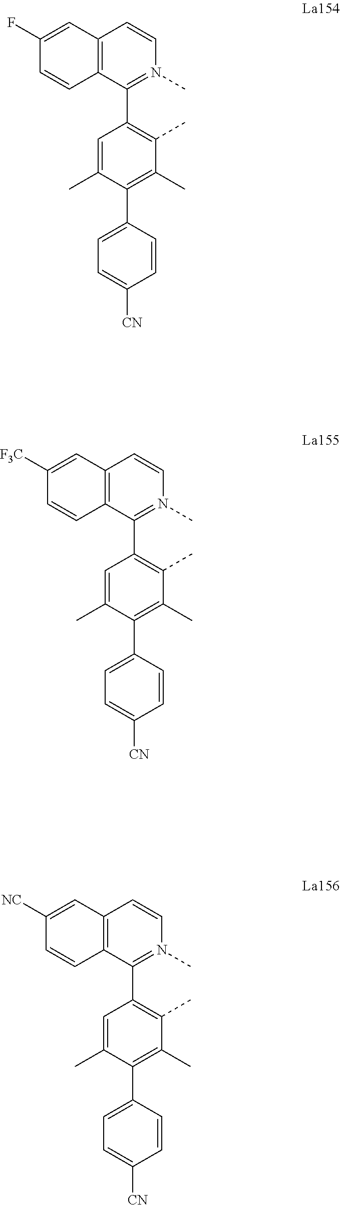

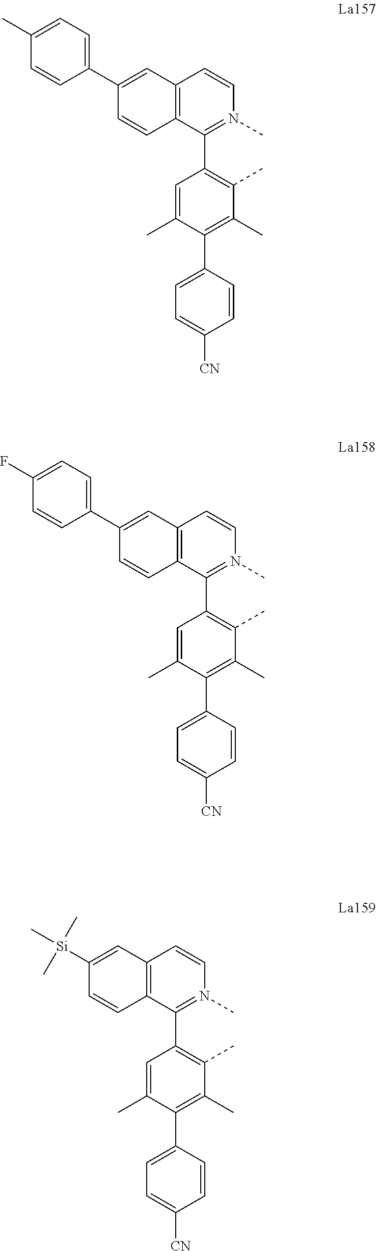

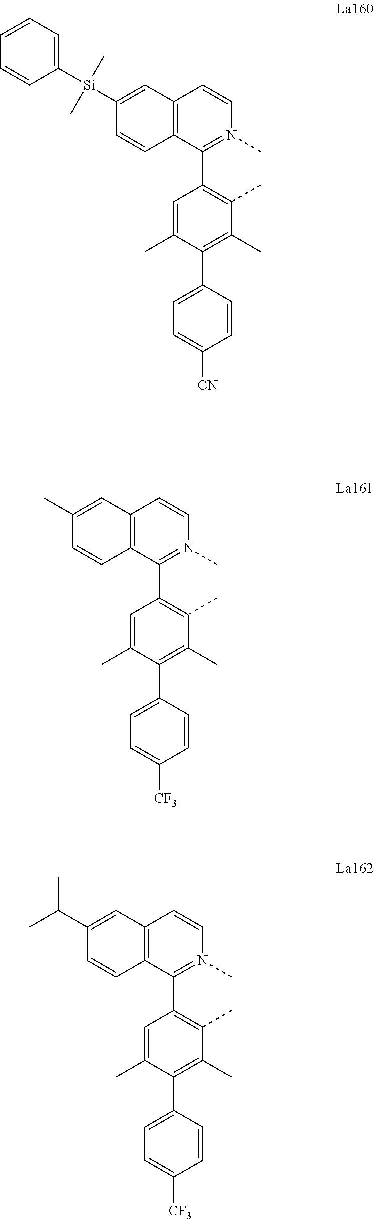

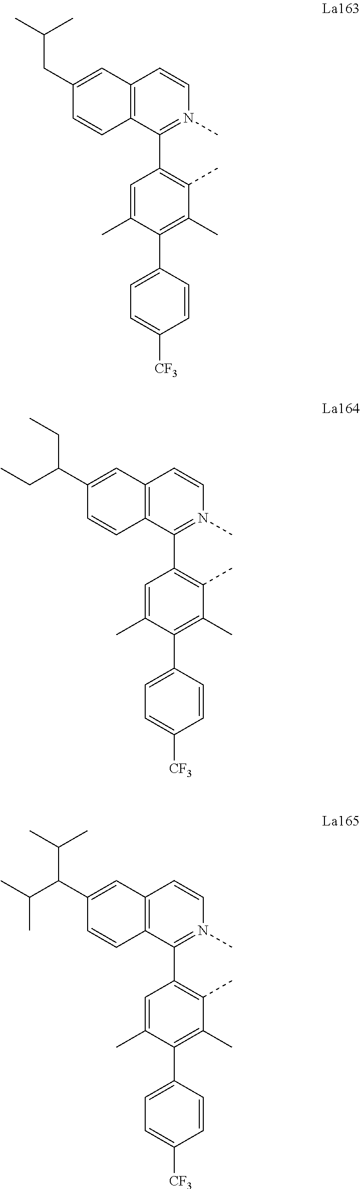

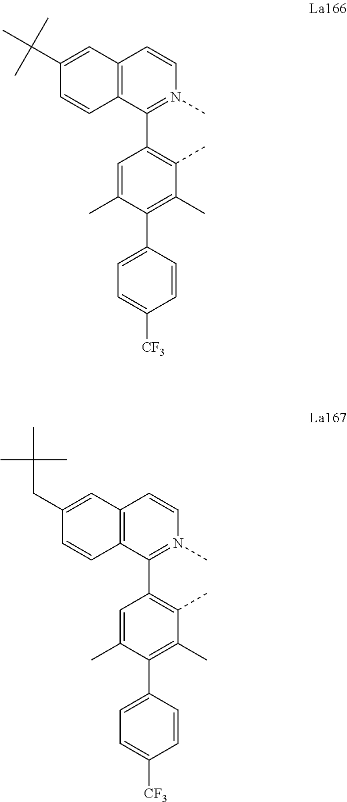

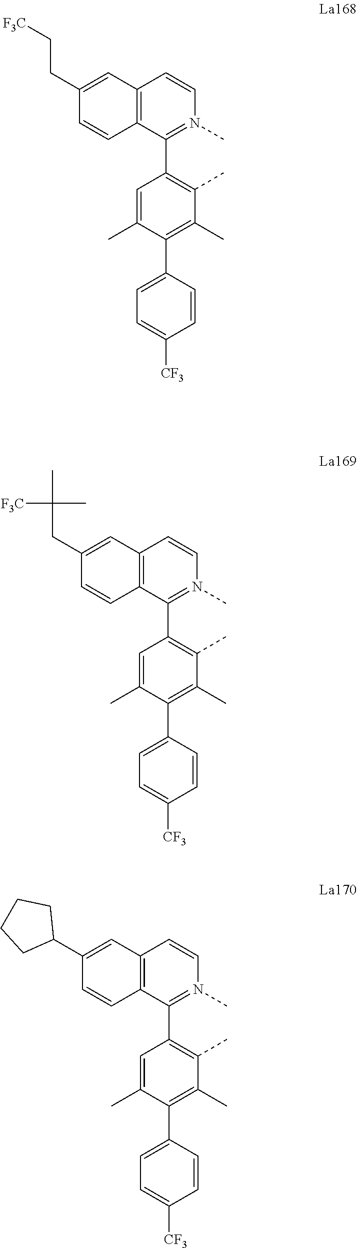

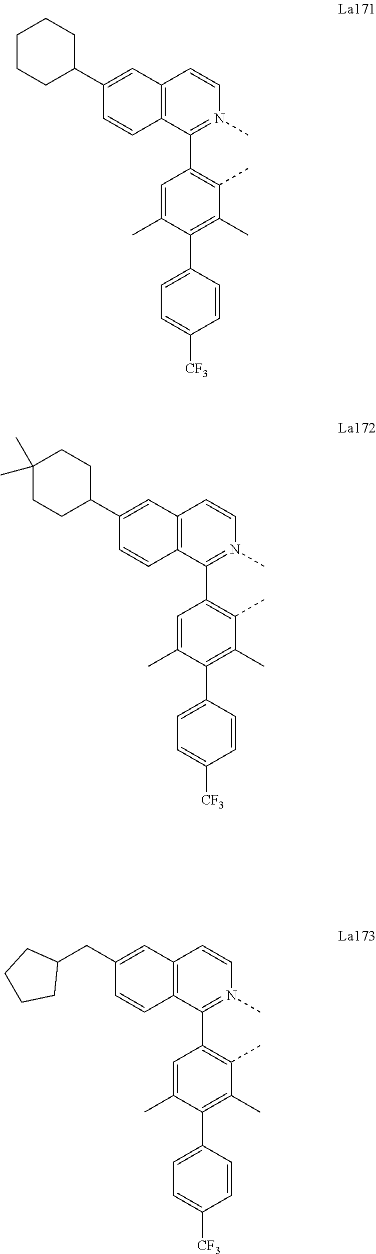

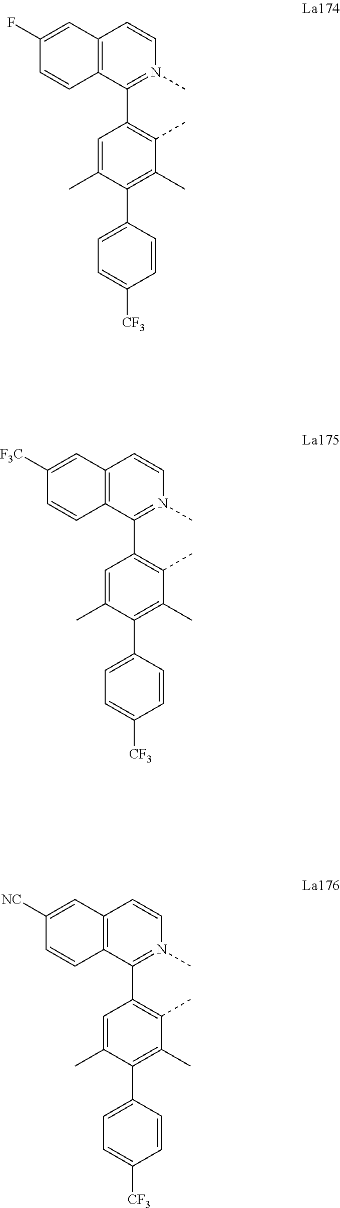

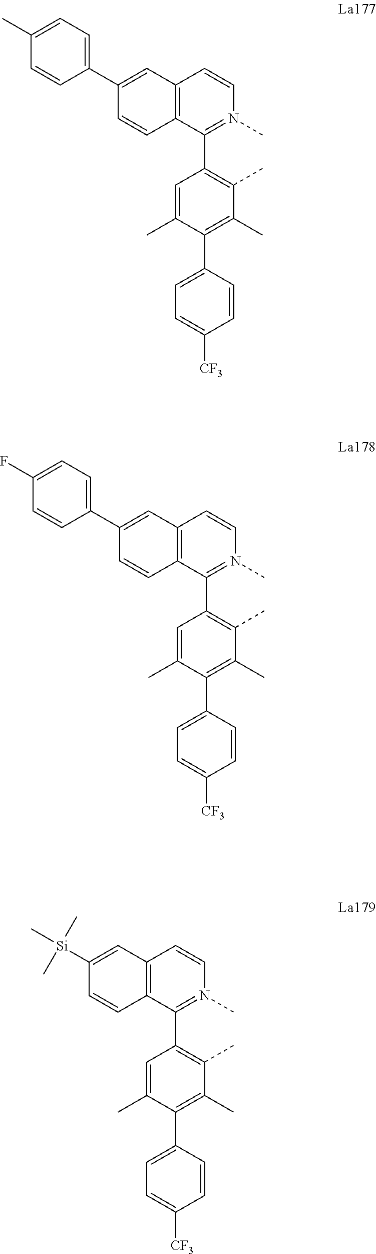

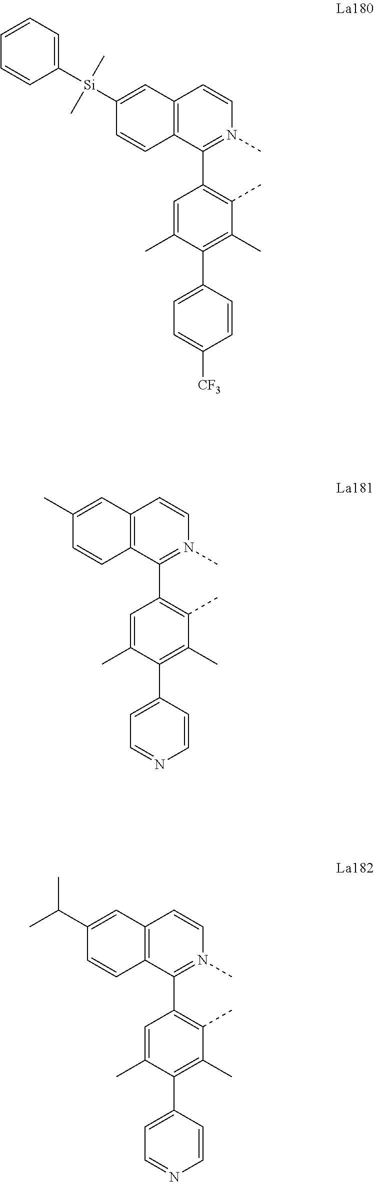

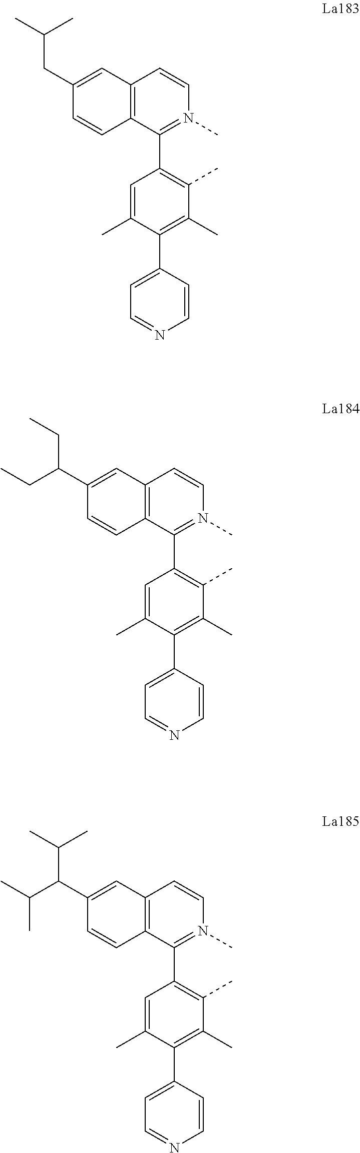

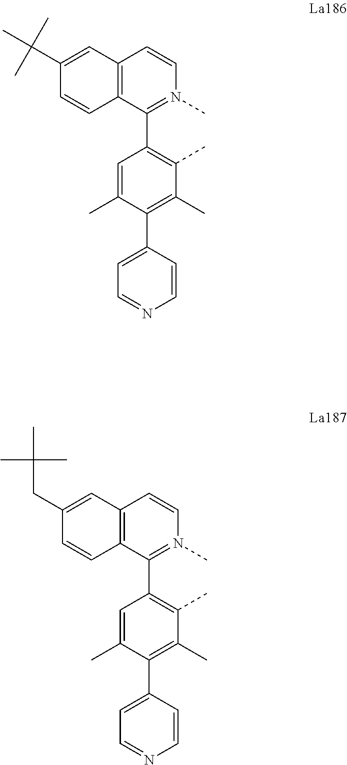

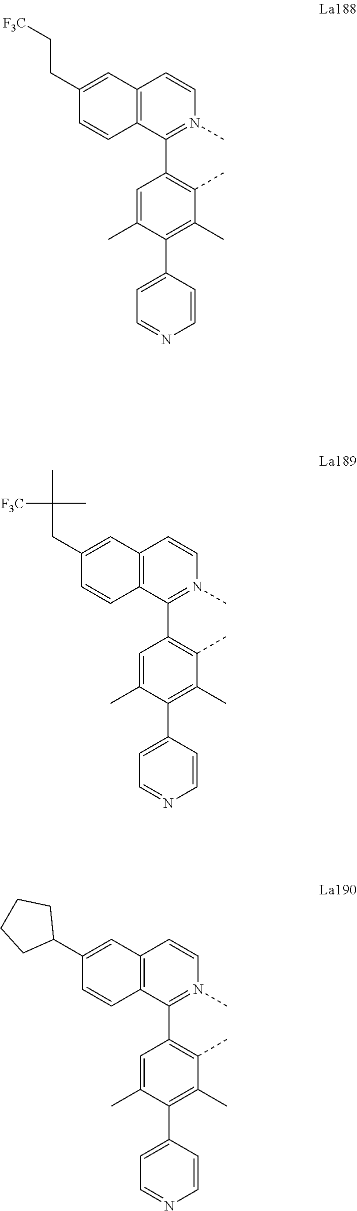

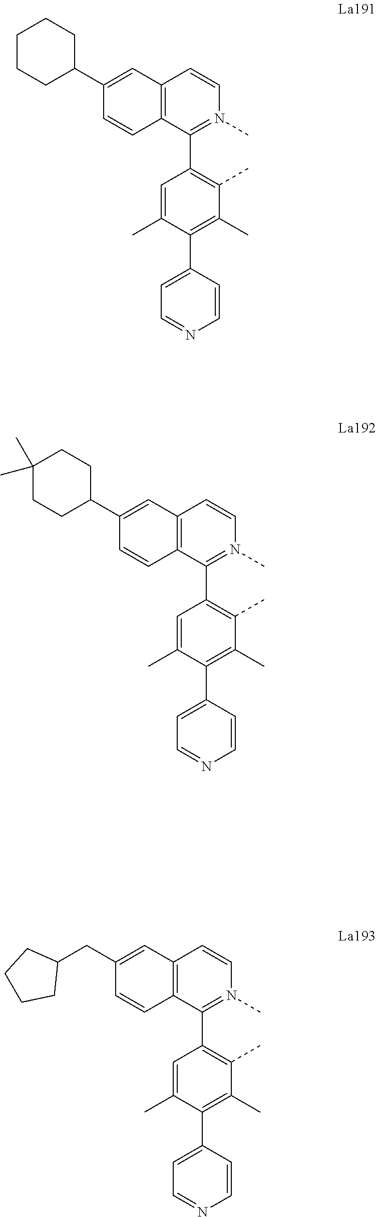

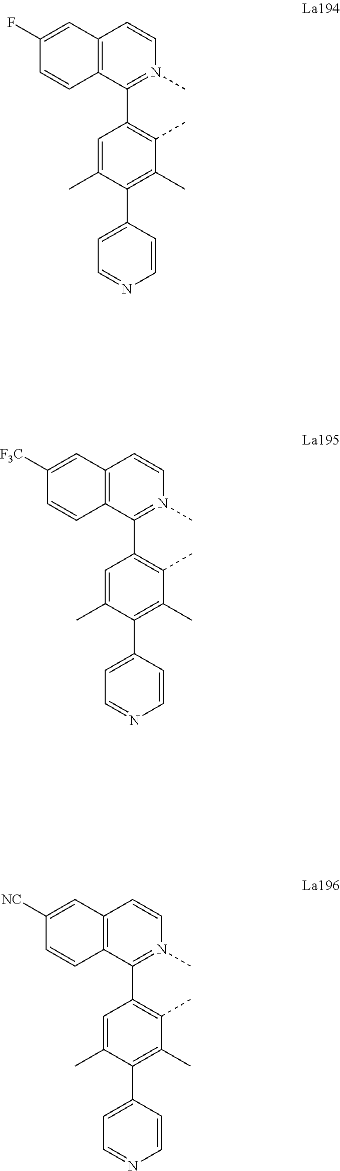

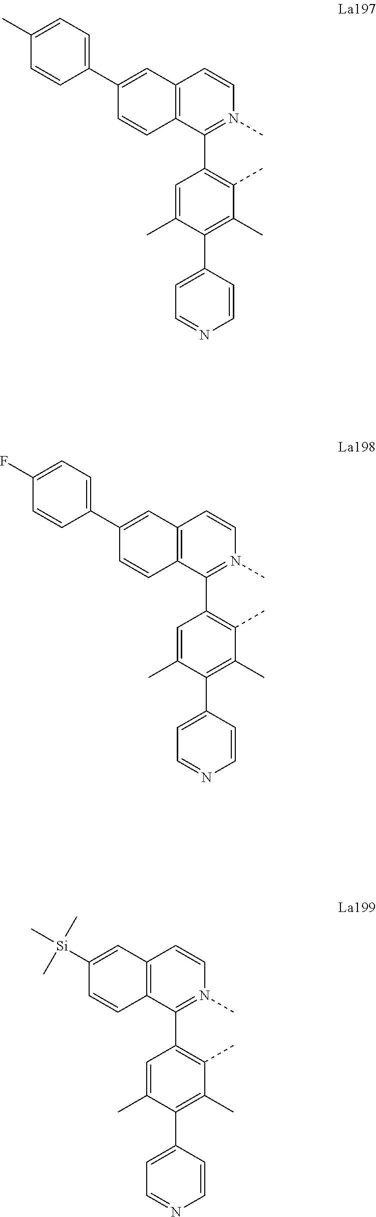

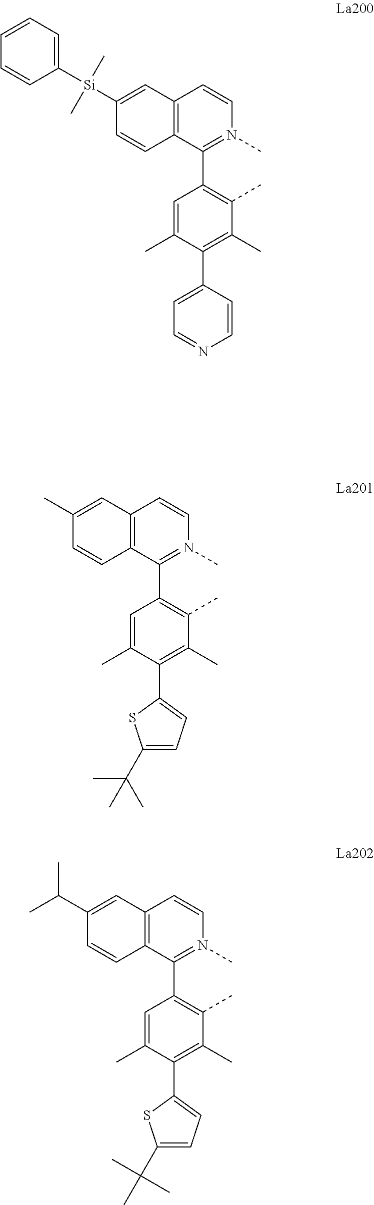

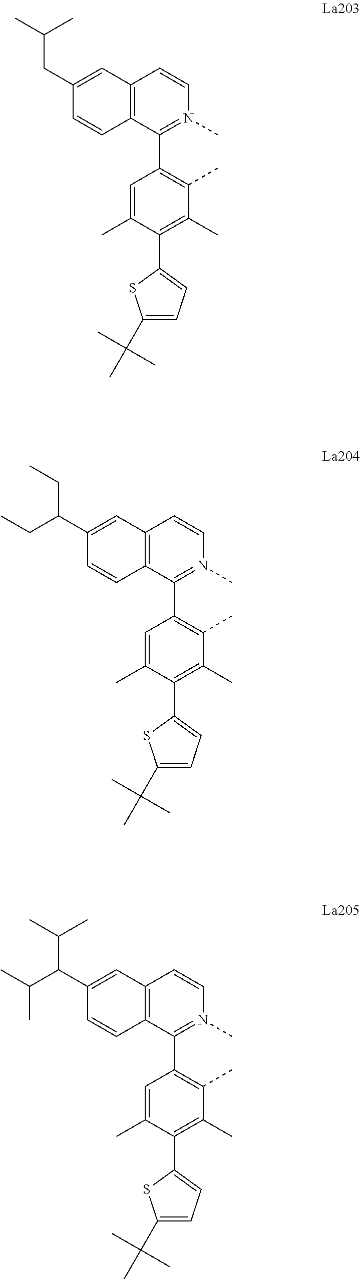

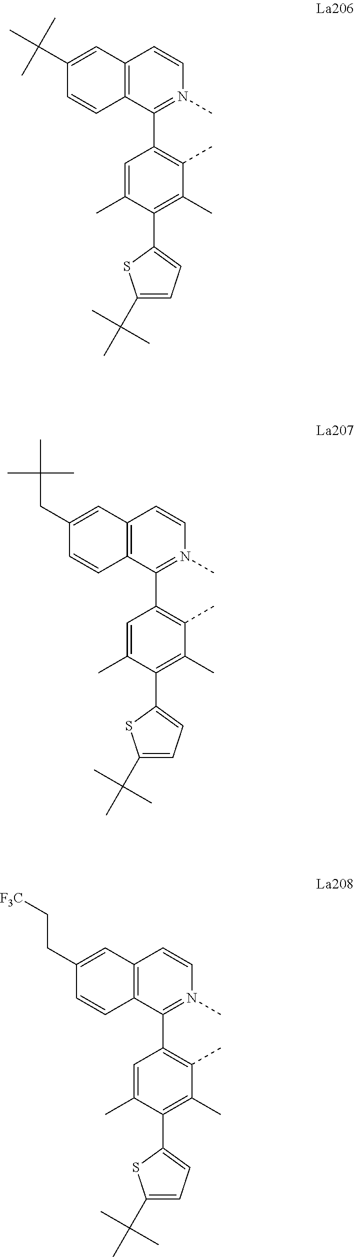

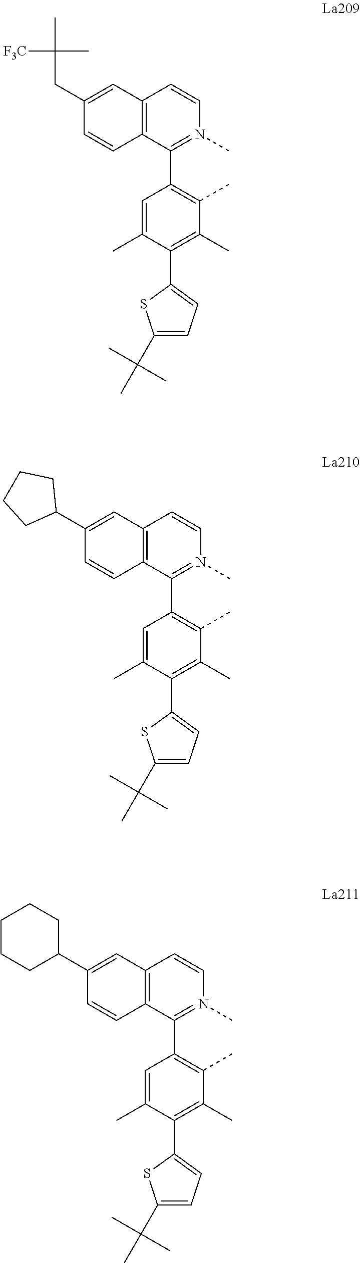

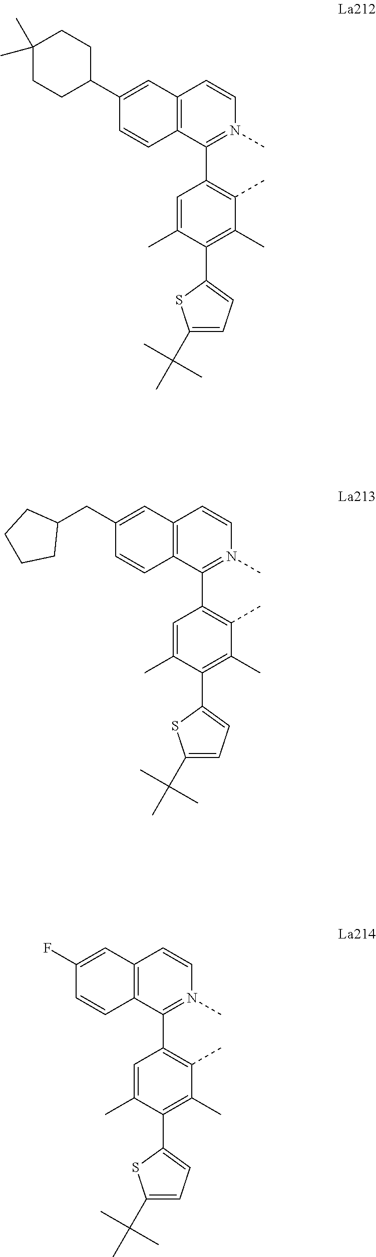

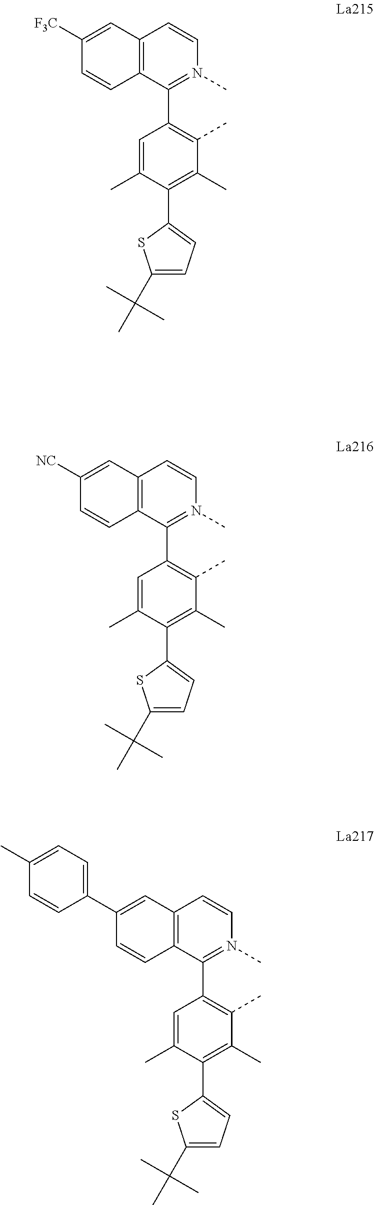

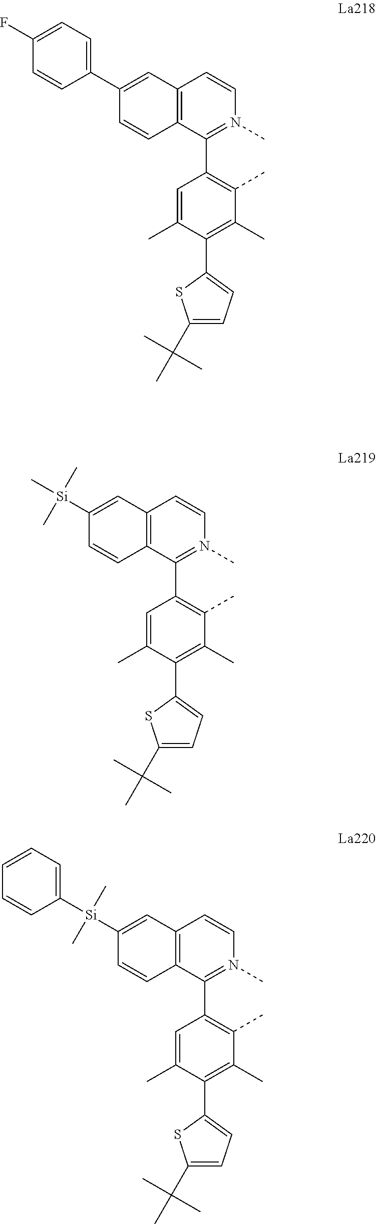

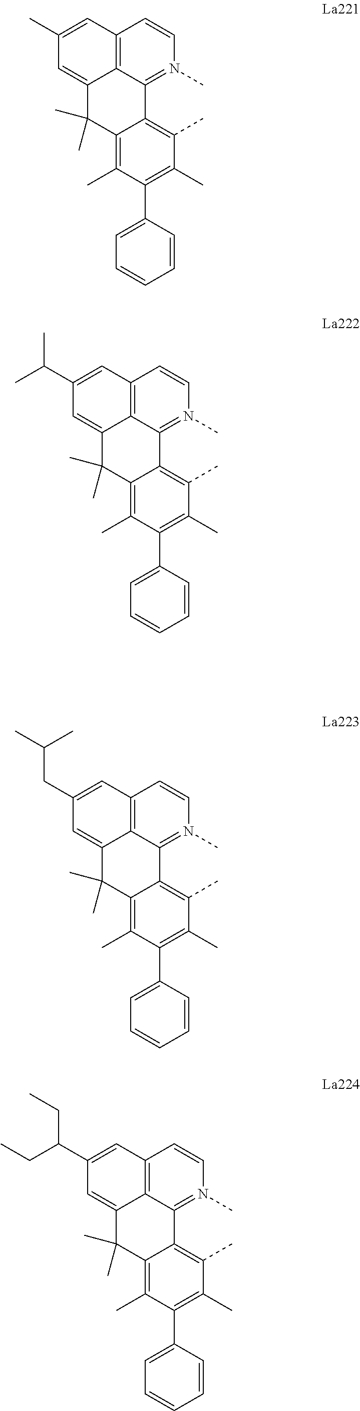

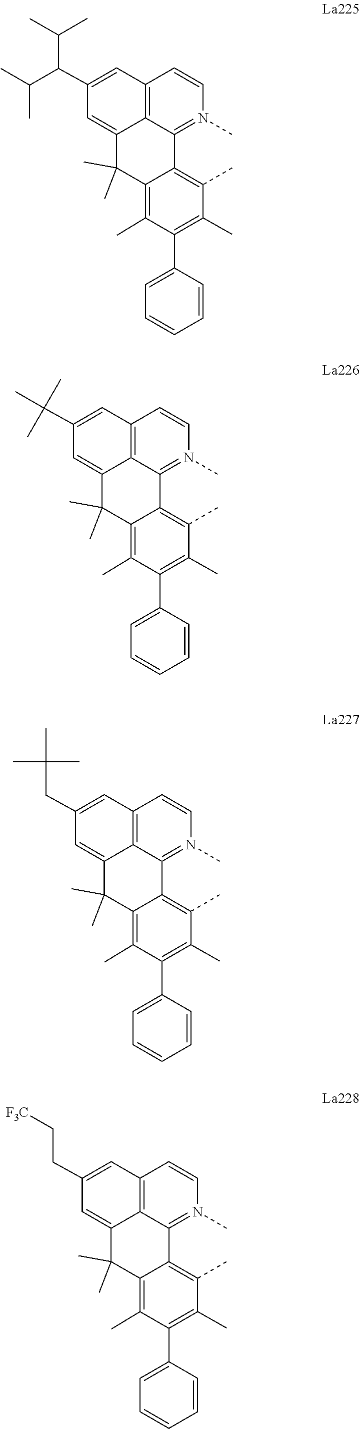

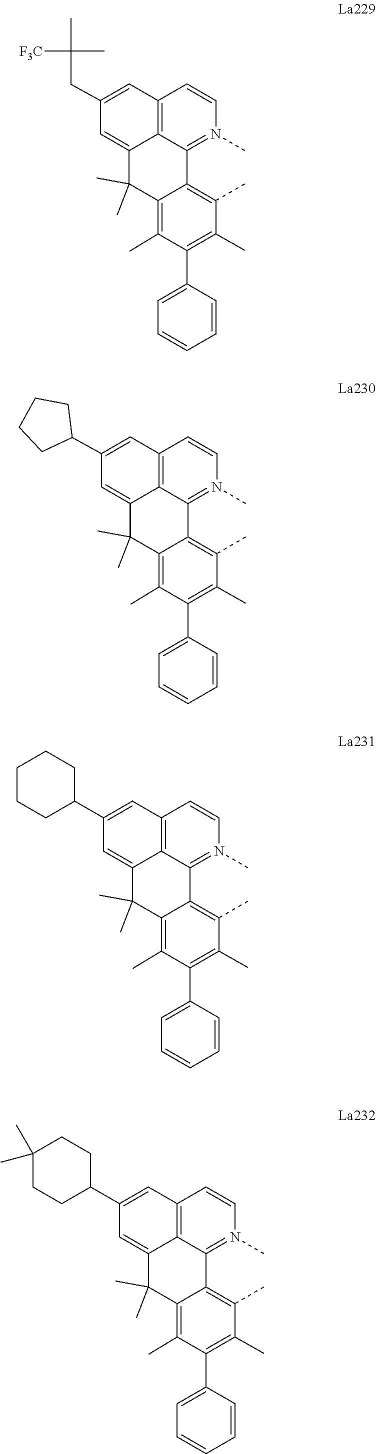

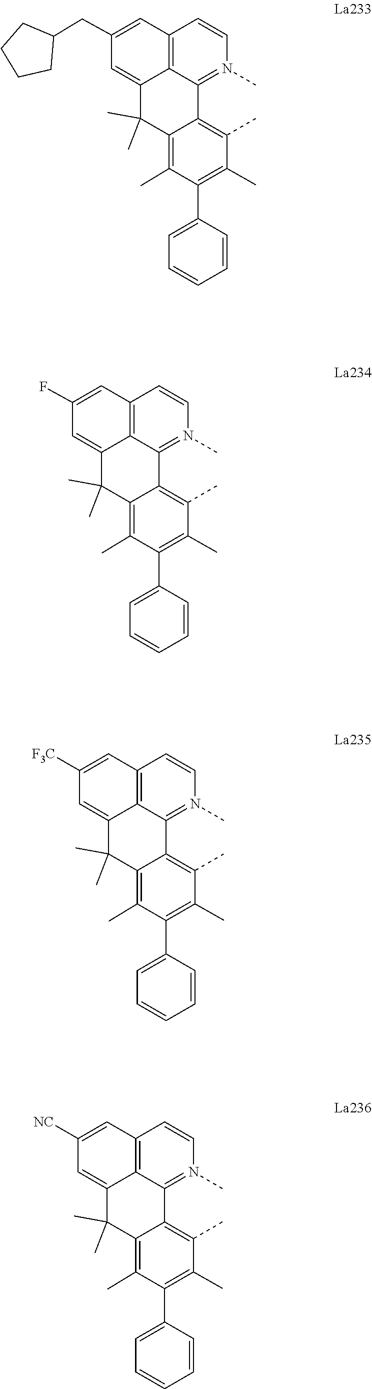

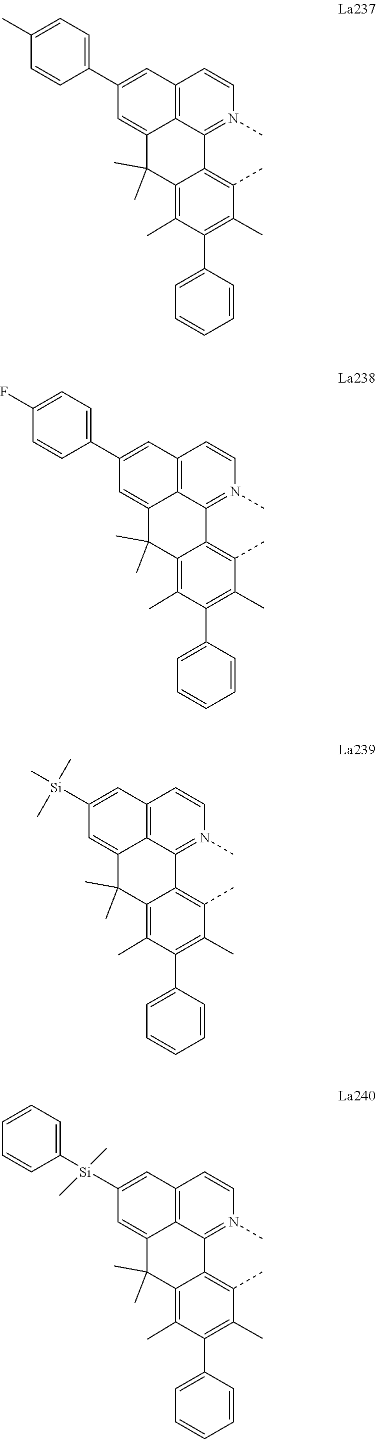

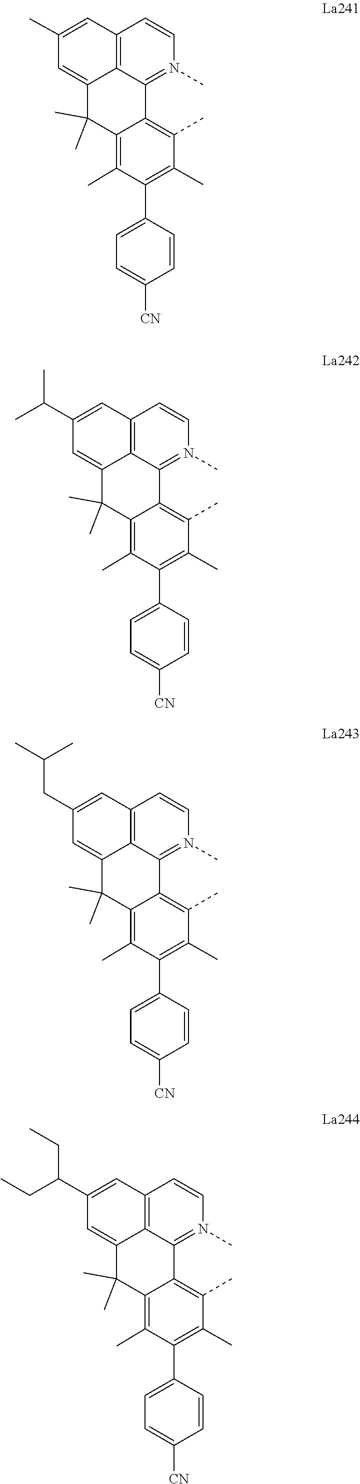

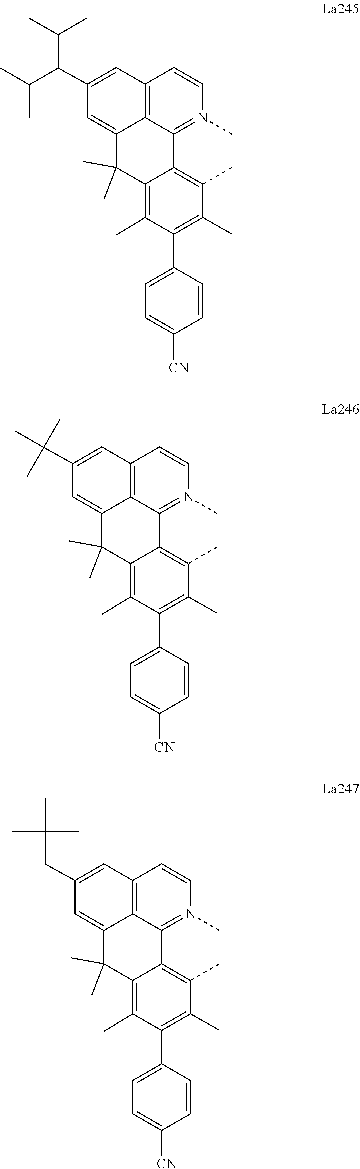

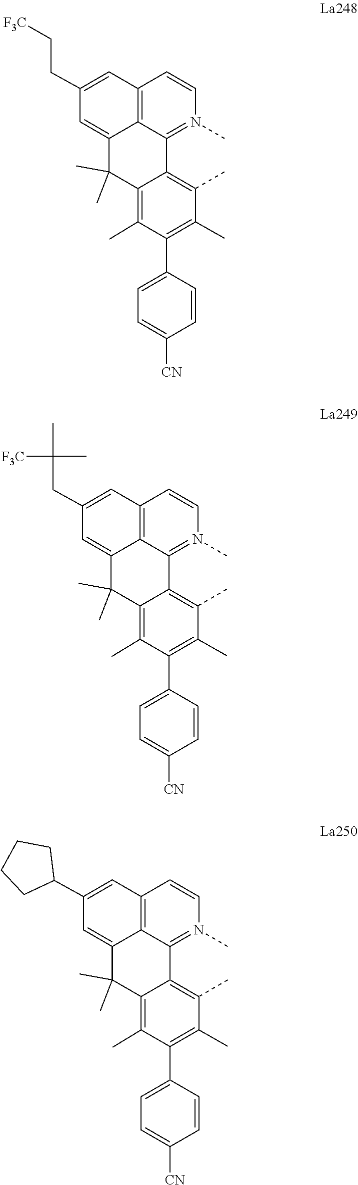

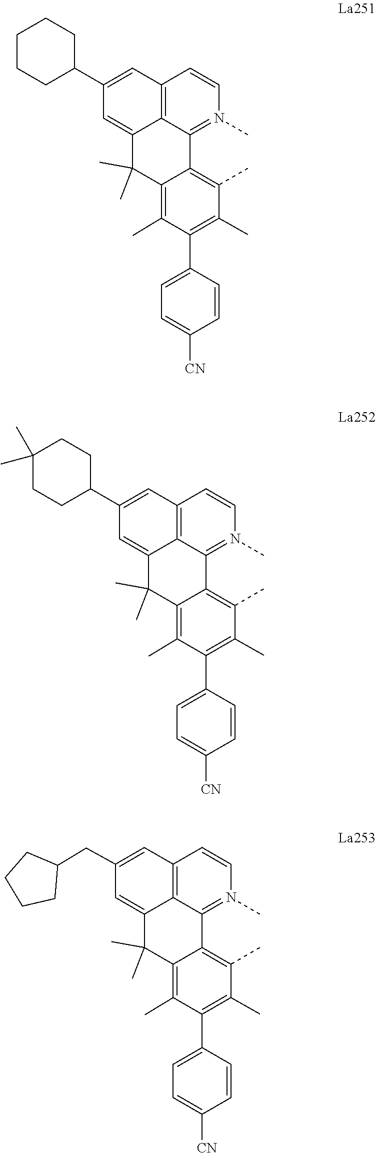

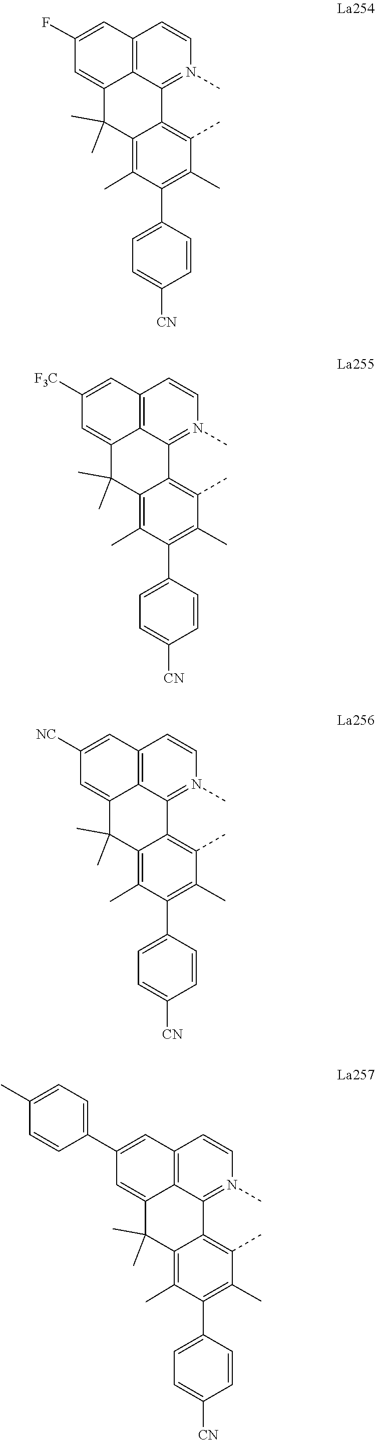

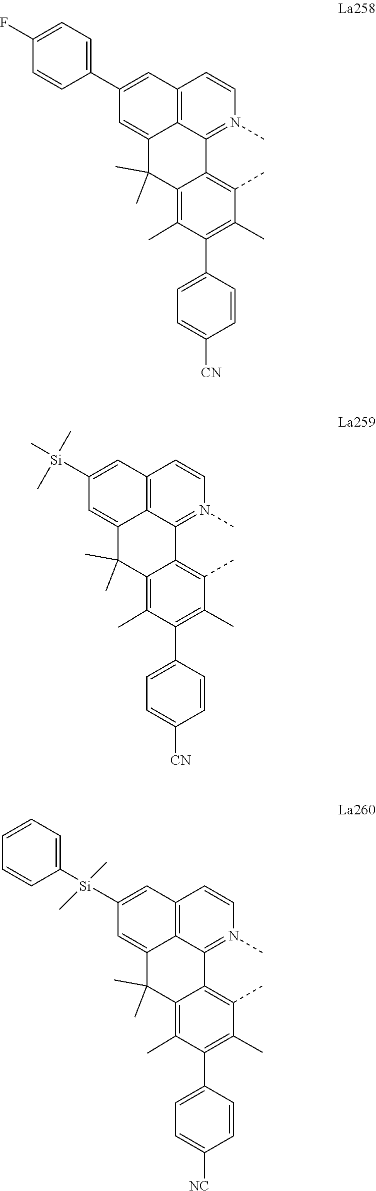

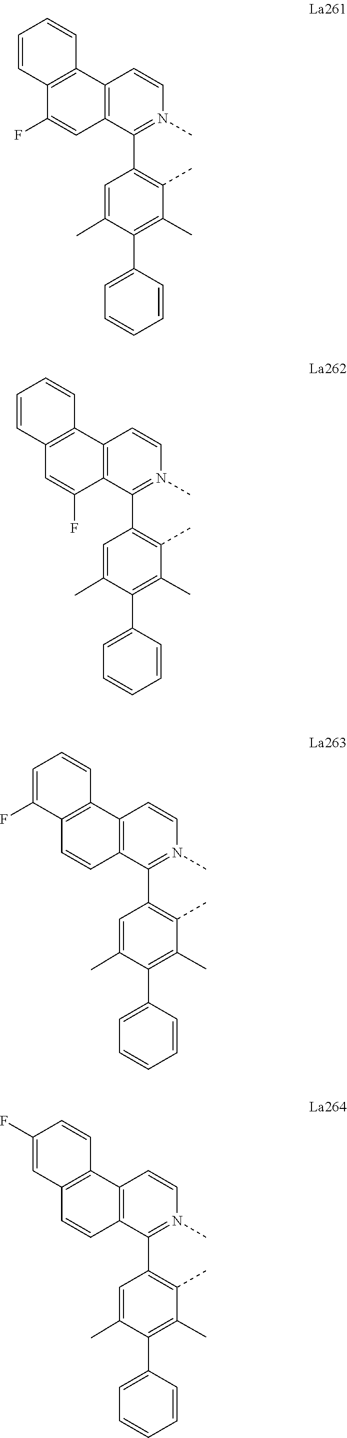

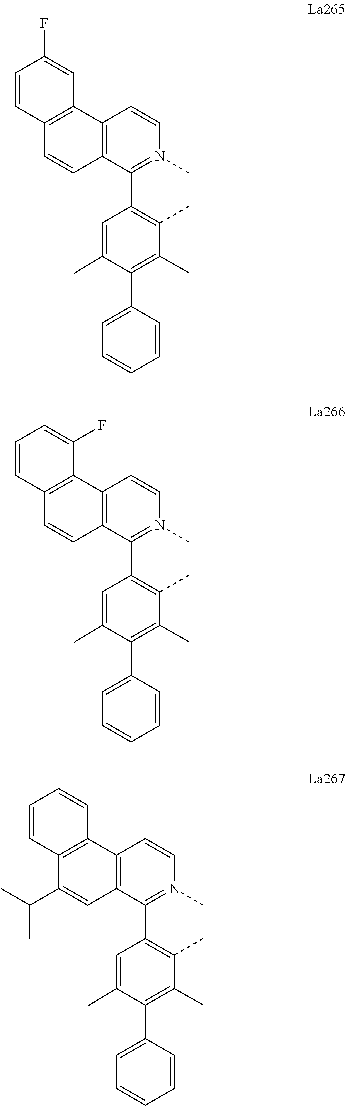

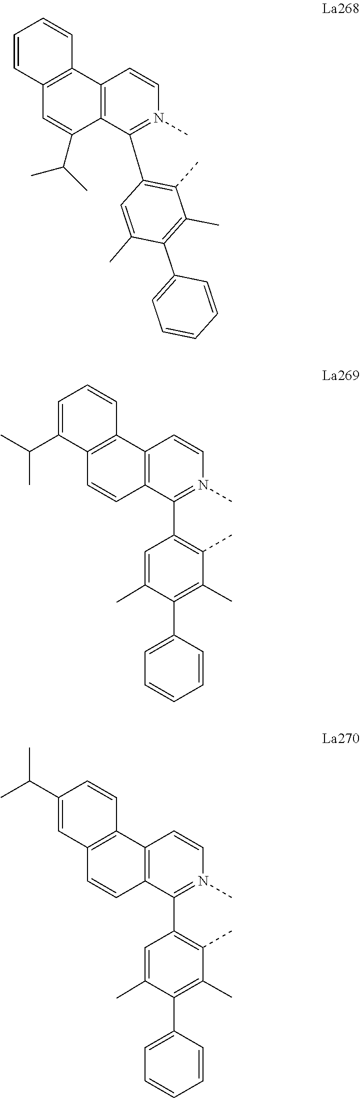

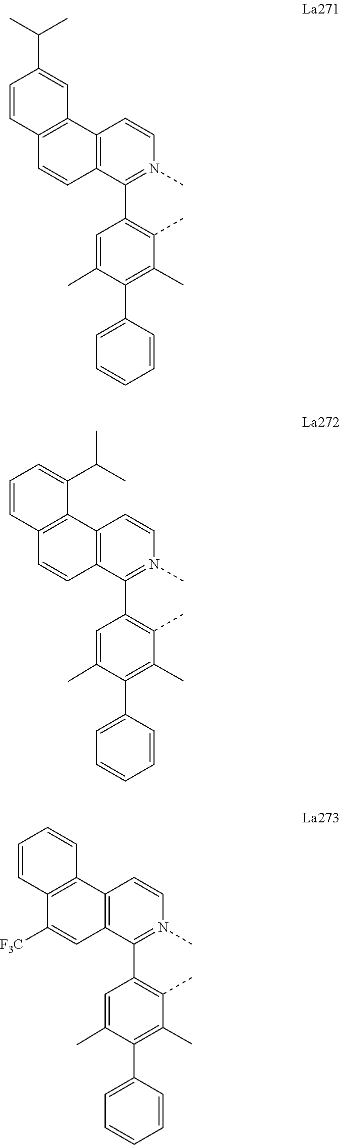

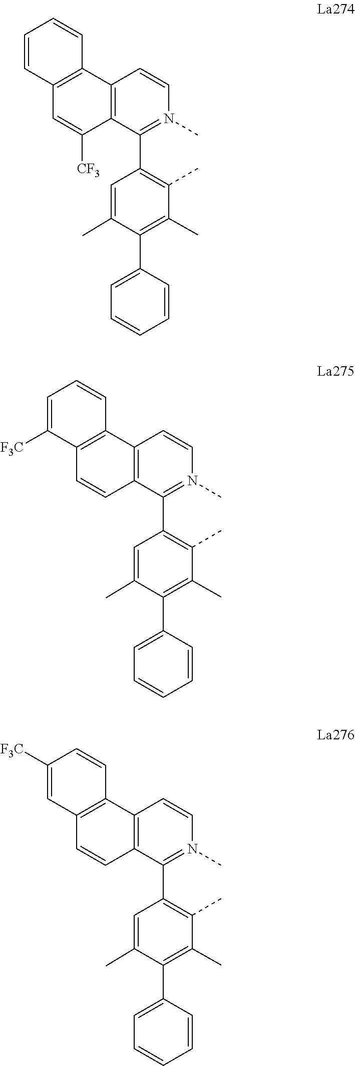

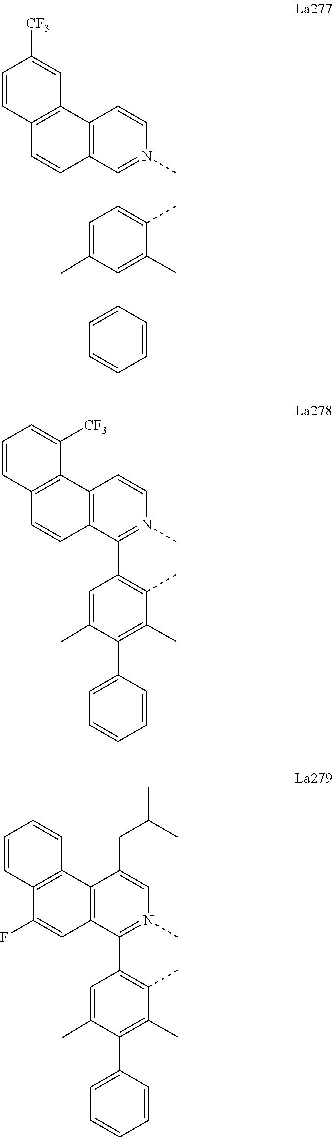

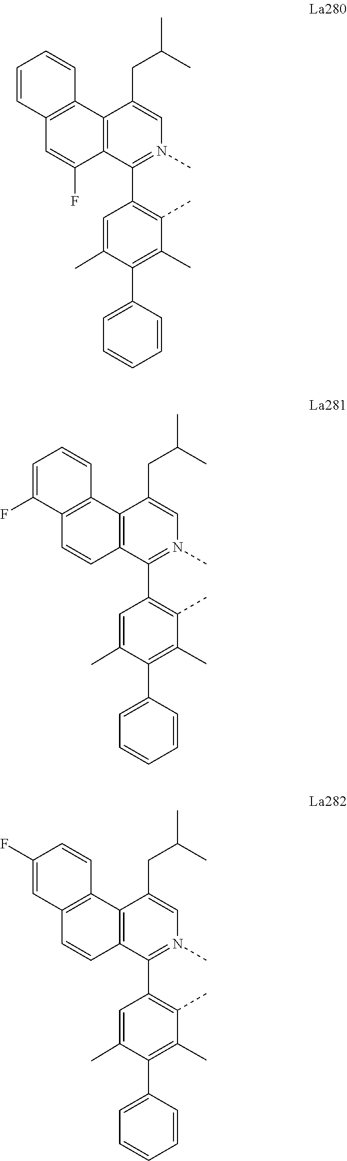

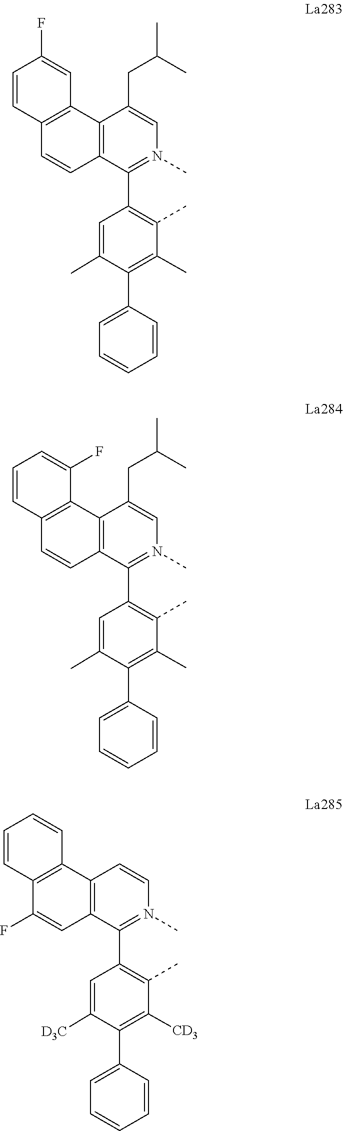

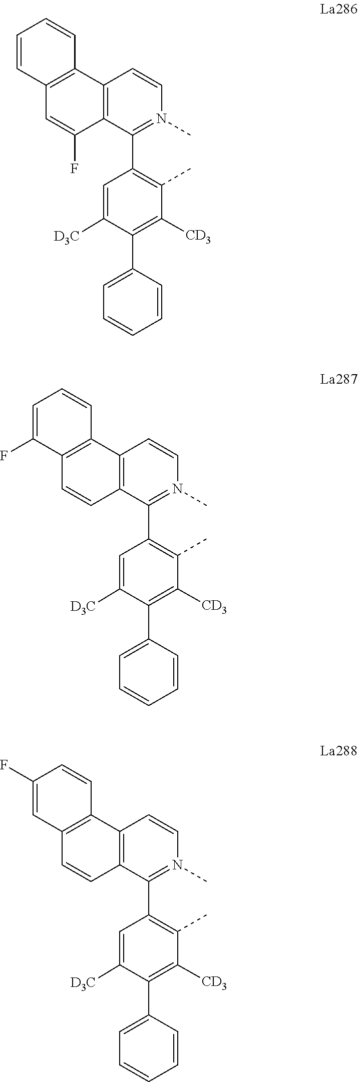

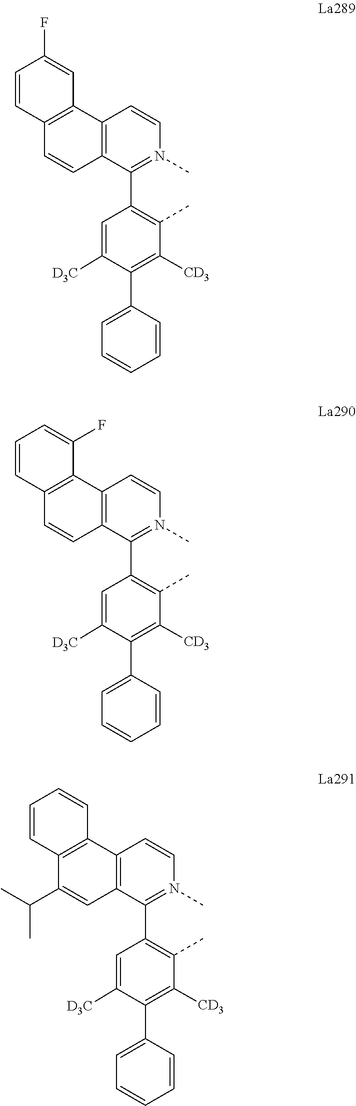

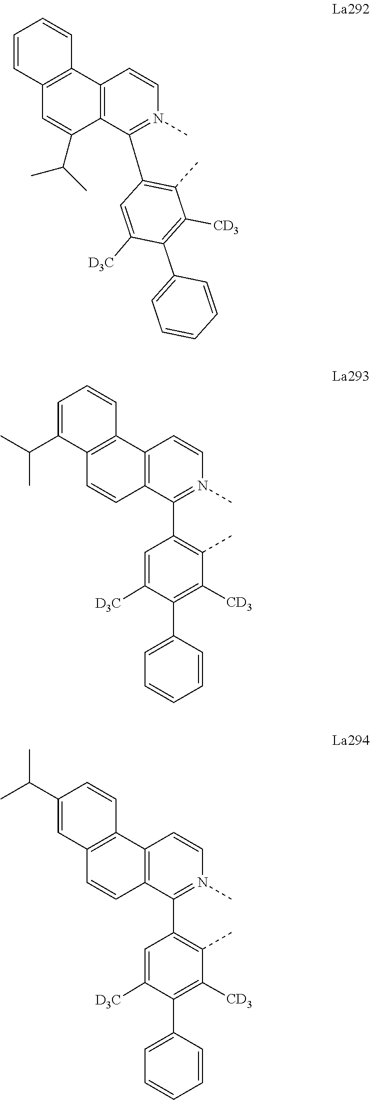

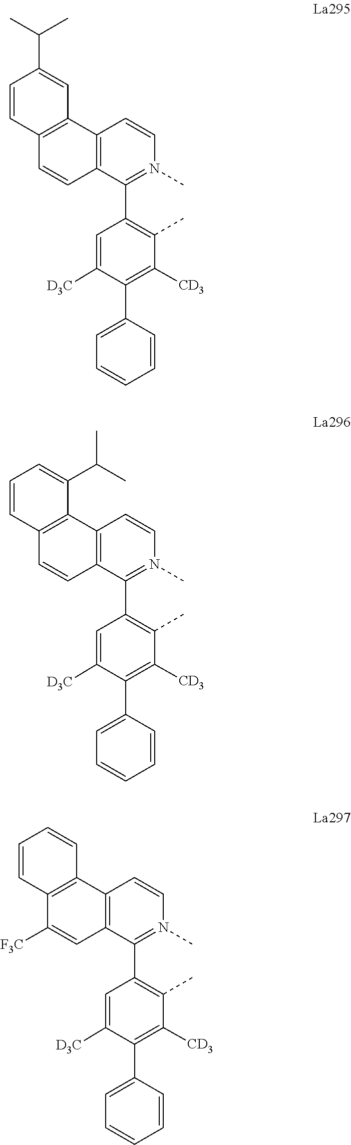

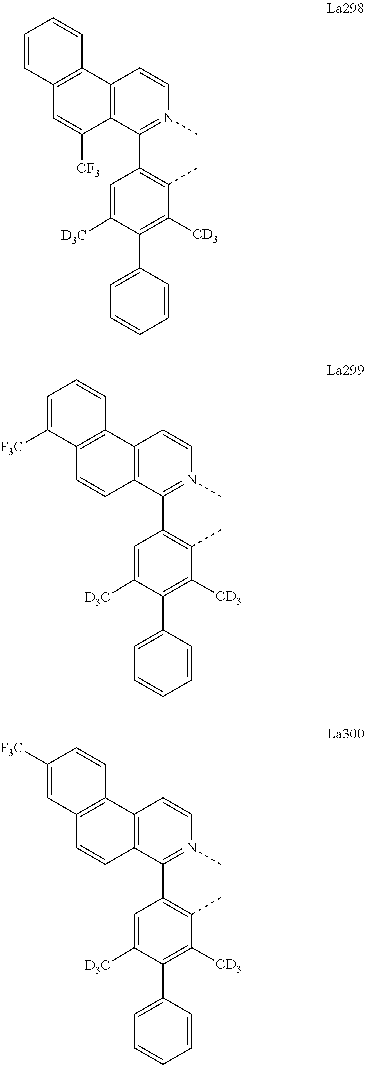

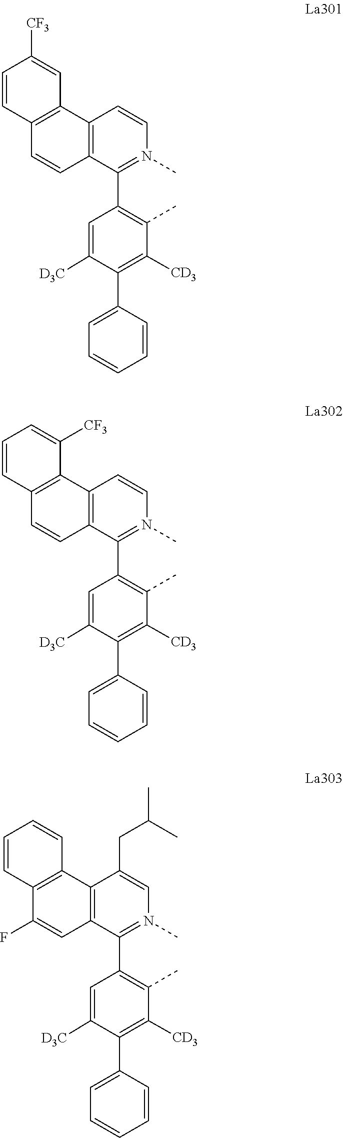

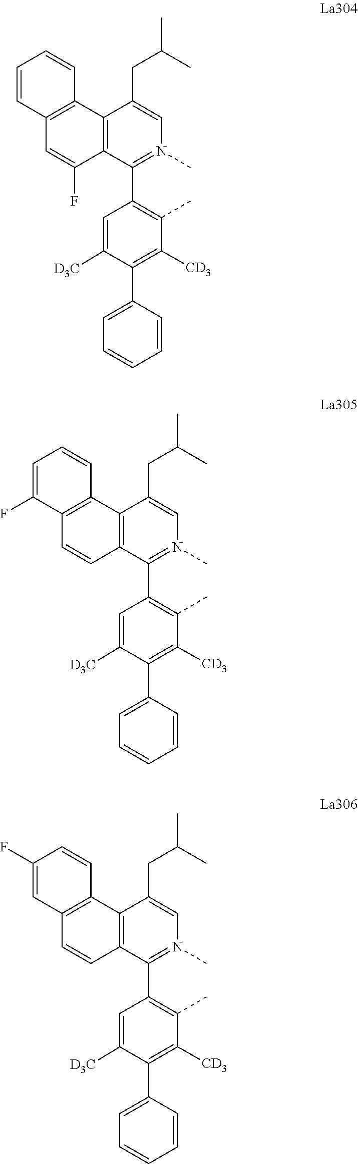

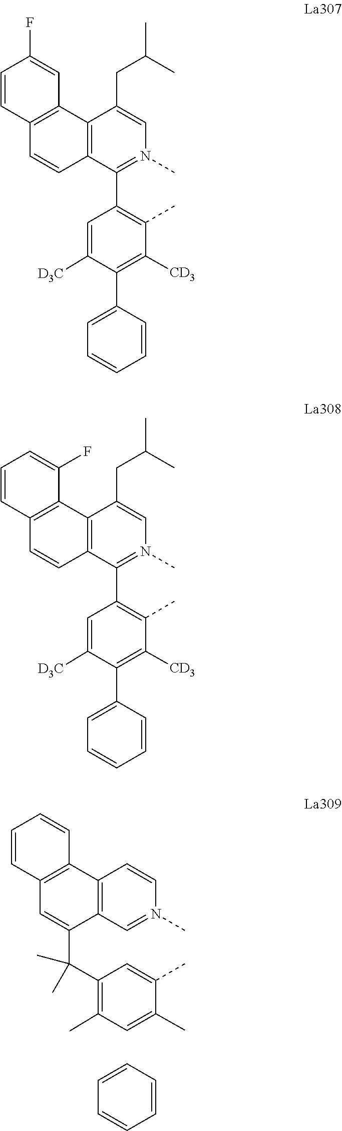

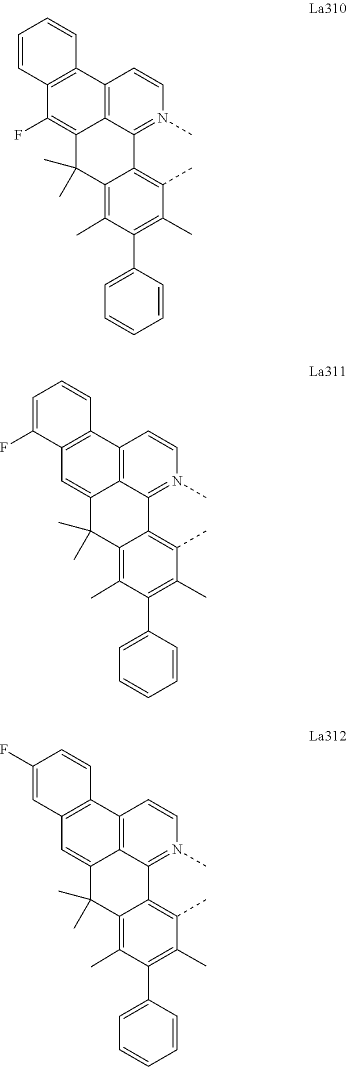

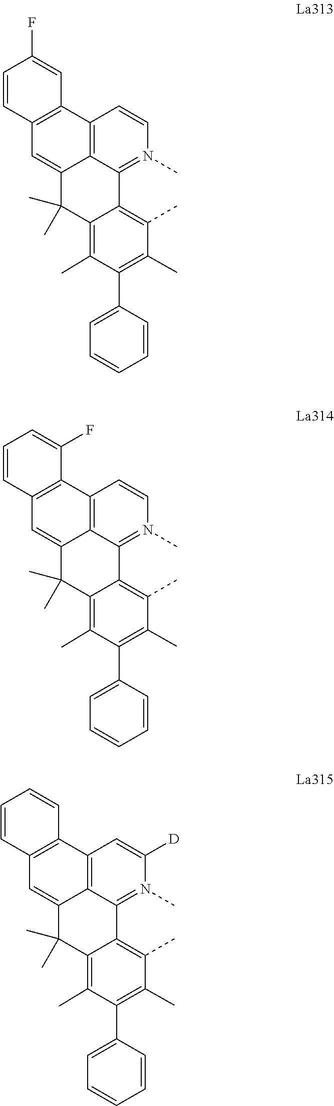

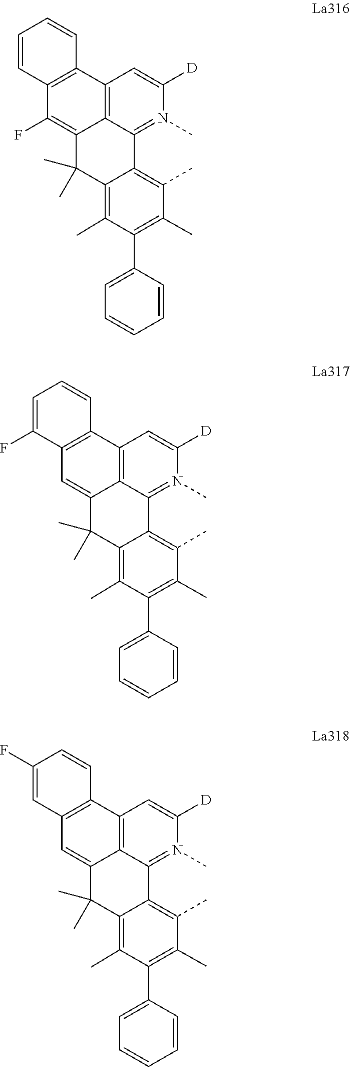

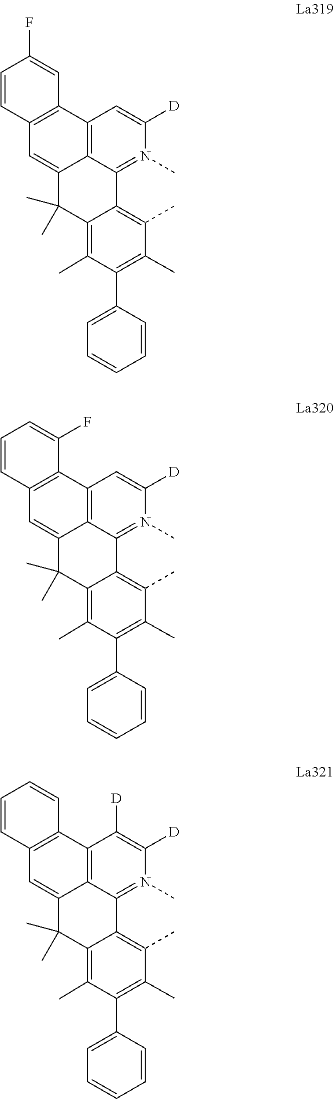

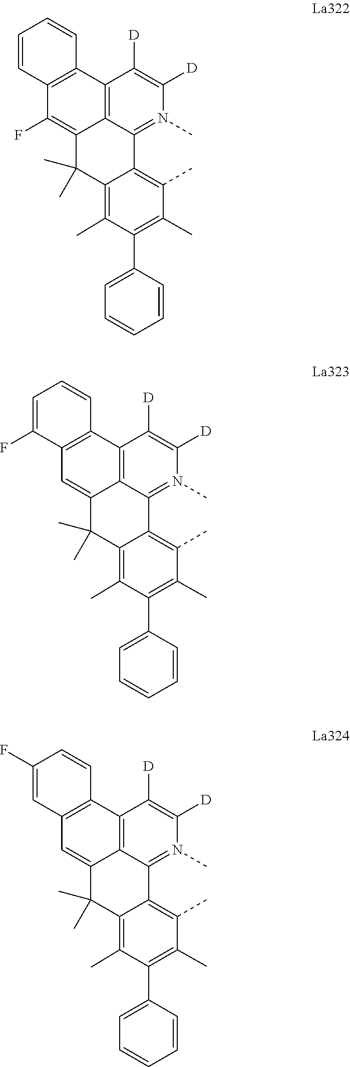

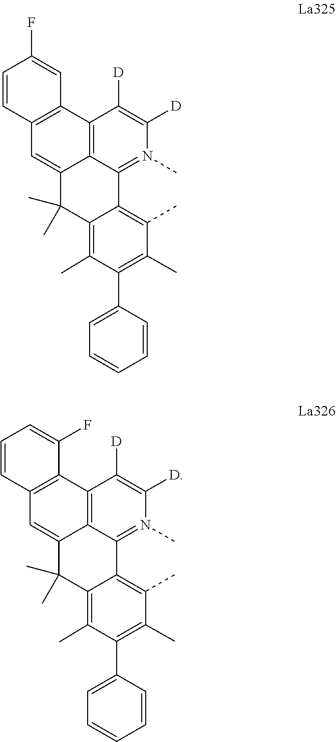

16. The metal iridium complex according to claim 1, wherein Lb is one of the following structural formulas, or is one of the following structural formulas in which hydrogen is partially or completely replaced by deuterium or fluorine,

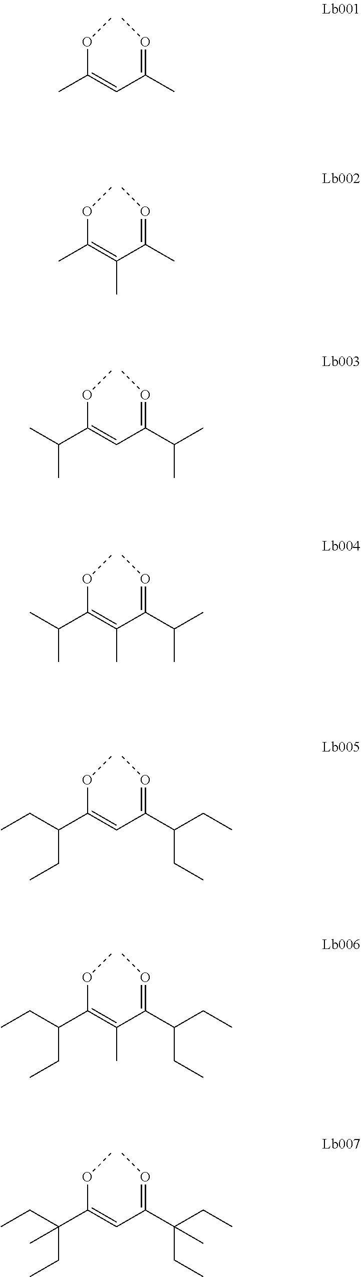

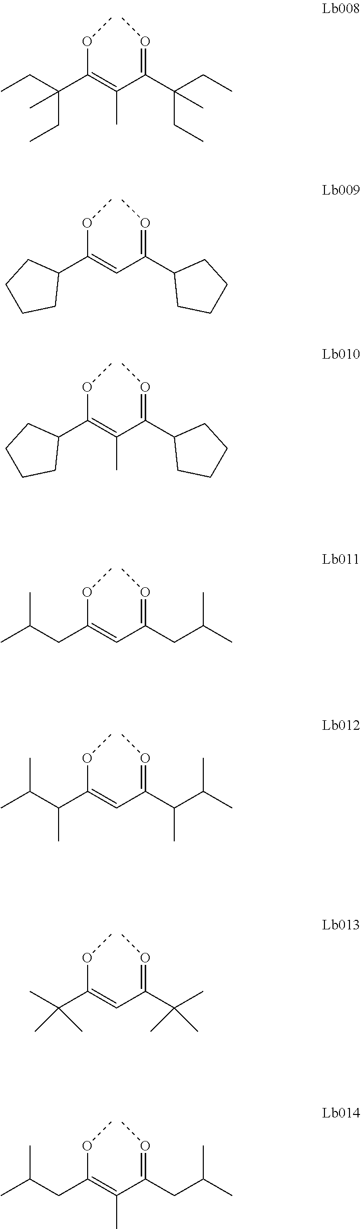

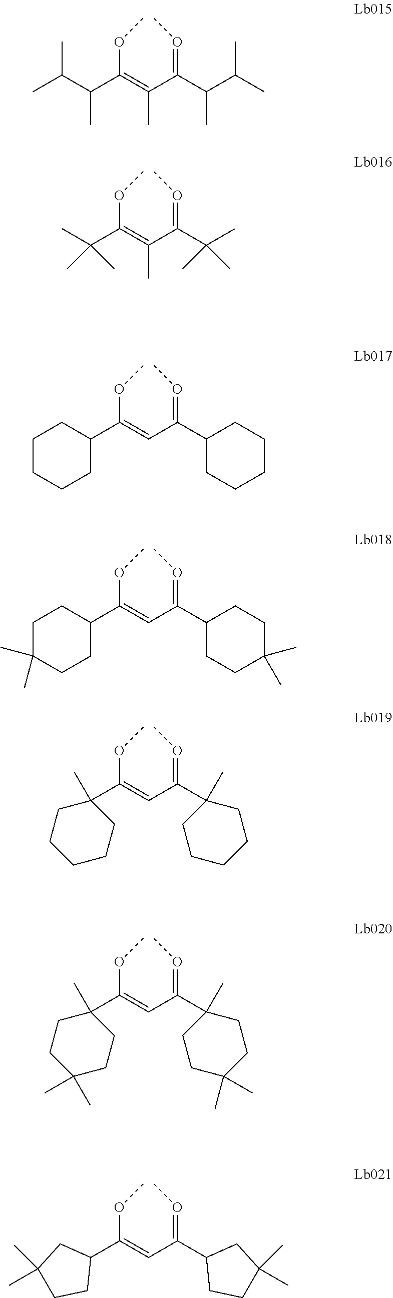

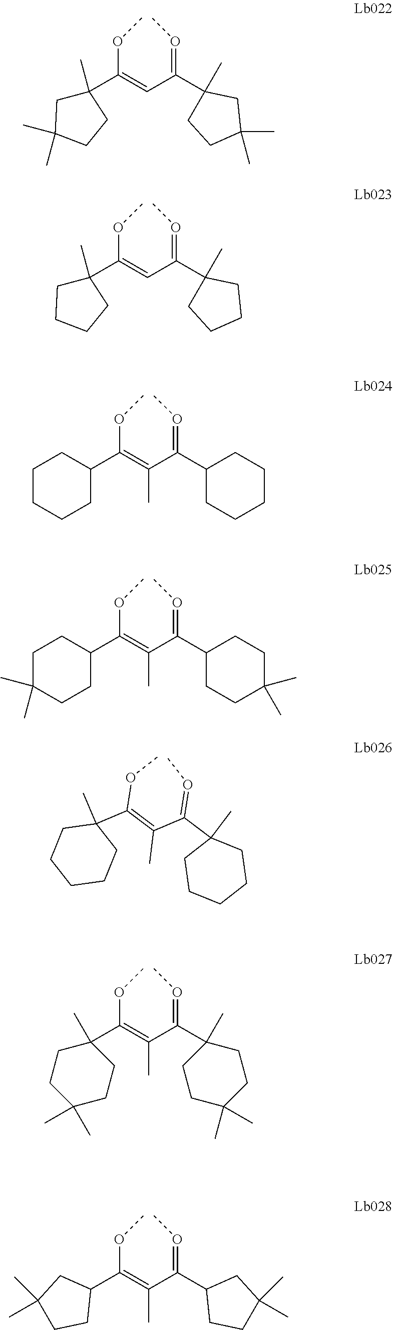

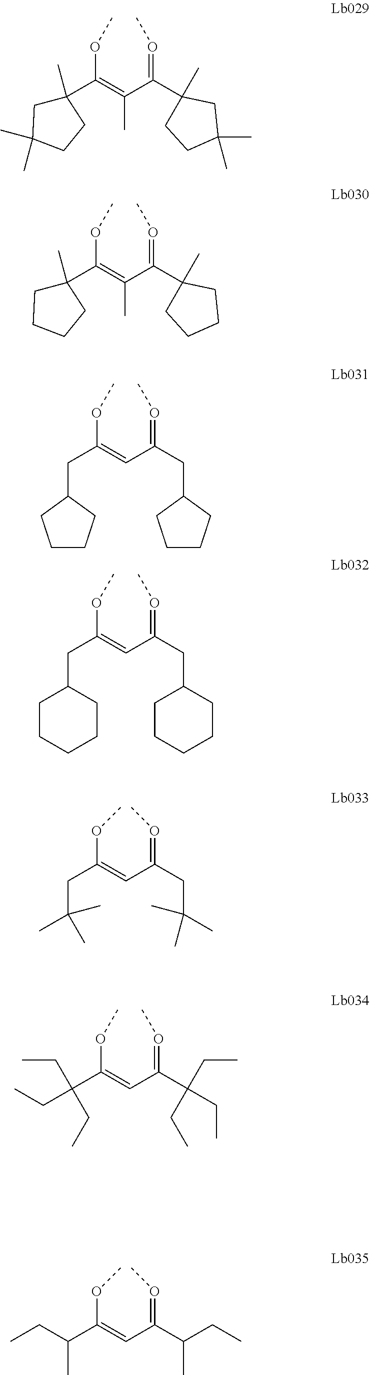

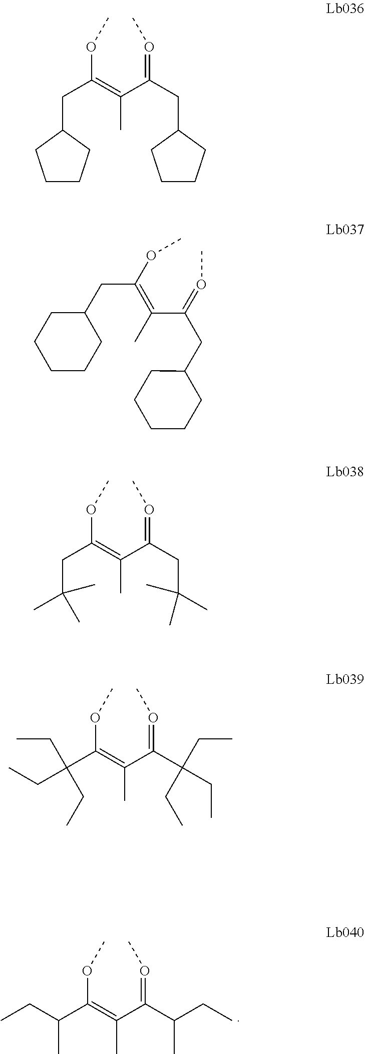

17. An electroluminescent device, comprising a cathode, an anode and an organic layer disposed between the cathode and the anode, wherein the organic layer comprises the metal iridium complex according to claim 1.

18. The electroluminescent device of claim 17, wherein the organic layer comprises a light-emitting layer, and the metal iridium complex is used as a red-emitting doping material of the light-emitting layer; or wherein the organic layer comprises a hole injection layer, and the metal iridium complex is used as a hole injection material in the hole injection layer.

19. A ligand La, having a structural formula as follows: 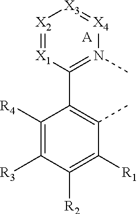
wherein R₁-R₄ and X₁-X₄ are as defined in claim 1.
